(19) United States
(12) Patent Application Publication
(10) Pub. No.: US 2005/0239052 A1
(43) Pub. Date: Oct. 27, 2005

(54) Cloning of rhadinovirus genome and methods for its use

(76) Inventors: Scott W. Wong, Beaverton, OR (US); Michael K. Axthelm, Hillsboro, OR (US)

Correspondence Address: KLARQUIST SPARKMAN, LLP, 121 SW SALMON STREET, SUITE 1600, PORTLAND, OR, 97204, US

(21) Appl. No.: 10/276,524

(22) PCT Filed: May 17, 2001
(86) PCT No.: PCT/US01/16274

§ 371 Date: Apr. 10, 2003

Related U.S. Application Data
(60) Provisional application No. 60/205,652, filed on May 18, 2000.

(51) Int. Cl. C12Q 1/70; A61K 39/12; C12N 7/00; C07K 14/005; C07H 21/04; C12N 15/86

(52) U.S. Cl. 435/5; 424/204.1; 435/456; 530/350; 530/388.3; 435/235.1; 536/23.72

(57) ABSTRACT

An isolated virus is provided (Japanese Macaque Herpesvirus, JMHV), as deposited with ATCC as deposit accession number PTA-1884, as are viral particles including this virus and host cells infected with this virus. A purified polypeptide is also provided that includes an amino acid sequence that has at least 95% sequence identity to an amino acid sequence as set forth as SEQ ID NO:3, SEQ ID NO:5, SEQ ID NO:8, SEQ ID NO:10, SEQ ID NO:12, SEQ ID NO:14, SEQ ID NO:17, SEQ ID NO:19, SEQ ID NO:21, SEQ ID NO:23, SEQ ID NO:25, and SEQ ID NO:27, as are nucleic acid molecules encoding these polypeptides. A method is provided for testing the efficacy of a drug in the treatment of a condition associated with infection with JMHV. A model for multiple sclerosis is provided, as is a method for testing the efficacy of a candidate vaccine against JMHV infection, or conditions associated with JMHV infection. In a further embodiment, a method is provided for detecting the presence of JMHV or a related virus in a biological specimen, by amplifying by polymerase chain reaction a JMHV nucleic acid sequence, or by using hybridization technology, if such sequence is present in the sample. A method is also provided for detecting the presence of JMHV in a biological specimen. Kits are provided including an antibody that binds to a JMHV polypeptide or an oligonucleotide that hybridizes to a JMHV nucleic acid sequence.

### PRIORITY CLAIM

This application is a continuation of U.S. Provisional Patent Application No. 60/205,652, filed May 18, 2000. This application is also related to PCT application US99/26260, which claims priority from U.S. Provisional Patent Application No. 60/109,409 and U.S. Provisional Patent Application No. 60/107,507. All of these applications are incorporated herein incorporated by reference.

### FIELD OF THE INVENTION

This application relates to a Japanese macaque herpesvirus (JMHV), specifically to the nucleic acid sequence of open reading frames in the virus and to amino acid sequences encoded by these sequences. Compositions and methods are provided for the production of animal models useful in assessing the efficacy of drugs for the treatment or prevention of conditions associated with infection by the virus, such as multiple sclerosis. In addition, methods are provided for the isolation of related viruses.

### BACKGROUND

Converging lines of evidence indicate that Kaposi's sarcoma-associated herpesvirus (KSHV) is the etiological agent responsible for Kaposi's sarcoma (KS) in individuals with and without HIV infection (Chang et al., 1994, Science 266:1865-9; Schalling et al., 1995, Nature Med. 7:707-8; Moore & Chang, 1995, N. Engl. J. Med. 332:1181-5; Whitby et al., 1995, Lancet 346:799-802; Ambroziak et al., 1995, Science 268:582-3; Dupin et al., 1995, Lancet 345:761-2; Chuck et al, 1996, J. Infect. Dis. 173:248-51; O'Neill et al., 1996, J. Clin. Pathol. 49:306-8; Gao et al., 1996, Nature Med. 2:925-8; Kedes et al., 1996, Nature Med. 2:918-24; Gao et al., 1996, N. Engl. J. Med. 335:233-41). In addition to KS, KSHV is also responsible for other acquired immunodeficiency syndrome (AIDS)-related and non-AIDS-related malignancies, such as primary effusion lymphomas (Cesarman et al., 1995, N. Engl. J. Med. 332:1186-91; Nador et al., 1996, Blood 88:645-56; Otsuli et al, 1996, Leukemia 10:1358-62), and multicentric Castleman's disease (MCD), a B cell proliferation disorder associated with overexpression of IL-6 activity (Soulier et al., 1995, Blood 86:1276-80; Yoshizaki et al., 1989, Blood 74:1360-7).

More recently, KSHV has been proposed to be involved in multiple myeloma, a B cell abnormality of monoclonal origin (Rettig et al., 1997, Science 276:1851-4; Said et al., 1997, Blood 90:4278-82; Parravicini et al., 1997, Science 278:1969-70; Masood et al., 1997, Science 278:1970-1; Whitby et al., 1997, Science 278:1971-2; Cottoni et al., 1997, Science 278:1972; Brousset et al., 1997, Science 278:290-4). Understanding how KSHV is involved in these malignancies is important for the generation of therapies against the spectrum of KSHV-associated diseases.

Testing the efficacy of therapeutics and vaccines against any disease, such as KHSV, is greatly facilitated by the availability of an animal model, such as a non-human primate model, because non-human primates are physiologically very similar to humans. Although such models have been developed for the study of HIV infection (for example, U.S. Pat. Nos. 5,212,084 and 5,543,131) none has yet been developed for KSHV infection.

Infection of animals with some herpesviruses, namely Herpesvirus saimiri and murine herpesvirus type 68, can cause a lymphoproliferative disorder (LPD). However, these animals are not adequate models of KSHV pathogenesis because they lack certain KSHV genes that may contribute to viral pathogenesis or influence HIV infection, such as Interleulin 6 (IL-6) and macrophage inflammatory protein 1 (MIP-1) (Albrecht et al., 1992, J. Virol. 66:5047-58; Virgin et al., 1997, J. Virol. 71:5894-904). Thus, so far the establishment of a non-human primate model for KSHV infection has remained elusive.

The present invention addresses this problem, and others, in the development of animal models for a variety of pathological conditions and diseases.

### SUMMARY OF THE DISCLOSURE

Rhesus macaques naturally harbor a virus related to KSHV, referred to as RRV, for rhesus rhadinovirus. The present disclosure also includes information about pathological conditions associated with RRV infection.

Japanese macaques can harbor a virus related to RRV, called Japanese Macaque Virus (JMHV).

An isolated virus is provided (Japanese Macaque Herpesvirus, JMHV) as deposited with ATCC as deposit accession number PTA-1884, deposited May 18, 2000, as are viral particles including this virus and host cells infected with this virus.

A purified polypeptide is also provided that includes an amino acid sequence that has at least 95% sequence identity to an amino acid sequence as set forth as SEQ ID NO:3, SEQ ID NO:5, SEQ ID NO:8, SEQ ID NO:10, SEQ ID NO:12, SEQ ID NO:14, SEQ ID NO:17, SEQ ID NO:19, SEQ ID NO:21, SEQ ID NO:23, SEQ ID NO:25, and SEQ ID NO:27, as are nucleic acid molecules encoding these polypeptides.

In another embodiment, a method is provided for testing the efficacy of a drug in the treatment of a condition associated with infection with JMHV. The method includes administering the drug to a non-human primate infected with an JMHV; and observing the non-human primate to determine if the drug prevents or reduces the presentation of one or more symptoms associated with JMHV infection.

In another embodiment, a method is provided for testing the efficacy of a candidate vaccine against JMHV infection, or conditions associated with JMHV infection. The method includes the steps of: (a) administering the vaccine to a non-human primate susceptible to infection with the JMJV; (b) inoculating the subject with the JMHV; and (c) observing the non-human primate to determine if the vaccine prevents or reduces an incidence of JMHV infection or a symptom associated with JMHV infection.

In a further embodiment, a method is provided for detecting the presence of JMHV or a related virus in a biological specimen, by amplifying by polymerase chain reaction a JMHV nucleic acid sequence, or by using hybridization technology, if such sequence is present in the sample.

In yet another embodiment, a method is provided for obtaining JMHV-related nucleic acid sequence using amplification or hybridization.

A method is also provided for detecting the presence of JMHV in a biological specimen, including contacting the biological specimen with an antibody that binds to a JMHV polypeptide, and detecting binding of the antibody to the biological specimen or a component thereof. Binding of the antibody to the biological specimen indicates the presence of JMHV.

Kits are provided that include an antibody that binds to a JMHV polypeptide or an oligonucleotide that hybridizes to a JMHV nucleic acid sequence.

The foregoing and other objects, features, and advantages of the invention will become more apparent from the following detailed description of several examples which proceeds with reference to the accompanying figures.

### BRIEF DESCRIPTION OF THE FIGURES

FIG. 1 shows a phylogenetic comparison of the gammaherpesviruses Epstein-Barr virus (EBV), Alcelaphine herpesvirus (AHV), Murine herpesvirus (MHV), Herpesvirus saimiri (HVS), Kaposi's sarcoma-associated herpesvirus (KSHV), and Rheusus rhadinovirus 17577 (RRV). It shows that among the known sequenced viruses, RRV is the closest relative to KSHV, using an accepted maximum parsimony method of evaluating evolutionary relationships.

FIG. 2 is a table showing the BamHI, EcoRI and HindIII restriction fragments of the RRV genome.

FIG. 3 is a schematic map of the 75 ORFs of RRV. Arrow direction represents direction of transcription.

FIG. 4 is a table showing the size, location and description (similarity to other proteins) of the proteins encoded by the ORFs of RRV.

FIG. 5 is a table showing a comparison of corresponding repeats in RRV and KSHV.

FIG. 6 is a table showing the comparison of interferon regulatory elements encoded by RRV and KSHV.

FIG. 7 is a table comparing the ORFs of RRV, KSHV and HVS. The table shows the start and stop points, the strand (+ or −) from which the ORF is transcribed, the size of the ORFs and the percentage similarity of KSHV and HVS when compared with RRV.

FIGS. 8A-8D are graphs showing CD20+lymphocytes, antibody response and RHKSHV isolation/detection in macaques infected with SIVmac239 and RRV (A) 18483 and (B) 18570 and macaques infected with SIVmac239 only (C) 18503 and (D) 18540. A “+” indicates positive for virus culture or viral DNA, as defined by PCR and Southern blot analysis; “−”, negative for virus culture or viral DNA.

FIG: 9 shows the result of the PCR analysis of PBLs and LNMCs from each of the macaques (18483, 18503, 18540 and 18570) for RRV DNA and β-globin in (A) graphical form and (B) digital form.

FIG. 10 shows the DNA sequence of the RRV ORF that encodes the MIP protein (nucleotides 22245-22592 of SEQ ID NO:1).

FIG. 11 shows the DNA sequence of the RRV ORF that encodes the IL-6 protein (nucleotides 19921-20544 of SEQ ID NO 1).

### SEQUENCE LISTING

The nucleic and amino acid sequences listed in the accompanying sequence listing are shown using standard letter abbreviations for nucleotide bases, and the code for amino acids. Only one strand of each nucleic acid sequence is shown, but the complementary strand is understood as included by any reference to the displayed strand.

SEQ ID NO 1 shows the nucleotide sequence of the RRV genome. RRV R1 corresponds to nucleotides 1353-2624 of SEQ ID NO 1. RRV ORF2 corresponds to the complement of nucleotides 2692-3258 of SEQ ID NO 1, which encodes dihydrofolate reductase, and which has some similarity to Kaposi's sarcoma-associated herpesvirus (KSHV) ORF 2. RRV ORF 4 corresponds to nucleotides 3676-5613 of SEQ ID NO 1, which encodes complement binding protein, and which has some similarity to KSHV ORF 4 RRV ORF 6, corresponds to nucleotides 6045-9443 of SEQ ID NO 1, which encodes ssDNA binding protein, and which has some similarity to KSHV ORF 6. RRV ORF 7 corresponds to nucleotides 9468-11528 of SEQ ID NO 1, which encodes a transport protein, and which has some similarity to KSHV ORF 7. RRV ORF 8, corresponds to nucleotides 11515-14004 of SEQ ID NO 1, which encodes glycoprotein B, and which has some similarity to KSHV ORF 8. RRV ORF 9, DNA polymerase protein, corresponds to nucleotides 14122-17166 of SEQ ID NO 1, which has some similarity to KSHV ORF 9. RRV ORF 10 corresponds to nucleotides 17261-18511 of SEQ ID NO 1, which has some similarity to KSHV ORF 10. RRV ORF 11 corresponds to nucleotides 18520-19749 of SEQ ID NO 1, which has some similarity to KSHV ORF 11. RRV R2 corresponds to the complement of nucleotides 19921-20544 of SEQ ID NO 1, which has some similarity to the Kaposi's sarcoma-associated IL-6 gene. RRV ORF 70, thymidylate synthase, corresponds to the complement of nucleotides 20777-21778 of SEQ ID NO 1, and which has some similarity to KSHV ORF 70. RRV R3 corresponds to the complement of nucleotides 22245-22592 of SEQ ID NO 1, which has some similarity to the KSHV K4 viral MIP gene. RRV ORF 16, a Bcl-2 homolog, corresponds to nucleotides 26846-27409 of SEQ ID NO 1, which has some similarity to KSHV ORF 16. RRV ORF 17 corresponds to the complement of nucleotides 27515-29125 of SEQ ID NO 1, which has some similarity to KSHV ORF 17. RRV ORF 18 corresponds to nucleotides 28998-29897 of SEQ ID NO 1, which has some similarity to KSHV ORF 18.

RRV ORF 19 corresponds to the complement of nucleotides 29905-31548 of SEQ ID NO 1, which encodes a tegument protein, and which has some similarity to KSHV ORF 19. RRV ORF 20 corresponds to the complement of nucleotides 31043-32095 of SEQ ID NO 1, and has some similarity to KSHV ORF 20. RRV ORF 21 corresponds to nucleotides 32094-33767 of SEQ ID NO 1, which encodes a thymdine kinase protein, and which has some similarity to KSHV ORF 21. RRV ORF 22 corresponds to nucleotides 33754-35868 of SEQ ID NO 1, and encodes a glycoprotein H protein, and which has some similarity to KSHV ORF 22. RRV ORF 23 corresponds to the complement of nucleotides 35865-37073 of SEQ ID NO 1, which has some similarity to KSHV ORF 23. RRV ORF 24 corresponds to the complement of nucleotides 37123-39321 of SEQ ID NO 1, and which has some similarity to KSHV ORF 24. RRV ORF 25, corresponds to nucleotides 39323-43459 of SEQ ID NO 1, which encodes a major capsid protein, and which has some similarity to KSHV ORF 25. RRV ORF 26 corresponds to nucleotides 43491-44408 of SEQ ID NO 1, which encodes a capsid protein, and which has some similarity to KSHV ORF 26. RRV ORF 27 corresponds to nucleotides 44433-45242 of SEQ ID NO 1, and which has some similarity to KSHV ORF 27. RRV ORF 28 corresponds to nucleotides 45408-45683 of SEQ ID NO 1, and which has some similarity to KSHV ORF 28. RRV ORF 29b corresponds to the complement of nucleotides 45733-46779 of SEQ ID NO 1, and which has some similarity to KSHV ORF 29b. RRV ORF 30 corresponds to nucleotides 46905-47135 of SEQ ID NO 1, and which has some similarity to KSHV ORF 30. RRV ORF 31 corresponds to nucleotides 47093-47746 of SEQ ID NO 1, and which has some similarity to KSHV ORF 31. RRV ORF 32 corresponds to nucleotides 47683-49077 of SEQ ID NO 1, and has some similarity to KSHV ORF 32. RRV ORF 33 corresponds to nucleotides 49049-50059 of SEQ ID NO 1, and which has some similarity to KSHV ORF 33. RRV ORF 29a corresponds to the complement of nucleotides 49977-50960 of SEQ ID NO 1, and has some similarity to KSHV ORF 29a. RRV ORF 34 corresponds to nucleotides 50959-51942 of SEQ ID NO 1, and has some similarity to KSHV ORE 34. RRV ORF 35 corresponds to nucleotides 51923-52372 of SEQ ID NO 1, has some similarity to KSHV ORF 35. RRV ORF 36, corresponds to nucleotides 52278-53585 of SEQ ID NO 1, which encodes a kinase, and which has some similarity to KSHV ORF 36. RRV ORF 37 corresponds to nucleotides 53566-55008 of SEQ ID NO 1, which encodes an alkaline exonuclease, and which has some similarity to KSHV ORF 37. RRV ORF 38 corresponds to nucleotides 54963-55172 of SEQ ID NO 1, and has some similarity to KSHV ORF 38. RRV ORF 39 corresponds to the complement of nucleotides 55255-56391 of SEQ ID NO 1, which encodes glycoprotein M, and which has some similarity to KSHV ORF 39. RRV ORF 40 corresponds to nucleotides 56526-57932 of SEQ ID NO 1, which encodes helicase/primase, and which has some similarity to KSHV ORF 40.

RRV ORF 41, corresponds to nucleotides 57917-58528 of SEQ ID NO 1, which encodes helicase/primase, and which has some similarity to KSHV ORF 41. RRV ORF 42 corresponds to the complement of nucleotides 58525-59343 of SEQ ID NO 1, which has some similarity to KSHV ORE 42. RRV ORF 43 corresponds to the complement of nucleotides 59297-61027 of SEQ ID NO 1, which encodes a capsid protein, and which has some similarity to KSHV ORF 43. RRV ORF 44 corresponds to nucleotides 60966-63338 of SEQ ID NO 1, which encodes helicase/primase, and which has some similarity to KSHV ORF 44. RRV ORF 4, corresponds to the complement of nucleotides 63379-64437 of SEQ ID NO 1, and which has some similarity to KSHV ORF 45. RRV ORF 46 corresponds to the complement of nucleotides 64479-65246 of SEQ ID NO 1, which encodes uracil DNA glucosidase, and which has some similarity to KSHV ORF 46. RRV ORF 47 corresponds to the complement of nucleotides 65222-65731 of SEQ ID NO 1, which encodes glycoprotein L, which has some similarity to KSHV ORF 47. RRV ORF 48 corresponds to the complement of nucleotides 65999-67168 of SEQ ID NO 1, and which has some similarity to KSHV ORF 48. RRV ORF 49 corresponds to the complement of nucleotides 67398-68303 of SEQ ID NO 1, and which has some similarity to KSHV ORF 49. RRV ORF 50 corresponds to nucleotides 68494-70038 of SEQ ID NO 1, which encodes a transactivator, and which has some similarity to KSHV ORF 50. RRV R4 corresponds to nucleotides 70355-70888 of SEQ ID NO 1. RRV R5 corresponds to nucleotides 71468-72160 of SEQ ID NO 1. RRV ORF 52 corresponds to the complement of nucleotides 72401-72820 of SEQ ID NO 1, and has some similarity to KSHV ORF 52. RRV ORF 5, corresponds to the complement of nucleotides 72884-73198 of SEQ ID NO 1, and has some similarity to KSHV ORF 53. RRV ORF 54 corresponds to nucleotides 73274-74146 of SEQ ID NO 1, which encodes a dUTPase, and which has some similarity to KSHV ORF 54. RRV ORF 55 corresponds to the complement of nucleotides 74207-74839 of SEQ ID NO 1, and has some similarity to KSHV ORF 55. RRV ORF 56 corresponds to nucleotides 74851-77337 of SEQ ID NO 1, which encodes a DNA replication protein, and has some similarity to KSHV ORF 56. RRV ORF 5, corresponds to nucleotides 77578-78906 of SEQ ID NO 1, which encodes an immediate-early gene product, and which has some similarity to KSHV ORF 57. RRV R6 corresponds to the complement of nucleotides 79266-80513 of SEQ ID NO 1, which has some similarity to KSHV vIRF K9 gene. RRV R7 corresponds to the complement of nucleotides 80686-81933 of SEQ ID NO 1, which has some similarity to KSHV vIRF K9 gene. RRV R8 corresponds to the complement of nucleotides 82262-83317 of SEQ ID NO 1, which has some similarity to KSHV vIRF K9 gene. RRV R9 corresponds to the complement of nucleotides 83491-84252 of SEQ ID NO 1, which has some similarity to KSHV vIRF K9 gene. RRV R10, corresponding to the complement of nucleotides 85052-86209 of SEQ ID NO 1, which has some similarity to KSHV vIRF K9 gene. RRV R11 corresponds to the complement of nucleotides 86355-87527 of SEQ ID NO 1, which has some similarity to KSHV vIRF K9 gene. RRV R12 corresponds to the complement of nucleotides 87894-88961 of SEQ ID NO 1, which has some similarity to KSHV ORF K9 gene. RRV R13 corresponds to the complement of nucleotides 89122-90216 of SEQ ID NO 1, which has some similarity to KSHV vIRF K9 gene. RRV ORF 58 corresponds to the complement of nucleotides 90462-91544 of SEQ ID NO 1, which has some similarity to KSHV ORF 58. RRV ORF 59 corresponds to the complement of nucleotides 91555-92739 of SEQ ID NO 1, which encodes a DNA replication protein, and which has some similarity to KSHV ORF 59. RRV ORF 60 corresponds to the complement of nucleotides 92868-93812 of SEQ ID NO 1, which encodes a small ribonucleotide reductase protein, and which has some similarity to KSHV ORF 60. RRV ORF 61 corresponds to the complement of nucleotides 93794-96160 of SEQ ID NO 1, which encodes a large ribonucleotide reductase protein, and which has some similarity to KSHV ORF 61. RRV ORF 62 corresponds to the complement of nucleotides 96163-97158 of SEQ ID NO 1, which encodes a assembly/DNA maturation protein, and which has some similarity to KSHV ORF 62. RRV ORF 63 corresponds to nucleotides 97157-99976 of SEQ ID NO 1, which encodes a tegument protein, and which has some similarity to KSHV ORF 63. RRV ORF 64 corresponds to nucleotides 99980-107626 of SEQ ID NO 1, which encodes a tegument protein, and which has some similarity to KSHV ORF 64. RRV ORF 6, corresponds to the complement of nucleotides 107637-108146 of SEQ ID NO 1, which encodes a capsid protein, and which has some similarity to KSHV ORF 65. RRV ORF 66 corresponds to the complement of nucleotides 108152-109498 of SEQ ID NO 1, which has some similarity to KSHV ORF 66. RRV ORF 67 corresponds to the complement of nucleotides 109524-110198 of SEQ ID NO 1, which encodes a tegument protein, and which has some similarity to KSHV ORF 67. RRV ORF 68 corresponds to nucleotides 110609-111982 of SEQ ID NO 1, which encodes a glycoprotein, and which has some similarity to KSHV ORF 68. RRV ORF 69 corresponds to nucleotides 112004-112897 of SEQ ID NO 1, which has some similarity to KSHV ORF 69. RRV ORF 71, corresponds to the complement of nucleotides 119211-119735 of SEQ ID NO 1, which encodes a FLIP protein, and which has some similarity to KSHV ORF 71. RRV ORF 72, corresponds to the complement of nucleotides 119794-120558 of SEQ ID NO 1, which encodes a cyclin D homolog, and which has some similarity to KSHV ORF 72. RRV ORF 73 corresponds to the complement of nucleotides 120866-122212 of SEQ ID NO 1, which encodes a latent nuclear antigen, and which has some similarity to KSHV ORF 73. RRV R15 corresponds to nucleotides 122866-123627 of SEQ ID NO 1, which has some similarity to KSHV K14 and α-2. RRV ORF 74 corresponds to nucleotides 123924-124952 of SEQ ID NO 1, which encodes a G protein coupled receptor, and which has some similarity to KSHV ORF 74. RRV ORF 75 corresponds to the complement of nucleotides 125057-128953 of SEQ ID NO 1, which encodes a tegument protein, FGARAT, and which has some similarity to KSHV ORF 75.

SEQ ID NO 2 shows the nucleic acid sequence of the JMHV ORF21.

SEQ ID NO 3 shows the deduced amino acid sequence of the JMHV ORF21.

SEQ ID NO 4 shows the nucleic acid sequence of another region of the JMHV ORF21.

SEQ ID NO 5 shows the deduced amino acid sequence of a portion JMHVORF21, as encoded by SEQ ID NO:4.

SEQ ID NO 6 shows the nucleic acid sequence of a portion of JMHV cosmid 3 fragment 5 T7, which has similarity to the nucleic acid sequence found in and to the right of RRV repeat unit rDL-B1.

SEQ ID NO 7 shows the nucleic acid sequence of the JMHV ORF 17, a capsid protein.

SEQ ID NO 8 shows the amino acid sequence of the JMHV ORF 17, deduced from SEQ ID NO:7.

SEQ ID NO 9 shows the nucleic acid sequence of JMHV ORF 21 thymidine kinase, which includes frameshift mutations.

SEQ ID NO 10 shows the amino acid sequence of a portion of JMHV ORF 21, which is the deduced amino acid sequence of the region between the framseshifts in SEQ ID NO:9.

SEQ ID NO 11 shows the nucleic acid sequence of JMHV ORF 24.

SEQ ID NO 12 shows the deduced amino acid sequence of JMHV ORF 24.

SEQ ID NO 13 shows the nucleic acid sequence of JMHV ORF 7 transport protein.

SEQ ID NO 14 shows the deduced amino acid sequence of the JMHV ORF 7 transport protein.

SEQ ID NO 15 shows the nucleic acid sequence of cosmid 3, fragment 1, sp6, which is similar to the sequence to the left of RRV repeat rDL-B1.

SEQ ID NO 16 shows the nucleic acid sequence of the JMHV ORF10.

SEQ ID NO 17 shows the deduced amino acid sequence of the JMHV ORF 10.

SEQ ID NO 18 shows the nucleic acid sequence of the ORF 9 DNA polymerase.

SEQ ID NO 19 shows the deduced amino acid sequence of the ORF 9 DNA polymerase.

SEQ ID NO 20 shows the nucleic acid sequence of JMHV ORF 10.

SEQ ID NO 21 shows the deduced amino acid sequence of the JMHV ORF 10.

SEQ ID NO 22 shows the nucleic acid sequence of the JMHV ORF 8 glycoprotein B.

SEQ ID NO 23 shows the deduced amino acid sequence of the JMHV ORF 8 glycoprotein B.

SEQ ID NO 24 shows the nucleic acid sequence of the JMHV ORF 9 DNA polymerase.

SEQ ID NO 25 shows the deduced amino acid sequence of the JMHV ORF9 DNA polymerase.

SEQ ID NO 26 shows the nucleic acid sequence of the JMHV ORF8 glycoprotein B.

SEQ ID NO 27 shows the deduced amino acid sequence of the JMHV ORF 8 glycoprotein B.

### ATCC Deposits

A Budapest Treaty deposit of RRV 17577 was made with the American Type Culture Collection (ATCC), Manassas, Va., on Mar. 12, 1998, and has been accorded ATCC Accession No. VR-2601.

A Budapest Treaty deposit of JMHV 17792 was made with the American Type Culture Collection (ATCC), Manassas, Va., on May 18, 2000, and has been accorded ATCC Accession No. PTA-1884.

### DETAILED DESCRIPTION OF SEVERAL EMBODIMENTS

### Abbreviations and Definitions

Animal: Living multicellular vertebrate organisms, a category which includes, for example, humans, non-human primates, mammals, and birds.

Cell: A plant, animal, insect, bacterial, or fungal cell.

Homologs: two nucleotide or amino acid sequences that share a common ancestral sequence and diverged when a species carrying that ancestral sequence split into two species. Homologs frequently show a substantial degree of sequence identity.

IL-6: Interleukin 6. IL-6 is a cytokine known to have pleiotropic immunological effects including anti-inflammatory and immunosuppressive effects (Human Cytokines, 1991, pg. 142-167, Blackwell Scientific Publications, Aggarwal and Gutterman, eds). Because IL-6 is a pleiotropic cytoline, IL-6 activity may be measured using a number of bioassays, including stimulation of immunoglobulin production in SKW6-C₁₄ cells as described by Hirano et al. (Nature 324:73-6, 1986) and stimulation of hybridoma cell growth as described by Matsuda et al., 1988 Eur. J. Immunol 18:951-956, both of which are incorporated by reference. As used herein, the term “IL-6 biological activity” refers to the ability of a protein to show activity in at least one of these assay systems

Immuno-compromised: Lacking a normal immune response. Immuno-compromised refers to a condition in which some or all of an animal's immune system is inoperative, leaving the animal with an increased susceptibility to infection or disease. An animal may be rendered immuno-compromised by a biological agent such as, in the case of non-human primates, Simian Immunodeficiency Virus (SIV). Many strains of SIV have been isolated and characterized; any SIV strain that produces an immuno-compromised state can be used in the present invention including, but not limited to, for example, SIVmac239 (Kestler et al., 1990, Science 248: 1109-12), SIVmac251 (Daniels et al., 1985, Science 228: 12014), SIVdeltaB670 (Murphy-Corb et al., 1986, Nature 321:435) and SIVmne (Benveniste et al., 1988, J. Virol. 62:2091-101). In addition, hybrid SIV/HIV chimeras as known in the field can be employed, as can HIV-2. Simian type D retroviruses (SRVs) which cause an AIDS-like disease in rhesus monkeys, can alternatively be used to immuno-compromise the animals in place of SIV. These viral agents are administered to the animal using conventional means, such as intravenous or intramuscular injection, or oral, intrarectal or intravaginal inoculation (also see Example 24). Either intact viral particles or viral DNA may be administered. As known in the field, plasmid constructs containing the entire SIV genome are infectious when inoculated into animals and so may be employed in place of purified viral DNA.

Alternatively, an animal may be rendered immuno-compromised by administration of agents that target the immune system, including but not limited to anti-CD3 antibody (CD3 being the T-cell receptor) either alone or conjugated with a toxic moiety, or immunosuppressive compounds including prednisone, azathioprine, cyclosporine A, and cyclophosphamide. Where an immunosuppressive compound such as cyclosporine is employed, an allogenic stimulus (such as a blood transfusion) may be administered with the subsequent administration of RRV to activate infection.

Alternatively, other methods of rendering an animal immuno-compromised may be used, including radiation treatment and surgical intervention.

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

JMHV: A virus having the virological, immunological or pathological characteristics of Japanese Macaque Herpesvirus (e.g. JMHV 17792). JMHV causes the symptoms of MS in Japanese macaque monkeys which are infected with the virus. In particular examples, the JMHV has at least 85% (for example at least 90%, 95% or 98%) sequence identity to the JMHV deposited with the virus deposited as ATTC Accession Number PTA-1884. In other examples, the JMHV has at least 90%, 91%, 92%, 93%, 94%, 95%, or 98% homology with an RRV (e.g. ATCC VR-2901). Without being bound by theory, the JMHV may be derived from an RRV (see the experimental examples).

JMHV 17792: Japanese macaque herpesvirus isolate 17792. A Budapest Treaty deposit of JMHV 17792 was made with the American Type Culture Collection, Manassas, Va. on May 18, 2001. This virus may be grown on primary Japanese macaque fibroblasts, using standard virological techniques. Alternatively, it may be grown on commercially available macaque cell lines. Infection of a non-human primate with JMHV 17792 can be accomplished using any standard method, including intravenous injection. In one embodiment, infection is achieved using 10⁶ plaque forming units of JMHV 17792.

KSHV: Kaposi's sarcoma-associated herpesvirus. KSHV is a herpesvirus associated with (and thought to be the etiological agent of) Kaposi's sarcoma in humans.

Lymphoproliferative Disorder: a group of disorders characterized by proliferation of lymphoid tissue, such a§ lymphocytic leukemia and malignant lymphoma, and characterized by such signs as lymphocytosis, lymphadenopathy, and splenomegaly.

MIP: macrophage inflammatory protein. The acronym MIP is used to describe a family of cytokines that includes MIP1 (Davatelis et al., 1989, Science 243: 1066-8) and MIP2 (U.S. Pat. No. 5,145,676). MIPs mediate pleiotropic immunological effects including activation of neutrophils to undergo an oxidative burst. MIPs are also intrinsically pyrogenic. MIP biological activity can be detected and quantified using bioassays as described in Kedal et al. (Science 277:1656-9, 1997) and Boshoff et al. (Science 278:290-4, 1997) that measure MIP concentrations using HIV inhibition and calcium mobilization, respectively. As used herein, the term “MIP biological activity” refers to the ability of a protein to show activity in at least one of these assay systems.

Multiple Sclerosis: A chronic, progressive disease of the central nervous system Currently, the exact cause of the disease is unknown and there is no cure. Multiple sclerosis refers to multiple areas of patchy scarring, or plaques, that result from demyelination (destruction of myelin, a fatty insulation covering the nerve fibers). When the myelin sheath is destroyed during the MS disease process, signals transmitted throughout the CNS are slowed or disrupted. In many cases, the body may compensate for the loss of myelin by increasing the density of the sodium channels so that action potentials can continue to be carried, in spite of loss of myelin. The nerves also retain the capacity to remyelinate. Unfortunately, the disease process often outpaces these corrective actions.

The symptoms, severity, and course of MS vary widely depending partly on the sites of the plaques and the extent of the demyelination. Experts generally group multiple sclerosis into four types: relapsing-remitting, primary-progressive, secondary-progressive, and progressive-relapsing MS. Relapsing-remitting multiple sclerosis generally occurs in younger people and is the most common form of MS. Symptoms flare up for several days and then go into remission over the next four to eight weeks. The latter three forms (primary-progressive, secondary-progressive, and progressive-relapsing MS) generally fall under the category of chronic-progressive MS. In chronic-progressive MS the symptoms of the disease continue to worsen slowly without remission. About 20% of multiple sclerosis patients (usually those patients whose first symptoms occur after age 45) have the chronic-progressive form of MS without first developing relapsing-remitting MS. Chronic-progressive MS may lead to serious speech problems and paralysis, and generally the symptoms continue to worsen over time.

Multiple sclerosis is defined as an autoimmune disease; that is, the body's immune system is damaged by genetic or environmental factors or both, causing it to attack its own tissues. In the case of MS, these tissues are the myelin covering the nerve fibers in the brain.

Although the exact agent that triggers MS is unknown, it has been proposed that viral antigens could trigger the autoimmune response in MS. One factor that seems to support this theory is the geographical distribution of the disease; the number of MS cases increases the further one gets from the equator in either direction. Epidemics of multiple sclerosis also seem to occur. For example, four separate MS epidemics happened between 1943 and 1989 in the Faroe Islands, located between Iceland and Scandinavia. During World War II, this region was occupied by British troops; each year for 20 years after the war, the incidence of MS increased, leading some researchers to think that the troops might have brought with them some disease-causing agent.

Although several virus have been associated with MS, no one causative agent has been identified. It has been proposed that HHV-6, a form of herpesvirus, may be the causative agent. However, other viruses have been implicated in the disease process including herpesviruses 1 and 2, varicella-zoster virus, cytomegalovirus, Epstein-Barr virus, measles virus, adenovirus, polyomavirus, and retroviruses, including HIV, HTLV-I, and HTLV-II. It has also been proposed that bacteria may be the causative agent. In one recent study, 17 patients who were admitted to a hospital and diagnosed with MS showed signs of infection with the microorganism Chlamydia pneumoniae, an atypical bacterium. However, the association of these any of these agents with MS is difficult, as infection in the general population is common.

Genetic factors are also believed to have a role in the development of MS. Children of MS patients have an increased risk 30 to 50 times that of the normal population. In addition, the odds of an MS sibling having the disease are about 20 times higher than in the normal population. A significant association between siblings with MS and the specific form of the disease, either relapsing-remitting or chronic progressive has been found. It is possible that a set of genes that somehow interact are responsible for the inherited risks. In addition, genetic variations that occur in different ethnic populations are also believed to result in a variable disease course amongst the different ethnic groups.

A symptom of MS is any physical characteristic associated with the disease process. For example, the first symptom of MS is often optic neuritis, the inflammation of the optic nerve. Vision, usually in one eye, becomes unclear or doubled, and there may be a shimmering effect Pain or nystagmus, involuntary jerking or movement of the eye, may also occur. In 20% of people with this condition, MS develops within two years; in 45% to 80% it develops within 15 years. Other early symptoms of multiple sclerosis include fatigue, heaviness or clumsiness in the arms and legs, tingling sensations, and poor coordination. Another indication of MS is a reaction known as Llermitte's sign, whereby bending the neck produces an electrical sensation that runs down the back and into the legs.

As the disease develops over months or even years, other symptoms may include spasticity, imbalance, tremors, incontinence, constipation, sexual dysfunction, hearing loss, vertigo, facial pain, and difficulties in swallowing. Problems in speech may occur because of difficulty in controlling the quality of the voice and articulating words. About half of patients display changes in mental function, including problems in concentration and problem solving. In about 10% of cases, there is severe mental dysfunction which resembles dementia. One of the primary symptoms of MS is spasticity, which is characterized by weakness, loss of dexterity, and the inability to control specific movements.

In general, MS itself is not fatal, although people who have this disease generally have a shorter life span than average. However, the negative emotional impact of this disease and its symptoms is considerable. The severity of the disease varies widely from patient to patient; MS sometimes remains asymptomatic or becomes only mildly symptomatic even long after initial plaque formation. About 20% to 35% of patients have a very mild form of the disease, with little if any disability, no need for medication, and a normal life expectancy. At the other extreme, between 3% and 12% of patients have a very serious, rapidly progressive form of MS. Most patients fall somewhere in the middle. Life-threatening complications may occur as a result of infections in the lungs or kidneys. As the muscles that control breathing weaken, the ability to cough is impaired and the patient is at higher risk for pneumonia and other complications in the lungs. Complications in the urinary tract also place the patient at risk for kidney infections. In very severe cases of MS, paralysis may occur. The severity of disability incurred over a lifetime is unpredictable.

About 1.1 million people worldwide have multiple sclerosis, and the incidence appears to be increasing. Between 250,000 and 350,000 Americans have the disease. Onset of symptoms typically occurs between the ages of 15 and 40 years, with a peak incidence in people in their 20s and 30s. Women are affected twice as often as men. Multiple sclerosis occurs worldwide but is most common in Caucasian people of northern European origin, especially those of Scottish descent It is extremely rare among Asians and Africans. In general, MS is more prevalent in temperate regions of the world than in the tropics. It is unclear whether this pattern is attributable to environmental factors or to genetics. A family history of the disease also puts people at risk, although the risk for someone inheriting all the genetic factors contributing to MS is only about 2% to 4%.

Myelin: A coating of nerve cells (neurons) made from layers of cell membranes that are produced in the brain and spinal cord by specialized cells called oligodendrocytes. Myelin coats lie in segments along the axons, the long filaments that carry electric impulses away from a nerve cell. The segments are separated from each other by tiny clusters called nodes of Ranvier, which house channels for sodium ions. These sodium ions are important for boosting the electrical charge required to pass signals from one nerve to another.

Non-human primate: Simian primates including chimpanzees, orangutans, baboons, and macaques. Any non-human primate may be used to produce a KSHV-disease animal model or a JMHV-disease animal model by the methods disclosed herein. Thus, in addition to the rhesus macaque and Japanese Macaque models described in detail below, pigtail and cynomologus macaques and baboons may also be used to produce KSHV-disease or JMHV-disease animal models by the methods disclosed herein.

Oligonucleotide: A linear polynucleotide sequence of up to about 200 nucleotide bases in length, for example a polynucleotide (such as DNA or RNA) which is at least 6 nucleotides, for example at least 15, 25, 50, 100 or even 200 nucleotides long.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

ORF: open reading frame. Contains a series of nucleotide triplets (codons) coding for amino acids without any termination codons. These sequences are usually translatable into protein.

PCR: polymerase chain reaction. Describes a technique in which cycles of denaturation, annealing with primer, and then extension with DNA polymerase are used to amplify the number of copies of a target DNA sequence.

Pharmaceutically acceptable carriers: The pharmaceutically acceptable carriers useful in this invention include conventional carriers. Remington's Pharmaceutical Sciences, by E. W. Martin, Mack Publishing Co., Easton, Pa., 15th Edition (1975), describes compositions and formulations suitable for pharmaceutical delivery of the viruses, nucleic acids and/or proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, ethanol, combinations thereof, or the like, as a vehicle. The carrier and composition can be sterile, and the formulation suits the mode of administration. For solid compositions (e.g., powder, pill, tablet, or capsule forms), conventional non-toxic solid carriers can include, for example, pharmaceutical grades of mannitol lactose, starch, sodium saccharine, cellulose, magnesium carbonate, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The composition can be a liquid solution, suspension, emulsion, tablet, pill, capsule, sustained release formulation, or powder. The composition can be formulated as a suppository, with traditional binders and carriers such as triglycerides.

Probes and primers: Nucleic acid probes and primers may readily be prepared based on the amino acid sequences provided by this invention. A probe is an isolated nucleic acid attached to a detectable label or reporter molecule. Typical labels include radioactive isotopes, ligands, fluorescent molecules, chemiluminescent molecules, and enzymes. In other embodiments, labels include co-factors, enzyme substrates; and haptens. Methods for labeling and guidance in the choice of labels appropriate for various purposes are discussed, e.g., in Sambrook et al., in Molecular Cloning A Laboratory Manual, Cold Spring (1989) and Ausubel et al., in Current Protocols in Molecular Biology, Greene Publishing Associates and Wiley-Intersciences (1987).

Primers are short nucleic acids, such as DNA oligonucleotides 10 nucleotides or more in length. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by the polymerase chain reaction (PCR) or other nucleic-acid amplification methods known in the art.

Methods for preparing and using probes and primers are described, for example, in Sambrook et al. (Molecular Cloning: A Laboratory Manual, 2^{nd} ed., vol. 1-3, Cold Spring Harbor, N.Y. 1989); Ausubel et al. (Current Protocols in Molecular Biology, Greene Publ. Assoc. & Wiley-Intersciences. 1987) and Innis et al. (PCR Protocols, A Guide to Methods and Applications, 1990, Innis et al. (eds.), 21-27, Academic Press, Inc., San Diego, Calif.). PCR primer pairs can be derived from a known sequence, for example, by using computer programs intended for that purpose such as Primer (Version 0.5, © 1991, Whitehead Institute for Biomedical Research, Cambridge, Mass.).

Probes and primers as used in the present invention typically comprise at least 15 contiguous nucleotides of the RRV genome sequence (SEQ ID NO 1), or 15 contiguous nucleotides of a JMHV sequence. One of skill in the art will appreciate that the specificity of a particular probe or primer increases with its length. Thus, for example, a primer comprising 20 consecutive nucleotides will anneal to a target with a higher specificity than a corresponding primer of only 15 nucleotides. Thus, in order to obtain greater specificity, probes and primers may be selected that comprise 20, 25, 30, 35, 40, 50 or more consecutive nucleotides. In order to enhance specificity, longer probes and primers may also be employed, such as probes and primers that comprise at least 20, 30, 40, 50, 60, 70, 80, 90, 100, or 150 consecutive nucleotides of the disclosed nucleic acid sequences.

Alternatively, such probes and primers may comprise at least 15, 20, 30, 40, 50, 60, 70, 80, 90, 100, or 150 consecutive nucleotides that share a defined level of sequence identity with the disclosed RRV or JMHV sequence, for instance, at least a 60%, 70%, 80%, 90%, 95% or 98% sequence identity. Alternatively, such probes and primers may be nucleotide molecules which hybridize under wash conditions of 70° C. and about 0.2×SSC for 1 hour, or alternatively under less stringent conditions of 65° C., 60° C., or 55° C. with from about 0.2 to 2×SSC (with, for instance, about 0.1% SDS) for 1 hour with a portion of the RRV sequence.

Purified: The term purified does not require absolute purity; rather, it is intended as a relative term. Thus, for example, a purified peptide preparation is one in which the peptide or protein is more enriched than the peptide or protein is in its natural environment within a cell. Preferably, a preparation is purified such that the protein or peptide represents at least 50% of the total peptide or protein content of the preparation.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

RRV 17577: Rhesus macaque rhadinovirus RRV isolate 17577. A Budapest Treaty deposit of RRV 17577 was made with the American Type Culture Collection, Manassas, Va., on Mar. 12, 1998, and has been accorded ATCC Accession No. VR-2601. This virus may be grown on primary rhesus fibroblasts, as described below (see Examples 1 and 14), using standard virological techniques. Alternatively, it may be grown on commercially available rhesus cell lines, including those available from ATCC, such as ATCC CRL-6306 and ATCC CL-160. Infection of a non-human primate with RRV 17577 may be accomplished using any standard method, including intravenous injection (see Examples 13, 23 and 24). Typically, infection is achieved by intravenous injection of around 10⁶ plaque forming units (PFUs) of RRV 17577.

RRV: A virus having the virological and immunological characteristics of RRV 17577, and which causes Kaposi's sarcoma in immunocompromised Rheusus monkeys which are infected with the virus. In particular examples, the RRV has at least 85% (for example at least 90%, 95% or 98%) sequence identity to SEQ ID NO 1.

Sequence Identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Homologs or orthologs of nucleic acid or amino acid sequences will possess a relatively high degree of sequence identity when aligned using standard methods. This homology will be more significant when the orthologous proteins or nucleic acids are derived from species which are more closely related (e.g., human and chimpanzee sequences), compared to species more distantly related (e.g., human and C. elegans sequences). Typically, orthologs are at least 50% identical at the nucleotide level and at least 50% identical at the amino acid level when comparing human orthologous sequences.

Methods of alignment of sequences for comparison are well known. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. Biosci. 8, 155-65, 1992; and Pearson et al., et al. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biotechnology Information (NCBI, Bethesda, Md.) and on the Internet, for use in connection with the sequence analysis programs blastp, blastn, blastx, tblastn and tblastx. Each of these sources also provides a description of how to determine sequence identity using this program.

Homologous sequences are typically characterized by possession of at least 60%, 70%, 75%, 80%, 90%, 95% or at least 98% sequence identity counted over the full length alignment with a sequence using the NCBI Blast 2.0, gapped blastp set to default parameters. Queries searched with the blastn program are filtered with DUST (Hancock and Armstrong, Comput. Appl. Biosci. 10:67-70, 1994). It will be appreciated that these sequence identity ranges are provided for guidance only; it is entirely possible that strongly significant homologs could be obtained that fall outside of the ranges provided.

Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that all encode substantially the same protein.

One indication that two nucleic acid sequences are substantially identical is that the polypeptide encoded by the first nucleic acid is immunologically cross reactive with the polypeptide encoded by the second nucleic acid.

An alternative indication that two nucleic acid molecules are closely related is that the two molecules hybridize to each other under stringent conditions, as described under “specific hybridization.”

Homologs of the disclosed RRV or JMHV nucleic acids typically possess at least 50% sequence identity counted over the length of one of the nucleic acids (the reference nucleic acid) using the NCBI Blast 2.0.6, gapped blastn set to default parameters. Nucleic acids showing substantial similarity when assessed by this method may show, for example, at least 50%, 60%, 70%, 80%, 90%, 95% or even 98% or greater sequence identity. When less than the entire sequence is being compared for sequence identity, substantially similar nucleotide sequences will typically possess at least 70% sequence identity over short windows of 30-90 nucleic acids, and may possess sequence identities of at least 80%, 90%, 95% or 98% or greater.

Homologs of the disclosed RRV or JMHV proteins typically possess at least 50% sequence identity counted over full-length alignment with the amino acid sequence of RRV using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least 50%, at least 55%, at least 60%, at least 70%, at least 75%, at least 80%, at least 90%, at least 95%, at least 98%, or at least 99% sequence identity. When less than the entire sequence is being compared for sequence identity, homologs will typically possess at least 70% sequence identity over short windows of 10-20 amino acids, and may possess sequence identities of at least 75%, at least 85% or at least 90%, at least 95% or 98% depending on their similarity to the reference sequence.

When comparing degrees of sequence identity between similar proteins, the degree of identity will be equal to or less than that the degree of similarity, due to the fact the similarity takes into account conservative amino acid substitutions. So, for instance, the degree of sequence identity between to substantially similar proteins may be at least 50%, 55%, 65%, 75%, 85%, 95%, 98% or more.

One of ordinary skill in the art will appreciate that these sequence identity ranges are provided for guidance only; it is entirely possible that strongly significant homologs could be obtained that fall outside of the ranges provided. The present invention provides not only the peptide homologs that are described above, but also nucleic acid molecules that encode such homologs.

An alternative indication that two nucleic acid molecules are closely related is that the two molecules hybridize to each other under stringent conditions, as described in Example 23.

Specific binding agent: An agent that binds substantially only to a defined target. As used herein, the term “RRV peptide specific binding agent” includes anti-RRV peptide antibodies and other agents that bind substantially only to the RRV peptide. Such “peptide specific binding agents” include anti-IL-6 and anti-MIP antibodies. Similarly, the term “JMHV peptide specific binding agent” includes anti-JMHV peptide antibodies and other agents that bind substantially only to the JMHV peptide. The antibodies may be monoclonal or polyclonal antibodies that are specific for an RRV or a JMHV peptide, as well as immunologically effective portions (“fragments”) thereof.

In one embodiment, the antibodies used in the present invention are monoclonal antibodies (or immunologically effective portions thereof) and may also be humanized monoclonal antibodies (or immunologically effective portions thereof). Immunologically effective portions of monoclonal antibodies include Fab, Fab′, F(ab′)₂, Fabc and Fv portions (for a review, see Better and Horowitz, Methods. Enzymol. 178:476-96, 1989). Anti-inhibitory peptide antibodies may also be produced using standard procedures described in a number of texts, including Antibodies, A Laboratory Manual by Harlow and Lane, Cold Spring Harbor Laboratory (1988).

Methods of making humanized monoclonal antibodies are well known, and include those described in U.S. Pat. Nos. 5,585,089; 5,565,332; 5,225,539; 5,693,761; 5,693,762; 5,585,089; and 5,530,101 and references cited therein. Similarly, methods of making and using immunologically effective portions of monoclonal antibodies, also referred to as antibody fragments, are well known and include those described in Better and Horowitz, 1989, Meth. Enzymol. 178:176-496; Better et al., 1990, Better and Horowitz, 1990, Advances in Gene technology: The Molecular Biology of Immune Disease & the Immune Response (ICSU Short Reports); Glockshuber et al., 1990, Biochemistry 29:1362-7; and U.S. Pat. Nos. 5,648,237; 4,946,778 and 5,455,030, and references cited therein.

The determination that a particular agent binds substantially only to an RRV peptide or a JMHV peptide may readily be made by using or adapting routine procedures. One suitable in vitro assay makes use of the Western blotting procedure (described in many standard texts, including Antibodies, A Laboratory Manual by Harlow and Lane). Western blotting may be used to determine that a given RRV peptide binding agent, such as an anti-IL-6 or MIP peptide monoclonal antibody, binds substantially only to the specific RRV protein.

Specific hybridization: Specific hybridization refers to the binding, duplexing, or hybridizing of a molecule only or substantially only to a particular nucleotide sequence when that sequence is present in a complex mixture (e.g. total cellular DNA or RNA). Specific hybridization may also occur under conditions of varying stringency.

Supernatant: The culture medium in which a cell is grown. The culture medium includes material from the cell. If the cell is infected with a virus, the supernatant can include viral particles.

Subject: This term includes both human and non-human subjects. Similarly, the term “patient” includes both human and veterinary subjects.

Transformed: A transformed cell is a cell into which has been introduced a nucleic acid molecule by molecular biology techniques. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Transgenic Cell: Transformed cells which contain foreign, non-native DNA.

Variants of Amino Acid and Nucleic Acid Sequences: The production of RRV or JMHV proteins can be accomplished in a variety of ways (for example see Examples 17, 21 and 25). DNA sequences which encode the protein, or a fragment of the protein, can be engineered such that they allow the protein to be expressed in eukaryotic cells, bacteria, insects, and/or plants. In order to accomplish this expression, the DNA sequence can be altered and operably linked to other regulatory sequences. The final product, which contains the regulatory sequences and the therapeutic protein, is referred to as a vector. This vector can then be introduced into the eukaryotic cells, bacteria, insect, and/or plant. Once inside the cell the vector allows the protein to be produced.

One of ordinary skill in the art will appreciate that the DNA can be altered in numerous ways without affecting the biological activity of the encoded protein. For example, PCR may be used to produce variations in the DNA sequence which encodes RRV or JMHV proteins, such as IL-6 or MIP. Such variants may be variants that are optimized for codon preference in a host cell that is to be used to express the protein, or other sequence changes that facilitate expression.

Two types of cDNA sequence variant may be produced. In the first type, the variation in the cDNA sequence is not manifested as a change in the amino acid sequence of the encoded polypeptide. These silent variations are simply a reflection of the degeneracy of the genetic code. In the second type, the cDNA sequence variation does result in a change in the amino acid sequence of the encoded protein. In such cases, the variant cDNA sequence produces a variant polypeptide sequence. In order to preserve the functional and immunologic identity of the encoded polypeptide, it is preferred that any such amino acid substitutions are conservative. Conservative substitutions replace one amino acid with another amino acid that has some homology in size, hydrophobicity, etc.

Such substitutions generally are conservative when it is desired to finely modulate the characteristics of the protein. For example, conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

Examples of amino acids which may be substituted for an original amino acid in a protein and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

The substitutions which in general are expected to produce the greatest changes in protein properties will be non-conservative, for instance changes in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histadyl, is substituted for (or by) an electronegative residue, e.g., glutamyl or aspartyl; or (d) a residue having a bulky side chain, e.g., phenylalanine, is substituted for (or by) one not having a side chain, e.g., glycine.

Variations in the DNA sequence that result in amino acid changes, whether conservative or not, should be minimized in order to preserve the functional and immunologic identity of the encoded protein. The immunologic identity of the protein may be assessed by determining whether it is recognized by an antibody to an RRV (or JMHV) protein; a variant that is recognized by such an antibody is immunologically conserved. Any DNA sequence variant will preferably introduce no more than 20, and preferably fewer than 10 amino acid substitutions into the encoded polypeptide. Variant amino acid sequences can, for example, be 80%, 90%, 95% or even 98% identical to the native amino acid sequence.

Vector: A nucleic acid molecule as introduced into a host cell, thereby producing a transformed host cell. A vector may include nucleic acid sequences that permit it to replicate in the host cell, such as an origin of replication. A vector may also include one or more selectable marker genes and other genetic elements known in the art.

Virion: A complete viral particle including envelope, capsid (if any), and nucleic acid elements.

The present invention utilizes standard laboratory practices for the cloning, manipulation and sequencing of nucleic acids, purification and analysis of proteins and other molecular biological and biochemical techniques, unless otherwise stipulated. Such techniques are explained in detail in standard laboratory manuals such as Sambrook et al. (Molecular Cloning: A Laboratory Manual, 2^{nd} ed., vol. 1-3, Cold Spring Harbor, N.Y. 1989) and Ausubel et al. (Current Protocols in Molecular Biology, Greene Publ. Assoc. & Wiley-Intersciences. 1987).

### EXAMPLE 1

### Isolation of RRV

This example describes how RRV was isolated from a rhesus macaque monkey. Fresh, dispersed bone marrow (BM) cells were isopynic gradient-purified (Ficoll-Paque, Pharmacia) from a 2 yr, 202 day old captive-reared rhesus macaque that was euthanized 503 days after intravenous infection with an SIVmac239 variant. Gradient-purified BM mononuclear cells were seeded into T-25 culture flasks and grown in the presence of Endothelial SFM media (GIBCO) supplemented with 10% fetal bovine serum, 1% L-glutamine, 1% penicillin-streptomycin-neomycin and 30 μg/mL endothelial cell growth supplement.

Cultures developing cytopathic effects (CPE) were rapidly frozen in liquid N₂ and thawed, and supernatants clarified by centrifugation and filtered through a 0.45 μ membrane. Filtered extracts were then used as inoculum on primary rhesus macaque fibroblast cultures. Fibroblast cultures developing CPE were scraped free into medium, pelleted at 400×g, washed in phosphate-buffered saline and suspended in cold Ito and Karnovsky's fixative (2.5% glutaraldehyde, 0.5% picric acid, 1.6% paraformaldehyde, 0.005% ruthenium red) in 0.1 M sodium cacodylate buffer, pH 7.4 for 2 hours. Fixed cells were washed in cacodylate buffer, post-fixed in 1% 0504 and 0.8% K₃Fe (Cn)₆ in cacodylate buffer for 1 hour, rinsed in distilled H₂O and pre-stained in 2% aqueous uranyl acetate for 1 hour. Fixed and pre-stained cells were dehydrated in a graded series of acetone imbedded in Epon 812 epoxy resin, polymerized at 60° C. and sectioned at 60 nm on an MT 5000 ultramicrotome. Copper grid mounted sections were stained with lead citrate and Uranyl acetate and viewed on a Phillips 300 electron microscope.

By electron microscopy, numerous herpesvirus particles were observed in the cells. This macaque developed LPD characterized as lymphocytic masses in myeloid and nonlymphoid tissues which were confirmed histopathologically as solid pleomorphic lymphoid masses.

### EXAMPLE 2

### Initial Characterization of RRV

Infectious virus was purified from infected primary rhesus fibroblast cultures exhibiting 100% CPE (see Example 1). Infected cells were harvested and disrupted by freeze-thawing and the cell debris removed by low speed centrifugation. Supernatants were centrifuged in a Beckman JA-14 rotor for 1 hour at 9000 rpm to pellet the virus, which was further purified through a six-step sorbitol gradient ranging from 20 to 70%, spun in a Beckman SW41 rotor for 2 hours at 18,000 rpm. Virus was diluted in balanced buffered salts solution and then pelleted through a 20% sorbitol cushion. Pelleted virus was resuspended in Tris-EDTA buffer (TE; 10 MM Tris-HCl, pH 8.0, and 1 mM EDTA) and lysed in TE with 0.6% SDS and proteinase K (200 μg) at 37° C. for 5 hours. Viral DNA was then isolated by CsCl₂ gradient centrifugation in a Beckman Ti 75 rotor at 38.4 K rpm for 72 hours, collected and dialyzed against TE.

The viral DNA was analyzed using degenerate primer polymerase chain reaction (PCR) amplification and Southern blot hybridization with a probe specific for the KSHV thyrmidylate synthase (TS) gene: The probe itself had the sequence of the KSHV TS (Orf 70 gene). Four genes were identified by these methods. A fragment encoding a portion of the viral DNA polymerase was obtained and DNA sequence analysis revealed that the virus was most likely a gamma herpesvirus, as amino acid sequence identity was highest among this class of herpesviruses. DNA sequence analysis of the viral DNA fragment found to hybridize to the KSHV TS probe revealed three open reading frames (ORFs) with homology to KSHV (Nicholas et al., 1997, Nature Med. 3:287-92; Russo et al.; 1996, Proc Natl Acad Sci USA 93:14862-7). One ORF encodes a homologue of macrophage inflammatory protein MIP-1 with amino acid sequence identity with KSHV MIP-II, the second ORF encodes a thyrmdylate synthase homologue and the third ORF encodes a homologue of interleukin-6 (IL-6) with homology to the rhesus IL-6 and KSHV IL-6. The presence of an IL-6-like cytokine and an MIP-1-like CC-chemokine flanking TS resembles the genomic organization of KSHV, indicating this virus is related to KSHV and is referred to herein as rhesus rhadinovirus (RRV).

To determine if RRV is present in tissue containing the lymphocytic masses, oligonucleotide PCR primers specific for the RRV MIP gene (see PCT Application No. U.S. 99/26260) were designed in an attempt to detect viral DNA in tissue from the macaque. By semi-quantitative PCR analysis, viral DNA sequences were detected in DNA samples from bone marrow at approximately 590 copies per 0.1 μg of tissue DNA. Because rhesus macaques held in captivity are reported to be naturally infected with a herpesvirus similar to KSHV, bone marrow DNA samples were isolated from normal and SIVmac239-infected macaques without LPD and analyzed by PCR. There was no evidence of viral DNA sequences. Additionally, since simian Epstein-Barr virus (EBV) has been found to be present in high copy number in lymphomas from SIV-infected macaques (Baslin et al., 1986, J. Natl. Cancer Inst. 77:127-39; Feichtingeretal., 1990, Amer. J. Pathol. 137:1311-5), the tissue samples from the macaque with disease were also analyzed by PCR for rhesus EBV (RhEBV) using oligonucleotide primers for RhEBV latent membrane protein 1. By this analysis, no signal for RhEBV was detected, suggesting that the RRV may be a contributing factor for LPD in this SIV-infected macaque.

### EXAMPLE 3

### Preparation of RRV DNA for Cloning

Primary rhesus fibroblasts grown in two 850 cm² roller bottles were infected with RRV at an MOI of 0.1 and the virus was harvested from the culture supernatant and the infected monolayers 10 to 12 days post-infection. Cellular debris was removed from the culture supernatant by centrifugation at 1,000×g for 10 minutes. Intracellular virus particles were released by sonication followed by centrifugation to pellet debris.

The two clarified supernatants were then combined and the virus was pelleted by centrifugation at 12,500×g for 1 hour at 4° C., and further purified through a six-step sorbitol gradient ranging from 20 to 70%. Gradients were centrifuged in a Beckman SW41 rotor for 2 hours at 18,000 rpm at 4° C. The interface containing the virus was collected and diluted with cold buffered saline solution. The virus was then pelleted by centrifugation in the SW41 for 50 minutes at 18,000 rpm. The virus pellet was resuspended in 9.2 ml of TE (see Example 2) before the addition of 0.6 ml of 10% sodium dodecylsulfate (SDS) and 0.2 ml of proteinase K (10 mg/mL) to release the viral DNA. Viral DNA was isolated by CsCl₂ gradient centrifugation in a Beckman Ti75 rotor at 38,400 rpm for 72 hours, collected, and dialyzed against TE.

To ensure that the DNA isolated contained all the necessary sequences required for RRV replication, DNA was transfected, in duplicate, into primary rhesus fibroblasts by the calcium phosphate method without dimethyl sulfoxide shock and observed for cytopathic effects (CPE). Control transfections, lacking viral DNA or calcium phosphate, did not develop CPE.

### EXAMPLE 4

### Construction of the Cosmid Library

Approximately 100 μg of purified RRV DNA (Example 3) was partially digested with Sau3A I. Aliquots taken at various time points were run on a 0.5% agarose gel and examined for the fraction which gave the desired range of fragments (30-42 kb). The selected fraction was dephosphorylated with calf intestinal alkaline phosphatase and 1 μg ligated into the cosmid vector SuperCos 1, prepared essentially as described by the manufacturer (Stratagene, La Jolla, Calif.). The resulting ligation product was packaged using GigaPack II Gold packaging extract (Stratagene) and grown for the isolation of recombinant cosmids.

Individual recombinant cosmids were grown in 3 ml cultures and the cosmid DNA was isolated by alkaline lysis. Cosmid DNA was digested with EcoR1 and the DNA fragments separated on a 0.8% agarose gel. The separated fragments were transferred to nitrocellulose and probed with various PCR amplification products corresponding to specific KSHV ORFs. Hybridization of the probes to the transferred recombinant cosmids was done under conditions of moderate stringency (2×SSC-0.1% SDS at 55° C.) with each of the KSHV-specific probes and at high stringency (0.2×SSC-0.1% SDS at 60° C.) with the RRV-specific probes. By this analysis and restriction endonuclease mapping, the recombinant cosmids were aligned and a set of recombinants was identified that represented the entire viral genome when compared to digested viral DNA.

### EXAMPLE 5

### Cloning and Sequencing

Ten micrograms of each purified recombinant cosmid (Example 4) were digested with EcoRI and the resulting fragments isolated from a 0.8% agarose gel using the QiaQuick gel extraction protocol (Qiagen). Recovered fragments were ligated into pSP73 (Promega). Individual clones were selected by restriction enzyme screening of DNA recovered by alkaline lysis from overnight cultures. Sequencing templates were prepared by alkaline lysis, followed by precipitation with 6.5% polyethylene glycol and 0.8 M NaCl. Templates were resuspended at a concentration of 0.1 μg/μl and end sequences were determined using primers corresponding to the SP6 and T7 promoters of pSP73. Internal sequences were determined using a combination of subcloning using convenient restriction sites and custom primers. DNA sequencing reactions were performed with the Applied Biosystems (ABI) PRISM Dye Terminator Cycle Sequencing Ready Reaction kits with AmpliTaq DNA polymerase per the manufacturer's instructions. Sequence data was acquired using an ABI 373A Sequencer in the Molecular Biology Core at the Oregon Regional Primate Research Center. The primary EcoRI fragments were sequentially arranged by sequencing across the EcoRI sites in the intact cosmids using custom primers. Except for those regions containing long, high GC repeat units, the entire viral DNA sequence was determined with a redundancy of 3- to 4-fold.

Sequences not accessible to custom primers or restriction subcloning were determined following deletion subcloning using the Exo Size Deletion kit (New England Biolabs). To accommodate this protocol, fragments were subcloned into vectors with restriction sites capable of generating the needed 3′ and 5′ overhanging ends. Double restriction digests to generate 3′ and 5′ overhanging ends were performed on 10 μg of recombinant plasmid DNA, which was then subjected to exonuclease III digestion. Aliquots were removed from the exonuclease m digests at empirically-determined time points, frozen on dry ice, then, after all the time points had been collected, incubated for 15 minutes at 65° C. to inactivate the enzyme. The DNA was then treated with Mung bean nuclease (MBN) for 30 minutes at 30° C. Prior to addition of 3 μl of MBN to the 12 μl exonuclease III product, the enzyme was diluted 1/25 to reduce nonspecific digestion. Nuclease-treated DNA was recovered using the Wizard prep system (Promega), then incubated for 30 minutes with 2.5 units of T4 DNA polymerase (Life Technologies) and 1 μM dNTPs at 37° C. The final product was ligated overnight with T4 DNA ligase and used to transform competent XL1 blue bacteria. Deletion products were size selected by restriction digests of DNA recovered from 3 ml cultures.

### EXAMPLE 6

### Assembly of the RRV Sequence, Assignment of ORFs, and Nomenclature

Factura (ABI) and Autoassembler (ABI) were used to assemble the final sequence from individual sequencing runs. Open reading frames in the RRV sequence were determined with the program MacVector (Oxford Molecular Group), using a setting of 100 or more amino acids. Putative ORFs were then translated and compared to a database of KSHV ORFs. RRV ORFs which matched KSHV ORFs were then compared to GenBank using BLASTP to verify the similarity, followed by a Gap analysis (Wisconsin GCG analysis package; Oxford Molecular Group) to determine the levels of similarity and identity between the RRV and KSHV proteins. When a gap in the genome of RRV corresponded to the location of a KSHV ORF with less than 100 amino acids, MacVector was reset to a lower limit. RRV ORFs were assigned the names of HVS ORFs when they showed similarity to KSHV ORFs with the same name.

The nucleotide sequence data from this study have been deposited in the GenBank, EMBL, and DDBJ nucleotide sequence databases under accession number AF083501 (SEQ ID NO 1).

### EXAMPLE 7

### Primary Structure Of the RRV Genome

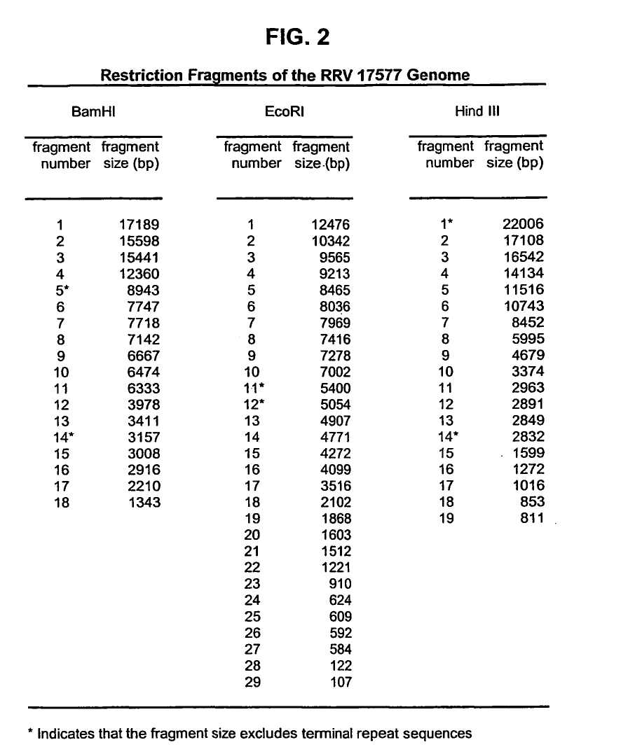

The genomic nucleotide sequence of the RRV genome (as shown in SEQ ID NO 1) was determined using twenty-nine EcoRI fragments (as shown in FIG. 2) from seven overlapping isolates of a partial Sau3A I cosmid library. Cosmids were selected by hybridization with PCR products from KSHV ORFs. EcoRI fragments from each cosmid were subcloned into pSP73 (Promega) and sequenced. The EcoRI fragments were arranged in the proper order by sequencing across the EcoRI junctions in the parent cosmids using custom primers. Greater than 98% of the viral genome was determined on both strands. The average sequencing redundancy was between 3 and 4, but three regions were sequenced on only one strand. One of these regions is a 106 bp segment of ORF 61 that was blocked on one side by an apparent hairpin. This segment was sequenced multiple times in one direction using templates derived from independent overlapping cosmids. The other two regions are 1 kb, high G+C, repetitive sequences. These segments, which are discussed in more detail below, were sequenced completely on one strand using a combination of custom primers and exonuclease III deletions.

Terminal repeats were identified on both the left and right ends of the genome and the sequence between them was designated as the LUR of the genome. The first base to the right of the left terminal repeat was designated base one. The LUR is 131,634 bp long (SEQ ID NO 1). The G+C content of RRV is 52.2%, which is comparable to the 53.5% G+C content of KSHV, but considerably higher than the 34.5% G+C content of the HVS genome. The CpG ratio is 1.11, which is substantially higher than the ratio found for other gamma-herpesviruses.

ORFs were identified by MacVector and compared to a database containing the full complement of known KSHV ORFs. Matches between RRV and KSHV proteins were verified by a BLASTP search of GenBank with the RRV proteins and then by Gap analysis. The initial screening for ORFs used a minimum size limit of 100 amino acids. This limit was reduced when smaller KSHV ORFs existed in locations corresponding to unassigned regions of RRV. Using this approach, 82 ORFs were identified, with 67 of these corresponding to ORFs found in both KSHV and HVS. In accordance with the standard nomenclature for rhadinovinises, these ORFs were labeled according to the HVS designation. The 15 ORFs not found in HVS were assigned labels beginning with R (for rhesus), indicating their presence in RRV, but not HVS. Some of these genes have counterparts in KSHV.

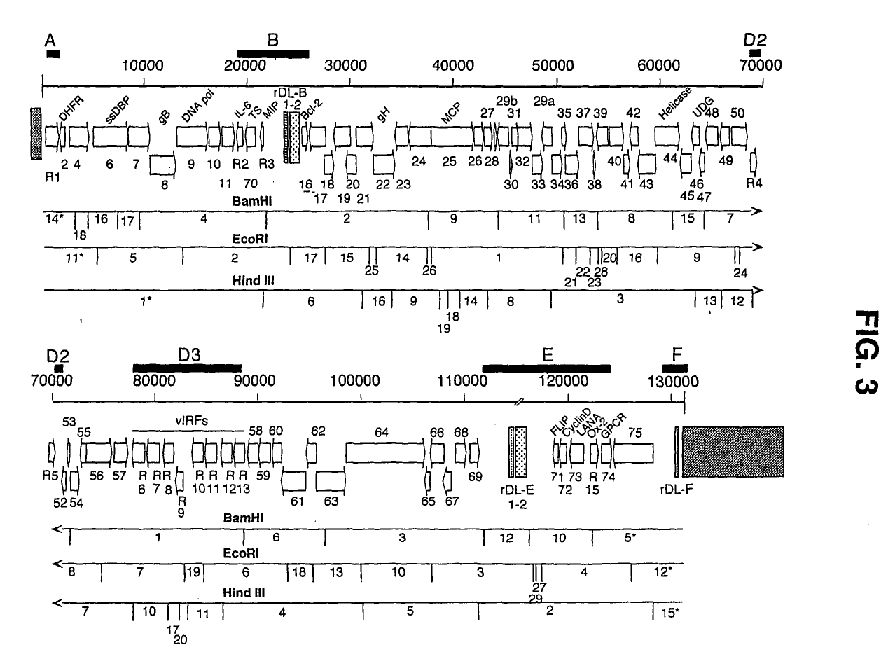

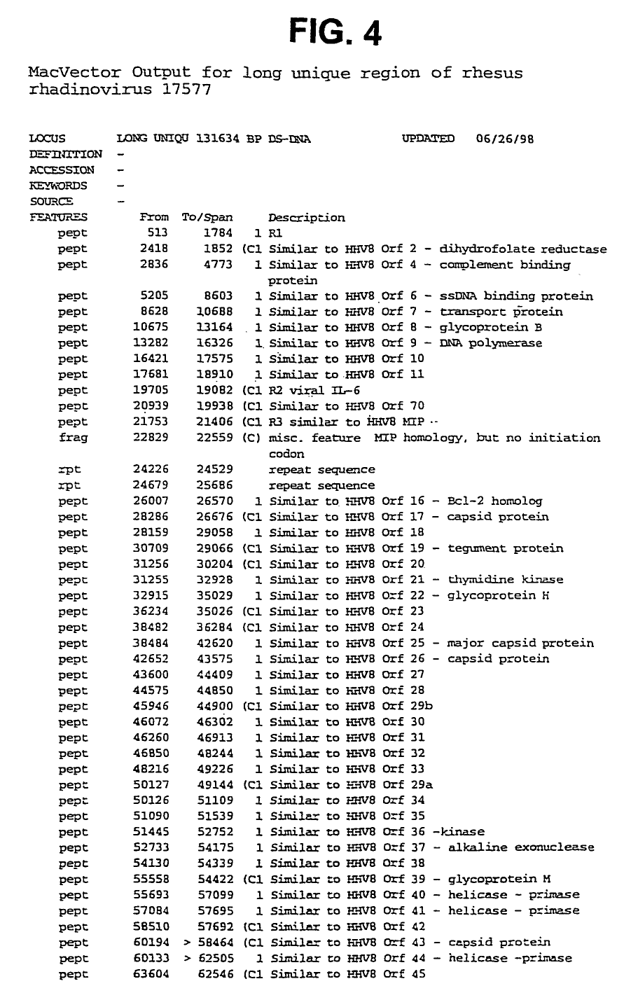

A map of the genome of RRV is presented in FIG. 3, with all identified ORFs and their orientations. The BamHI, EcoRI, and HindIII restriction sites in relation to the genome are shown in FIG. 2. The BamHI and Hind III maps were generated from the final compiled sequence. The EcoRI map was also generated from the final compiled sequence, but it was further characterized by sequencing across the EcoRI junctions in the parent cosmids. Fragment sizes for each restriction map are presented in FIG. 4.

### EXAMPLE 8

### Genomic Organization of RRV

The overall genomic organization of RRV matches the general structure of gamma-herpesviruses, with blocks of shared ORFs interrupted at specific locations (referred to as divergent loci) where the viral genomes code for acquired cellular genes. The primate rhadinoviruses form a subset of the gamma-herpesviruses and their genomes are correspondingly more similar to each other than to other members of the family.

The genomic sequence of RRV is presented in SEQ ID NO 1. FIG. 3 shows a schematic representation of the ORFs of RRV with a corresponding restriction map. FIG. 4 shows the location, size and description of the RRV ORFs.

### EXAMPLE 9

### Comparison of RRV and KSRV ORFs

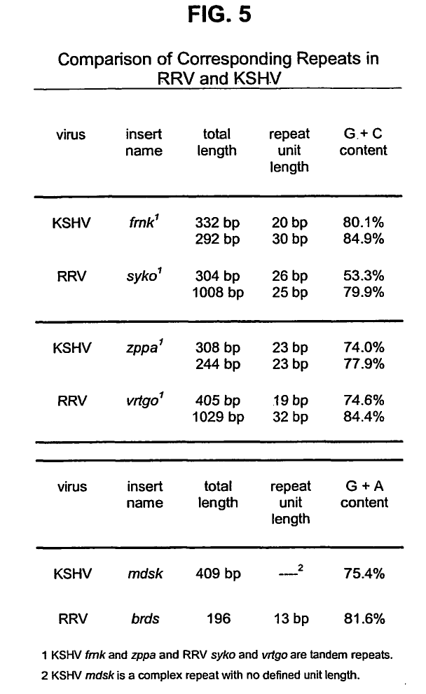

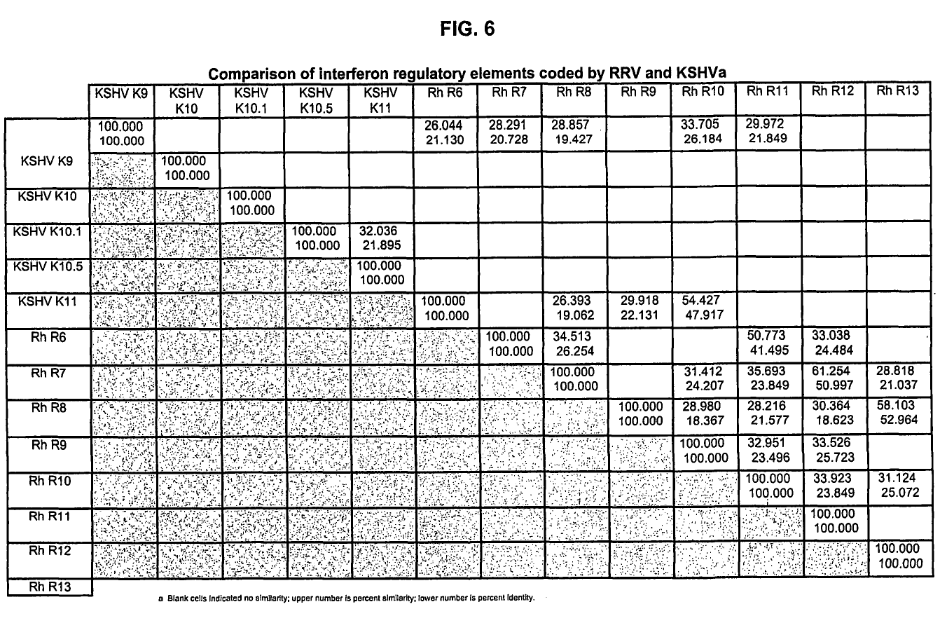

A comparison of corresponding repeats in RRV and KSHV is shown in FIG. 5. In addition, FIG. 5 presents data for RRV ORFs along with the results of the Gap analysis of ORFs shared by RRV, KSHV, and HVS. All HVS-like ORFs found in KSHV are found in RRV. A comparison table of interferon regulatory elements encoded by the RRV and KSHV genomes is shown in FIG. 6.

### EXAMPLE 10

### Comparison of RRV and HVS ORFs

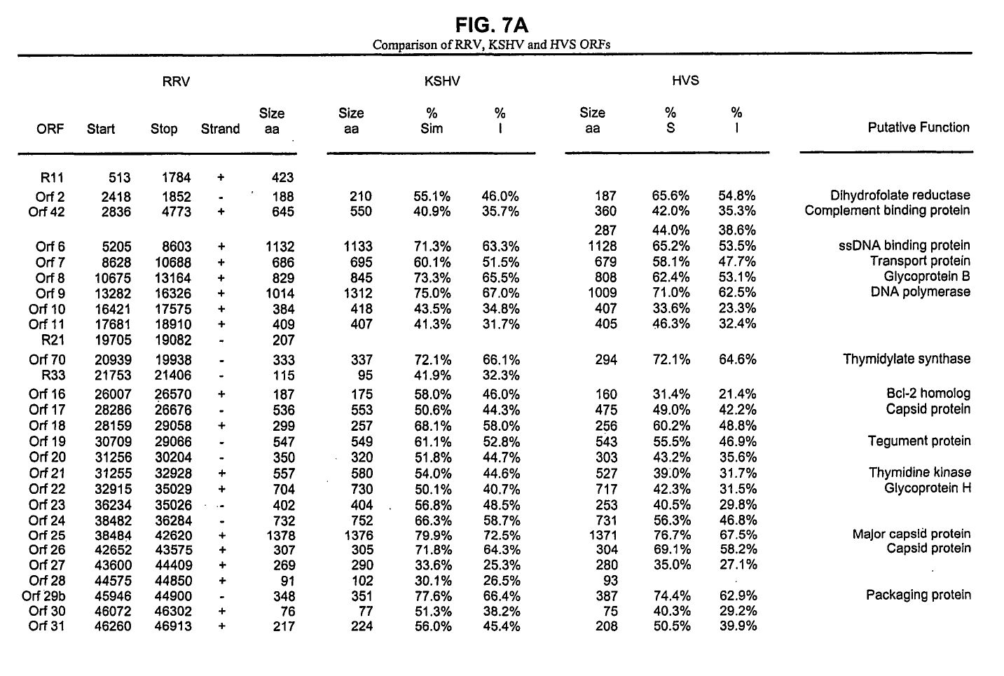

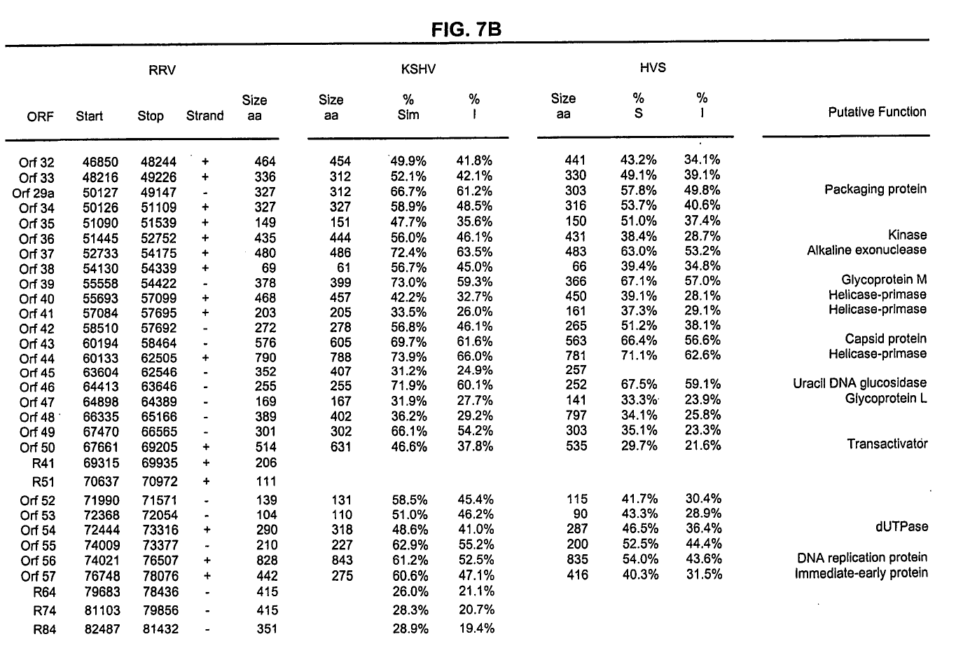

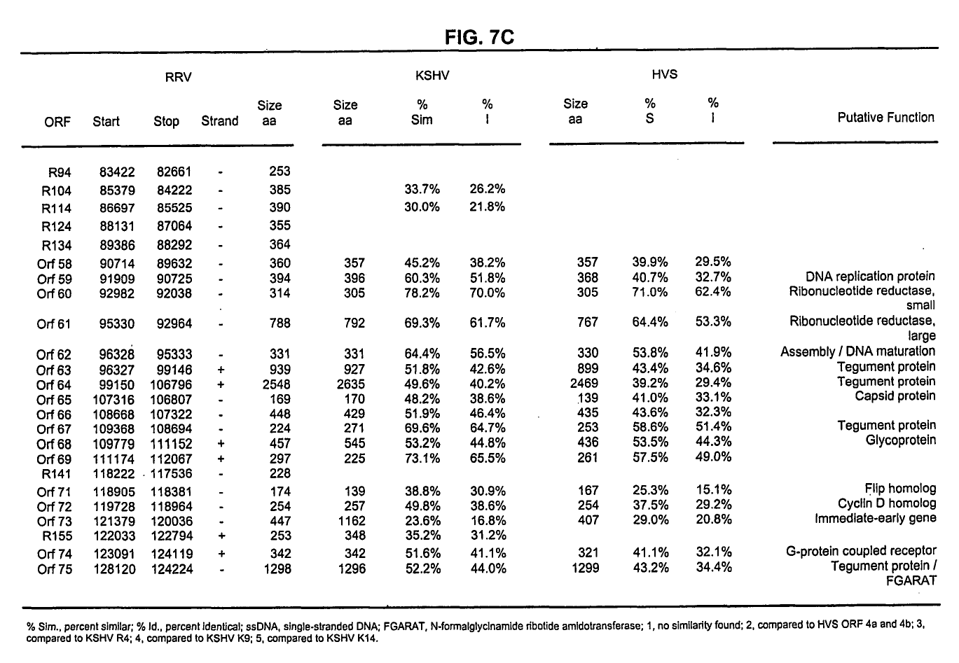

FIG. 7 shows the results of the Gap analysis of ORFs shared by RRV, KSHV, and HVS. In general, RRV and HVS ORFs are highly similar when the corresponding RRV and KSHV ORFs are highly similar, although the Gap values are generally lower.

### EXAMPLE 11

### ORFs Unique to RRV and KSHV

RRV includes 14 genes which are not found in HVS (R1; R2, R3, R4; R5; R6; R7; R8; R9; R10; R11; R12, R13; and R15 (see U.S. 99/26260). These are designated in FIG. 3 as “R” ORFs. Of these fifteen genes, 11 have counterparts in the genome of KSHV. R2 and R3 are cytokine genes. R2 has functional homology to K2, the vIL-6 gene of KSHV. Gap analysis of the vIL-6 genes from KSHV and RRV shows no notable similarity, but both possess four conserved cysteines found in cellular IL-6. In addition, RRV vIL-6 has IL-6-like activity in cell culture. R3 has a small, but clear, similarity to KSHV K4, a vMIP1β gene. It is the only vMIP gene in RRV, as compared to the three vMIP genes found in KSHV.

RRV R6 through R13 are vIRFs as are KSHV K9 through K11 (FIG. 6). K9, the most studied of the KSHV vIRFs, does not have a DNA binding domain, but has been demonstrated to inhibit the endogenous cellular interferon response pathways. Five of the RRV vIRFs (R6, R7, R8, R10, and R11) are similar to K9, though only R10 has a similarity greater than 30%. The remaining similarities fall between 26% and 30%. There is no measurable similarity between any RRV vIRF and any KSHV vIRF other than K9. There is, however, a pattern of higher similarity between members of the RRV vIRF family. R6, R7, R8, and R9 are most similar to R10, R11, R12, and R13, respectively, with the similarities falling between 50% and 62%. The pattern of similarity suggests a single, possibly recent, gene duplication event for RRV which increased the number of vIRFs in the genome from four to eight.

The final RRV gene with a unique KSHV counterpart is R15, which has some similarity to K14, a viral NCAM Ox-2 homologue. The similarity between R15 and K14 (35.2%) is relatively low compared to most other shared proteins.

A number of genes in RRV appear to be truly unique. R1 colocalizes with, but has no similarity to, K1, a KSHV gene that has been demonstrated to have in vivo transforming ability. K1 and R1 both colocalize with ORF1, or STP (saimiri transforming protein), although both K1 and R1 are in opposite orientations compared to STP. A BLASTP search of GenBank using R1 reveals a limited amino-terminal similarity to a series of Fc receptors, including a potential transmembrane domain. These data suggest that R1, like K1 and STP, may have transforming potential via transmembrane signaling.

R4 and R5 are located between ORF 50 and 52, the same location as K8 and K8.1 in KSHV;
however, there is no similarity between either R4 or R5 and the KSHV proteins. A BLASTP search of GenBank failed to show any significant alignments with either R4 or R5, so their functions are unknown.

RRV has no confirmed ORFs in the region corresponding to K12, the ubiquitously expressed kaposin gene. A large ORF exists to the right of ORF 71, but it has no apparent control regions (TATA box or polyadenylation signal), so it has not been designated as a true ORF, pending identification of transcripts from this region. No ORFs corresponding to KSHV K15 have been identified.

### EXAMPLE 12

### Co-localization of Repeat Units in RRV and KSHV

The RRV genome contains three highly repetitive regions, which correspond to three of the repetitive regions of KSHV: frnk, zppa, and mdsk (FIG. 5). KSHV frnk and zppa, and the corresponding RRV repetitive regions, rDL-B and rDL-E, respectively, are tandem repeats.

The first element of the RRV syko repeat is much lower in G+C content than the corresponding KSHV element, although the sizes are comparable (FIG. 5). The second element is over 700 bp longer than the corresponding KSHV element. The first element of the RRV vrtgo repeat is 30% longer than the corresponding KSHV element, and the second RRV element is over four times as long as the second KSHV element. There is no sequence similarity between the various elements of the two viruses nor is there any similarity between any two repeat sequences in RRV.

Not all repeat elements found in KSHV have corresponding repeats in RRV. This includes the KSHV vnct and waka/jwka repeats. This also includes the moi repeat, which is located in the center of the KSHV ORF 73 and is responsible for the divergent lengths of RRV and KSHV ORF 73. Moi is described in the annotations to the KSHV GenBank entry as having 15 different 11-16 bp repeats. The result of this repeat element is the presence in ORF 73 of a highly acidic central domain, with a large number of glutamate residues coded by a repeating GAG codon. KSHV ORF 73 is a potential leucine zipper protein, with a number of leucine zipper sites in the repeat region. RRV lacks the moi repeat and its concomitant acidic domain. It also lacks any evidence for a leucine zipper, indicating that the biology of ORF 73 in RRV may be substantially different than the biology of ORF 73 in KSHV.

### EXAMPLE 13

### Production of Simian Kaposi's Sarcoma (KS) and Lymphoproliferative Disorders Model

This example describes how the RRV cloned above can be used to produce a non-human primate model for Kaposi's sarcoma and lymphoproliferative disorders. Four rhesus macaques (identification numbers 18483, 18503, 18540 and 18570) that were approximately 1.5 years old, and PCR— and seronegative for RRV were selected. To perform the antibody analysis, infected cells were solubilized with 0.5% Nonidet P40 and 1% sodium deoxycholate in phosphate buffered saline, and clarified in a Beckman SW28 rotor at 23,500 rpm for 1 hour at 4° C. The clarified supernatant was used as antigen for coating enzyme-linked immunosorbent assay (ELISA) plates (500 ng/well). ELISAs were then performed essentially as described by Kodama et al. (AIDS Res Hum Retoviruses 5:33743, 1989).

All of the animals were then inoculated intravenously with cell-free supernatants containing the equivalent of 5 ng of p27 prepared from COS-1 cells transfected with an SIVmac239 molecular clone (Endres et al., 1995, SW. J. Med. Primatol. 24:1414). The PBMCs from all macaques were prescreened for in vitro susceptibility to virus infection as described by Naidu et al. (J. Virol. 62:4691-6, 1988). All inoculations and animal manipulations were performed according to institutional guidelines at the Oregon Regional Primate Research Center (Beaverton, Oreg.). Every 34 days for 4 weeks, then at 2-week intervals, macaques were sedated with ketamine hydrochloride (10 mg per kilogram of body weight) and examined for fever, weight loss, cutaneous signs, lymphadenopathy, and hepatomegaly or splenomegaly. At these times, venipuncture was performed and blood specimens collected. Plasma was monitored for virus during the first 4 weeks with the SIV p27 enzyme-linked immunosorbent assay (ELISA) (Coulter Corp. Hialeah, Fla.). T cell subsets and B cells were measured by flow cytometry with the OKT4 (CD4, Ortho), B9.11 (CD8, Coulter), and B-Ly-1 (CD20, Coulter) monoclonal antibodies.

At 8 weeks post-SIV infection, rhesus macaques 18483 and 18570 were inoculated intravenously with 5×10⁶ plaque forming units of gradient purified RRV that was grown and titered by plaque assay on primary rhesus fibroblasts. The two remaining macaques (18503 and 18540) were kept as SIV-infected controls. Every 3-4 days for 2 weeks, once a week for 4 weeks, then at 2 week intervals, the macaques were examined and blood samples collected and analyzed. Virus isolations were performed by cocultivation of 2×10⁵ PBMCs from each of the macaques with primary rhesus fibroblasts in duplicate. Cell cultures were monitored every 2-3 days for 3-4 weeks for cytopathic effects characteristic of RRV. PBLs were also analyzed by PCR for the presence of viral DNA. PCR analysis for RRV was performed with the following oligonucleotide primers: vMIP-1 and vMIP-2 (see PCT U.S. 99/26260). The conditions for PCR were 94° C. for 2 minutes (I cycle); 94° C. for 0.5 minutes, 50° C. for 0.5 min, 72° C. for 0.5 minutes (30 cycles); 72° C. extension for 5 minutes (1 cycle). Each PCR reaction used 0.1 Fg of total DNA, 50 pmole of each primer, 1 U of Vent polymerase, 40 μM each of deoxynucleotide triphosphate, 10 mM KCl, 10 mM Tris-HCl (pH 8.8), 10 MM (NH₄)₂SO₄, 2 MM MgSO₄ and 0.1% Triton X-100 in a final volume of 50 μL. The PCR reactions were run out on a 1% agarose gel, transferred to nitrocellulose, and probed with a ³²P-ATP-labeled oligonucleotide primer specific for vMIP-3 (see PCT U.S. 99/26260). Hybridizations were performed overnight at room temperature in 6×SSC, 0.1% SDS and 10 μg/nL E. Coli tRNA. Southern blots were then washed with 2×SSC and 0.1% SDS twice at room temperature followed by two washes for 1 hour in 2×SSC and 0.1% SDS at 47° C. Bound probe was visualized by exposing NEN duPont reflection film to the washed membrane at 80° C. with an NEN duPont Reflection screen.

Infectious RRV was recovered from the peripheral blood mononuclear cells (PBMCs) of both RRV macaques injected with RRV as early as 4 weeks after inoculation for one macaque (18570) and 8 weeks for the other macaque (18483), but not from the control macaques. The peripheral blood leukocytes (PBL) from both macaques were also shown to harbor viral DNA as determined by PCR and Southern blot analysis for the viral MIP gene, as early as 4 weeks after inoculation for one macaque (18483) and as late as 14 weeks for the second macaque (18570). Additionally, antibody responses to RRV were observed as measured by ELISA in the RRV-infected macaques beginning 4 weeks post-infection, but not in the control macaques.

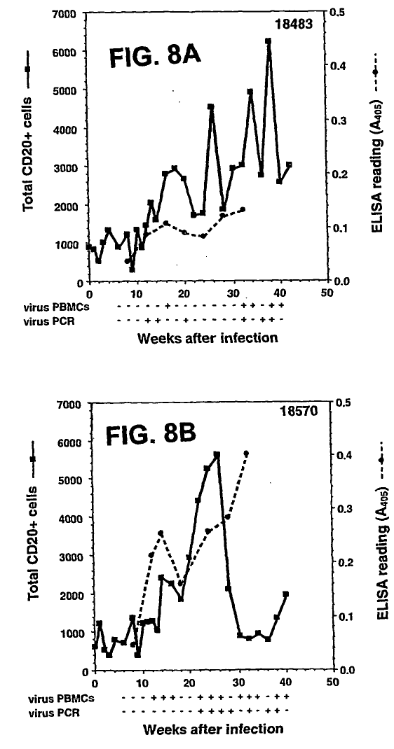

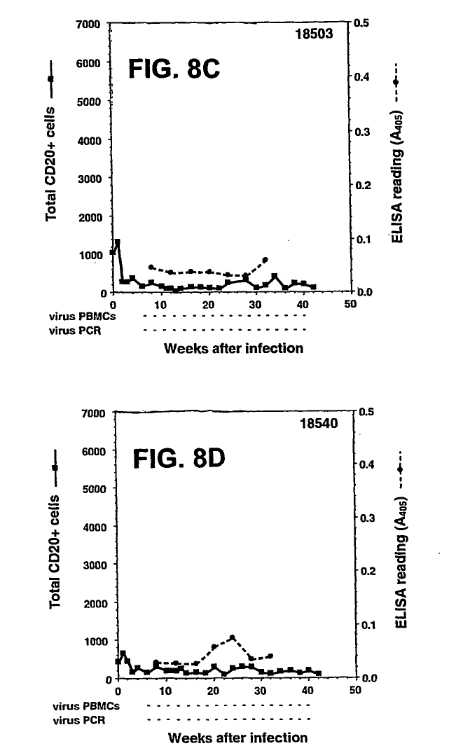

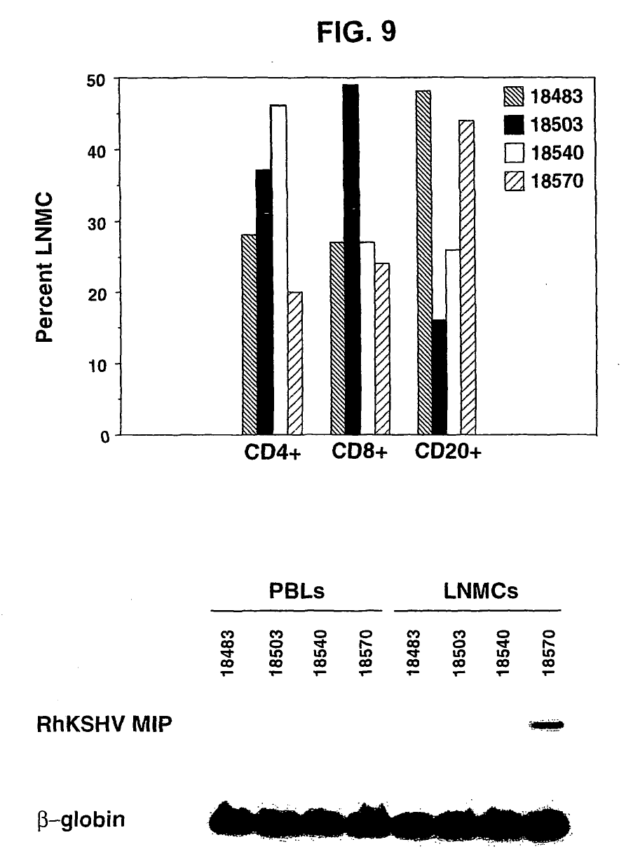

Flow cytometry analysis (FACS) of PBLs at the indicated weeks post-infection (FIGS. 8A-8D) showed there was limited CD4+ lymphocyte depletion after SIV infection in both groups of macaques followed by a rebound and sustained CD4+ lymphocyte counts. However, examination of CD20+B lymphocytes revealed significant differences between the two groups. The two control macaques exhibited a dramatic and sustained decline in CD20+B lymphocytes (FIGS. 8C and 8D), whereas both co-infected macaques exhibited a transient increase in B lymphocytes beginning 6 weeks after RRV infection (FIGS. 8A and 8B). The increase in CD20+B lymphocytes correlated with the isolation and/or detection of RRV in both macaques; however, viral load did not appear to correlate with the increase in CD20+B lymphocytes when all samples from each macaque were analyzed simultaneously. It has been reported that CD23, a B cell activation marker, is induced by RhEBV infection of macaques (Moghaddam et al. 1997, Science 276:2020-33). FACS analysis of PBMCs from RRV-infected macaques revealed no detectable CD23+ cells. This would suggest that the mechanism responsible for increased numbers of CD20+ B lymphocytes following RRV infection differ from the activation of B lymphocytes by RhEBV.

Routine physical examinations were performed on all four macaques, and early symptoms of SIV infection were observed in all four macaques by 2 weeks, including fever, rash and malaise. However, 11 weeks after inoculation with RRV, macaques 18483 and 18570 developed marked lymphadenopathy and splenomegaly, estimated to be enlarged 10 to 20 times the size of a normal spleen. In contrast, there was only slight lymph node enlargement in the control macaques not infected with RRV and no detectable enlargement of the spleen. Lymph node biopsies of the RRV-infected macaques revealed almost identical histology, characterized by a predominately follicular lesion with giant germinal centers and paracortical hyperplasia with increased vascularity, resembling angiofollicular lymph node hyperplasia associated with KSHV in Castleman's disease (Lachant et al. 1985, Am. J. Clin. Pathol. 83:27-33). In contrast, the lymph nodes of the control macaques exhibited atrophied lymphoid follicles and paracortical depletion characteristic of SIV-induced lymphoid atrophy (Chalifoux et al., 1987, Am. J. Pathol. 128:104-10; Ringler et al., 1989, Am. J. Pathol. 134:373-83; Wyand et al, 1989, Am. J. Pathol. 134:385-93). By FACS analysis, the majority of the lymph node mononuclear cells were CD20+B lymphocytes in RRV-infected macaques, whereas CD4+ and CD8+ T lymphocytes predominated in the control macaques.

The presence of viral DNA was determined by PCR analysis on DNA derived from PBLs. Detection of antibodies to RRV was determined by enzyme-linked immunosorbent assay (ELISA) on plates coated with extracts derived from RRV-infected cells. By PCR analysis, RRV sequences were more prevalent in the lymph nodes than in the peripheral blood of RRV-infected macaques, whereas control macaques were negative for RRV sequences (FIGS. 9A and 9B).

Additional disease manifestations were also observed in the RRV-infected macaques that parallel clinical features and B cell abnormalities observed in AIDS patients. Hypergammaglobulinemia was observed in the RRV-infected macaque that the virus was derived from, as well as in the macaques experimentally infected with RRV, whereas the two control macaques had gammaglobulin levels similar to those before SIV infection. In addition, one of two RRV-infected macaques (18570) developed severe autoimrnune hemolytic anemia 30 weeks after RRV infection, a condition frequently observed in MCD patients (Parravicini et al., 1997, Am. J. Pathol. 151:1517-22).

The second of the two RRV-infected macaques developed other unique clinical manifestations that paralleled those of AIDS patients with KS. At 60 weeks post-RRV infection it developed a distended abdomen that was clinically evident upon physical examination. Palpation revealed a pronounced fluid accumulation in the peritoneal cavity. This animal was euthanized due to persistent fluid accumulation and hyperbilirubinemia. Necropsy analysis on this animal revealed an abundance of ascites fluid, which was comprised predominately of CD20 B cells, as identified by FACS analysis. In addition, this animal exhibited a mesenchymal proliferative lesion throughout the viscera, that was identified by histopathological examination to be retroperitoneal fibromatosis (RF). RF is an abnormal highly vascularized mesenchymal proliferative lesion that exhibits histological features resembling Kaposi's Sarcoma. Analysis of DNA isolated from the ascites and RF lesion by PCR with RRV MIP primers (given in Example 2) revealed a high viral load, implying RRV infection was responsible for these abnormal proliferations.

### EXAMPLE 14

### Other Methods to Prepare RRV Nucleic Acid Sequences

Obtaining the RRV Viral Genome

The RRV genome (SEQ ID NO 1) can be procured by de novo isolation from a viral culture. A biological sample of the virus (accession number VR-2601) may be obtained from the ATCC in Manassas, Va. This virus can be grown in vitro using primary rhesus fibroblasts (see Example 1). The virus is harvested from the culture supernatant and the infected host cells. Cellular debris is removed by centrifugation and intracellular virus particles may be released by sonication followed by centrifugation to pellet debris. The virus is then pelleted by centrifugation and further purified through a six-step sorbitol gradient. The interface containing the virus is collected and the virus then pelleted by centrifugation, and the viral DNA released by SDS disruption. Viral DNA may be isolated by CsCl₂ gradient centrifugation.

Obtaining Selected Polynucleotides from the Viral Genome

The isolated viral genome can be used as a source of polynucleotides as identified by the sequence as disclosed herein (SEQ ID NO 1). The polymerase chain reaction (PCR) may be used to amplify any polynucleotide selected from the known viral sequence using the viral genome as a source of template DNA. The template DNA may also be provided in the form of one or more cosmids that contain fragments of the viral genome. Alternately, cDNA, produced by reverse transcription of RNA extracted from RRV infected host cells, may be used as a template in a reverse-transcription PCR (RT-PCR) reaction. Methods and conditions for PCR and RT-PCR amplification are described in Innis et al. (PCR Protocols, A Guide to Methods and Applications. 1990, Inns et al. (eds.), 21-27, Academic Press, Inc., San Diego, Calif.).

The selection of PCR primers may be made according to the portions of the genome to be amplified. Primers may be chosen to amplify small fragments of the genome, ORFs or fragments including many contiguous genes from the genome. Variations in amplification conditions may be required to accommodate primers of differing lengths, and such considerations are well known in the art and are discussed in Innis at al. (PCR Protocols, A Guide to Methods and Applications. 1990, Innis et al. (eds.), 21-27, Academic Press, Inc., San Diego, Calif.), Sambrook et al. (Molecular Cloning: A Laboratory Manual, 2^{nd} ed., vol. 1-3, Cold Spring Harbor, N.Y. 1989) and Ausubel et al. (Current Protocols in Molecular Biology, Greene Publ. Assoc. & Wiley-Intersciences. 1987). For example, the ORF corresponding to the MIP gene may be amplified from an RRV genomic (or appropriate cosmid) template using a pair of primers (see PCT U.S. 99/26260). Likewise, the ORF corresponding to the IL-6 gene may be amplified from an RRV genomic (or appropriate cosmid) template using a pair of primers (see PCT U.S. 99/26260).

Many different primers may be selected from the sequence disclosed and used in PCR amplification reactions to amplify DNA sequences of interest from the RRV genome.

Polynucleotides that may be obtained by the above methods include, for example: the entire polynucleotide genome of RRV as shown in SEQ ID NO 1; ORFs of this genome; oligonucleotides comprising at least 15, 20, 30, 40, 50, 70, 100 and 150 consecutive nucleotides of the genome sequence as shown in SEQ ID NO 1; nucleic acid sequences defined by nucleotides 1 to 11031 of SEQ ID NO 1 and nucleotides 21625 to 131634 of SEQ ID NO 1; and ORFs selected from these nucleic acid sequences. It is readily apparent that fragments of any length may be made using the above methods and information.

### EXAMPLE 15

### Therapeutic and Diagnostic Uses of the RRV or JMHV IL-6 Protein

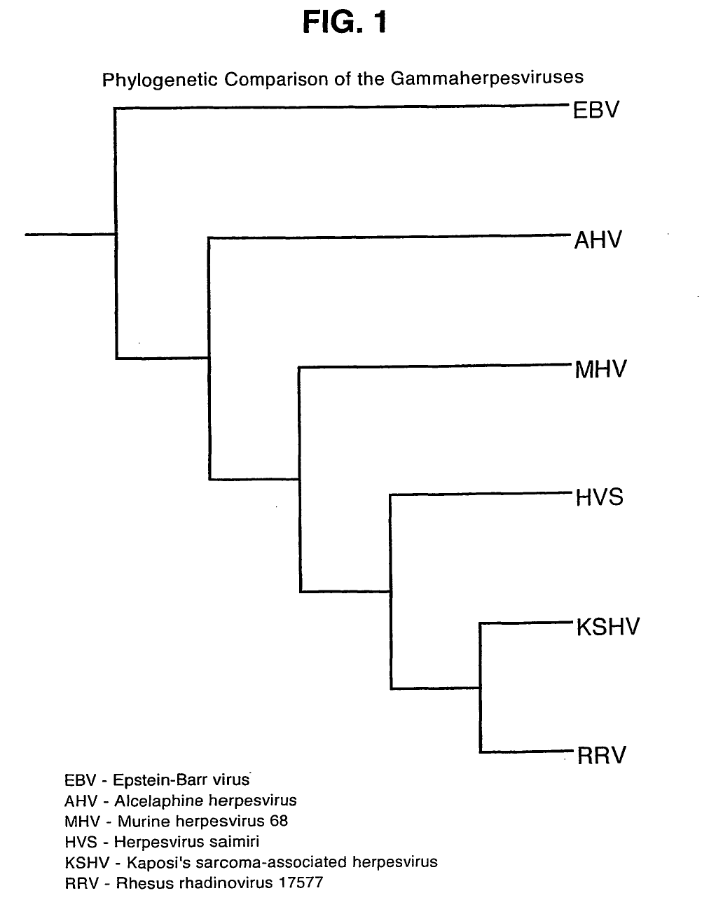

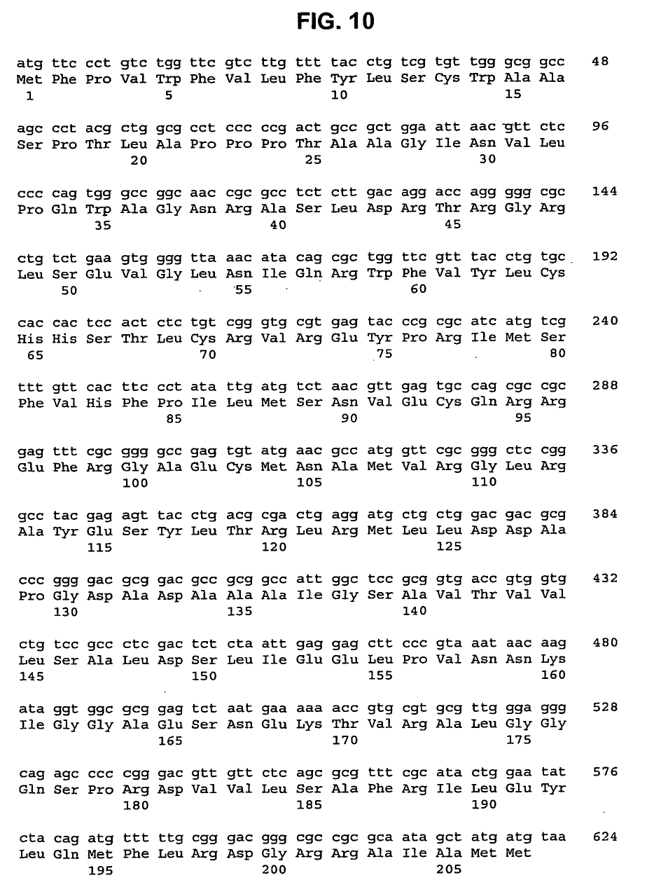

As disclosed herein, the genome of RRV possesses an IL-6 gene (FIG. 1), and the genome of JMHV posses an IL-6 gene, similar to that found in KSHV. The IL-6 and MIP proteins of KSHV are thought to be important in disease pathology, such as in Kaposi's sarcoma. The primary structure of the RRV IL-6 protein is shown in FIG. 10 (SEQ ID NO 21). Given the sequence information, one can readily make derivative proteins of RRV IL-6 or JMHV IL-6. In one specific, non-limiting example, such derivative proteins include proteins that differ from the primary amino acid sequence as shown in FIG. 10 (SEQ ID NO 21) by one or more conservative amino acid substitutions. Examples of such conservative substitutions are given in the DEFINITIONS section of the specification. Derivative proteins also include proteins consisting of an amino acid sequence that has a defined degree of amino acid similarity with the RRV IL-6 or JMHV IL-6 protein. For instance, such derivative proteins will typically have at least 50% sequence similarity (and may have at least 60%, 70%, 80%, 90%, 95%, 98% or even 99% sequence similarity) with the RRV IL-6 protein. Such derivative proteins will not only share sequence similarity with KSHV IL-6 but will also possess IL-6 biological activity.

IL-6 is a cytokine known to have pleiotropic immunological effects including anti-inflamatory and immunosuppressive effects, and may be used in several therapeutic and diagnostic applications. RRV IL-6 or JMHV IL-6 of the invention may be likewise be used. For instance, IL-6 may be used to induce stimulation of hematopoietic stem cells, and to encourage proliferation, differentiation and terminal maturation of erythroid cells from hematopoietic cells. Thus, for instance, RRV IL-6 or JMHV IL-6 may be used in vivo or ex vivo to treat diseases that involve leukopenia and thrombocytopenia. Such uses include stimulation of hematopoietic cells of radiotherapy patients or people exposed to radiation accidentally. IL-6 may be used in such applications in conjunction with GM-CSF (granulocyte-macrophage stimulating factor) (see U.S. Pat. Nos. 5,610,056 and 5,620,685, herein incorporated by reference). IL-6 can also be used to stimulate growth of megakaryocytes and platelets, and for the inhibition of tumor growth (see U.S. Pat. No. 5,620,685, herein incorporated by reference). IL-6 can also be used for the treatment of leukemias, such as chronic myeloid leukemia (CML) and acute myeloid leukemia, by inducing terminal differentiation of cells with IL-6 (see WO 90/01943, herein incorporated by reference). RRV IL-6 or JMHV II=6 may be used for all such applications.

Therapeutic applications may involve the administration of RRV IL-6 or JMHV IL-6 in a number of ways. RRV IL-6 or JMHV IL-6 may be administered in vivo, e.g., by injection systemically or locally, for instance, into a subject. Many other forms of in vivo administration are possible including intravenous, subcutaneous, across a mucous membrane (anally, vaginally or sublingually), transdermal or by direct injection. Additionally, it may be administered ex vivo, by the removal of cells from a subject, the treatment of these cells in vitro with RRV IL-6 or JMHV IL-6, and the replacement of these cells into the subject. Another recently developed method of delivery of a protein drug is by introducing the gene coding for the drug into a subject, for instance within the genome of a virus, such as an adenovirus or a retrovirus, whereby the protein is expressed in the subject. Other modes of administration are provided in Example 25.

Such examples are provided for illustrative purposes only and it will be seen that RRV IL-6 or JMHV IL-6 may be used in a variety of topical and systemic immunological treatments where it would be desirable to stimulate cell proliferation or to induce anti-inflammatory or immunosuppressive effects. Additionally, IL-6 of the invention may be used for research and diagnostic purposes as discussed generally herein. For instance, IL-6 may be used to produce antibodies for diagnostic purposes to diagnose diseases characterized by increased or decreased production of IL-6, and the nucleic acid sequence encoding IL-6 may be used to produce probes and primers for diagnostic and research purposes or for gene therapy applications. The IL-6 could also be used as a targeting molecule for identifying cells with receptors for IL-6, and for directing therapeutic agents to these cells, for example by linking detector or therapeutic molecules to IL-6.

### EXAMPLE 16

### Therapeutic and Diagnostic Uses of the RRV or JMHV MIP Protein

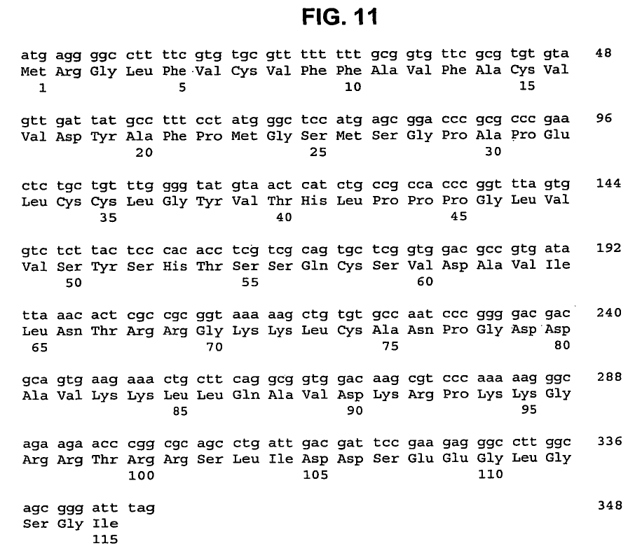

The genome of RRV as disclosed herein possesses an MIP gene (FIGS. 1 and 11) similar to that found in KSHV. The primary structure of the RRV MIP protein is shown in FIG. 11 (SEQ ID NO 25). Similarly, JMHV encodes a MIP protein (see below). Given the sequence information, one can readily make derivative proteins of RRV or JMHV MIP. Such derivative proteins include proteins that differ from the primary amino acid sequence as shown in FIG. 11 (SEQ ID NO 25), or from a JMHV MIP protein, by one or more conservative amino acid substitutions. Derivative proteins also include proteins consisting of an amino acid sequence that has a defined degree of amino acid similarity with the RRV or JMHV MIP protein. Typically such derivative proteins will have at least 50% sequence similarity with the RRV or JMHV MIP protein, and may have at least 60%, 70%, 80%, 90%, 95%, 98%, or even 99% sequence similarity. Such derivative proteins will not only share sequence similarity with KSHV MIP but will also possess MIP biological activity. MIP biological activity can be detected and quantified using bioassays as described in Kedal et al. (Science 277:1656-9, 1997) and Boshoff et al. (Science 278:2904, 1997) that measure MIP concentrations using HIV inhibition and calcium mobilization, respectively.

MIP is a cytokine that activates neutrophils to undergo an oxidative burst and is also intrinsically pyrogenic. The MIP genes and proteins of the invention may be used in several therapeutic and diagnostic ways. The RRV MIP protein may be used for the same applications as other MIP proteins. Treatment of wounds to promote healing by application of MIP to the wound site is discussed in U.S. Pat. No. 5,145,676. U.S. Pat. No. 5,474,983 (herein incorporated by reference) discusses various methods of treatment of inflammatory diseases including asthma, allergies and dermatitis. U.S. Pat. No. 5,656,724 (herein incorporated by reference) discloses the use of MIP to suppress proliferation of dividing myeloid cells e.g., for the treatment of neutropenia. Use of MIP to inhibit HIV is discussed by Kedal et al. (Science 277:1656-9, 1997). RRV or JMHV MIP may be used for all such applications.

As illustrated for IL-6 above, MIP may be administered in various ways to provide a therapeutic effect including in vivo, ex vivo and by gene therapy.

Such examples are provided for illustrative purposes only and it will be seen that MIP may be used in a variety of topical, systemic, in vivo and ex vivo immunological treatments where it would be desirable to activate neutrophils or to induce fever. Additionally, MIP of the invention may be used for diagnostic purposes as discussed generally herein. For instance, MIP may be used to produce antibodies for diagnostic purposes to diagnose diseases characterized by increased or decreased production of MIP, and the nucleic acid sequence encoding MIP may be used to produce probes and primers for diagnostic and research purposes, or for gene therapy applications.

The MIP could also be used as a targeting molecule for identifying cells with receptors for MIP, and for directing therapeutic agents to these cells, for example by linking detector or therapeutic molecules to MIP.

Although Examples 15 and 16 provide examples of therapeutic uses of the RRV or JMHV IL-6 and MIP proteins, any of the other proteins encoded by the RRV or JMHV can also be administered therapeutically, or diagnostically. For example, RRV or JMHV proteins that induce pathological or physiological conditions in a recipient can be administered to stimulate that condition for study, or to provide an animal or human model of the condition. That model can then be used to study the condition, or treatments for it.

### EXAMPLE 17

### Expression of RRV or JMHV cDNA Sequences

With the provision of the RRV genomic (SEQ ID NO 1), and the provision of JMHV sequences, the expression and purification of any of the RRV proteins, or JMHV proteins, by standard laboratory techniques, is now enabled. Fragments amplified as described herein can be cloned into standard cloning vectors and expressed in commonly used expression systems consisting of a cloning vector and a cell system in which the vector is replicated and expressed. Purified proteins may be used for functional analyses, antibody production, diagnosis, and patient therapy. Furthermore, the DNA sequences of the RRV cDNAs or JMHV cDNAs can be manipulated in studies to understand the expression of RRV genes or JMHV genes and the function of their products. Mutant forms of RRV or JMHV may be isolated based upon information contained herein, and may be studied in order to detect alteration in expression patterns in terms of relative quantities, and functional properties of the encoded mutant RRV protein. Partial or full-length cDNA sequences, which encode for the protein, may be ligated into bacterial expression vectors. Methods for expressing large amounts of protein from a cloned gene introduced into E. coli may be utilized for the purification, localization and functional analysis of proteins. For example, fusion proteins consisting of amino terminal peptides encoded by a portion of the E. coli lacZ or trpE gene linked to RRV protein or JMHV protein may be used to prepare polyclonal and monoclonal antibodies against this protein. Thereafter, these antibodies may be used to purify proteins by immunoaffinity chromatography, in diagnostic assays to quantitate the levels of protein and to localize proteins in tissues and individual cells by immunofluorescence and microscopy.

Intact native protein may also be produced in E. coli in large amounts for functional studies. Standard prokaryotic cloning vectors may also be used, for example pBR322, pUC18 or pUC19 as described in Sambrook et al. (Molecular Cloning: A Laboratory Manual, 2^{nd} ed., vol. 1-3, Cold Spring Harbor, N.Y. 1989). Nucleic acids of RRV or JMHV may be cloned into such vectors which may then be transformed into bacteria such as E. coli which may then be cultured so as to express the protein of interest. Other prokaryotic expression systems include, for instance, the arabinose-induced pBAD expression system that allows tightly controlled regulation of expression, the IPTG-induced pRSET system that facilitates rapid purification of recombinant proteins and the IPTG-induced pSE402 system that has been constructed for optimal translation of eukaryotic genes. These three systems are available commercially from Invitrogen and, when used according to the manufacturer's instructions, allow routine expression and purification of proteins.

Methods and plasmid vectors for producing fusion proteins and intact native proteins in bacteria are described in Sambrook et al. (Molecular Cloning: A Laboratory Manual, Cold Spring Harbor, N.Y., 1989, Chapter 17). Such fusion proteins may be made in large amounts, are easy to purify, and can be used to elicit antibody response. Native proteins can be produced in bacteria by placing a strong, regulated promoter and an efficient ribosome binding site upstream of the cloned gene. If low levels of protein are produced, additional steps may be taken to increase protein production; if high levels of protein are produced, purification is relatively easy. Suitable methods are presented in Sambrook et al. (Molecular Cloning: A Laboratory Manual, Cold Spring Harbor, N.Y., 1989) and are well known in the art. Often, proteins expressed at high levels are found in insoluble inclusion bodies. Methods for extracting proteins from these aggregates are described by Sambrook et al. (Molecular Cloning: A Laboratory Manual, Cold Spring Harbor, N.Y., 19S9, Chapter 17).

Vector systems suitable for the expression of lacZ fusion genes include the pUR series of vectors (Ruther and Muller-Hill, 1983, EMBO J. 2:1791), pEX1-3 (Stanley and Luzio, 1984, EMBO J. 3:1429) and pMR100 (Gray et al., 1982, Proc. Natl. Acad. Sci. USA 79:6598). Vectors suitable for the production of intact native proteins include pKC30 (Shimatake and Rosenberg, 1981, Nature 292:128), pKK177-3 (Amann and Brosius, 1985, Gene 40:183) and pET-3 (Studiar and Moffatt, 1986, J. Mol. Biol. 189:113). The RRV or JMHV fusion protein may be isolated from protein gels, lyophilized, ground into a powder and used as an antigen. The DNA sequence can also be transferred to other cloning vehicles, such as other plasmids, bacteriophages, cosmids, animal viruses and yeast artificial chromosomes (YACs) (Burke et al., 1987, Science 236:80612). These vectors may then be introduced into a variety of hosts including somatic cells, and simple or complex organisms, such as bacteria, fungi (Timberlake and Marshall, 1989, Science 244:1313-7), invertebrates, plants (Gasser and Fraley, 1989, Science 244:1293), and mammals (Pursel et al., 1989, Science 244:1281-8), which cell or organisms are rendered transgenic by the introduction of one or more heterologous RRV DNAs and/or JMHV DNAs.

Various yeast strains and yeast-derived vectors are commonly used for expressing and purifying proteins, for example, Pichia pastoris expression systems are available from Invitrogen (Carlsbad, Calif.). Such systems include suitable Pichia pastoris strains, vectors, reagents, transformants, sequencing primers and media.

Non-yeast eukaryotic vectors can also be used for expression of the RRV or JMHV proteins. Examples of such systems are the well known Baculovirus system, the Ecdysone-inducible mammalian expression system that uses regulatory elements from Drosophila melanogaster to allow control of gene expression, and the Sindbis viral expression system that allows high level expression in a variety of mammalian cell lines. These expression systems are available from Invitrogen.

For expression in mammalian cells, the cDNA sequence may be ligated to heterologous promoters, such as the simian virus SV40, promoter in the pSV2 vector (Mulligan and Berg, 1981, Proc. Natl. Acad. Sci. USA 78:2072-6), and introduced into cells, such as monkey COS-1 cells (Gluznan, 1981, Cell 23:175-82), to achieve transient or long-term expression. The stable integration of the chimeric gene construct may be maintained in mammalian cells by biochemical selection, such as neomycin (Southern and Berg, 1982, J. Mol. Appl. Genet. 1:32741) and mycophoenolic acid (Mulligan and Berg, 1981, Proc. Natl. Acad. Sci USA 78:2072-6).

DNA sequences can be manipulated with standard procedures such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate or with the use of specific oligonucleotides in combination with PCR

The cDNA sequence (or portions derived from it) or a mini gene (a cDNA with an intron and its own promoter) may be introduced into eukaryotic expression vectors by conventional techniques. These vectors are designed to permit the transcription of the cDNA eukaryotic cells by providing regulatory sequences that initiate and enhance the transcription of the cDNA and ensure its proper splicing and polyadenylation. Vectors containing the promoter and enhancer regions of the SV40 or long terminal repeat (LTR) of the Rous Sarcoma virus and polyadenylation and splicing signal from SV40 are readily available (Mulligan and Berg, 1981, Proc. Natl. Acad. Sci. USA 78:2072-6; Gorman et al., 1982, Proc. Natl. Acad Sci USA 78:6777-81). The level of expression of the cDNA can be manipulated with this type of vector, either by using promoters that have different activities (for example, the baculovirus pAC373 can express cDNAs at high levels in S. frugiperda cells (Summers and Smith, 1985, Genetically Altered Viruses and the Environment, Fields et al. (Eds.) 22:319-328, Cold Spring Harbor Laboratory Press, Cold Spring Harbor, N.Y.) or by using vectors that contain promoters amenable to modulation, for example, the glucocorticoid-responsive promoter from the mouse mammary tumor virus (Lee et al., 1982, Nature 294:228). The expression of the cDNA can be monitored in the recipient cells 24 to 72 hours after introduction (transient expression).

In addition, some vectors contain selectable markers such as the gpt (Mulligan and Berg, 1981, Proc. Natl. Acad. Sci. USA 78:2072-6) or neo (Southern and Berg, 1982, J. Mol. Appl. Genet. 1:32741) bacterial genes. These selectable markers permit selection of transfected cells that exhibit stable, long-term expression of the vectors (and therefore the cDNA). The vectors can be maintained in the cells as episomal, freely replicating entities by using regulatory elements of viruses such as papilloma (Sarver et al., 1981, Mol Cell Biol 1:486) or Epstein-Barr (Sugden et al., 1985, Mol. Cell Biol. 5:410). Alternatively, one can also produce cell lines that have integrated the vector into genomic DNA. Both of these types of cell lines produce the gene product on a continuous basis. One can also produce cell lines that have amplified the number of copies of the vector (and therefore of the cDNA as well) to create cell lines that can produce high levels of the gene product (Alt et al., 1978, J. Biol. Chem. 253:1357).

The transfer of DNA into eukaryotic, in particular human or other mammalian cells, is now a conventional technique. The vectors are introduced into the recipient cells as pure DNA (transfection) by, for example, precipitation with calcium phosphate (Graham and vander Eb, 1973, Virology 52:466) or strontium phosphate (Brash et al., 1987, Mol. Cell Biol. 7:2013), electroporation (Neumann et al., 1982, EMBO J. 1:841), lipofection (Felgner et al., 1987, Proc. Natl. Acad Sci USA 84:7413), DEAE dextran (McCuthan et al., 1968, J. Natl. Cancer Inst. 41:351), microinjection (Mueller et al., 1978, Cell 15:579), protoplast fusion (Schafner, 1980, Proc. Natl. Acad. Sci. USA 77:2163-7), or pellet guns (Klein et al., 1987, Nature 327:70). Alternatively, the cDNA can be introduced by infection with virus vectors. Systems are developed that use, for example, retroviruses (Bernstein et al., 1985, Gen. Engrg. 7:235), adenoviruses (Ahmad et al., 1986, J. Virol. 57:267), or Herpes virus (Spaete et al., 1982, Cell 30:295).

These eukaryotic expression systems can be used for studies of RRV or JMHV genes and mutant forms of these genes, and the RRV or JMHV proteins and mutant forms of these proteins.

Such uses include, for example, the identification of regulatory elements located in the 5′ region of RRV genes or JMHV genes on genomic clones that can be isolated from genomic DNA libraries, such as human or mouse libraries, using the information contained in the present invention. The eukaryotic expression systems may also be used to study the function of the normal complete protein, specific portions of the protein, or of naturally occurring or artificially produced mutant proteins. Naturally occurring RRV or JMHV wild-type or mutant proteins may exist in a variety of cancers or diseases, while artificially produced mutant proteins can be designed by site directed mutagenesis as described above. These latter studies may probe the function of any desired amino acid residue in the protein by mutating the nucleotide coding for that amino acid.

Using the above techniques, the expression vectors containing RRV or JMHV genes or cDNA sequence or fragments or variants or mutants thereof can be introduced into human cells, mammalian cells from other species or non-mammalian cells as desired. The choice of cell is determined by the purpose of the treatment. For example, monkey COS cells (Gluzman, 1981, Cell 23:175-82) that produce high levels of the SV40 T antigen and permit the replication of vectors containing the SV40 origin of replication may be used. Similarly, Chinese hamster ovary (CHO), mouse NIH 3T3 fibroblasts or human fibroblasts or lymphoblasts may be used.

One method that can be used to express RRV or JMHV polypeptides from the cloned RRV or JMHV cDNA sequence in mammalian cells is to use the cloning vector, pXT1. This vector is commercially available from Stratagene, contains the Long Terminal Repeats (LTRs) and a portion of the GAG gene from Moloney Murine Leukemia Virus. The position of the viral LTRs allows highly efficient, stable transfection of the region within the LTRs. The vector also contains the Herpes Simplex Thymidine Kinase promoter (TK), active in embryonal cells and in a wide variety of tissues in mice, and a selectable neomycin gene conferring G418 resistance. Two unique restriction sites BglII and XhoI are directly downstream from the TK promoter. RRV or JMHV cDNA, including the entire open reading frame for an RRV protein or JMHV protein such as IL-6 and the 3′ untranslated region of the cDNA is cloned into one of the two unique restriction sites downstream from the promoter.

The ligated product is transfected into mouse NIH 3T3 cells using Lipofectin (Life Technologies, Inc.) under conditions outlined in the product specification. Positive transfectants are selected after growing the transfected cells in 600 μg/ml G418 (Sigma, St. Louis, Mo.). The protein is released into the supernatant and may be purified by standard immunoaffinity chromatography techniques using antibodies raised against RRV proteins (see Example 18).

Expression of RRV or JMHV proteins in eukaryotic cells can be used as a source of proteins to raise antibodies. The RRV or JMHV proteins may be extracted following release of the protein into the supernatant as described above, or, the cDNA sequence may be incorporated into a eukaryotic expression vector and expressed as a chimeric protein with, for example, β-globin. Antibody to β-globin is thereafter used to purify the chimeric protein. Corresponding protease cleavage sites engineered between the β-globin gene and the cDNA are then used to separate the two polypeptide fragments from one another after translation. One useful expression vector for generating β-globin chimeric proteins is pSG5 (Stratagene). This vector encodes rabbit β-globin.

The present disclosure thus encompasses recombinant vectors which comprise all or part of RRV or JMHV genome or cDNA sequences, for expression in a suitable host. The RRV or JMHV DNA is operatively linked in the vector to an expression control sequence in the recombinant DNA molecule so that a RRV or JMHV polypeptide can be expressed. The expression control sequence may be selected from the group consisting of sequences that control the expression of genes of prokaryotic or eukaryotic cells and their viruses and combinations thereof. The expression control sequence may be specifically selected from the group consisting of the lac system, the trp system, the tac system, the trc system, major operator and promoter regions of phage lambda, the control region of fd coat protein, the early and late promoters of SV40, promoters derived from polyoma, adenovirus, retrovirus, baculovirus and simian virus, the promoter for 3-phosphoglycerate kinase, the promoters of yeast acid phosphatase, the promoter of the yeast alpha-mating factors and combinations thereof.

The host cell, which may be transfected with the vector of this invention, may be selected from the group consisting of: E. coli, Pseudomonas, Bacillus subtilis, Bacillus stearothermophilus or other bacilli; other bacteria; yeast; fingi; plant; insect; mouse or other animal; or human tissue cells.

It is appreciated that for mutant or variant RRV or JMHV DNA sequences, similar systems are employed to express and produce the mutant or variant product.

### EXAMPLE 18

### Production of Antibodies to RRV and RRV Proteins or JMHV and JMHV Proteins

Polyclonal or monoclonal antibodies (including humanized monoclonal antibodies) and fragments of monoclonal antibodies such as Fab, F(ab′)2 and Fv fragments, as well as any other agent capable of specifically binding to an RRV or JMHV protein, may be produced to the RRV virion, the JMHV virion, or any of the RRV or JMHV proteins (for example odd-numbered SEQ ID Nos 3-165). Optimally, antibodies raised against an RRV or JMHV protein would specifically detect the RRV or JMHV protein of interest (or a virion containing the protein of interest). That is, such antibodies would recognize and bind the protein and would not substantially recognize or bind to other proteins found in human or other cells. The determination that an antibody specifically detects the RRV or JMHV protein is made by any one of a number of standard immunoassay methods; for instance, the Western blotting technique (Sambrook et al., 1989, Molecular Cloning: A Laboratory Manual, Cold Spring Harbor Laboratory, Cold Spring Harbor, N.Y.).

To determine that a given antibody preparation (such as one produced in a mouse) specifically detects the RRV or JMHV protein by Western blotting, total cellular protein is extracted from murine myeloma cells and electrophoresed on a SDS-polyacrylamide gel. The proteins are then transferred to a membrane (for example, nitrocellulose) by Western blotting, and the antibody preparation is incubated with the membrane. After washing the membrane to remove non-specifically bound antibodies, the presence of specifically bound antibodies is detected by the use of an anti-mouse antibody conjugated to an enzyme such as alkaline phosphatase; application of the substrate 5-bromo-4-chloro-3-indolyl phosphate/nitro blue tetrazolium results in the production of a dense blue compound by immuno-localized alkaline phosphatase. Antibodies which specifically detect an RRV or JMHV protein will, by this technique, be shown to bind to the RRV or JMHV protein band (which will be localized at a given position on the gel determined by its molecular weight). Non-specific binding of the antibody to other proteins (such as serum albumin) may occur and may be detectable as a weak signal on the Western blot. The non-specific nature of this binding will be recognized by one skilled in the art by the weak signal obtained on the Western blot relative to the strong primary signal arising from the specific antibody-VIAP protein binding.

A substantially pure virion can be obtained, or substantially pure RRV or JMHV protein suitable for use as an immunogen is isolated by purification or recombinant expression. Concentration of protein in the final preparation is adjusted, for example, by concentration on an Amicon filter device, to the level of a few micrograms per milliliter. Monoclonal or polyclonal antibody to the protein can then be prepared as described by Harlow and Lane (Antibodies, A Laboratory Manual Cold Spring Harbor Press. 1988).

Alternatively, antibodies may be raised against synthetic RRV or JMHV peptides synthesized on a commercially available peptide synthesizer (see Example 26) based upon the predicted amino acid sequence of the RRV or JMHV protein (Harlow and Lane, Antibodies, A Laboratory Manual, Cold Spring Harbor Press. 1988).

Another method of raising antibodies against RRV or JMHV proteins is by subcutaneous injection of a DNA vector which expresses the RRV protein into laboratory animals, such as mice. Delivery of the recombinant vector into the animals may be achieved using a hand-held form of the Biolistic system (Sanford et al., 1987, Particulate Sci. Technol. 5:27-37) as described by Tang et al. (Nature 356:152-4, 1992). Expression vectors suitable for this purpose may include those which express the RRV protein under the transcriptional control of either the human β-actin promoter or the cytomegalovirus (CMV) promoter.

Monoclonal Antibody Production by Hybridoma Fusion

Monoclonal antibody to epitopes of the RRV or JMHV protein identified and isolated as described can be prepared from murine hybridomas according to the classical method of Kohler and Milstein (Nature 256:495, 1975) or derivative methods thereof. Briefly, a mouse is repetitively inoculated with a few micrograms of the selected protein over a period of a few weeks. The mouse is then sacrificed, and the antibody-producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall (Enzymol. 70:419, 1980), and derivative methods thereof. Selected positive clones can be expanded and their monoclonal antibody product harvested for use. Detailed procedures for monoclonal antibody production are described in Harlow and Lane (Antibodies: A Laboratory Manual. 1988, Cold Spring Harbor Laboratory, New York).

Polyclonal Antibody Production by Immunization

Polyclonal antiserum containing antibodies to heterogeneous epitopes of a single protein can be prepared by immunizing suitable animals with the expressed protein (e.g. see Example 17), which can be unmodified or modified to enhance immunogenicity. Effective polyclonal antibody production is affected by many factors related both to the antigen and the host species. For example, small molecules tend to be less immunogenic than others and may require the use of carriers and adjuvant. Also, host animals vary in response to site of inoculations and dose, with both inadequate or excessive doses of antigen resulting in low titer antisera. Small doses (ng level) of antigen administered at multiple intradermal sites appears to be most reliable. An effective immunization protocol for rabbits can be found in Vaitukaitis et al. (J. Clin. Endocrinol. Metab. 33:988-91, 1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony et al. (In: Handbook of Experimental Immunology, Wier, D. (ed.). Chapter 19. Blackwell. 1973). Plateau concentration of antibody is usually in the range of 0.1 to 0.2 mg/ml of serum (about 12 μM). Affinity of the antisera for the antigen is determined by preparing competitive binding curves, as described, for example, by Fisher (Manual of Clinical Immunology, Chapter 42, 1980).

Labeled Antibodies

Antibodies of the present invention can be conjugated with various labels for their direct detection (see Chapter 9, Harlow and Lane, Antibodies: A Laboratory Manual. 1988). The label, which may include, but is not limited to, a radiolabel, enzyme, fluorescent probe, or biotin, is chosen based on the method of detection available to the user.

### EXAMPLE 19

Diagnostic Methods

An embodiment of the present invention is a method for screening a subject to determine if the subject has been infected with RRV or JMHV. One major application of the RRV sequence information presented herein is in the area of diagnostic testing for predisposition to a disease (such as Kaposi's Sarcoma and lymphoproliferative disorders for RRV, or for multiple sclerosis for JMHV) that develops in at least a sub-set of hosts infected with RRV or JMHV. The gene sequence of the RRV genes, including intron-exon boundaries is also useful in such diagnostic methods. The method includes providing a biological sample obtained from the subject, in which sample includes DNA or RNA, and providing an assay for detecting in the biological sample the presence of any of the RRV or JMHV genes or proteins. Suitable biological samples include samples obtained from body cells, such as those present in peripheral blood, urine, saliva, tissue biopsy, surgical specimen, fine needle aspirate specimen, amniocentesis samples and autopsy material. The detection in the biological sample may be performed by a number of methodologies, as outlined below.

The foregoing assay may be assembled in the form of a diagnostic kit and preferably comprises either: hybridization with oligonucleotides; PCR amplification of the gene or a part thereof using oligonucleotide primers; RT-PCR amplification of the RNA or a part thereof using oligonucleotide primers; or direct sequencing of any of the RRV genes present in a subject using oligonucleotide primers. The efficiency of these molecular genetic methods should permit the rapid identification of patients infected with RRV.

One embodiment of such detection techniques is the polymerase chain reaction amplification of reverse transcribed RNA (RT-PCR) of RNA isolated from cells (for example lymphocytes) followed by direct DNA sequence determination of the products. The presence of one or more RRV genes is taken as indicative of a potential RRV infection. The presence of one or more JMHV genes is taken an indicative of potential JMHV infection.

Alternatively, DNA extracted from lymphocytes or other cells may be used directly for amplification. The direct amplification from genomic DNA would be appropriate for analysis of an entire RRV of JMHV gene including regulatory sequences located upstream and downstream from the open reading frame. Recent reviews of direct DNA diagnosis have been presented by Caskey (Science 236:1223-1228, 1989) and by Landegren et al. (Science 242:229-37, 1989).

Further studies of RRV or JMHV genes isolated from subjects may reveal particular mutations, deletions or alterations in gene sequences, which occur at a high frequency within particular populations of individuals. In this case, rather than sequencing the entire RRV gene, it may be possible to design DNA diagnostic methods to specifically detect the most common RRV mutations, deletions or alterations in gene sequences.

The detection of specific DNA mutations or alterations in gene sequences may be achieved by methods such as hybridization using specific oligonucleotides (Wallace et al., 1986, Cold Spring Harbor Symp. Quant. Biol. 51:257-61), direct DNA sequencing (Church and Gilbert, 1984, Proc. Natl. Acad. Sci. USA. 81:1991-5), the use of restriction enzymes (Flavell et al., 1978, Cell 15:25; Geever et al., 1981, Proc. Natl. Acad. Sci USA 78:5081), discrimination on the basis of electrophoretic mobility in gels with denaturing reagent (Myers and Maniatis, 1986, Cold Spring Harbor Symp. Quant. Biol. 51:275-284), RNase protection (Myers et al., 1985, Science 230:1242), chemical cleavage (Cotton et al., 1985, Proc. Natl. Acad. Sci. USA 85:4397-401), and the ligase-mediated detection procedure (Landegren et al., 1988, Science 241:1077).

Oligonucleotides specific to normal, mutant or alterative sequences are chemically synthesized using commercially available machines, labeled radioactively with isotopes (such as ³²P) or non-radioactively, with tags such as biotin (Ward and Langer et al., 1981. Proc. Nat. Acad. Sci. USA 78:6633-57), and hybridized to individual DNA samples immobilized on membranes or other solid supports by dot-blot or transfer from gels after electrophoresis. The presence of these specific sequences are visualized by methods such as autoradiography or fluorometric (Landegren et al., 1989, Science 242:229-37) or calorimetric reactions (Gebeyehu et al., 1987, Nucleic Acids Res. 15:4513-34). The absence of hybridization would indicate a mutation in the particular region of the gene, or that the patient is not infected with RRV or JMHV.

Sequence differences between disclosed and other forms of RRV or JMHV genes may also be revealed by the direct DNA sequencing method of Church and Gilbert (Proc. Natl. Acad. Sci. USA 81:1991-5, 1988). Cloned DNA segments may be used as probes to detect specific DNA segments. The sensitivity of this method is greatly enhanced when combined with PCR (Wrichnik et al., 1987, Nucleic Acids Res. 15:52942; Wong et al., 1987, Nature 330:384-6; Stoflet et al., 1988, Science 239:491-4). In this approach, a sequencing primer which lies within the amplified sequence is used with double-stranded PCR product or single-stranded template generated by a modified PCR. The sequence determination is performed by conventional procedures with radiolabeled nucleotides or by automatic sequencing procedures with fluorescent tags.

Sequence alterations may occasionally generate fortuitous restriction enzyme recognition sites or may eliminate existing restriction sites. Changes in restriction sites are revealed by the use of appropriate enzyme digestion followed by conventional gel-blot hybridization (Southern, 1975, J. Mol. Biol. 98:503). DNA fragments carrying the site (either normal, mutant, or alternative) are detected by their reduction in size or increase of corresponding restriction fragment numbers. Genomic DNA samples may also be amplified by PCR prior to treatment with the appropriate restriction enzyme; fragments of different sizes are then visualized under UV light in the presence of ethidium bromide after gel electrophoresis.

Screening based on DNA sequence differences may be achieved by detection of alteration in electrophoretic mobility of DNA fragments in gels with or without denaturing reagent. Small sequence deletions and insertions can be visualized by high-resolution gel electrophoresis. For example, a PCR product with small deletions is clearly distinguishable from a normal sequence on an 8% non-denaturing polyacrylamide gel (WO 91/10734; Nagamine et al., 1989, Am. J. Hum. Genet. 45:337-9). DNA fragments of different sequence compositions may be distinguished on denaturing formamide gradient gels in which the mobilities of different DNA fragments are retarded in the gel at different positions according to their specific “partial-melting” temperatures (Myers et al., 1985, Science 230:1242). Alternatively, a method of detecting a mutation comprising a single base substitution or other small change could be based on differential primer length in a PCR. For example, an invariant primer could be used in addition to a primer specific for a mutation. The PCR products of the normal and mutant genes can then be differentially detected in acrylamide gels.

In addition to conventional gel-electrophoresis and blot-hybridization methods, DNA fragments may also be visualized by methods where the individual DNA samples are not immobilized on membranes. The probe and target sequences may be both in solution, or the probe sequence may be immobilized (Saiki et al., 1989, Proc. Nat. Acad. Sci. USA 86:62304). A variety of detection methods, such as autoradiography involving radioisotopes, direct detection of radioactive decay (in the presence or absence of scintillant), spectrophotometry involving calorigenic reactions and fluorometry involved fluorogenic reactions, may be used to identify specific individual genotypes.

If more than one mutation or alternative sequence is frequently encountered in one or more RRV or JMHV genes, a system capable of detecting such multiple mutations would be desirable. For example, a PCR with multiple, specific oligonucleotide primers and hybridization probes may be used to identify all possible mutations or alternative sequences at the same time (Chamberlain et al., 1988, Nucl. Acids Res. 16:1141-55). The procedure may involve immobilized sequence-specific oligonucleotides probes (Saiki et al., 1989, Proc. Nat. Acad. Sci. USA 86:6230-4).

### EXAMPLE 20

### Quantitation of RRV or JMHV Proteins

An alternative method of determining if a subject has been infected with RRV or JMHV is to quantitate the level of one or more RRV (or JMHV) proteins in the cells of a subject. This diagnostic tool would be useful for detecting the levels of RRV proteins which result from, for example, infection by RRV. This diagnostic tool would also be useful for detecting the levels of the JMHV proteins which result from infection by JMHV. These diagnostic methods, in addition to those described in EXAMPLE 19, provide an enhanced ability to diagnose susceptibility to diseases caused by RRV or JMHV infection.

The determination of RRV or JMHV protein levels would be an alternative or supplemental approach to the direct determination of the presence of one or more RRV (or JMHV) genes by the methods outlined above in EXAMPLE 19. The availability of antibodies specific to one or more of the RRV (or JMHV) proteins (for example those described in Example 18) will facilitate the quantitation of cellular RRV or JMHV proteins by one of a number of immunoassay methods which are well known in the art and are presented in Harlow and Lane (Antibodies, A Laboratory Manual, Cold Spring Harbor Laboratory, New York. 1988).

Such assays permit both the detection of RRV or JMHV proteins in a biological sample and the quantitation of such proteins. Typical methods involve: providing a biological sample of the subject in which the sample contains cellular proteins, and providing an immunoassay for quantitating the level of RRV or JMHV protein in the biological sample. This can be achieved by combining the biological sample with an RRV or JMHV specific binding agent, such as an antibody (e.g. monoclonal or polyclonal antibodies that bind an RRV or JMHV protein), so that complexes form between the binding agent and the viral protein present in the sample, and then detecting or quantitating such complexes.

In particular forms, these assays may be performed with the RRV specific binding agent or the JMHV specific binding agent immobilized on a support surface, such as in the wells of a microtiter plate or on a column. The biological sample is then introduced onto the support surface and allowed to interact with the specific binding agent so as to form complexes. Excess biological sample is then removed by washing, and the complexes are detected with a reagent, such as a second anti-RRV protein antibody, or anti-JMHV protein antibody, that is conjugated with a detectable marker.

In an alternative assay, the cellular proteins are isolated and subjected to SDS-PAGE followed by Western blotting, for example as described in Example 18. After resolving the proteins, the proteins are transferred to a membrane, which is probed with specific binding agents that recognize any of the RRV proteins or JMHV proteins. The proteins are detected, for example with HRP-conjugated secondary antibodies, and quantitated.

In yet another assay, the level of one or more RRV or JMHV proteins in cells is analyzed using microscopy. Using specific binding agents which recognize RRV or JMHV, samples can be analyzed for the presence of one or more RRV or JMHV proteins. For example, frozen biopsied tissue sections are thawed at room temperature and fixed with acetone at −200° C. for 5 minutes. Slides are washed twice in cold PBS for 5 minutes each, then air-dried. Sections are covered with 20-30 μl of antibody solution (15-45 μg/ml) (diluted in PBS, 2% BSA at 15-50 μg/ml) and incubated at room temperature in humidified chamber for 30 minutes. Slides are washed three times with cold PBS 5 minutes each, allowed to air-dry briefly (5 minutes) before applying 20-30 μl of the second antibody solution (diluted in PBS, 2% BSA at 15-50 μg/ml) and incubated at room temperature in humidified chamber for 30 minutes. The label on the second antibody may contain a fluorescent probe, enzyme, radiolabel, biotin, or other detectable marker. The slides are washed three times with cold PBS 5 minutes each then quickly dipped in distilled water, air-dried, and mounted with PBS containing 30% glycerol. Slides can be stored at 4° C. prior to viewing.

For samples prepared for electron microscopy (versus light microscopy), the second antibody is conjugated to gold particles. Tissue is fixed and embedded with epoxy plastics, then cut into very thin sections (˜1-2 em). The specimen is then applied to a metal grid, which is then incubated in the primary anti-RRV antibody, washed in a buffer containing BSA, then incubated in a secondary antibody conjugated to gold particles (usually 5-20 nm). These gold particles are visualized using electron microscopy methods.

For the purposes of quantitating the RRV or JMHV proteins, a biological sample of the subject, which sample includes cellular proteins, is required. Such a biological sample may be obtained from body cells, such as those present in which expression of the protein has been detected. The expression of RRV or JMHV proteins in peripheral blood leukocytes is clearly the most accessible and convenient source from which specimens can be obtained. Specimens can be obtained from peripheral blood, urine, saliva, tissue biopsy, amniocentesis samples, surgical specimens, fine needle aspirates, and autopsy material, particularly cancer cells. Quantitation of RRV or JMHV proteins would be made by immunoassay and compared to levels of the protein found in non-RRV or non-JMHV expressing cells, or to the level of RRV proteins in non-RRV infected cells (cells of the same origin that are not infected), or to the level of JMHV proteins in non-JMHV infected cells (cells of the same origin that are not infected). A significant (preferably 50% or greater) increase in the amount of one or more RRV proteins in the cells of a subject compared to the amount of one or more RRV proteins found in non-RRV infected cells or that found in normal cells, would be taken as an indication that the subject may have been infected with RRV. Similarly, a significant (preferably 50% or greater) increase in the amount of one or more JMHV proteins in the cells of a subject compared to the amount of one or more JMHV proteins found in non-JMHV infected cells or that found in normal cells, would be taken as an indication that the subject may have been infected with JMHV.

### EXAMPLE 21

### Sequence Variants of RRV or JMHV

Sequence information for JMHV is shown in Example 31, and in the attached sequence listing. The amino acid sequence of JMHV proteins now facilitates the creation of DNA molecules, and thereby proteins, which are derived from those disclosed by vary in their precise nucleic acid or amino acid sequence from those disclosed. Such variants may be obtained through a combination of standard molecular biology laboratory techniques and the nucleotide sequence information disclosed by this invention.

Variant DNA molecules include those created by standard DNA mutagenesis techniques, for example, M13 primer mutagenesis. Details of these techniques are provided in Sambrook et al. (In: Molecular Cloning: A Laboratory Manual, Cold Spring Harbor, N.Y., 1989, Chapter 15). By the use of such techniques, variants may be created which differ in minor ways from those disclosed. DNA molecules and nucleotide sequences which are derivatives of those specifically disclosed herein and which differ from those disclosed by the deletion, addition or substitution of nucleotides while still encoding a protein which possesses the functional characteristics of the RRV proteins are comprehended by this invention. Also within the scope of this invention are small DNA molecules which are derived from the disclosed DNA molecules. Such small DNA molecules include oligonucleotides suitable for use as hybridization probes or polymerase chain reaction (PCR) primers. These small DNA molecules will comprise at least a segment of the RRV or cDNA molecules or the RRV gene and, for the purposes of PCR, will comprise at least a 15 or a 20-50 nucleotide sequence of the RRV cDNAs (even-numbered SEQ ID Nos 2-164) or the RRV genes (i.e., at least 20-50 consecutive nucleotides of the RRV cDNA or gene sequences). Alternatively, these small DNA molecules will comprise at least a segment of the JMHV or cDNA molecules or the JMHV gene and, for the purposes of PCR, will comprise at least a 15 or a 20-50 nucleotide sequence of the JMHV cDNAs or the JMHV genes (i.e., at least 20-50 consecutive nucleotides of a nucleic acid encoding a JMHV protein). DNA molecules and nucleotide sequences which are derived from the disclosed DNA molecules as described above may also be defined as DNA sequences which hybridize under stringent conditions to the DNA sequences disclosed, or fragments thereof.

Hybridization conditions resulting in particular degrees of stringency will vary depending upon the nature of the hybridization Method of choice and the composition and length of the hybridizing DNA used. Generally, the temperature of hybridization and the ionic strength (especially the Na⁺ concentration) of the hybridization buffer will determine the stringency of hybridization Calculations regarding hybridization conditions required for attaining particular degrees of stringency are discussed by Sambrook et al. (In: Molecular Cloning: A Laboratory Manual, Cold Spring Harbor, N.Y., 1989 ch. 9 and 11), herein incorporated by reference. By way of illustration only, a hybridization experiment may be performed by hybridization of a DNA molecule (for example, a deviation of the RRV cDNA) to a target DNA molecule (for example, the RRV cDNA) which has been electrophoresed in an agarose gel and transferred to a nitrocellulose membrane by Southern blotting (Southern, J. Mol. Biol. 98:503, 1975), a technique well known in the art and described in Sambrook et al. (Molecular Cloning: A Laboratory Manual, Cold Spring Harbor, N.Y., 1989). Hybridization with a target probe labeled with [³²P]-dCTP is generally carried out in a solution of high ionic strength such as 6×SSC at a temperature that is 20-25° C. below the melting temperature, Tₘ, described below. For such Southern hybridization experiments where the target DNA molecule on the Southern blot contains 10 ng of DNA or more, hybridization is typically carried out for 6-8 hours using 1-2 ng/ml radiolabeled probe (of specific activity equal to 10⁹ CPM/μg or greater). Following hybridization, the nitrocellulose filter is washed to remove background hybridization. The washing conditions should be as stringent as possible to remove background hybridization but to retain a specific hybridization signal. The term Tₘ represents the temperature above which, under the prevailing ionic conditions, the radiolabeled probe molecule will not hybridize to its target DNA molecule. The Tₘ of such a hybrid molecule may be estimated from the following equation (Bolton and McCarthy, Proc. Natl. Acad. Sci. USA 48:1390, 1962): Tₘ=81.5° C.-16.6(log₁₀[Na⁺])+0.41(% G+C)-0.63(% formamide)-(600/1); where I=the length of the hybrid in base pairs.

This equation is valid for concentrations of Na⁺ in the range of 0.01 M to 0.4 M, and it is less accurate for calculations of Tₘ in solutions of higher [Na⁺]. The equation is also primarily valid for DNAs whose G+C content is in the range of 30% to 75%, and it applies to hybrids greater than 100 nucleotides in length (the behavior of oligonucleotide probes is described in detail in Ch. 11 of Sambrook et al. (Molecular Cloning: A Laboratory Manual, Cold Spring Harbor, N.Y., 1989).

Thus, by way of example, for a 150 base pair DNA probe derived from the open reading frame of the RRV cDNA (with a hypothetical % GC=45%), a calculation of hybridization conditions required to give particular stringencies may be made as follows: For this example, it is assumed that the filter will be washed in 0.3×SSC solution following hybridization, thereby: [Na⁺]=0.045M; % GC=45%; Formamide concentration=0; 1=150 base pairs; Tₘ=81.5-16.6(log₁₀[Na⁺])+(0.41×45)−(600/150); and so Tₘ=74.4° C.

The Tₘ of double-stranded DNA decreases by 1-1.5° C. with every 1% decrease in homology (Bonner et al., J. Mol. Biol. 81:123, 1973). Therefore, for this given example, washing the filter in 0.3×SSC at 59.4-64.4° C. will produce a stringency of hybridization equivalent to 90%; that is, DNA molecules with more than 10% sequence variation relative to the target RRV cDNA will not hybridize. Alternatively, washing the hybridized filter in 0.3×SSC at a temperature of 65.4-68.4° C. will yield a hybridization stringency of 94%; that is, DNA molecules with more than 6% sequence variation relative to the target RRV cDNA molecule will not hybridize. The above example is given entirely by way of theoretical illustration. One skilled in the art will appreciate that other hybridization techniques may be utilized and that variations in experimental conditions will necessitate alternative calculations for stringency.

In particular embodiments of the present invention, stringent conditions may be defined as those under which DNA molecules with more than 25%, 15%, 10%, 6% or 2% sequence variation (also termed “mismatch”) will not hybridize.

The degeneracy of the genetic code further widens the scope of the present invention as it enables major variations in the nucleotide sequence of a DNA molecule while maintaining the amino acid sequence of the encoded protein. In one specific, non-limiting example, the eleventh amino acid residue of the RRV MIP protein is alanine. This is encoded in the RRV cDNA by the nucleotide codon triplet GCG. Because of the degeneracy of the genetic code, three other nucleotide codon triplets, GCT, GCA and GCC, also code for alanine. Thus, the nucleotide sequence of the RRV DNA could be changed at this position to any of these three codons without affecting the amino acid composition of the encoded protein or the characteristics of the protein. Based upon the degeneracy of the genetic code, variant DNA molecules may be derived from the DNA molecules disclosed herein using standard DNA mutagenesis techniques as described above, or by synthesis of DNA sequences. DNA sequences which do not hybridize under stringent conditions to the DNA sequences disclosed by virtue of sequence variation based on the degeneracy of the genetic code are herein also comprehended by this invention.

The invention also includes DNA sequences that are substantially identical to any of the DNA sequences disclosed herein, where substantially identical means a sequence that has identical nucleotides in at least 75%, 80%, 85%, 90%, 95%, 98%, or even 99% of the aligned sequences.

One skilled in the art will recognize that the DNA mutagenesis techniques described above may be used not only to produce variant DNA molecules, but will also facilitate the production of proteins which differ in certain structural aspects from the RRV or JMHV proteins, yet which proteins are clearly derivative of this protein and which maintain the essential characteristics of the RRV or JMHV proteins. Newly derived proteins may also be selected in order to obtain variations on the characteristic of the RRV or JMHV proteins, as described above. Such derivatives include those with variations in amino acid sequence including minor deletions, additions and substitutions.

While the site for introducing an amino acid sequence variation is predetermined, the mutation per se need not be predetermined. For example, in order to optimize the performance of a mutation at a given site, random mutagenesis may be conducted at the target codon or region and the expressed protein variants screened for the optimal combination of desired activity. Techniques for making substitution mutations at predetermined sites in DNA having a known sequence as described above are well known.

Amino acid substitutions are typically of single residues; insertions usually will be on the order of about from 1 to 10 amino acid residues; and deletions will range about from 1 to 30 residues. Deletions or insertions preferably are made in adjacent pairs, i.e., a deletion of two residues or insertion of two residues. Substitutions, deletions, insertions or any combination thereof may be combined to arrive at a final construct Obviously, the mutations that are made in the DNA encoding the protein must not place the sequence out of reading frame and preferably will not create complementary regions that could produce secondary mRNA structure.

Substitutional variants are those in which at least one residue in the amino acid sequence has been removed and a different residue inserted in its place. Such substitutions generally are made conservatively, as defined above.

The effects of these amino acid substitutions or deletions or additions may be assessed for derivatives of the JMHV proteins by assays in which DNA molecules encoding the derivative proteins are transfected into cells using routine procedures. These JMHV proteins are expressed recombinantly (for example see Example 17), purified, and analyzed for their ability to cause symptoms associated with JMHV infection.

### EXAMPLE 22

### Cloning Virus in Other Species

The genomic sequence of the rhesus macaque RRV (SEQ ID NO 1) and sequences of JMHV, facilitates the identification of DNA molecules, and thereby proteins, which are the RRV or JMHV homologs in other species. These other homologs can be derived from those sequences disclosed, but which vary in their precise nucleotide or amino acid sequence from those disclosed. Such variants may be obtained through a combination of standard molecular biology laboratory techniques and the nucleotide and amino acid sequence information disclosed by this invention.

The Japanese macaque virus isolate was isolated from a lesion that was minced and co-cultured with primary rhesus fibroblasts. The isolate was then cloned by limiting dilution and a stock of virus generated from this clone. Total cellular DNA was harvested from virus infected cells and the DNA subjected to degenerate PCR for viral DNA polymerase, exactly as described above for RRV. Once confirmed, a cosmid library of this virus was made from purified viral DNA (as described for RRV) and then a portion of the protein genes was cloned and sequenced.

Results for this analysis are shown in the following Table 1:

TABLE 1
RRV Sequences from Japanese Macaque

Total number of amino acid residues inferred: 972
Number of differences compared to RRV: 29
Percent identity: 97.02%
Japanese Macaque Data
These are end sequences. For ORFs represented
twice, section I is from one plasmid, section
II is from another plasmid. These are non-over-
lapping sections.
Orf 7 section I (see SEQ ID NO:14)
GLFNSIDDTINALSRDCSVTFFQQANYTNVMRKQNELFTRLNSILCQGSA
GSQKPATPSEPRTATVAATAASDVIKDAQYRKEQYMKKVARDGFKKLTEC
LQTQSAVLANALCMRVWGGVQYGEASELVNHFLLRRRFVALPWEARCRSN
QILFENSKYIKNSLYSQRLSREHVEIITLQFYGLITGPLTRQSDLFPGPA
NVVLAQCFEAAGMLPHHKMLVSEMIW
Orf 7 section II (see SEQ ID NO:14)
PIESLFCGGLFNSIDDTINALSRDCSVTFFQQANYTNXMRKQNELFTRLN
SILCQGSAGSXKPATPSEPRTATVXATAASDVIKDAQYRXEQYMKKVARD
XFKKLTECLQTQSAVLANALCMRRMGGRRI
Orf 8 (see SEQ ID NO:24 and SEQ ID NO:27)
YRKVATSVTVYRGWTETAVTGKQEVIRPVPQYEINHMDTTYQCFSSMRVN
VNGIENTYTDRDFTNQTVFLQPVEGLTDNIQRYFSQPVLYTTPGWFPGIY
RVRTTVNCEIVDMIARSAEPYSYFVTALGDTVEVSPFCLNDSTCSVADKA
ENGLGVRVLTNYTIVDFATRTPTTETRVFADSGEYTVSWKAEDPKSAVCA
LTLWKTFPRAIQTTHESQLPLCGQRR
Orf 9 section I
(see SEQ ID NO:20 and SEQ ID NO:25)
VPSRFQTDIIPSGTVLKLLGRTENGTSVCVNVFRQQVYFYAKVPAGVNVT
HVLQQALKNTAGRAACGFSTRRVTKKILKTYDVAEHPVTEITLSSGSMLS
TLSDRLVACGCEVFESNVDAVRRFVLDHGFTTFGWYSCARATPRLAXRDA
RTALEFDCSWEDLSV
Orf 9 section II
(see SEQ ID NO:20 and SEQ ID NO:25)
MDFFNPYLGPRGPRPPSHKCTDAPAPAGAVQPPPDVCRLIPACLRTPGAG
GMIPVTIPFPPTYFENGARGDVLLAHERSMWTARGQRPVVPDPQDQSITF
HAYDVVETTYAADRCAEV
Orf 10 (see SEQ ID NO:18)
AQMKIIYAPGDPNAEIVLGQSGPVLPTHTGGRVLGVYADAEKTIQPGSSA
EVRVQLIFPTGSAARGDLAFLVAGVAPEPLFIVTPTLLLSGCTT
HLRLFNPNGT
Orf 29b (SEQ ID NO:28)
NVAVEGNSSQDAGVAIATVLNEICSVPLSFLHHADKNTLIRSPIYMLGPE
KAKAFESFIYALNSGTFSASQTVVSHTIKLSFDPVAYLIDQIKAIRCIPL
KDGGHTYCAKQKTMSDDVLVATVMAHYMATNDKFVFKSLE

The clones containing the Japanese macaque gamma2 herpesvirus was subsequently sequenced. The nucleic acid sequence form the Japanese macaque gamma2 herpesvirus was then compared with the sequence of the rhesus macaque gamma2 herpesvirus 17577. Based on a comparison of 2328 amino acid residues form fourteen independent regions (average length 166±71), the percent similarity between the two viruses was calculated to be an average of 94.12% at the polypeptide level (range 75.652% to 99.422%) when JMHV and RRV sequences were compared (see Example 22).

Similarly, this invention now also facilitates the identification of other DNA molecules, and thereby proteins, which are the JMHV homologs in other species. These other homologs are derived or related to those sequences disclosed, but vary in their precise nucleotide or amino acid sequence. Such variants may be obtained through a combination of standard molecular biology laboratory techniques and the JMHV nucleotide and amino acid sequence information disclosed by this invention.

In one specific, non-limiting example, a human homolog of JMHV is isolated from a human MS lesion lesion that is minced and co-cultured with primary fibroblasts, using the methods described herein. In one specific, non-limiting example, primary tissue explant cultures and cocultivation of leukocytes with traget cells are used to enhance the isolation of a human virus. Fresh tissue (e.g. spinal chord) is dissected from visible human MS lesion identified during necrosy. Tissue explant cultures are established from small tissue framents cultured in flasks (e.g. T-25 flasks) in media, such as Dulbecco's Modified Eagle Medium (DMEM) supplemented with serum, L-glutamine and antibiotics. In one example, 20% fetal calf serum is utilized. In another example, human serum is utilized. The explant cultures are fed by partial medium replacement, and passaged at confulence. Confulent monlayer explant cultures are rinsed in calcium and Magnesium-free phospahte buffered saline, pH 7,4, and digested with trypsin-EDTA at 37° C., washed with grwoth medium and seeded into flasks. These first passage explant cultures are fed by complet medium replacement, and watched for the development of cytopathic effects. One cytopathic effects are noted, the cells are rapidly frozen in liquid nitrogen and thawed. Cell-fee supernatants are clarified by centrifugation, fileted, and frozen at −80° C.

Peripheral blood mononuclear cells (PBMC) are then isopycnic gradient-purified (Ficoll-Paque, Pharmacia) from fresh ammonium heparin anti-coagulated blood obtained from a human MS patient at necropsy. Gradient-purified PBMC are washed in calcium- and magnesium-free phosphate-buffered saline and approximately 3×10⁶ PBMC are cocultured with 1×10⁵ primary human fibroblasts in T-25 culture flasks containing DMEM growth medium supplemented with serum, 1% L-glutamine and antibiotics (e.g. 1% penicillin-streptomycin-neomycin). The PBMC-fibroblast cocultures are fed by partial medium replacement after several days in culture, and passaged at confluence at approximately day 9. Confluent PBMC-fibroblast cocultures are rinsed in calcium- and magnesium-free phosphate-buffered saline, pH 7.4, digested with trypsin-EDTA at 37° C., washed with growth medium and then seeded into culture flasks. First passage PBMC-fibroblast cultures are fed by complete medium replacement at 3 to 5 day intervals and exhibit cytopathic effects (CPE) one to two weeks after passage. Once cytopathic are noted the cultures are rapidly frozen in liquid N₂ and thawed. Cell-free supernatants are clarified by centrifugation, filtered through a 0.45 micron membrane and stored frozen at −8° C.

To prepare virus stocks, cell-free filtered supernatants are harvested from the explant cultures and the PBMC-fibroblast cocultures and separately inoculated onto primary human fibroblast cultures. Fibroblast cultures inoculated with explant supernatant and PBMC-fibroblast coculture supernatant develop CPE after inoculation. The cultures are rapidly frozen in liquid N₂ and thawed and cell-free supernatants are clarified by centrifugation, filtered through a 0.45 micron membrane and stored frozen at −80° C.

The isolate is then cloned by limiting dilution, and a stock of virus is generated from this clone. Total cellular DNA is harvested from virus infected cells. The harvested DNA is then subjected to degenerate PCR for viral DNA polymerase, exactly as described above for JMHV and RRV. Once confirmed, a cosmid library of this virus is made from purified viral DNA (as described above for JMHV and RRV). The viral genes included in the cosmid library are subseqwuently cloned and sequenced.

### EXAMPLE 23

### Screening Assays for Pharmaceutical Agents of Interest

The present disclosure provides a virus that is involved in the causation or progression of certain diseases, such as KS or multiple sclerosis, and therefore provides an animal model and assays directed to identifying potential pharmaceutical agents that inhibit the biological activity of the virus. Drug screening assays which determine whether or not a drug has activity against the virus can include incubating a compound to be evaluated for use in treatment of the condition with cells which express the RRV or JMHV proteins or peptides, and determining the effect of the compound on the activity of the virus. In vitro assays in which the virus is maintained in suitable cell culture are preferred, though in vivo animal models would also be effective.

In vitro assays include infecting cells such as rhesus or Japanese macaque fibroblasts, neuronal cells, peripheral blood leukocytes or susceptible T cell lines such as MT-4 with the agent of interest in the presence of varying concentrations of compounds targeted against viral replication. These compounds include, but are not limited to nucleoside analogs, chain terminators, antisense oligonucleotides and random polypeptides. (Asada et al., J. Clin. Microbiol. 27:2204, 1989; Kikuta et al., Lancet 7:861, 1989). Infected cultures and their supernatants can be assayed for the total amount of virus, including the presence of the viral genome, by quantitative PCR, by dot blot assays, or by using immunologic methods. For example, a culture of susceptible cells could be infected with the RRV or JMHV in the presence of various concentrations of drug, fixed on slides after a period of days, and examined for viral antigen by indirect immunofluorescence with monoclonal antibodies to viral polypeptides (Kikuta et al, supra). Alternatively, chemically adhered MT4 cell monolayers can be used for an infectious agent assay using indirect immunofluorescent antibody staining to search for focus reduction (Higashi, J. Clin. Microbiol. 27:2204, 1989, incorporated by reference).

As an alternative to whole cell in vitro assays, purified enzymes isolated from the RRV or JMHV can be used as targets for rational drug design to determine the effect of the potential drug on enzyme activity, such as thymidylate sunthase or DNA polymerase. The genes for these two RRV enzymes are provided herein. A measure of enzyme activity indicates an effect on the infectious agent itself. Drug screens using herpes viral products are known and have been previously described in EP 0514830 (herpes proteases) and WO 94/04920 (U_{L} 13 gene product).

In particular embodiments, this invention provides an assay for screening anti-KS or anti-MS chemotherapeutics. Infected cells can be incubated in the presence of a chemical agent that is a potential chemotherapeutic against KS (e.g. acyclo-guanosine). The level of virus in the cells is then determined after several days by IFA for antigens or Southern blotting for viral genome or Northern blotting for mRNA and compared to control cells. This assay can quickly screen large numbers of chemical compounds that may be useful against KS. This invention also provides an assay system that is employed to identify drugs or other molecules capable of binding to the DNA molecule or proteins, either in the cytoplasm or in the nucleus, thereby inhibiting or potentiating transcriptional activity. This assay would be useful in the development of drugs that are specific against particular cellular activity, or that would potentiate such activity, in time or in level of activity. Also included are drugs identified by this assay which have an anti-viral activity, and an effect against conditions associated with RRV infection, such as KS.

Similarly, this invention provides an assay for secreening anti-multiple sclerosis (MS) therapeutics. Infected cells can be incubated in the presence of a chemical agent that is a potential anti-inflammatory agent or other agent of use in treating MS. The level of virus in the cells is then determined by IFA for antigens, Southern blotting for the viral genome, Northern blotting for mRNA, or PCR, and compared to control cell. This assay can quickly screen large numbers of agents that may be useful in the treatment of MS. This invention also provides an assay system that is employed to identify drugs or other molecules capable of binding to the DNA molecule or proteins, either in the cytoplasm or in the nucleus, thereby inhibiting or potentiating transcriptional activity. This assay would be useful in the development of drugs that are specific against particular cellular activity, or that would potentiate such activity, in time or in level of activity. Also included are drugs identified by this assay which have an anti-viral activity, and an effect against conditions associated with JMHV infection, such as MS.

### EXAMPLE 24

### Generating Animal Models

Animal models are useful for resolving a number of fundamental problems of infectious diseases that include, but are not limited to, determinants of virulence of the organism, mechanisms of host resistance, mechanisms of pathogenicity, establishment and regulation of chronic infection, and antimicrobial and chemotherapeutic actions of drugs on infectious agents. Variables that are commonly manipulated to address fundamental problems include, but are not limited to, the strain of infectious agent, the infecting dose of infectious agent and the route of administration of the infectious agent, the species or subspecies of animal, the age of animal, and the genetic background of the animal (Viral pathogenesis, N. Nathanson, Lippincot-Raven, Philadelphia, 1997).

In an embodiment in which one or more RRV strains are employed for generating an animal model, the RRV used may be naturally occurring variant isolates recovered from rhesus macaques and other non-human primate species, molecular clones generated from these naturally occurring variant isolates and recombinant viruses with introduced mutations, deletions or recombined genomes designed to address function of specific genes.

By manipulating the infecting dose and route of RRV administration virus-host interactions dependent upon dose and tissue or organ-specific disease manifestations can be explored. Thus, the present invention includes various doses of RRV administered by oral, inhalation, intratracheal, intravaginal, intrarectal and parenteral routes including, but not limited to intravenous, intraarterial, intradermal, subcutaneous, intramuscular, intraperitoneal and organ-specific administration routes such and intracerebral and intraocular administration.

Many disease manifestations with a given infections agent are highly influenced by age and species or subspecies of the host and the particular genetic makeup of the host. The present disclosure provides a virus that is involved in the causation or progression of certain diseases, such as KS, in the rhesus macaque, but is also useful for the study of and discovery of disease manifestations that are host species, age and genetic background dependent. In particular embodiments, one skilled in the art may vary the species of animal to which the RRV is administered to produce or discover a particular disease manifestation, or similarly vary the genetic background of the animal to produce or discover a particular disease manifestation, even including the use of genetically engineered animals.

In another embodiment, in which one or more JMHV strains are employed for generating an animal model, the JMHV used may be naturally occurring variant isolates recovered from Japanese macaques and other non-human primate species, molecular clones generated from these naturally occurring variant isolates and recombinant viruses with introduced mutations, deletions or recombined genomes designed to address function of specific genes.

By manipulating the infecting dose and route of JMHV administration virus-host interactions dependent upon dose and tissue or organ-specific disease manifestations can similarly be explored. Thus, the present invention includes various doses of JMHV administered by oral, inhalation, intratracheal, intravaginal, intrarectal and parenteral routes including, but not limited to intravenous, intraarterial, intradermal, subcutaneous, intramuscular, intraperitoneal and organ-specific administration routes such and intracerebral and intraocular administration.

Many disease manifestations with a given infections agent are highly influenced by age and species or subspecies of the host and the particular genetic makeup of the host. The present disclosure provides a virus that is involved in the causation or progression of certain diseases, such as MS, in the Japanese macaque, but is also useful for the study of and discovery of disease manifestations that are host species, age and genetic background dependent. In particular embodiments, one skilled in the art may vary the species of animal to which the JMHV is administered to produce or discover a particular disease manifestation, or similarly vary the genetic background of the animal to produce or discover a particular disease manifestation, even including the use of genetically engineered animals.

### EXAMPLE 25

### Pharmaceutical Compositions and Modes of Administration

Various delivery systems for administering pharmaceutical proteins from the RRV or JMHV include encapsulation in liposomes, microparticles, microcapsules, expression by recombinant cells, receptor-mediated endocytosis (see Wu and Wu, J. Biol. Chem. 1987, 262:4429-32), and construction of a therapeutic nucleic acid (such as an anti-sense molecule) as part of a retroviral or other vector. Methods of introduction include, but are not limited to, intradermal, intramuscular, intraperitoneal, intravenous, subcutaneous, intranasal, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, the pharmaceutical compositions may be introduced into the central nervous system by any suitable route, including intraventricular and intrathecal injection; intraventricular injection may be facilitated by an intraventricular catheter, for example, attached to a reservoir, such as an Ommaya reservoir.

The use of liposomes as a delivery vehicle is another delivery method of the present invention. The liposomes fuse with the target site and deliver the contents of the lumen intracellularly. The liposomes are maintained in contact with the target cells for a sufficient time for fusion to occur, using various means to maintain contact, such as isolation and binding agents. Liposomes may be prepared with purified proteins or peptides that mediate fusion of membranes, such as Sendai virus or influenza virus. The lipids may be any useful combination of known liposome forming lipids, including cationic lipids, such as phosphatidylcholine. Other potential lipids include neutral lipids, such as cholesterol, phosphatidyl serine, phosphatidyl glycerol, and the like. For preparing the liposomes, the procedure described by Kato et al. (J. Biol. Chem. 1991, 266:3361) may be used

The present invention also provides pharmaceutical compositions which include a therapeutically effective amount of one or more RRV, or one or more JMHV proteins, or RRV or JMHV DNA, alone or with a pharmaceutically acceptable carrier.

The pharmaceutical compositions or methods of treatment may be administered in combination with other therapeutic treatments, such as other antineoplastic or antitumorigenic therapies.

Administration of Nucleic Acid Molecules

In an embodiment in which one or more RRV or JMHV nucleic acids are employed for generating an animal model, the analog may be delivered intracellularly (e.g., by expression from a nucleic acid vector or by receptor-mediated mechanisms). In a specific embodiment where the therapeutic molecule is a nucleic acid, administration may be achieved by an appropriate nucleic acid expression vector which is administered so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 1991, 88:1864-8). Alternatively, the nucleic acid can be introduced intracellularly and incorporated within host cell DNA for expression, by homologous recombination.

The vector pCDNA, is an example of a method of introducing the foreign cDNA into a cell under the control of a strong viral promoter (CMV) to drive the expression. However, other vectors can be used. Other retroviral vectors (such as pRETRO—ON, Clontech), also use this promoter but have the advantages of entering cells without any transfection aid, integrating into the genome of target cells ONLY when the target cell is dividing (as cancer cells do, especially during first remissions after chemotherapy) and they are regulated. It is also possible to turn on the expression of the RRV or JMHV nucleic acid by administering tetracycline when these plasmids are used. Hence these plasmids can be allowed to transfect the cells, then administer a course of tetracycline with a course of chemotherapy to achieve better cytotoxicity.

Other plasmid vectors, such as pMAM-neo (also from Clontech) or pMSG (Pharmacia) use the MMTV-LTR promoter (which can be regulated with steroids) or the SV 10 late promoter (pSVL, Pharmacia) or metallothionein—responsive promoter (pBPV, Pharmacia) and other viral vectors, including retroviruses. Examples of other viral vectors include adenovirus, AAV (adeno-associated virus), recombinant HSV, poxviruses (vaccinia) and recombinant lentivirus (such as HIV). All these vectors achieve the basic goal of delivering into the target cell the cDNA sequence and control elements needed for transcription. The present invention includes all forms of nucleic acid delivery, including synthetic oligos, naked DNA, plasmid and viral, integrated into the genome or not.

Also contemplated are inhibitory nucleic acid therapeutics which can inhibit the activity of RRV, for example in subject with KS or other diseases associated with RRV infection. Similarly, inhibitory nucleic acid therapeutics are provided which can inhibit the activity of JMHV, for example in subject with MS or other diseases associated with JMHV infection. Inhibitory nucleic acids may be single-stranded nucleic acids, which can specifically bind to a complementary nucleic acid sequence. By binding to the appropriate target sequence, an RNA-RNA, a DNA-DNA, or RNA-DNA duplex or triplex is formed. These nucleic acids are often termed “antisense” because they are usually complementary to the sense or coding strand of the gene, although recently approaches for use of “sense” nucleic acids have also been developed. The term “inhibitory nucleic acids” as used herein, refers to both “sense” and “antisense” nucleic acids.

By binding to the target nucleic acid, the inhibitory nucleic acid can inhibit the function of the target nucleic acid. This could, for example, be a result of blocking DNA transcription, processing or poly(A) addition to mRNA, DNA replication, translation, or promoting inhibitory mechanisms of the cells, such as promoting RNA degradation. Inhibitory nucleic acid methods therefore encompass a number of different approaches to altering expression of RRV or JMHV genes. Cleavage, and therefore inactivation, of the target nucleic acids may be effected by attaching a substituent to the inhibitory nucleic acid which can be activated to induce cleavage reactions. The substituent can be one that affects either chemical, or enzymatic cleavage. Alternatively, cleavage can be induced by the use of ribozymes or catalytic RNA. In this approach, the inhibitory nucleic acids would include either naturally occurring RNA (ribozymes) or synthetic nucleic acids with catalytic activity.

The inhibitory nucleic acid therapies can be used to target nucleic acids to sequences of RRV or JMHV for use in treating conditions caused by the RRV or JMHV, or proteins of the RRV (or JMHV), for example for treating KS or KS-like syndromes, or MS or MS-like syndromes.

Administration of Antibodies

Therapeutic, intravenous, polyclonal or monoclonal antibodies has been used as a mode of passive immunotherapy of herpesviral diseases, such as infection with CMV. Immune globulin from subjects previously infected with the RRV and bearing a suitably high titer of antibodies against the virus can be given in combination with antiviral agents (e.g. ganciclovir), or in combination with other modes of immunotherapy that are currently being evaluated for the treatment of KS, which are targeted to modulating the immune response (i.e. treatment with copolymer-1, antiidiotypic monoclonal antibodies, T cell “vaccination”). In one embodiment, antibodies specific for an epitope expressed on cells infected with the RRV are utilized and can be obtained as described above. Similarly immune globulin from subjects previously infected with the JMHV and bearing a suitably high titer of antibodies against the virus can be given in combination with antiviral agents (e.g. ganciclovir), or in combination with other modes of immunotherapy that are currently being evaluated for the treatment of MS, which are targeted to modulating the immune response. In one embodiment, antibodies specific for an epitope expressed on cells infected with the JMHV, obtained as described above, are utilized.

The present invention also provides pharmaceutical compositions which include a therapeutically effective amount of the antibody, and a pharmaceutically acceptable carrier or excipient.

### EXAMPLE 26

### Vaccines

This invention provides substances suitable for use as vaccines for the prevention of diseases associated with RRV infection, such as KS, and diseases associated with JMHV infection, or infection by related viruses which cause disease such as MS, and methods for administering them. Particular vaccines are directed against RRV ore related viruses, and may include antigens obtained from RRV or those related viruses, or the vaccines are directed against JMHV or related viruses, and may include antigens obtained from JMHV or its related viruses. In one embodiment, the vaccine contains attenuated RRV or JMHV, or related viruses found in humans. In another embodiment, the vaccine contains killed RRV or JMHV. In another embodiment, the vaccine contains a nucleic acid vector encoding RRV or JMHV, or a surface protein of RRV or JMHV, such as a capsid protein. In another embodiment, the vaccine is a subunit vaccine containing an RRV or JMHV subunit, such as glycoprotein B, major capsid protein, or other gene products found to elicit appropriate humoral and/or cell mediated immune responses.

This invention also provides a method of vaccinating a subject against Kaposi's sarcoma and lymphoproliferative disorders, comprising administering to a susceptible subject an effective amount of the peptide or polypeptide encoded by an isolated DNA molecule encoding a polypeptide or combination of polypeptides expressed by the DNA molecule, and a suitable acceptable carrier. In one embodiment, naked DNA is administered to the subject in an effective amount to vaccinate the subject against Kaposi's sarcoma and lymphoproliferative disorders, or other disease associated with RRV infection.

In another embodiment, a method is provided for vaccinating a subject against multiple sclerosis, comprising administering to a susceptible subject an effective amount of the peptide or polypeptide encoded by an isolated DNA molecule encoding a polypeptide or combination of polypeptides expressed by the DNA molecule, and a suitable acceptable carrier. In one embodiment, naked DNA is administered to the subject in an effective amount to vaccinate the subject against multiple sclerosis, or other disease associated with JMHV infection.

The vaccine can be made using synthetic peptide or recombinantly-produced polypeptide described above as antigen. Typically, a vaccine will include from about 1 to 50 micrograms of antigen, for example from about 15 to about 45 micrograms. Typically, the vaccine is formulated so that a dose includes about 0.5 milliliters. The vaccine may be administered by any route known in the art, for example parenteral, subcutaneous or intramuscular.

There are a number of strategies for amplifying an antigens effectiveness, particularly as related to the art of vaccines. For example, cyclization of a peptide can increase the peptide's antigenic and immunogenic potency. See U.S. Pat. No. 5,001,049. More conventionally, an antigen can be conjugated to a suitable carrier, usually a protein molecule. This procedure can allow multiple copies of an antigen, such as a peptide, to be conjugated to a single larger carrier molecule. Additionally, the carrier may possess properties which facilitate transport, binding, absorption or transfer of the antigen.

For parenteral administration, such as subcutaneous injection, examples of suitable carriers are the tetanus toxoid, the diphtheria toxoid, serum albumin and lamprey, or keyhole limpet, hemocyanin because they provide the resultant conjugate with minimum genetic restriction. Conjugates including these universal carriers can function as T cell clone activators in individuals having very different gene sets. The conjugation between a peptide and a carrier can be accomplished using one of the methods known in the art. Specifically, the conjugation can use bifunctional cross-linkers as binding agents as detailed, for example, by Means and Feeney, “A recent review of protein modification techniques,” Bioconjugate Chem. 1:2-12 (1990).

Vaccines against RRV can be made from the RRV envelope glycoproteins, and vaccines against JMHV can be made from the JMHV envelope glycoproteins. These proteins can be purified and used for vaccination (Lasky, L. A., 1990, J. Med. Virol. 31:59). MHC-binding peptides from cells infected with the human herpesvirus can be identified for vaccine candidates per the methodology of Marloes, et al., 1991, Eur. J. Immunol. 21:2963-2970. The RRV or JMHV antigen may be combined or mixed with various solutions and other compounds as is known in the art. For example, it may be administered in water, saline or buffered vehicles with or without various adjuvants or immunodiluting agents. Examples of such adjuvants or agents include aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, Corynebacterium parvum (Propionibacterium acnes), Bordetella pertussis, polyribonucleotides, sodium alginate, lanolin, lysolecithin, vitamin A, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.). Other suitable adjuvants are Amphigen (oil-in-water), Alhydrogel (aluminum hydroxide), or a mixture of Amphigen and Alhydrogel. Only aluminum is approved for human use.

The proportion of antigen and adjuvant can be varied over a broad range so long as both are present in effective amounts. For example, aluminum hydroxide can be present in an amount of about 0.5% of the vaccine mixture (Al₂O₃ basis). On a per-dose basis, the amount of the antigen can range from about 0.1 μg to about 100 μg protein per subject, for example about 1 μg to about 50 μg per dose, or about 15 μg to about 45 μg. A suitable dose size is about 0.5 ml. Accordingly, a dose for intramuscular injection, for example, would comprise 0.5 ml containing 45 μg of antigen in admixture with 0.5% aluminum hydroxide. After formulation, the vaccine may be incorporated into a sterile container which is then sealed and stored at a low temperature, for example 4° C., or it may be freeze-dried. Lyophilization permits long-term storage in a stabilized form.

The vaccines may be administered by any conventional method for the administration of vaccines including oral and parenteral (e.g., subcutaneous or intramuscular) injection. Intramuscular administration is preferred. The treatment may consist of a single dose of vaccine or a plurality of doses over a period of time. Also, the antigen could be a component of a recombinant vaccine which is adaptable for oral administration. Vaccines of the invention may be combined with other vaccines for other diseases to produce multivalent vaccines. A pharmaceutically effective amount of the antigen can be employed with a pharmaceutically acceptable carrier such as a protein or diluent useful for the vaccination of mammals, particularly humans. Other vaccines may be prepared according to methods well-known to those skilled in the art.

Those of skill will readily recognize that it is only necessary to expose a mammal to appropriate epitopes in order to elicit effective immunoprotection. The epitopes are typically segments of amino acids which are a small portion of the whole protein. Using recombinant genetics, it is routine to alter a natural protein's primary structure to create derivatives embracing epitopes that are identical to or substantially the same as (immunologically equivalent to) the naturally occurring epitopes. Such derivatives may include peptide fragments, amino acid substitutions, amino acid deletions and amino acid additions of the amino acid sequence for the viral polypeptides from the human herpesvirus. For example, it is known in the protein art that certain amino acid residues can be substituted with amino acids of similar size and polarity without an undue effect upon the biological activity of the protein. The human herpesvirus proteins have significant tertiary structure and the epitopes are usually conformational. Thus, modifications should generally preserve conformation to produce a protective immune response.

### EXAMPLE 27

### Peptide Synthesis and Purification

The peptides provided by the present invention can be chemically synthesized by any of a number of manual or automated methods of synthesis known in the art. For example, solid phase peptide synthesis (SPPS) is carried out on a 0.25 millimole (mmole) scale using an Applied Biosystems Model 431A Peptide Synthesizer and using 9-fluorenylmethyloxycarbonyl (Fmoc) amino-terminus protection, coupling with dicyclohexylcarbodiimide/hydroxybenzotriazole or 2-(1H-benzo-triazol-1-yl)-1,1,3,3-tetramethyluronium hexafluorophosphate/hydroxybenzotriazole (HBTU/HOBT), and using p-hydroxymethylphenoxymethylpolystyrene (HMP) or Sasrin resin for carboxyl-terminus acids or Rink amide resin for carboxyl-terminus amides.

Fmoc-derivatized amino acids are prepared from the appropriate precursor amino acids by tritylation and triphenylmethanol in trifluoroacetic acid, followed by Fmoc derivitization as described by Atherton et al. (Solid Phase Peptide Synthesis, IRL Press: Oxford, 1989).

Sasrin resin-bound peptides are cleaved using a solution of 1% TFA in dichloromethane to yield the protected peptide. Where appropriate, protected peptide precursors are cyclized between the amino- and carboxyl-termini by reaction of the amino-terminal free amine and carboxyl-terminal free acid using diphenylphosphorylazide in nascent peptides wherein the amino acid sidechains are protected.

HMP or Rink amide resin-bound products are routinely cleaved and protected sidechain-containing cyclized peptides deprotected using a solution comprised of trifluoroacetic acid (TFA), optionally also comprising water, thioanisole, and ethanedithiol, in ratios of 100:5:5:2.5, for 0.5-3 hours at room temperature.

Crude peptides are purified by preparative high pressure liquid chromatography (HPLC), for example using a Waters Delta-Pak C18 column and gradient elution with 0.1% TFA in water modified with acetonitrile. After column elution, acetonitrile is evaporated from the eluted fractions, which are then lyophilized. The identity of each product so produced and purified may be confirmed by fast atom bombardment mass spectroscopy (FABMS) or electrospray mass spectroscopy (ESMS).

### EXAMPLE 27

### Spontaneous Demyleniating Encelpholmyletis in Japanes Macaques

Multiple sclerosis (MS) is a chronic, debilitating inflammatory disease limited to central nervous system (CNS) white matter for which there are very few effective treatments. The disease may not be etiologically homogeneous, but rather a complex set of diseases that have in common pathogenic mechanisms that involve genetically predisposed individuals, and infectious agents as initiators and diverse mechanisms of inflammatory white matter destruction (Hafler, J Clin Invest 104:527-529, 1999). Genetic studies in families with MS-affected members have revealed that MS is a complex trait, that the contribution of individual genes to susceptibility is probably small, and that differences are possible between familial and sporadic forms of the disease (Kalman amd Lublin, Biomed Pharacother 53:358-370, 1999.).

The inflammatory response in MS is characterized by T lymphocyte-mediated demyelination of the CNS and by autoimmune responses to myelin proteins such as myelin basic protein (MBP), proteolipid protein (PLP), and myelin oligodendrocyte glycoprotein (MOG) (e.g. Wekerle, Curr Opin Neurobiol 3:779-784, 1993). No aspect of the inflammatory response in MS has been identified as being specific for the disease (Prineas and McDonald, In: Graham D I, Lantos P L (eds.), Greenfield's Neuropathology, 6^{th} Edn. Oxford Univ. Press, New York, pp. 813-896, 1997). The lesions are vasocentric and initially characterized by leukocyte investment of small blood vessels, endothelial cell activation, edema and myelin swelling. Secondarily, there is macrophage and microglial cell activation and selective myelin destruction. Chronic-active lesions are characterized by ongoing myelin breakdown and foamy macrophages are actively engaged in removing and digesting myelin. Oligodendroglial cells are typically reduced in number, astrocytes in the demyelinated zone are reactive and axons are, for the most part, preserved. Chronic plaques are, in general, quiescent glial scars. There is extensive variability in the clinical course of the disease and heterogeneity in the distribution and character of the lesions and there are five, and possibly six, clinical syndromes that seem to belong to the narrow group of immune-mediated demyelinating diseases considered to be MS or MS variants (Hickey, J Neuroimmunol 98:37-44, 1999).

Epidemiological evidence strongly supports the hypothesis that MS may be initiated by a virus infection. However, no single virus has been consistently found in MS lesions and no fewer than 16 infectious agents have been associated with the disease (Johnson, Viral Infections of the Nervous System, 2^{nd} Ed, Lippincott-Raven, Philadelphia, 1998). The JMHV, and related human viruses, are believed to be involved in the pathogenesis of this constellation of diseases.

Determining the mechanisms by which infections trigger autoimmune sequelae is an area of great interest, and may provide important clues about the pathogenesis and regulation of autoimmune diseases. While there is no evidence of an active cytolytic viral infection in the CNS in MS, viruses may participate in the pathogenesis of MS in one or more ways. First, viruses are postulated to share sequence homologies with myelin epitopes and, through the mechanism of molecular mimicry, activate autoreactive T cells (e.g. see Fujinami and Oldstone, Science 230:1043-1045, 1985). In general, there is little direct evidence to support this mechanism. Second, viral antigens expressed in the CNS as a result of chronic, non-cytolytic infection may serve as targets for virus-specific T cells entering into the CNS resulting in non-specific inflammatory, or bystander damage to myelin and myelin-producing oligodendrocytes (Miller and Gerety, Semin Virol 1:263-272, 1990). Third, CNS viral infections that initially cause cytolytic or bystander damage to myelin secondary to virus-specific T cells may promote induction of autoimmunity to the myelin proteins released by viral damage to the CNS by epitope spreading (Lehmann et al., Nature 558:155-157, 1992).

Several virus-induced and autoimmune models have been used to study the underlying mechanisms of myelin destruction in MS. These models include demyelinating diseases induced by infection with mouse hepatitis virus, Sindbis virus, Semliki Forest virus, herpesvirus, or Theiler's murine encephalomyelitis virus, as well as immunization with CNS autoantigens. Although these models have provided a wealth of knowledge about mechanisms of inflammatory demyelination and are attractive parallel models for human MS, their direct relevance to MS is unknown.

Demyelinating encephalomyelitis with morphologic features similar to multiple sclerosis was first observed in the Oregon Regional Primate Research Center Japanese macaque troop in July, 1986. The troop originated from 55 founder animals imported from Japan in 1965 and has been maintained as a close group. No additional animals have been introduced (Eaton, Adv Behav Biol 11:287-297, 1974). The incidence of the disease peaked the following year, affecting 3% of the population. Subsequently, approximately 1% of the population developed demyelinating encephalomyelitis annually. The age of the affected animals ranges from 97 days to 20 years, with approximately equal sex distribution (20 males and 18 females). The mean age at disease onset is 5 years, 22 days and young animals are over-represented among the 38 cases identified to date (median age=3 years, 284 days).

Clinically, the disease is characterized by acute onset paresis or paralysis involving one or more limbs in robust, healthy animals that lack evidence of chronic disease. The severity is variable ranging from limb weakness to tetraparesis. Cerebrospinal fluid analysis generally reveals moderate to marked pleocytosis dominated by lymphocytes and elevated protein (Table 2). Regardless of the initial severity, the disease is uniformly progressive and necessitates euthanasia of the animal. Treatment with high doses of corticosteroids delays progression for several days to weeks. However, all animals treated with corticosteroids to date have manifested relapsing, progressive disease.

TABLE 2
Japanese Macaque Demyelinating
Encephalomyelitis, CSF Analysis

| | Glucose | Total Protein | | | |
|---|---|---|---|---|---|
| ID No. | (mg %) | (mg/dL) | WBC (□L) | Lymph (%) | PMN (%) |
| 13762 | 58 | 20 | 1 | 100 | 0 |
| 14871 | nd | nd | 336 | 9 | 4 |
| 12800 | nd | 107 | 186 | 9 | 4 |
| 13754 | nd | 112 | 2,710 | 100 | 0 |
| 07804 | nd | nd | 58 | 100 | 0 |
| 09179 | nd | 11 | 0 | | |
| 08234 | nd | 104 | 11 | 100 | 0 |
| 08420 | 52 | 113 | 30 | 100 | 0 |
| 12133 | 41 | 32 | 0 | | |
| 16376 | 42 | 29 | 204 | 95 | 5 |
| 15997 | nd | 71 | 83 | 90 | 10 |
| 13768 | 56 | 56 | 112 | 100 | 0 |
| 16387 | 54 | 48 | 70 | 85 | 15 |
| 13744 | 46 | 12 | 8 | 100 | 0 |
| 15306 | 33 | 195 | 12 | 100 | 0 |
| 19400 | nd | 144 | 341 | 93 | 7 |
| Range | 33-58 | 11-195 | 0-2,710 | | |
| (Mean) | (48) | (75) | (260) | (89) | (5) |

Pathogically, Japanese macaque demyelinating encephalomyelitis is characterized by multifocal dull yellow or pale tan plaques that range from 0.2 to 1.0 cm in greatest dimension distributed primarily in the cerebellar and spinal cord white matter. Gray matter is exquisitely spared. Most animals lack lesions in the mid- and forebrain. Histologically, they are vasocentric and vary considerably in age. Acute lesions are dominated by perivenous lymphocytic cuffs that frequently track perforating venules from pia-arachnoid surfaces into adjacent white matter. The infiltrating lymphocytes frequently have large, activated nuclei as do endothelial cells lining the venules within the lymphocytic cuffs. The adjacent myelin is edematous, vesiculated and sparsely infiltrated with lymphocytes and macrophages. Occasionally, small focal hemorrhages are present in the edematous myelin. Subacute lesions are densely infiltrated with macrophages and lymphocytes and there is conspicuous myelin damage and phagocytosis of myelin debris by macrophages. Necrosis and microcystic spaces are dominant features in some subacute lesions. Chronic lesions consist of crisp plaques composed principally of fibrous astrocytes with foamy macrophages and occasional lymphocytes entrapped in the meshwork of glial fibers. The propensity for these lesions to occur only in white matter, their inflammatory demyelinating character and variable age are recognized features of MS. The acute fulminant course despite the histologic evidence of both acute and chronic lesions, the presence of necrosis in some subacute lesions and the predilection for the cerebellum and spinal cord are most consistent with the neuromyelitis optica or Devic's disease variation of MS (Prineas and McDonald, In: Graham D I, Lantos P L (eds.), Greenfield's Neuropathology, 6′ Edn. Oxford Univ. Press, New York, pp. 813-896, 1997; Raine, In: Davis R L, Robertson DM (eds.), Textbook of Neuropathology, 3^{rd} Edn. Williams and Wilkins, New York, pp. 627-714, 1997). While Devic's disease has clinical and pathologic features that are distinct from those of classical MS, individuals that survive the acute episode often progress to develop a disease course typical of classical MS, suggesting that the underlying process is similar or identical.

### EXAMPLE 28

### Isolation of Japanese Macaque Herpesvirus 17792

Attempts to isolate bacteria from cerebrospinal fluid and lesioned brain in these animals were negative in all cases, as were initial attempts to isolate viruses by inoculating cell cultures with homogenized brain. Thus, primary tissue explant cultures and cocultivation of leukocytes with target cells were used to enhance the probability of isolating highly cell-associated agents. Fresh tissue was dissected from visible spinal cord lesions identified during necropsy examination of Japanese macaque 17792 on May 22, 1995. Spinal cord tissue explant cultures were established from small spinal cord tissue fragments cultured in T-25 cultures flasks in Dulbecco's Modified Eagle Medium (DMEM, Gibco) supplemented with 20% fetal bovine serum, 1% L-glutamine and 1% penicillin-streptomycin-neomycin. Spinal cord explant cultures were fed by partial medium replacement every 7-10 days and passaged at confluence after 30 days in culture. Confluent monolayer explant cultures were rinsed in calcium- and magnesium-free phosphate-buffered saline, pH 7.4, digested with trypsin-EDTA at 37° C., washed with growth medium and seeded into T-25 culture flasks. First passage spinal cord explant cultures were fed by complete medium replacement at 5 to 7 day intervals and developed cytopathic effects (CPE) 7 days after passage. Cytopathic effects spread through the first passage spinal cord explant cultures after 14 days in culture and they were rapidly frozen in liquid N₂ and thawed. Cell-free supernatants were clarified by centrifugation, filtered through a 0.4511 membrane and stored frozen at −80° C.

Peripheral blood mononuclear cells (PBMC) were isopycnic gradient-purified (Ficoll-Paque, Pharmacia) from fresh ammonium heparin anti-coagulated blood obtained from Japanese macaque 17792 at necropsy. Gradient-purified PBMC were washed in calcium- and magnesium-free phosphate-buffered saline and 3×10⁶ PBMC were cocultured with 1×10⁵ primary rhesus macaque fibroblasts in T-25 culture flasks containing DMEM growth medium supplemented with 10% fetal bovine serum, 1% L-glutamine and 1% penicillin-streptomycin-neomycin. The PBMC-fibroblast cocultures were fed by partial medium replacement after 3 days in culture and passaged at confluence at day 9. Confluent PBMC-fibroblast cocultures were rinsed in calcium- and magnesium-free phosphate-buffered saline, pH 7.4, digested with trypsin-EDTA at 37° C., washed with growth medium and seeded into T-25 culture flasks. First passage PBMC-fibroblast cultures were fed by complete medium replacement at 3 to 5 day intervals and exhibited CPE 12 days after passage. Cytopathic effects spread through the first passage PBMC-fibroblast cultures after 15 days in culture and the cultures were rapidly frozen in liquid N₂ and thawed. Cell-free supernatants were clarified by centrifugation, filtered through a 0.45 micron membrane and stored frozen at −80° C.

To prepare virus stocks, cell-free filtered supernatants harvested from the spinal cord explant cultures and the PBMC-fibroblast cocultures were separately inoculated onto primary rhesus macaque fibroblast cultures. Fibroblast cultures inoculated with spinal cord explant supernatant and PBMC-fibroblast coculture supernatant developed CPE 5 days post-inoculation. The cultures were rapidly frozen in liquid N₂ and thawed and cell-free supernatants were clarified by centrifugation, filtered through a 0.45 micron membrane and stored frozen at −80° C. Using similar techniques, vero cells (American Type Culture Collection ATCC CCL-81) were shown to be susceptible to infection and suitable for propagation of virus.

For transmission electron microscopy, passage primary spinal cord explant cultures exhibiting CPE and fibroblast cultures developing CPE following inoculation with cell-free filtered supernatant from primary spinal cord explant cultures or PBMC-fibroblast cocultures were scraped free into medium, pelleted at 400×g, washed in calcium- and magnesium-free phosphate-buffered saline and suspended in cold ITO and Karnofsky's fixative (2.5% glutaraldehyde, 0.5% picric acid, 1.6% paraformaldehyde, 0.005% ruthenium red) in 0.1 M sodium cacodylate buffer, pH 7.4, for 2 hours. Fixed cells were washed in cacodylate buffer, post-fixed in 1% OsO₄ and 0.8% K₃Fe(Cn)₆ in cacodylate buffer for 1 hour, rinsed in distilled H₂O and pre-stained in 2% aqueous uranyl acetate for 1 hour. Fixed and pre-stained cells were dehydrated in a graded series of acetone imbedded in Epon 812 epoxy resin, polymerized at 60° C. and sectioned at 60 nm on an MT 5000 ultramicrotome. Copper grid mounted sections were stained with lead citrate and uranyl acetate and viewed in a Phillips 300 electron microscope. Numerous herpesvirus particles were observed in the primary spinal cord explant cells and in fibroblasts inoculated with cell-free filtered supernatant from primary spinal cord explant cultures and the PBMC-fibroblast cocultures.

### EXAMPLE 29

### Japanese Macaque Herpesvirus Epidemiology

To retrospectively and prospectively evaluate the relationship between Japanese macaque herpesvirus (JMHV) infection and encephalomyelitis, animals were identified in the troop that were actively infected with JM herpesvirus by cocultivating PBMC with rhesus macaque fibroblasts (see above), the seroconversion rate using an enzyme-linked immunoassay (ELISA) constructed with strain 17792 JM herpesvirus was established as follows. JM herpesvirus strain17792-infected primary rhesus macaque fibroblasts were solubilized with 0.5% Nonidet P40 and 1% sodium deoxycholate in phosphate-buffered saline, pH 7.2, and clarified in a Beckman SW28 rotor at 23,500 rpm for 1 hour at 4° C. The clarified supernatant was used as antigen for coating 96-well Maxisorp plates (2 ng/well) (Nunc, Roskilde, Denmark). ELISAs were then performed essentially as described (Kodama et al., AIDS Res Hum Retroviruses 5:337-343 1989).

In 1995-1996, 70-75% of the infected animals were less than 7 years of age. Analysis of samples from these animals revealed that 16% of the animals in the troop had culturable simian spumaretrovirus (simian foamy virus; SFV) and 8% of the animals in the troop had JM herpesvirus in peripheral blood leukocytes (PBL). Virus isolation from PBL was repeated for 199 animals in 1997; SFV was recovered from 11 (5.5%), the JM herpesvirus was recovered from 120 (60.3%), and 5 (0.25%) had dual infections.

The number of JMHV-positive animals decreased with age; 97% of animals less than 1 year of age and 27% of animals greater than 10 years of age were isolation-positive. One hundred percent of the animals greater than 300 days of age were seropositive for JM herpesvirus. Eight animals developed demyelinating encephalomyelitis in 1995-1997 and all were seropositive for the JM herpesvirus, and 6 of 8 were animals previously identified as JM herpesvirus isolation-positive. Two of the JM herpesvirus-infected animals were co-infected with SFV. SFV was isolated from 1 of 2 JM herpesvirus isolation-negative animals and no virus was isolated in the remaining animal. Brain and spinal cord were available for virus isolation from 7 of the 8 animals with demyelinating encephalomyelitis; MRV was isolated from lesioned brain or spinal cord explant cultures from 3, both MRV and SFV were recovered from 2, 1 yielded only SFV and virus was not isolated from 1. Virus was not recovered from explant cultures of normal appearing white matter from the cerebrum in any animal.

### EXAMPLE 30

### JMHV Sequence

The Japanese macaque virus isolate was isolated from a lesion that was minced and co-cultured with primary rhesus fibroblasts. The isolate was then cloned by limiting dilution and a stock of virus generated from this clone. Total cellular DNA was harvested from virus infected cells and the DNA subjected to degenerate PCR for viral DNA polymerase, exactly as described above for RRV. Once confirmed, a cosmid library of this virus was made from purified viral DNA (as described for RRV) and then a portion of the protein genes was cloned and sequenced.

Cosmid 3 is an isolate from a Japanese macaque virus library. It contains about 35,000 bp of viral sequence and 7000 bp of vector. The viral sequence was sub-cloned using Eco R1. The fragments below are these EcoR1 fragments. The terms T7 and SP6 are the names of universally used primers for sequencing—they sequence opposing ends of the viral fragment. Because the lengths of the viral inserts vary, and may be quite large, the open reading frame found at one end of the insert may or may not differ from the open reading frame found at the other end of the insert. Protein sequences are in the standard single letter code.

Cosmid 3 Fragment 7 T7 Sequence
Orf 21 - Thymidine kinase
DNA sequence

(SEQ ID NO:2)

GAATTCATATTGCATATGTCCTCTATGGACACAGTTTGTACACACAACTG
AACCATTTGTTCCGGTGTGAGATATTGCATCATCACCCACGTACAGAACA
CGGCGTGGTACGCCCACGCTACCTGTCGCAAGTAGTTTTGCGTGATTCCT
TTTTCTTCCTTGCGGCCTCGACTTTGAATGCGCCTGTGCGCCTCCGAACT
GTTGAGCGTTAGCAAAACGACCACATCACCGTCGTGTGCCGAGAAAAGAG
ATAGGATTTGAAACAAATGATCCGGCGTTAGCCTGTTGTACTTTACGTGA
ACCAGCGGAAAAACAACGGTGGCGGACAGTAGGTGTCTGTCAAAAACAAT
CCAATTCGCTGGCTTTGTGCTACCGTTTCCCACTAGCCACGGTTGCAGGT
TGCGACCGATGCCGGCGGCCGTTGCA
Deduced amino acid sequence

(SEQ ID NO:3)

ATAAGIGRNLQPWLVGNGSTKPANWIVFDRHLLSATVVFPLVHVKYNRLT
PDHLFQILSLFSAHDGDVVVLLTLNSSEAHRRIQSRGRKEEKGITQNYLR
QVAWAYHAVFCTWVMMQYLTPEQMVQLCVQTVSIEDICNMN
Cosmid 3 Fragment 7 SP6 sequence
Orf 21 - Thymidine kinase
DNA sequence

(SEQ ID NO:4)

GAATTCAATGACTGGCATGGTGCCGCAGGAAAACGTCTTAAGCTGTCCAG
AGCCCATGAAATTTTGGACGTGTGTTTATTCAAATTGCCTTAAAGAGCAG
CGCAGCATAGTTAAGCAAGGCACCCACGGGAAATCGATCACTTCTGCTCG
CGTATACGCGTGTCAGAGCAAGTTTGCGCTACCTTTTCGTGCAACGGCCG
CCGGCATCGGTCGCAACCTGCAACCGTGGCTAGTGGGAAACGGTAGCACA
AAGCCAGCGAATTGGATTGTTTTTGACAGACACCTACTGTCCGCCACCGT
TGTTTTTCCGCTGGTTCACGTAAAGTACAACAGGCTAACGCCGGATCATT
TGTTTCAAATCCTATCTCTTTTCTCGGCACACGACGGTGATGTGGTCGTT
TTGCTAACGCTCAACAGTTCGGAGGCGCACAGGCGCATTCAAAGTCGAGG
CCGCAAGGAAGAAAAAGGAATCACGCAAAACTACTTGCGACAGGTAGCGT
GGGCGTACCACGCCGTGTTCTTGT
Deduced amino acid sequence

(SEQ ID NO:5)

NSMTGMVPQENVLSCPEPMKFWTCVYSNCLKEQRSIVKQGTHGKSITSAR
VYACQSKFALPFRATAAGIGRNLQPWLVGNGSTKPANWIVFDRHLLSATV
VFPLVHVKYNRLTPDHLFQILSLFSAHDGDVVVLLTLNSSEAHRRIQSRG
RKEEKQITQNYLRQVAWAYHAVFL
Cosmid 3 Fragment 5 T7
No ORF associated, but highly similar to the DNA
sequence found in and to the right of RRV repeat
unit rDL-B1.
DNA sequence

(SEQ ID NO:6)

AGCGAGTCMGTGAGCGAGGAAGCGGAAGAGCGCCCAATACGCAAACCGCC
TCTCCCCGCGCGTTGGCCGATTCATTAATGCAGGTTAACCTGGSKTRTCG
AAATTAATACGACTCACTATAGGGAGACCGGCCTCGAGCAGCTGAAGCTT
GCATGCCTGCAGGTCGACTCTAGAGGATCCCCGGGTACCGAGCTCGAATT
CCAGACAGGTAAGATTTTAGCTCCTAATGTTTGCCTTGCCGCCTAGCTCC
TAATGTTTGCCTTGCCGCCTAGCTCCTAATGTTTGCCTTGCCGCCTAGCT
CCTAATGTTTGCCTTGCCGCCTAGCTCCTAATGKTTGCCTTGCCGCCTAG
CTCCTAATGTTTGCCTTGCCGCCTAGCTCCTAATGTTTGCCTTGCCGCCT
AGCTCCTAATGTTTGCCTTGCCGCCTAGCTCCTAATGTTTGCCTTGCCGC
CTAGCTCCTAATGTTTGCCCACGTTTATGTTTAAGCACACTAAAATTTAA
AAACGTTTGTGTTGGTTTTTATGACCGGCTTGGTACAAAACCTGCTGGTG
ATTTTTTACCCAACAAAAATAATAAATAAAAAAGTTAAAACTTATTTCTG
TTGTCTGTGGTAATTGCGTTCCCCGAGGGT
Cosmid 3 Fragment 5 SP6
Orf 17 - a capsid protein
DNA sequence

(SEQ ID NO:7)

GAATTCGCAGCCCCTCTTGAAACACTGATGGCCAAGGCGATCGACGCCGG
ATTTATCCGAGATCGCACAGACCTGCTCAAAACAGATAAAGGCGTGGCCA
GAGTCGCGAGAAGTACGTATTTAAAGGCCAGCCAGTCTCCCTCCTCTCAG
CACGGCGGTAACCGCGACACCCAAACCATGAGCGCCCTCCCGGATGACAA
CATCACCATTCCCAAGAGCACCTTTCTAACCATGGTGCAGAGCAGTCTCG
ATCACATGCGCAACCAGGGCCAACGCGCGTACGTTTCCGCGCCACCCTCG
ATGCCGGCAACGGCGGCGTATCCCTCGTGGATACCGCCACCAGAACTGAC
CGTCCCGTCGTACGCGCCGCCCGTAGCGCCACCGTTCCCTTTTCAGTCGG
CGTTTG
Deduced amino acid sequence

(SEQ ID NO:8)

EFAAPLETMAKAIDAGFIRDRTDLLKTDKGVARVARSTYLKASQSPSSQH
GGNRDTQTMSALPDDNITIPKSTFLTMVQSSLDHMRNQGQRAYVSAPPSM
PATAAYPSWIPPPELTVPSYAPPVAPPFPFQSAF
Cosmid 3 Fragment 3 T7
Orf 21 Thymidine kinase
DNA sequence, includes frameshift mutations

(SEQ ID NO:9)

GRGCGCAGCGAGTCAGTGAGCGAGGNAAGCGGAAGAGCGCCCAATACGCA
AACCGCCTCTCCCCGCGCGTTGGCCGATTCATTAATGCAGGTTAACCTGG
STTATCGAAATTAATACGACTCACTATAGGGAGACCGGCCTCGAGCAGCT
GAAGCTTGCATGCCTGCAGGTCGACTCTAGAGGATCCCCGGTACCGAGCT
CGAATTCCAGATTGACTCATCGGTTTCTAACCCTAACAAAGTTGCATGAA
CAGAGTATGATACCAATGGTGGTAGAAATGTTAGCAGCGGTTAAAGAACA
CGTGACCTTAATGGAGGTCTGTTTGGGCCTCTTTAAAGAGCTACGAAAGC
TTCAGATTTTAATTGTTGACGCGGGAGAACATTTAGATGATACGTGTGGC
CTTTGGGGAAATATTTATGGGCAGGTAATGTCAAATGAGGCTATTAAACC
ACGAGCCGTGAACTGGCCAGCCCTTGAAAGCTACATTCAAACGCTAACCA
GCTTGGAAAGCAATGCAGCCAATTAACCACGGTTTGTGTTTGTTGGTAAT
TACATTCTCTGTAATTAATGGATATGAATATAATGAAGAAAATGTACCTG
GACTTGAAATAGTTCTTTTTACCCCTGCC
Deduced amino acid sequence - region between
frameshifts

(SEQ ID NO:10)

SLHACRSTLEDPRVPSSNSRLTHRFLTLTKLHEQSMIPMVVEMLAAVKEH
VTLMEVCLGLFKELRKLQILIVDAGEHLDDTCGLWGNIYGQVMSNEAIKP
RAVNWPALESYIQTLTSLESNAAN
Cosmid 3 Fragment 3 SP6
Orf 24 Unknown function
DNA sequence

(SEQ ID NO:11)

GAATTCAATACGCTCCAAAAGATAGAGATCACAATTTTATTTTTGACGCC
AATCAAAACCCAGATCGACATAAACAAGTGCACCACGACCACCAGACCGA
ACCGCTTCCAGACATGTTCGATCCAGTAAAGCACCTAAGTTTACACAACT
TCAAAATCTCAGTTTTCAATACCAATATGGTAATTAATACCAAAATCACG
TGCCGGTCTCTCACCGGTACCTTCGAGTCAATCATCGACATCCCCAGGCT
CACAAATAACTTTGTAATGAAAAAATTCTCCGTGAAAGAACCGTCATTCA
CTGTGAGCGTGTTCTACTCCGACAACCTGTGCAACGGGGCTGCGATTAAC
GTTAACATAAGCGGGGACATGCTGCACTTTATGTTCGCTATGGGAAACCT
GAGGTGCTTTTTGCCGGTGAAGCACATTTTCCCGGTTTCGATTGCGAACT
GGAACTCTACGTTAGACCTCCACGGGCTCGAAAACCAATACATAGTTAGA
CGGGGGCGGCGAGACGTTTTCTGG
Deduced amino acid sequence

(SEQ ID NO:12)

IQYAPKDRDHNFIFDANQNPDRHKQVHHDHQTEPLPDMFDPVKHLSHNFK
ISVFNTNMVINTKITCRSLTGTFESIIDIPRLTNNFVMKKFSVKEPSFTV
SVFYSDNLCNGAAINVNISGDMLHFMFAMGNLRCFLPVKHIFPVSIANWN
STLDLHGLENQYIVRRGRRDVFW
Cosmid 3 Fragment 1 T7
Orf 7 Transport protein
DNA sequence

(SEQ ID NO:13)

GATCCCATAGAGTCCCTGTTCTGCGGCGGTCTTTTTAACTCTATAGACGA
TACCATTAACGCACTGAGCCGGGATTGTTCGGTAACGTTCTTTCAACAGG
CAAACTATACCAACGTCATGCGAAAACAAAACGAGCTGTTTACCAGACTC
AACAGCATATTGTGTCAGGGGAGCGCGGGATCGCAAAAACCGGCCACCCC
CTCGGAGCCACGGACCGCCACCGTCGCGGCGACCGCGGCAAGCGACGTCA
TTAAAGACGCACAATATCGCAAAGAACAGTACATGAAAAAGGTGGCCAGG
GACGGCTTTAAAAAACTAACAGAGTGTCTGCAGACGCAAAGCGCGGTGTT
GGCAAACGCGCTCTGCATGCGCGTATGGGGGGGCGTCGCATACGGCGA
Deduced amino acid sequence

(SEQ ID NO:14)

DPIESLFCGGLFNSIDDTINALSRDCSVTFFQQANYTNVMRKQNELFTRL
NSILCQGSAGSQKPATSEPRTATVAATAASDVIKDAQYRKEQYMKKVARD
GFKKLTECLQTQSAVLANALCMTVWGGVAYG
Cosmid 3 Fragment 1 SP6
No ORF. Similar to sequence to the left of the
RRV repeat rDL-B1
DNA sequence

(SEQ ID NO:15)

GAATTCACACCCGAACAAATCTAAAAGCAAAATTACACAAATTAACAAAA
ATACAACTATAACACAGTTAAAAAATTCAATACATAATTACAATGGTAAA
ATTCACGCGTACATAATTACAATTTTAAATTCAATTTAAAAATTCACGTG
TACACAATTACAAAATTCACACAATTTTAAAATCCTTTTAACCATAAAAT
TAGAACGGCACTGTATATATGCAGCGTATTGCAAACTGGCAGCTTTGTGT
TAAAGACCACGGCATTAAATTTTAAATTCGCCAGGTCGCCAAAAGTACTG
GTTACGTTAGTTCATCTAAAAGCTTAACGTGCTTGTGCTTAAATTTACGC
CGTGCCATTGCTCCATTTTAAATTAAAATTTAAAATGTAGTGCGAGCGAG
CTAGAAACAGAAACCGCGACGCGGGGCAGGTTGCGTAGGTTCGCCGTTTT
TGGCTCCAGTGACAATCCAAAAGCTGCGGTTTACGAGCCATTGTTTTTGT
CAACCACTAAACCGAAAGCATGCG
Large EcoR1 fragments were then further reduced
using Kpn 1
Cosmid 3 EcoR1 Fragment 1 Kpn 1 fragment 1 T7
Orf 10 Unknown protein
DNA sequence

(SEQ ID NO:16)

GGTACCCCCACGACTATAAAAAGAGACACCCTGGTGGCCGCCGCCGCGCC
CTGCCCCGTGGTGCGAGTAAGCTCCGCCGACGACGCGCCGCGGGATCTCG
TCGCGTCACCAGACACCGGGGCGCTCTCCATTGACGCGTTCACAATCCCG
GTCGGTCTCCCCGGGGTGGTCTCGGCGGAGTGTCACGTGTCTATGCGCGA
CAACGGGGTCCACGAACGCATGAGCCATTAACGGCGACAATGGGAACACC
GGTGCGTTTCTTTCGCGGCGAGTGGCAAACCTCGAGTCTGGTGGACAACG
GCACGCCACGGTACAGCTCCCTGGTGTGGGCCGCCACTATCCACGACGGC
TACCTGACACTGGTGAACAGGTCGGAGCTGTGCGTCACGGAGCGGTCTCC
GTGTCTGCCGGCGTGCCCCAGCATCGGGAGACTGGTCG
Deduced amino acid sequence C-terminus of protein

(SEQ ID NO:17)

GTPTTIKRDTLVAAAAPCPVVRVSSADDAPRDLVASPDTGALSIDAFTIP
VGLPGVVSAECHVSMRDNGVHERMSH
Cosmid 3 EcoR1 Fragment 1 Kpn 1 fragment 2 SP6
Orf 9 DNA polymerase
DNA sequence

(SEQ ID NO:18)

GGTACCGAGCCGCTTCCAGACGGACATTATTCCGAGCGGAACCGTGCTCA
AGCTCCTGGGGCGAACCGAGAACGGCACCAGCGTGTGCGTGAACGTGTTC
CGTCAACAGGTATATTTCTACGCGAAGGTCCCAGCCGGCGTCAACGTCAC
CCACGTCCTCCAGCAGGCCCTCAAGAACACCGCCGGCAGGGCCGCGTGCG
GCTTCTCGACCAGACGAGTAACCAAAAAAATTCTCAAAACGTACGACGTC
GCGGAGCATCCCGTCACTGAAATCACGCTATCGTCCGGTTCCATGCTCTC
GACCCTCAGCGACCGCCTCGTCGCGTGCGGGTGCGAGGTGTTCGAGTCAA
ACGTGGACGCCGTTCGCCGGTTCGTCCTGGATCACGGGTTTACCACGTTC
GGGTGGTACTCGTGCGCGCGTGCCACGCCCCGCCTAGCGGNCAGAGATGC
CAGAACGGCCCTGGAGTTTGACTGCAGCTGGGAGGACCTCAGCGTTC
Deduced amino acid sequence

(SEQ ID NO:19)

VPSRFQTDIIPSGTVLKLLGRTENGTSVCNVVFRQQVYFYAKVPAGVNVT
HVLQQALKNTAGRAACGFSTRRVTKKILKTYDVAEHPVTEITLSSGSMLS
TLSDRLVACGCEVFESNVDAVRRFVLDHGFTTFGWYSCARATPRLAXRDA
RTALEFDCSWEDLSV
Cosmid 3 EcoR1 Fragment 1 Kpn 1 fragment 2 T7
Orf 10 Unknown protein
DNA sequence

(SEQ ID NO:20)

GGTACCGTTGGGGTTGAATAGGCGCAGGTGTGTTGTGCAACCGGAAAGCA
AAAGTGTTGGGGTGACGATGAATAGGGGCTCCGGTGCCACGCCCGCAACT
AGAAACGCCAGATCGCCCCGAGCGGCGCTCCCTGTTGGAAAGATTAACTG
AACCCGGACTTCCGCGGAGCTTCCAGGTTGGATGGTTTTTCGGCGTCGGC
GTACACCCCCAATACGCGGCCGCCGGTGTGGGTGGGCAGGACGGGTCCGG
ACTGGCCCAAGACTATCTCGGCGTTGGGGTCCCCCGGAGCGTAAATGATT
TTCATTTGCGCCG
Deduced amino acid sequence

(SEQ ID NO:21)

AQMKIIYAPGDPNAEIVLGQSGPVLPTHTGGRVLGVYADAEKTIQPGSSA
EVRVQLIFPTGSAARGDLAFLVAGVAPEPLFIVTPTLLLSGCTT
HLRLFNPNGT
Cosmid 3 EcoR1 Fragment 1 Kpn fragment 3 SP6
Orf 8 Glycoprotein B
DNA sequence

(SEQ ID NO:22)

GGTACCGCAAGGTGGCCACCTCGGTAACCGTCTATCGAGGGTGGACCGAG
ACCGCCGTGACCGGTAAGCAAGAGGTCATTCGACCGGTGCCGCAGTACGA
GATTAACCACATGGACACGACCTACCAGTGTTTCAGCTCCATGCGCGTGA
ACGTTAACGGCATCGAAAACACCTACACGGACAGGGACTTCACTAACCAG
ACCGTGTTCCTGCAACCGGTCGAGGGGCTCACGGATAACATTCAGCGATA
CTTCAGTCAGCCGGTGCTGTACACGACACCGGGATGGTTTCCTGGAATCT
ACAGGGTCCGAACCACGGTCAACTGCGAGATCGTGGACATGATCGCGCGT
TCGGCGGAACCGTACTCTTACTTTGTCACCGCCCTGGGAGACACGGTAGA
GGTATCGCCGTTCTGCTTAAACGACTCGACGTGCTCCGTCGCTGATAAAG
CCGAAAACGGCCTCGGCGTGCGCGTGCTTACAAATTACACCATTGTTGAC
TTCGCTACCCGCACGCCCACCACCGAAACGCGAGTTTTCGCAGACTCGGG
AGAATACACCGTATCGTGGAAGGCGGAAGACCCTAAGTCGGCAGTCTGTG
CGCTGACGCTCTGGAAAACCTTTCCCAGGGCCATACAGACGACGCACGAA
AGCCAGCTACCACTTTGTGGCCAACGACGTG
Deduced amino acid sequence

(SEQ ID NO:23)

YRKVATSVTVYRGWTETAVTGKQEVIRPVPQYEINHMDTTYQCFSSMRVN
VNGIENTYTDRDFTNQTVFLQPVEGLTDNIQRYFSQPVLYTTPGWFPGIY
RVRTTVNCEIVDMIARSAEPYSYFVTALGDTVEVSPFCLNDSTCSVADKA
ENGLGVRVLTNYTIVDFATRTPTTETRVFADSGEYTVSWKAEKPKSAVCA
LTLWKTFPRAIQTTHESQLPCGQRR
Cosmid 3 EcoR1 Fragment 1 Kpn 1 fragment 3 T7
Orf 9 DNA polymerase
DNA sequence

(SEQ ID NO:24)

GGTACCTCGGCGCACCTGTCCGCCGCGTACGTCGTTTCAACGACGTCGTA
CGCGTGAAACGTAATGGATTGGTCTTGGGGGTCCGGGACGACTGGCCGTT
GGCCGCGCGCCGTCCACATGGACCGTTCGTGGGCGAGCAGCACGTCTCCG
CGCGCGCCGTTTTCAAAGTACGTCGGCGGGAACGGGATCGTGACCGGGAT
CATCCCGCCTGCCCCCGGCGTTCGGAGGCAGGCGGGAATGAGCCTGCAGA
CGTCTGGTGGCGGCTGAACGGCGCCGGCGGGGGCGGGCGCATCGGTGCAT
TTGTGTGAGGGGGGGCGTGGTCCTCGAGGACCCAGGTACGGGTTAAAAAA
ATCCATGCTCGATACAGAACGCTGACGCCCGGGGGCGCC
Deduced amino acid sequence

(SEQ ID NO:25)

GAPGRQRSVSSMDFFNPYLGPRGPRPPSHKCTDAPAPAGAVQPPPDVCRL
IPACLRTPGAGGMIPVTIPFPPTYFENGARGDVLLAHERSMWTARGQRPV
VPDPQDQSITFHAYDVVETTYAADRCAEV
Cosmid 3 EcoR1 Fragment 1 Kpn 1 fragment 4 T7
Orf 8 Glycopeotein B
DNA sequence

(SEQ ID NO:26)

GGTACCGCCTAACCTTAAAGATGTGGGGGACGATATTTTTTTTAAACACC
ATTAGAATGCCTTCTTGGTGCGTTTTGNCCTCGGTGCCAGGACACGTCTT
TTCCAGGTTAAACCTGAAGAGTTCGCCGGTGGCCGAGGCGCTGCACACGC
GAAACTTGAACGCCTCGGCCCTGGGTGGGTTTTCGGGAGGTGTGGGCGTC
GACGGGCCCGGCGTGGACTTTGCAGTGGTGGTCACGCCCTTTGGGGTGGT
GACGTTTTCGCCAACCGGCGCGCCAATCGCGATTATCACGACCCACGCCC
GCAGCAGACGACGCGTCCGGTTAGGTATCA
Deduced amino acid sequence

(SEQ ID NO:27)

IPNRTRRLLRAWVVIIAIGAPVGENVTTPKGVTTTAKSTPGPSTPTPPEN
PPRAEAFKFRVCSASATGELFRFNLEKTCPGTEXKTHQEGILMVFKKNIV
PHIFKVRRY

The sequence of the Japanese macaque gamma2 herpesvirus were compared with the rhesus macaque gamma2 herpesvirus 17577. The results are shown in FIGS. 12-25. Based on a comparison of 2328 amino acid residues from 14 independent regions (average length of 166+71 amino acids), the percent similarity between the two viruses at the protein level was found to be 94.12%. The range among the 14 regions was 75.652% to 99.422%. The lowest identity was found in the MIP region of the two genomes, while the highest identity was found in ORF 21. The calculations are shown below in Table 3:

TABLE 3
Calculation of % Similarity and % Identity

| Gene | Length of | | | | |
|---|---|---|---|---|---|
| Region | Alignment | % similarity | # similar | % identity | # identical |
| IL6 | 207 | 94.203% | 195 | 91.304% | 178 |
| MIP | 120 | 80.870% | 97 | 75.652% | 73 |
| Orf 07 | 226 | 98.230% | 222 | 98.230% | 218 |
| Orf 08 | 109 | 94.495% | 103 | 94.495% | 97 |
| Orf 08 | 226 | 97.235% | 220 | 96.774% | 213 |
| Orf 09 | 120 | 94.915% | 114 | 93.220% | 106 |
| Orf 09 | 165 | 98.182% | 162 | 96.364% | 156 |
| Orf 10 | 104 | 94.231% | 98 | 93.269% | 91 |
| Orf 10 | 76 | 94.737% | 72 | 93.421% | 67 |
| Orf 17 | 135 | 94.074% | 127 | 93.333% | 119 |
| Orf 21 | 174 | 99.422% | 173 | 99.422% | 172 |
| Orf 24 | 174 | 95.977% | 167 | 94.253% | 157 |
| Orf 29b | 140 | 98.571% | 138 | 98.571% | 136 |
| TS | 352 | 93.994% | 331 | 93.093% | 308 |

These results were then used to estimate the % similarity and identity across the viral genome, as shown below in Table 4:

TABLE 4
Estimated % Similarity and Identity Across Entire Viral Genome

| | similarity | identity |
|---|---|---|
| Mean | 95.011% | 93.716% |
| Std. Error | 1.199% | 1.532% |
| n | 14 | 14 |
| Avg. length | 166.3 | 166.3 |
| Std dev | 70.5 | 70.5 |

Over the entire genome, the JMHV and the RRV17577 are estimated to have 95% similarity. The present invention includes the entire MHV genome, and each of the peptides (including variants and fragments thereof), as well as DNA sequence which encodes them, such as the OFRs set forth in this specification.

### EXAMPLE 31

### Reactivity of Human MS Patient Serum to Japanese Macaque

To determine if there was evidence for JM herpesvirus or a closely related virus in MS patients, serum antibodies were measured that reacted with strain 17792 JM herpesvirus. A panel of 15 coded human serum samples that contained serum from normal control and MS patients were used. The results of this study are summarized in Table 5. Six of 15 samples yielded positive O.D. values (>0.150), 5 of the 6 positive samples were from MS patients. The 9 samples with O.D. values of <0.150 included 5 MS patients and 4 controls. However, when the O.D. values were ranked, 8 of 10 samples with the highest O.D. values segregated to MS patients, suggesting that some MS patients have a humoral immune response to JM herpesvirus or a related human virus.

The present invention includes the antibodies that recognize JM herpesvirus, as well as antigens recognized by these antibodies. Thus viral antigen (such as a viral antigens expressed in humans) which are recognized by an anti-JMHV antibody, are included herein. Examples of such antigens iclude antigens encoded by a human virus that is associated with the development of MS. A human viral antigen that is encoded by a human virus can have, for example, 80%, 90%, 95%, or 98% sequence homology with a JMHV antigen encoded by the virus deposited as ATCC accession no. PTA-1884.

Variants of the JMHV include variants that infect humans, and which are associated with the development of MS, or pathological lesions (such as demyelination) that are associated with MS in humans. Such variants can be isolated using the techniques disclosed in this specification for obtaining viruses with similar nucleic acid sequences, or the viruses can be recognized by the specific binding of simian antibodies against JMHV to the human variant, as illustrated in Table 5.

TABLE 5
MS and Control Patient Serum Reactivity
to Japanese Macaque Herpesvirus

| Rank | ID No. | O.D. Value | Status |
|---|---|---|---|
| 1 | 4 | 0.047 | MS |
| 2 | 11 | 0.052 | Control |
| 3 | 8 | 0.065 | Control |
| 4 | 1 | 0.082 | Control |
| 5 | 3 | 0.106 | MS |
| 6 | 2 | 0.112 | Control |
| 7 | 10 | 0.112 | MS |
| 8 | 7 | 0.120 | MS |
| 9 | 5 | 0.122 | MS |
| 10 | 12 | 0.169 | MS |
| 11 | 15 | 0.214 | MS |
| 12 | 14 | 0.253 | MS |
| 13 | 13 | 0.415 | MS |
| 14 | 9 | 0.419 | Control |
| 15 | 6 | 0.430 | MS |

Having illustrated and described the principles of cloning the RRV and JMHV nucleic acid molecules, cDNA, proteins encoded by the cDNA, and modes of use of these biological molecules, it should be apparent to one skilled in the art that the invention can be modified in arrangement and detail without departing from such principles. In view of the many possible embodiments to which the principles of our invention may be applied, it should be recognized that the illustrated embodiments are only examples of the invention and should not be taken as a limitation on the scope of the invention. Rather, the scope of the invention is in accord with the following claims. We therefore claim as our invention all that comes within the scope and spirit of these claims.

1. An isolated virus (Japanese Macaque Virus, JMHV) as deposited with ATCC as deposit accession number PTA-1884.

2. A host cell infected with the virus of claim 1.

3. A purified polypeptide comprising an amino acid sequence that has at least 95% sequence identity to an amino acid sequence as set forth as SEQ ID NO:3, SEQ ID NO:5, SEQ ID NO:8, SEQ ID NO:10, SEQ ID NO:12, SEQ ID NO:14, SEQ ID NO:17, SEQ ID NO:19, SEQ ID NO:21, SEQ ID NO:23, SEQ ID NO:25, and SEQ ID NO:27.

4. A purified polypeptide of claim 3, wherein the polypeptide has at least 98% sequence identity to an amino acid sequence as set forth as SEQ ID NO:3, SEQ ID NO:5, SEQ ID NO:8, SEQ ID NO:10, SEQ ID NO:12, SEQ ID NO:14, SEQ ID NO:17, SEQ ID NO:19, SEQ ID NO:21, SEQ ID NO:23, SEQ ID NO:25, and SEQ ID NO:27.

5. The purified polypeptide of claim 2, wherein the polypeptide has an amino acid sequence as set forth as SEQ ID NO:3, SEQ ID NO:5, SEQ ID NO:8, SEQ ID NO:10, SEQ ID NO:12, SEQ ID NO:14, SEQ ID NO:17, SEQ ID NO:19, SEQ ID NO:21, SEQ ID NO:23, SEQ ID NO:25, and SEQ ID NO:27.

6. An antibody that binds to the purified polypeptide of claim 2.

7. An isolated nucleic acid sequence encoding the polypeptide of claim 3.

8. An isolated nucleic acid sequence comprising a sequence having at least 95% sequence identity to one of SEQ ID NO: 2, SEQ ID NO: 4, SEQ ID NO: 6, SEQ ID NO: 7, SEQ ID NO: 9, SEQ ID NO:11, SEQ ID NO:13, SEQ ID NO:15, SEQ ID NO:16, SEQ ID NO: 18, SEQ ID NO: 20, SEQ ID NO: 22, SEQ ID NO: 24, or SEQ ID NO: 26.

9. The isolated nucleic acid sequence of claim 8, wherein the nucleic acid comprises a sequence as set forth as SEQ ID NO: 2, SEQ ID NO: 4, SEQ ID NO: 6, SEQ ID NO: 7, SEQ ID NO: 9, SEQ ID NO: 11, SEQ ID NO: 13, SEQ ID NO: 15, SEQ ID NO: 16, SEQ ID NO: 18, SEQ ID NO: 20, SEQ ID NO: 22, SEQ ID NO: 24, or SEQ ID NO: 26.

10. An isolated nucleic acid sequence comprising a promoter operably linked to the nucleic acid sequence of claim 7.

11. A vector comprising the nucleic acid sequence of claim 7.

12. A host cell transformed with the vector of claim 11.

13. The vector of claim 11, wherein the vector is a viral vector.

14. A viral particle comprising the viral vector of claim 12.

15. An isolated nucleic acid molecule that hybridizes under highly stringent conditions with a nucleic acid molecule encoding the polypeptide of claim 5.

16. A method for testing the efficacy of a drug in the treatment of a condition associated with infection with Japanese macaque herpesvirus (JMHV), the method comprising:
(a) administering the drug to a non-human primate infected with a Japanese macaque herpesvirus (JMHV); and
(b) observing the non-human primate to determine if the drug prevents or reduces the presentation of one or more symptoms associated with Japanese macaque herpesvirus (JMHV) infection.

17. The method of claim 16, wherein the drug is a drug used to treat multiple sclerosis.

18. The method of claim 16, wherein the non-human primate is a Japanese macaque monkey.

19. A method for testing the efficacy of a candidate vaccine against Japanese macaque herpesvirus (JMHV) infection, or conditions associated with Japanese macaque herpesvirus (JMHV) infection, the method comprising:
(a) administering the vaccine to a non-human primate susceptible to infection with the Japanese macaque herpesvirus (JMHV);
(b) inoculating the subject with the Japanese macaque herpesvirus (JMHV), and
(c) observing the non-human primate to determine if the vaccine prevents or reduces an incidence of Japanese macaque herpesvirus (JMHV) infection or a symptom associated with Japanese macaque herpesvirus (JMHV) infection.

20. The method of claim 19, wherein the non-human primate is a Japanese macaque monkey.

21. The method of claim 19, wherein the symptom is associated with multiple sclerosis.

22. A method of detecting the presence of Japanese macaque herpesvirus (JMHV) or a related virus in a biological specimen, comprising:
(1) amplifying by polymerase chain reaction a Japanese macaque herpesvirus (JMHV) nucleic acid sequence, if such sequence is present in the sample, using two or more oligonucleotide primers comprising 20 contiguous nucleotides of the nucleic acid sequence of claim 7; and
(2) determining whether an amplified sequence is present.

23. The method of claim 22, wherein the step of determining whether an amplified sequence is present comprises one or more of:
(a) electrophoresis and staining of the amplified sequence; or
(b) hybridization to a labeled probe of the amplified sequence.

24. The method of claim 23, wherein the amplified sequence is detected by hybridization to a labeled probe.

25. The method of claim 24, wherein the probe comprises a detectable non-isotopic label chosen from the group consisting of:
a fluorescent molecule;
a chemiluminescent molecule;
an enzyme;
a co-factor;
an enzyme substrate; and
a hapten.

26. The method of claim 23, wherein the biological specimen is a primate specimen.

27. The method of claim 26, wherein the primate specimen is a non-human primate specimen.

28. A method of detecting the presence of Japanese macaque herpesvirus (JMHV) in a biological specimen, comprising:
exposing the biological specimen to a probe that hybridizes to a Japanese macaque herpesvirus (JMHV) nucleic acid sequence of claim 7, if the sequence is present in the sample to form a hybridization complex; and
determining whether the hybridization complex is present.

29. The method of claim 28, wherein the biological specimen is a primate specimen.

30. The method of claim 29, wherein the primate specimen is a non-human primate specimen.

31. A method of obtaining a Japanese macaque herpesvirus (JMHV)-related nucleic acid sequence comprising:
(a) amplifying a for rhesus rhadinovirus (RRV) nucleic acid sequence using two or more oligonucleotide primers comprising 20 contiguous nucleotides of the nucleic acid sequence of claim 7 to generate a product amplified nucleic acid sequence; and
(b) detecting the presence of a product amplified nucleic acid sequence; and
(c) isolating the product amplified nucleic acid sequence.

32. The method of claim 31, wherein amplifying the for rhesus rhadinovirus (RRV) nucleic acid sequence comprises polymerase chain reaction amplification.

33. The method of claim 31, wherein the detection of an amplified sequence is present comprises detecting the amplified sequence using one or more of:
(a) electrophoresis and staining of the amplified nucleic acid sequence;
(b) hybridization of a labeled probe to the amplified nucleic acid sequence; or
(c) sequencing the amplified nucleic acid sequence.

34. The method of claim 33, wherein the amplified sequence is detected by hybridization.

35. A method of obtaining a Japanese macaque herpesvirus (JMHV)-related nucleic acid sequence from a sample comprising
(a) contacting a nucleic acid of the sample with a probe or primer that hybridizes under moderately stringent hybridization conditions to the nucleic acid of the sample, wherein the probe or primer comprises 15 consecutive nucleotides of the nucleic acid sequence of claim 7; and
(b) isolating the nucleic acid of the sample to which the probe hybridizes.

36. The method of claim 35, further comprising sequencing the nucleic acid of the sample to which the probe hybridizes.

37. A method of detecting the presence of Japanese macaque herpesvirus (JMHV) in a biological specimen, comprising:
contacting the biological specimen with the antibody of claim 6,
detecting binding of the antibody to the biological specimen or a component thereof, wherein binding of the antibody to the biological specimen indicates the presence of a Japanese macaque herpesvirus (JMHV).

38. The method of claim 37, wherein the antibody is detectably labeled.

39. The method of claim 37, wherein the probe comprises a detectable non-isotopic label chosen from the group consisting of:
a fluorescent molecule;
a chemiluminescent molecule;
an enzyme;
a co-factor;
an enzyme substrate; and
a hapten.

40. A kit comprising a container means comprising an oligonucleotide primer comprising at least 15 contiguous nucleotides of the nucleic acid sequence of claim 8.

41. A kit comprising a container means comprising an antibody of claim 6.

42. A virus comprising a nucleic acid sequence that has 95% sequence identity with a nucleic acid sequence of the virus of claim 1.

43. The virus of claim 42, wherein infection with virus in a subject results in demyelinating encephalomylelitis in the subject.

44. The virus of claim 42, wherein the virus is a human virus.

45. A nucleic acid sequence obtained by the method of claim 35.

46. The nucleic acid sequence of claim 45, wherein the sequence is a viral sequence.

47. The nucleic acid sequence of claim 46, wherein the viral sequence infects human cells.

48. A non-human primate model for multiple sclerosis, comprising a non-human primate infected with a Japanese macaque herpesvirus (JMHV), wherein the non-human primate exhibits a symptom or a pathological feature of multiple sclerosis.

49. The non-human primate model of claim 48, wherein the symptom is acute onset paresis or paralysis involving one or more limbs.

50. The non-human primate model of claim 48, wherein the pathological feature is myelin destruction in a central nervous system.


SEQUENCE LISTING (28 sequences)

SEQ ID NO: 1 - DNA, 133719 bp, Macaca mulatta rhadinovirus 17577
gatcgggaaaacgcgaggggagcgggggacaggggacggcgtgtgcgtgcttgtgagacaccgggtacggctgcctgcctgctcgctggcctgcttgctgaggggacagtaggcctgcttgctcgctggcctgcttgctgaggggacagtaggcctgcttgctgaggggacagtaggcctgcttgctcgctggcctgcttgctgaggggacagtagggctgctggcttgctagtagggctgctcgctggcctgcttgcttgctcgctggcctgcttgcttgctcgctggcctgcttgcttgctcgctggcctgcttgcttgctcgctggcctgcttgcttgctcgctggcctgcttgcttgctcgctggcctgcttgcttgctcgctggcctgcttgcttgctcgctggcctgcttgctgaggggacagtagggctgcttgcttgctaaggggacggtacgcctgcctgatggcttgatagtagggctgctgggctgctagtagggctgctgggctgctagtagggctgctgggctgctagtagggctgctgggctgctagtagggctcctgggctgctagtagggctgctgggctgctagtagggctcctgggctgctagtagggctgctgggctgctagtagggctgctgggctgctagtagggctgctgggctgctagtagggctgcctgctggcttgcttgcttgcttgctagtggggccgcttgcctgctactagggctgctgtgcagctgggagaacagagtagggctgccggccagctgcgtgcgagggcgtccgagggccagacgaggacacgggacccgggcctctcccccaggcacaaagcagtagggctggccaggggaaacagtaaggctgattgcttgctgaaaaacagtagggctgctggtttgttgctaacggaaaaaggggaggtgtgtgtatgcttttgtgactttccagcggagggggtacagtgcacggccaagttacaagcacctgcttaacttgctttggctctgtgcggttttgttgctaggtactagtagtaacacatagtatttcatcgcatggcgcctgcatacgctccacagcatccgaaacacgttatttctatagactaactttagtgttcttcttggttggtaccattttacgaagtttgctccgttgaataataagaaatccgttgtggttacaatacacctgccaacgtattggatggttccttttgccatcaacctttgcttgcattctaacattgaattttcacattacaccctagtcttctcatgcaattaggtatgtctttgctcgtgtttaattttctctacacatttctgtggaaatgtttgtgttggttttatttatgttattgcaacctgtatcggtggagcttttgcctgcaaaattaacttctgttccaacgtggtgtccaccacatcccggagatacctacttgctaacctgccgcgggacgtctacggccagagaccagcgaagcacacaatggtttcgcaacaacacgcttatgcgtgggagtaatttctacggcagactggtatctgtgactcccaatgctacgatatctgaccggtatgcgtgtcaaacaaaaacaacaacgcggagtaacaacatcgattttcgggtaagctcatcgcgcctcacgctccaagaacggtgctcttcatacggctatacttacgcgaataacacaagggtattgaggtgttactctggtggaaacgtaactttaagaaacgttgtctttcatttaaacggtacagcggtcatcaacggtactacaacaaacatacatacatttgtgttaacagaaaagacaggagggacgtatttctgttctgcgtttattgggaatgaaaaattctattctcagacaattaatgtgttttttacttcatttacctttaaacctacaaacgacattcccaatgagtcacattttaataaaactgggcaaatacaacaaacagctagtgtacaacatcctgaaaactacgttgtgttctctgttcccgttttttctattggcgttttaacaggtattgcaatatcgttgattatgtgttggttatttacaatacgctgcaacgagaactctgaatcatcaactaatagttatgcaagccagacaagctacattcaaccctcccataatcagcgttccaatactaatgaatgtagtcgccatacctacagaaatgctcatcaagaagagagtattgaagaactaccaaaccaacacacaagtgaaactgattcttgctgtcaattagttttacttgaagtgaaaaatgtagcctacgatggaccgcaggaaaacacaattaacgaagttatggaacagtatgatgatgtggttgtaaaaaatatagaacaaacatcatatgaggataatgttgagcacatggactatagtgatactataaatcccaattttaattactacagtggactaatattggaagaagtagatgaagttttttacaatgaactagaaaatcaatatcatggattaatactggagaatttagatcacaatgagtacaatcatttaaatgaattaaacatgatagaacaatatgattggttagaataaataacttgtgggttatttttaaattaaaacataaacaacattaaagcagcatttttgtgtaaatcctttatttattaaaatttttttcatatacctgaaacttatatttaattccattttcctcaaagtttgtatctttgcccggtatctctgataacatggtgtattcggtaaaattaattgatggaaaaaatacatcacaatcaaaactctccataatgcgcgtaatatatagttttaagggacacttatagttaagaacactctcataaacagattttcctccaataacccagaccgtgtttaattgttcttttagtttgtattgtctatagaaattaaacgcatcgtctagcgttctcgctagaaagtgtgctccgtgcggtggttcacgtaattctctgctcaaaataatattaattctgttcaccagagggcgcttcttttcagggatggaaaaccatgttcttttccccataataaccacattcttttcacctacaaccgatggcgtagacgtcattttctgaaaatacatcatttcgtttctaaggtacggccaaggcattgttccgtttttaccaattcctaattgttcatcaactgcaacgatgcagttaactgtaatgtccatggttttagttgccactgacgggtttaacacagaagtatttcacaattataaacaaataacccacgtgacatgtacttactaatgtaagtacgtaacgtgatatagttaatcatattcgctttcacataatggacaaaacgaaaaaatgttacctgtgttaagcctaccttgtttggatggatttatggttacaatgaaaaataaaaatatatatataacgttttttacgtaaaacatattgtaatttaatcacgtgatgtttatgttggctactaacacacaatgtttgcataaaagcacttatgttagtgagcttcaatctttatattttattcaaattgtttaaagaggcatgtgaatatatttaacacgcattcagaaataggcgcaacgctgtgccgtataactggtaaaaacatgacgtttaaactttttcctctgtttttattacacgccataatgtacgtccactgcgatgaaaactgtaaacctccacatttcacggaatatcgcgtcaagtctaacacagaaaaggacttatatagtgttggagaaacagctgaattaatttgtcgtcctggttatgttacaaatacaaaaataataacaacagaatgtttacaaaatggtacgtggtcaacaccaaattttccatgcgacagaaaaagatgtcccacacctgctgacttgctgaatggagccgtgcacattcacgggggagataatgccttaaaatttggatccaatatttcctatgagtgtaatgaaggttatgatttaattggtagtaatgttcgtttttgtattttacaagacacagaaaacgtaaattgggattcaaatgaaccagtctgtgaaattcagaaatgtattaaaccaccggcagtggaacatggggactacctacctaaccaagatgtttataactatggagatgcaattacatttaaatgttcattgtcgtatacactcgttggatcaacaacattagtatgcacgtcaaacaaaaagtggtcaaactctttcccaacgtgtttaatgctcgtatgtgaaagtccacaaatagacaatgggtacatagacattggcttgtccagaagatacaaccatggacaatcaattactgtaaagtgtagcgacgggtacaacattgttgggcctgaaacattaacgtgcacaaacacaacttgggttccaccattacctaagtgtgtattagttacaaataacccaagcacacccatgccagaaacacccatgccagaaacacccacgccagattatcaaaaaataaatttgtcaaccgctaaaactgcaacaacaccaaatgcgtttgttacaactgttgtttctccagagaaagacgacgttacttgtgtaaagcctcattttgagcgattcatggtaaaggctgaaaatgacaaggaaaaatacagtgttggtgcgagcgttgagctaatatgtcgaccaggatttactaaaatgcagtctacagtttctgttgaatgtttgtccaacggaacatggactgctccaaatgccaagtgtcatagaaaaaaatgtccaacccctcaagaacttttaaacggagagtatatagttacaagcggagaagatgcttttaagtacggaacaaatataacatataaatgtaatgaaggttatcaacttttaggaagtatggtgcggatttgtatgcttaaagacgatttaaaaacagttgactgggagccaaaagcgcctatatgtgatattgaaaaatgtaagccaccgccacaaattacaaacggaaaataccatccggtgaaagacttttatcagtatttggacaccgtaacattttcgtgcaatcgtgacttttctttagttggagatgaaatgacaacgtgtataagtaatacgtggaataaaccgtttccaagatgtgaacaaatcacttgcagcgctcctaatattgcacacggaaagctgctaacaggttcttcaagcgtttacaaatacggtcaatctgttaccattggttgtgaaactggatttactctaattggcagtgaaatttctacatgcaaggattcatcgtgggatccaccacttcctacgtgcgtgccagctgtttcaatgccttctgacacacctaaaccagaaaccaaaaaaccaaacacgccaacgccagaagcacccaaaccaaacaccccaaacgttggaacacatacaccattcaaaccaccaccacaaaatccaccaatagcacccccaatgagtaaatggaaaaggcatgtcgtgttagttctttttgcaagtgtcgcgtccttgttattcgtacttgctgccctttattgttgttttctaaaataactgttttttgtcttcagcaggttcgccaggcaaactcgcacgcattaaccaatctgccaaccgccgttgattccggaattaagtttacattattcaaggttgccaataaaggtggtttaaaaatattctattggtgttcattgttttatgttgacccgtttatagttatcgcgccaccttgtggctacattatatagcacgatcactttccacgttatactttcacgtactatgactcatacgccttaacgtcacgtggcgtgcgattgtggccggggctgaaaataacacaaggggtacataatccatccaggcggcacacattagacacggtttataaaactatatcggatgcgccaacaatcactgtcgctagcgacactgatagaaaaacattttaacgtttgtttagcgaacttgaataacacataatggcttccaaaggcaacgccggacaacccctggaagataatcaggggtctcgtgccccgataggtgcgtgcggatacgtgtacgcgtattcgaaacaagactttccctttgccgaggcgtccatactcggcaacagaccatctggatctggcgttttctcgctaccaatcctttacggacttacagttgaacacgaattccctctcaccgtaaaagccgcatacaaaaaagttgacaccacgacgctcgccgttaaggtgacgtgctttcacagagaggttattgtgtttcacaatgcaagtttattcaggccggtgtttgacggtaccggtcttaacgaactatgcgaggaagccagggctctctttgggtacacgcagtttatagaaccgggtccacctcacagcatatggaaccctctggaatgtccgcagttaccggacaaggatgagatgtttcttggcgttgttgttacggaagggtttaaggaaagactgtggaggggctgtctcgttcccgcggtgttccagacccagcaggtgcagattgccggacgccaggcgtttaaagtgccgttgtacgacgaagacctgtttgcacctcacggtcatagaatgccaaggttttaccataaagacgttagcgcgtacctctacgactccctctttaccagcatcgcccaggccctgagactcaaagacgtgacggcggtcatccacgccacagaaaagcaattcatgcaggaccattacaaaattgccaagatagtgcaggcaaaacagttttcaacgacgctgccgaaaacgacagacgggtcgtcccacatgattgtggacagcgtcgtcgccgagctcgcccttagttacggctgtatgtttctcgagtgtccccaggacgcgtgcgagttgctgaactacgatagctggcccatatttgatggttgtgactcaccagaggctagggttaacgcgttagagcgctggtcggccgaacaggccgttcacgtggcgggtcaactgttcgctgccaattcggtgctgtacctaactaaagtgcagaagcaagcgcccaggggacaaaagggagacgtaaacgtgtacaactcctttttcctccaacacggactggggtttttaaatgaggccacgatcaaggaaaacggcagcgaagcctttaagggcgtaccctcaaacgccctcgatggttcttcgttcacgccgtatcacctggcctacgccgcgtctttctcgccccatctgctggcgaagttatgttattacatgcagttcttgcaacaccacaaaagctccacgaaccaggcgtttaacatggtccattatgtcggcaccgccgccaactcagagatgtgcacgctatgtcacggcaacacgccggcaacgtgcctcaacacgctgttctatagactgaaggataggtttcccgccgtaaccacccctcagcgcagggacccctacgtggtgaccggaacagccgggacctttaacgacctggagattctgggcaacttcgcgagctttagagaccgcgaagaggacggaaacccggccgacgagcacccaaagtacacgtactggcagctatgtcagaccgtgacagaaaagctatccgcgattggaatcaccgaagaccacgataatcacgtgaacctcatcaccaacatccaaagttttctcagggtgttcaagggtatcgactcaattgtggacggagaggtcatgaagttcgttaattcgatgattaaaaataactttaatttccgcgagcacgtcaaatcggtccatcacatactccagttctgctgcaacgtgtattggcaggcgccgtgcgcggtgttcctgaatctgtactacaaatccctgctgtggatcattcaggatatctgcctgccgtactgcatgatctacgaacaagataatccggcgatgggcatcctcccctccgagtggctaaagatgcattttcagacgttgtggacgaactttaaagcggcgtgtctcgaccgcggggtcctcacggggtgcgaactgaaaatcgtacaccgggacatgttctgcgacttcttcgacaccgacgcggggtccaacggcttaatggccccctttaaaatgcaggttagaatagcccgagccatgatggtcgttccgaaatcaattaaaataaaaaatagaatcattttttccaacaccgcgggatccgaggcggtgcagtcggggttcgtcaaaccgacgggaaccagggacacttacgtggtggccggaccgtacatgaagtttctcaactcgctgcatcgcgcgctgttccccgacaccaagaccgccgcgctgtacctgtggcacaagatctcccagaccaacaaaaccccagttctgaaagacgtcccggacgacgagctggcggagctggtgtcgtacgtaaagaccaacagcctcgcgttcgaggaaacgaacgtgctggacgtggttccggattcactcatgtcgtacgcgaggatcaaactgaacggggccattctaagggcatgtggccagattcagttctacgccacgacgctgcactgcctcacgccggtgctacagacgatcgatgccgaggaatacccccacgtgctgggctccgcggcaatcgccacaccggtggcttacctggcagaaatacgcggccgcaccgccctcaccgtccagacgacggcgcgtcagccggtcgccgccacagggcgcctgcgtcccgtgataaccgttcccatggtagtcaacaaatacacgggggtcaacgggaacaacaacgttttccactgcggaaacctggggtacttcgcggggcgcggcgtggaccgcaacctgtggccggaaagctccccctttaagaaaacgggcgtcagcgccatgctaagaaagagacacgtcatgatgacccccattatcgaccgcctaataaagcgagccgcgggacagacaatcagcacgttcgaggcggaaagcgttaaaaggagcgtgcaggcgctgttagaggataaggacaaccctaacctattgaagtcggtaatcttggagcttatacgacacctggggaagggctgccaggacttaagctccgaggacgtgcaatattacctcggtgactattgtatgttgacggacgaggttttatttacgttggataatatagcacagtcaggcgtgccgtggactatcgaggacgcgggtgccctaatagaggatcgccaggacgcagacgatcttcagttcgtagacagcgacgatatcgccaccgcttcctgtcagccccccgaggaacagctaccgacccctagcgccggcgccctactggccgggaagaagcgaaaaattaacgcgctgctgagcgatctagacctttaggaaaaccgtggcaggcgggcaacaatggccagggaactcgcagcattatacgcgcagctgtcggccctcgccgtcgacttgagtctggttatctttgcggacccgcgaagtatcgacggtgcccgcattctaaaaacaaaaacacagatagagaacctgaaccgcgaccttctgccgctgctacgcgagcaaaactcggtagagacgtccagcctgtcgctcgaagtggagcacctggccaaaaacatcgaggacaaactcggcgagctggagcgcagtctgcggcagagatattcgagccgagagcattttgaaacactacacctgagacccgaatgtcactatcactctacggttacttttcagttttacgggggcgggttaatagatgtaaacatgtgcctaataaacgatgtagaactgctgtgtaaaagactagggagtgtgttttattgcatcggtgcaaacgaagctctgtccggattgaaccgggttctgacgtttctgtcaacactgcggggtatctccccgatcccgcacccagacctatacgtcacgtcagtgccttgcgtacagtgcctgagggaaatcgaactcgtaccaaatcaggggtccagtttactcgcggtgttggcagaccgacactgcgatcacctctgtaagaaggttagggcggagccaatacacggcctgtttgagacagaactgagccagctgggtctaaaagtaacaaaacgttcggacgccacgcagcacggcgtccggtcctctgcagatcagttaagggagtcgtcgctggcggccatacaagatcacaatatattcaaacgggtgtccgcgtcaatcatggaactatccaatctaatttattggaacgccgggcaaaccggcctccagaccgggaccgaaaacgagtgctcacaaatggccagactgctaacacacgaggccgatatgcacgagcaccgtgcgctaataacacccaaactaagcgcgactcacttctacgactgtttccgaccggatcccatagaatccctgttctgcggcggtctttttaactctatagacgacaccataaacgcactgagccgggattgctccgtgacgttctttcaacaggcaaactataccaacgttatgcgaaaacaaaacgagctgttcaccagactcaatagcatcctgcgtcaggggagcgcgggatcgcaaaaaccggccaccccctcggagccacggaccaccaccgtggcggcaaccgcggcaagcgacgtcatcaaagacgcacagtatcgcaaagaacagtacatgaaaaaggtggccagggacggctttaaaaaactaacagagtgtctgcagacgcagagcgcggtgttggcaaacgcactctgcatgcgcgtatgggggggcgtcgcctacggcgaggcgtccgagctggtgaaccattttctcctcaggcggcgcttcgtcgcgcttccctgggaggcgcgctgccgctcggatcagattttattcgaaaactcaaagtacattaaaaactcactatattcccagcgcctcagtcgcgaacacgtagagattatcacgctgcagttttacggcctgataaccggccccctgacgcgccagagcgatctctttcccggccccgccaacgtcgcgctggcccagtgtttcgaggcggccggaatgcttccgcatcacaagatgctggtgtcagagatgatatggccccagattcaaccgaaagactggatagaccagacatttaatcgtttttaccaacttcccgaaggtgatctcaacgcggtacaaaagtccgcctggtgctttatacgagagctcgtcctctcggtggcgctttataatcgcacgtgggaaaagacgctgcggatattttccctagcgcgcgagaaactctccatctccaacctagacgttaaaggcctgacgtccggcctgtatctaacgtacgagcaagacgcgccgctcgttctaatttctcaaaataccggctggatatttaaagacctgtacgctcttctgtaccatcacctgcaactgtccgacggccatgatgataactaaccgaacgcgtcgtctcctgcgggcgtgggtcgtgataatcgcgatcggcacggcggttggcgaaaacgtcaccacccccaagggcgcgaccaccaccgcgaagccaacgccgggcccgtcgacgcccacacctcccgagaacccacctagggccgaggcgtttaagtttcgcgtgtgcagcgcctcggccaccggcgaactcttcaggtttaacctggaaaaaacgtgtccgggcaccgaggacaagacgcaccaagaaggcatcctgatggtgtttaaaaaaaatattgtcccgcacatctttaaggtcagacggtaccgcaaggtggccacctcggtgaccgtctatcgagggtggaccgagaccgccgtgaccggcaagcaagaggtcatccgaccggtgccgcagtacgagatcaaccacatggacacgacctaccagtgtttcagctccatgcgcgtaaacgtcaacggcatagtaaacacctacacggacagggacttcactaaccagaccgtgtttctgcaaccggtcgaggggctcacggataacatccagcgatacttcagtcagccggtgctgtacacgacaccgggatggtttccgggaatttacagggtcagaaccacggtcaactgcgagatcgtggacatgatcgcgcgttcggcggaaccgtactcgtattttgtcaccgccctgggagacacggtagaggtgtccccgttctgccacaacgactcaacgtgctcggtcgcggagaaaaccgaaaacggcctcggcgcccgcgtgctcacaaattacaccatcgtcgacttcgcgacccgccagcccaccaccgaaacgcgggtcttcgccgactcgggagaatacaccgtatcgtggaaggcggaggaccccaagtcggcggtctgcgcgctgacgctctggaaaaccttccccagggcgatacagacgacgcacgaggccagctaccacttcgtggccaacgacgtgacggcgaccttcacgtccccgctctcccaggtaactaacttcacgggcacgtacccctgcctcaatgatgttattcagaaaaccctcaacgccaccatcaagaagctgtccgatacccacgcaacaaacggatcggagcagtactacgaaaccgaggggggtctgtttctcctgtggcagccgttaacgccgctaagcctagctgacgagatgcgcgaattaaacggcaccacgccagcaccccccaccacaacctcaaccgccaaccgcgttcgaagaagcgtcggtacgaacgagcaggcaacggacgacctagcggcgccccagctgcagttcgcctacgacaagctccgcgcgagcatcaacaaggtgctggaggagctctccagggcgtggtgccgagaacaggtgagggacacctacatgtggtacgaactgagcaagattaaccccaccagcgtaatgacggcgatatacgggcggccggtgtcggccaagttcgtgggcgacgccatctccgtgacggactgcgtggcggtggaccaggcgtccgtcagcatccacaagagcctccgcacgtccaccccggggatctgctactcgcgccccccggtcacgttcaggttcctcaacagcaccacgctgttcaagggccagctgggacccagaaacgagatcatactgacggacaaccaggtggaggcgtgcaaagagacgtgcgaacactacttcatagcgagcaacgtaacctactactacaaagactacgtcttcgtgaaaaaaattaacacctccgagatatccaccctcggtacgttcatcgccctgaacctgtcgtttatagagaacatagatttcagggtcatcgagctgtacagccgcgcggagaaaaagctgtccgggagcgttttcgatatagaaaccatgttcagggaatacaactactacacgcaacgcctggcgggactccgggaggacctggacaacacgatcgacctgaaccgcgaccgcctggcccgcgacctgtccgagatagtcgcggacctgggcgatgtcggccgcacggtcgttaacgtggccagtagcgtgataaccctgttcggatcaatcgtgagcgggttcattaactttataaagagtccgttcgggggcatgctcatgatcctggtgattgtggcggtcgtcctgatcgtgtttgcgctaaaccggcgcaccaacgccatcgcccaggcccccatcaggatgatctaccccgacatagacaaaatgcagccctctggcggtaaagtcgaccaggagcagattaaaaacattctcgccggcatgcaccagctacagcaggaagagcgtaggcggttagacgaacagcagaggtcagcgccctcgcttttccggcgcgcgtcagacggactaaaacgtcgctttaggggatataaaccgctggaaaacgaagaggctcaagagtatgaaatgagcaaataaccacacccacacgcctgtacttgccgcccgccagagccgcgcggccaatcgattcgcgcacccggccggtccccgacaccctctagcgcccccgggcgtccgcggtctgtatcaatcatggatttctttaacccgtacctgggccctcgcggaccacgcccccactcacacagaggcaccgatgctcccgcccctgccggcgccggagccgttcagccgccaccagacgtttgcaggctcatccccgcctgcctccgaacgccaggggcaggcgggatgatcccggtcacgatcccgttcccgccaacgtacttcgagaacggtgctcgcggagacgtgctgctcgccaacgaacggtccatgtggacggcgcgcgaccgcaagcccgtcgccccggacccccaagaccaatccatcacgtttcacgcgtacgacgtcgttgaaacaacgtacgcggcggacaggtgtgccgaggtacctagccgcttccaaacggacattatcccaagcggaaccgtgctcaagctcctggggcgaaccgaggacggcaccagcgtgtgcgtgaacgtgttccgtcaacaggtatatttctacgcgaaggttccagccggcattaacgtcacccacatcctccagcaggccctcaagaacacagccggccgtgccgcgtgcggcttctcgaccagaagagtgaacaaaagaattctcaaaacgtacgacgtcgcggagcatcccgtcacggaaatcacgctatcgtccggttccatgctctcgaccctcagcgaccgcctcgtcgcgtgcgggtgcgaggtgttcgagtcaaacgtggacgccgttcgccggttcgttctggatcacgggtttaccacgttcgggtggtactcgtgcgcgcgcgccacgccccgcctggcggccagagatgccaggacggccctggagtttgactgcagctgggaggacctcagcgttcaagcggaccgcagcgactggcccccgtaccgcatcgtggcctttgatatcgagtgcactggagaggcgggatttccgtgcgccacgcgcgacggcgacgcggtgatccagatctcctgcgtcttctacacgaccagggaaggcgcgcccaatccgccaaacatactgttcagcgtcgggacgtgcgaccccatcccggacaccgacgttttggagtttccgtcggaatatgacatgctggtgtcgttcttcgccatgatccgcgacttcgaggtggactttttaaccggctataacatctcaaacttcgatctcccgtacctaatcacgcgagcgtcccaggtgtacaaccttcgattaaacgaatacacaaaaataaaaaccggctccatctttgaagttcacgagccccgtggcgggggaggggggttcatgaggtcggtctcaaaaattaaaatagcgggcatcgtccccatagacatgtaccaggtgtgtcgcgaaaagctcagcctctccgactacaaactggacacggtggccaggcagtgtctgggtgggaaaaaagaggacgtatcgtacaaggacattccccctctgtttcgctcaggtccgggcggcagggctaaggtgggcagctattgcgtgatggactcggtcctggtgatggacctcttaaaaatgtttatgatacacgtggagatttcggagatagccaagctggccaagattcaggccaggcgcgtcctgacggacggccaacagctccgcgtgttctcctgcctgctggaggccgcggccagggagaactttatcctcccggttccaacgcccgagggacaggggggctatcagggcgcgacggtgatcaaccccattccggggttttacgacgagccggtcctggtggtcgattttgccagcctgtacccgagcatcatccaggcgcacaacctgtgctactccaccatgatacacggacgagacctgcacctgcaccccaacctgacgccggacgactacgagacgttcgtgctgagcggcggaccggtacattttgtaaaaaaacacaagcgggagtctctgctgggaagactgctaaccgtgtggttagaaaagcgaagggcgatccggcgcaccctggcggcgtgcgatgacccgtcgctaaaaaccatcttagataaacaacagctggccatcaaggtgacatgtaacgcggtttacgggttcaccggggtggccagcggcctcctcccatgcattaacatagcggaaaccgtgacgctccgggggcgcacgatgctggagatgtcaaagtcttacgtggaggccctgacgacggaagacctgcgaacgcgtctcggtcgcgaggtgaccgcccgtcacggcgcgcggtttcgcgtcgtctacggtgacaccgactccctctttatcgcgtgcgacggttattccgcggaagccgtttccgctttctgtgacgatctggccgccaggatcactgcggacctgttccccccacccattaagctagaggcggaaaagacgttcaagtgtctgctgctgctgacgaaaaagcgctacatcggggtcctattgaacgacaaaatggtcatgaaaggggtcgacctcattcgcaaaacggcctgcaagtttgtccaggagcgatgccgcgccatcctggacctggtgctccacgatccggaggtcaaggctgcggcgcggctgttgtgcaagcggccgccgcacgcggtatacgaggaggggctgccggctggctttataaaaatcgtagaggtcctcaacgcgagctatctggacctccgaaacagcgtcgtgcccatcgagcagttaacgttctccaccgagctcagccgccccgtctgcgattacaagaccaccaacctgccccacctggcggtgtaccaaaagctggcgagcaggtgcgaggagctgccccaggtgcacgatagaatcccctacgtgttcgttgacgcgcccgggtccctaaagtcggacctggccgaacacccggattacgtcagacagcaccagattcccgtcgcggtcgacctatatttcgacaaactggtgcacggcgcggccaacatcctccagtgtctgttcggcaacaacgcggacaccacggtggccatcctctacaattttctcaacgtcccgtataagctgttctcgtgaacgccaattggagaacgccaacataagacgccgcgccagcggagtccgcagggagagctcgagcggcgaggagcgaccaacggagaccgccaccatgctggttaacgaactgtcggtggtcctcggcgactgggaggtgacttttcaccggggtagattcagcttcgtcaacctcacccgcctgcaaacgttcaagggccacgggggctacgccagggtccgactccccttctcgctcgaccagttactccaccaacatttcgcgttcggactcgtgacgcgtctcaaggaactgccccccttctccgactgcgtggcccttatcgccccgttggattccggcggcgacgcggacgcggcgcgcgtggcccccgggttcgtgctggactcctctcgcccgctgaccgtgtgggtaaacgcgagcgggcggcacacgatccggttctgcctcctctttctaaagccgatcgacctggagcgcgcggtcacgtacgtcttcggcgagaacggcggcgcgcgctcggagggcaccccaaagcccacctgcgcgaccgaaagcctgcccggtggacccctgcgcgtctccggcgaggcgtctcagacgtcgccccattctttcgttgcgtattttcccacggccaactcggtggcctgcctaagcctgttgcggttacaggtgaggccgttttcggatgacgcggcgcacagggacgcgcggatctccccgaaatacgtcacgtttagtaactccgggggtaacgtctgcaaggcgtccgttcacacgctgtccccgtcgcggtgtaaaacggcgcaaatggaaatcatctacgctcccggggaccccaacgccgagatagtcctgggccagtccggacccgtcctgcccacccacaccggcggccgcgtattgggggtctacgccgacgccgaaaaaaccatccaacctggaagctccgcggaagtccgggttcagttaatcttccaacagggagcggccgctcggggcgatctggcgtttctggtcacgggcgtggcaccggagcccctattcgtcgtcaccccggcactcttgctttccggttgcacaacccacctgcgcctattcaaccccaacggtacccccacgactataaaaagagacacccttgtggccgccgccgcgccctgccccgtggtgcgattaagctccgccgacgacgcgccgcgagacctcgtcgcgtcaccagacaccggggcgctctccattaacgcgttcacaatcccggtcggtttcccaggggtggtctcggcggagtgtcacgtgtccctacgcgacaacggggtccacgaacgcatgaaccattgacggcaacgatgggaacaccggtgcgtttctttcgcggcgagtggcagacctcgagtctagtggacaacggcacgccacggtacagctccctggtgtgggccgccactattcacgacggctacctgacactggtgaacaggtcagagctgtgcgtcacggagaggtctccgtgtctgccggcatgccccagcatcgggagactggtcgggaagaggtttcccggcttcgcctttgccagcgccactctgggcgatcggggaacacgcaccgtgttctacgcgttcggtcaccgcgacaacccactggacatagtacccgccgtggtcgagcgcgcggatcgcgagctggtgctgcgggttcacgctccgcaaacaacgcgggtgtcgcgatacggacttaaggtattcgtggcgatcgttacggtggtgcgcccgcccggggtgttcctacactttccacaagaccgcgttccgatcgcgctgacagacgcgtgcagccaggagggctccaggctaacctctgaagagccgtggataaaaattcaaggctttcccgtcctatctgacgagaccgcgcacccatttctcttaacccagaagaccaagccctttaccgagcgaaagttttgccgcctgatcatggacaacgaccagcgcagcgccgtcaacaccgtctacctgggaaagcagcacgtgagggtgaccgtgacccgccccccggaaacaatcgtcaccgacggccccgtgacggcgaccctgtccctcaccggtaatgcgccaatcgcctttcgccacaacccatactttgaactcccgtggtcgtccacaacggcgatattcacgcccgtggtgtacgtgggcctgaccgtgtgcatcccacccaactgtagcaaattcgtaaggtacggtaacacctacgtctcggcatttaaccgcaagctgacggcgattattagcaatcacgcccacaacggcgggttccggattcaggactgcgagtggccaccgaaccgggagatagagattttggtaaccaacgtgtcccaggccccggtgtacatcagcaccgggacgcagctggggcaagccatcttcgtgttcgcgccgcggttcggtggcccggcgaaactgcggcagctcctcggccaccgatcgcgcgccctggagctgccgggcggggtgacagtggacagccaaaaactgtgtaggtttgagaccatgtacctgttttccacgtaaattactaataaaccgtttgctcgtatcgctcacacaacgccaaaccgtctctcattctcggggtcgcgcgcctcgcgaacacacaaggtggctcaaacacccccctcccgcaccctcgccacacaaaaccagttaacgccttccgttagatgcagtttatttattattttattacatcatagctattgcgcggcgcccgtcccgcaaaaacatctgtagatattccagtatgcgaaacgcgctgagaacaacgtcccgggggctctgccctcccaacgcacgcacggttttttcattagactccgcgccacctatcttgttatttacgggaagctcctcaattagagagtcgagggcggacagcaccacggtcaccgcggagccaatggccgcggcgtccgcgtccccgggcgcgtcgtccagcagcatcctcagtcgcgtcaggtaactctcgtaggcccggagcccgcgaaccatggcgttcatacactcggccccgcgaaactcgcggcgctggcactcaacgttagacatcaatatagggaagtgaacaaacgacatgatgcgcgggtactcacgcacccgacagagagtggagtggtggcacaggtaaacgaaccagcgctgtatgtttaaccccacttcagacaggcgccccctggtcctgtcaagagaggcgcggttgccggcccactgggggagaacgttaattccagcggcagtcgggggaggcgccagcgtagggctggccgcccaacacgacaggtaaaacaagacgaaccagacagggaacatgaccggttaaattactcgcttacaatcgcgggcggcggccggtcaacgccaggtccattaaaaacacaccggccgccaacccccaacgcggggccgcgccctggaacgcggttccttccaatcgcaaagaaccgcgtcacaaaaagggctcgttttgaacccattttgtgccatcgggtttcgttttcagatacggaaacggctcgtccaaaaacacccaacgggggtgtgctcaaacggccatctccatctctatgtggggatgggggtcgtagccctcgagactcagatccgcgcgcgtaaagtcctccagacgcgccacctttctcaaaatcttcagccgcggaaacggacgcggggtcctccgcagctgaagcagcaggggatcaacgtggttgttgtaaacgtgggcgtcacccaaggtgtgcacaaagtctcccggggtcaggcccgtgacgtgagcgatcagatacgtcaggagggcgtagctggcgatgttaaacgggaccccgaggcccatgtcggcggacctctggtacagctggcaggacagctccccccgagccacgtaaaactgacacaaaacgtgacaaggagggagagccatccgcgcgaggtccgcggggttccacgcgcacataacgatgcgccgatcgtggggccgcctgttaattagatccaccacgtaacgcagctggtccaccccctgaccctcgtagttggcgtcggcccccctgtactccgccccaaaatgtctccactggaacccgtacaccggccccagatcgccctcgcggcggtccccgaagccctgcgccgccaaaaaggcgcgggacccgtgcgcgtcccaaatttttacgccgcggcgcgacagttcggtggagtcggtggagcccctgataaaccacagcaactcctccacgacgcccctccaaaacacccttttggtggttaacagaggaaactcgtccctgaggttatatcgggcctgaagcccgaacacggacctggtgcccacgcccgtcctgtcctccctctgcacgccgtgtttaataattaaatccaggtgcgccaagtactgcagctcgccgtggtcgccgcggcacgcgcagcggacggccgcggcaccttcgggtgcggaaaaaacccggagtcgtgcggccgcgattccagccacgcaaacgggtataatttttttaacgtagcagataccgagatgcaccaggacaatcataattacaaccgaggtgctttacaactataaccgtaaacggctgaagacgaaacttattttaaaggcagcttgggcggggccgaccacaggcgcggcccggctgttagcgagtaataaacccacgtggtctccccaagcgtctccccgagcgtgagcgtctccccaaacgccaaaaacaacacagataataaaataaataacatgtttattttttataaacttaacgcgcgattatgcttcttaacaggggccaatagaagcaacgtgcgaggcggcattcgcgcgctcaaaggcaaccgcaacaccggcgctcgtaacagcgtaaacaaacaactattagccattcgtaaccgtaaaatccaaccctctgcgtcccgagccattcgcccagctgcgtctacgtcgagggtgtttctaagtcgcattttgagcccttggcacggccgcgtctgcgttaatggtgtttctaaatcccgctgccaaggccctcttcggaatcgtcaatcaggctgcgccgggttcttctgccctttttgggacgcttgtccaccgcctgaagcagtttcttcactgcgtcgtccccgggattggcacacagctttttaccgcggcgagtgtttaatatcacggcgtccaccgagcactgcgacgaggtgtgggagtaagagaccactaaaccgggtggcggcagatgagttacataccccaaacagcagagttcgggcgcgggtccgctcatggagcccataggaaaggcataatcaactacacacgcgaacaccgcaaaaaaaacgcacacgaaaaggcccctcatggtgacaaaaacacaaaacaaagacggggtcgctcgtgcgttgccaccagcgccacgctcgccgtgctcggccacggttgacggcgtccattaatacccgcggcccgggcccgcctcgacggaaaccaggcgtggttagccacctgacgcacgtgcctcccgtccagatgtggcattgcgagtcccaaaacgggcgtgtctaactacggtcgccccggggttcaaagaacggggatcccccaaacaggtaaaaagctttttgtccaaacagaaagccaacagcaaccgaaaggaatctccaaaacaagcaagggtcccgtgccggcgataaacggaaaatttccctcacctaaacacacgcgccgcgtaaactgttaaaacacgcaatacttcctaggcctgtgtttaataaaacacacaagggttgtaatttcgcggggccctttgggccccgcgcgtctgggggtgcgttgtctcgttgtggttcgcaatcaccaccccgcctgcccgaaaacagggcaacaaccccctggcctagttttttaaaaacttaacaccggcaaggggagagggagaaggggtgcggctaaatgggctgttagcagccaaccgcagcggggaaaaaagggggcaaacgccgggttaacacaaacagcaacaggcgccaaacccaatataacacaacgcatattccgtgtgtttcgcgggccggggtcggtacggctaacccgcgttgggcgtttgcacaagcacgcccccgccctgcacccggctccccgaggtcaccgaagggtcaggaaacatagttttcacggtcgccagtcgcgcgcgtggcaacatctttcggtgccctccgaacgacccggaaaaatccccgccgcgccgggtcgttcggagggcaccgaaagatatccccaaacgcaacctaaagcatcatgtttggggtttcggtgacgcgcgccgcagaggaggccggtggtggcgctggcgaagatagtggccgcagagcacgggtttcggtagtgacgcgcctggacggctgccacctggcagcaggccaaaaacacgggcgccgcgagcggccggcaggtggcgccgcgctttcccccacgccgcgatttggcaaaatggcggaccggcagcgccggttggacgcggcggacgtcaatcacgacggagagaagcgcagttggtcagttgtcccgtcaatcaccgagggccgcgccggttggcggataatatgaaaccggcgcggccattggacgctggcggcagccaatgggcgcgttgggttttgtttacaagttccctatatatattatattataccttccctgttaagggaatcccatgttcttaaatctaatcagctgggttatataattagaatatcagtaattactattattactgtattatttattaaattatttattttattatttatttaataaattaattcttgcgttattatgtaatcagatactgggaaatctactcccagtttaataaaaataaataattaattctattatggtgggggttctaaatattgtacaaaacaacaatattttttaaatttattcactaaaaatgctcccattttacttgtgacgtacatgttaggaatgtgggagtggtttgtaactatgttttcaaacaacacccttttgggccgtctgaaactcatataagcggaaggctcggcgttaattgccactggccgctaaccagtccaccttgccagttggagttttattgctgcttatggctttacctcgcattccgggcacaagctgatttacgtagcacgcattgggggtttaacccggttgaatttagtggtttgcacgtgtaaaaaagcccaccgaggcttgggtttaagcgtttaaagcgttgctggttttataacatgcgatttaattgctatgggagctagcttgtattccgcatgctttcggtttagtagttgtacaaaacagaattgtagcccgcaaattgttactggtacccaaaacggcaagcccgcttaacacgccccgcgctgcggcttttgttcctagcccgctcgcaatacaatcgagattttaatttaaaatggggcaatggcgtagcgcaaatttaagcgcgtcaaaaatttaaaatggactaacgtaaccggtgcttttagtgacctatggcgaattttaaaatttaaagccgtgggttttaacacagagctgccagcttgtaatacgttgcatatacagtgccgttgtgattttatggttaagtggattttaacattgaattttgtaatggtgtacacgtggatttttaaattgaatttaaaattgtaattacgtgcgcgtggattttaaaattgtatttatgtaacattgttaaattttaactgcgttatggttgtattttggttaatttatgtaattttgcttttagatttgttcgggtgtgaattccagacaggtaagagcctagctcctaatgtttgccttgccgcctagctcctaatgtttgccttgccgcctagctcctaatgtttgccttgccgcctagctcctaatgtttgccttgccgcctagctcctaatgtttgccttgccgcctagctcctaatgtttgccttgccgcctagctcctaatgtttgccttgccgcctagctcctaatgtttgccttgccgcctagctcctaatgtttgccttgccgcctagctcctaatgtttgccttgccgcctagctcctaatgtttgccttgccgcctagctcctaatgtttaacaacattaatgtttaagcacactaaaatttaaagacgtttgtgttggtttttatgaccagcttggtacaaaacctgctggtgattttttacccaacaaataataaataaaaaagttaaaacttatttctgttgtctgtggtaattgggttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgagggtcccgggctccccgttccccgattcaacgttaaagtggtttatttagcaaacacacgttttttgccgcggtttgggttgtacatacacgcgggtgcgcattggcccgcccatgcaaaaaaataacgttgggtttccgaaacgcgtaggtgctttagtgtttcatggcacgttttcgtgtcacggtaggagtggttagttttttggtagcgaatgcaaccaggtagctcgcctgaccgggaaagggaacgttttaaaccgcagtgttaccgcggtgcggcatacggtttaacgtgcaactgccggtcccgcgcgtggtaccttaagtttacgtgtagcaattatggcggctgttcagggccctccgccgccccctgaggaagaaaatgaaaattctttgccagttgacgtttatgctatagagggcatctttctttactgtgggctcgggcaggcggagtacttgcaccatcccgtgtttagccctattaaggaatttatcagcgcctttctcaaggacagcgctcgcctgtacgagaggctcttgcgccacaccgattaccgctctctgcggggactaaacgccataggccaagggatgctgcaaataaacacggacggacgccacaactggggtcgcgctttggccgtgttgggtcttggtgcgtatgtggtggataaggttaaagacgacgagcgtcttttaacgttcgccatagccgttctacccgtgtacgcgtacgaggcgctggagtctcagtggtttcgttcacacggcgaatgggagggactcaggaattactgcgagcgaatactgaggcatcgccgcaacgcgaggagacacatgtgctacggagttgcggctggtcttctggcgctagtggcgctgtttgccatcaggcgataggtgtgcgtttaaccgcgtattcccccaccctgaccttaagtgagccgattgtatatgagaccaataaaacaaaacaagctaacgtggtattcgttggaacattttttatttgagcagttcctcgcagaacattttttgtatctgtgacacgggggccggctgtgcgctggcctccaccggggcccgcgcggactgtccgcattctggatcgaggggcgggcacgcgccagcgcggggggcagaggctgaaggaatggcgtttgacattacagattcttgggcggcgggggctggagttgccggctgcgtggcgagaccggcgtggtggctcgccggctgcgggggcggtggttgcgtttgctggatgccctgcgcgctctggtacggctgtggggcgtggagatgggtgtccgttccggaataccactgcggctggtacgctaggtacggcgcagacgcgtaggtctgtggccacgagaactgaacgggcggtggttgcggtcgttggtcggcgtcctgtgcgccggaggtctgcgacgcggctcgttttaggtccctcagatccgcctgaatgtccaatatgtttttggacagcgccatgacgtccttgtgtatgccgacctcctcgcctggaaacacgggctcgtcggaaagctccacgtcccgtttgcgcttctggtggcgagacggtgccggagcgtagccgtacgtcggagaatagtacgtggccgcatacggagagggttggggggcaaacgccgactggaaagggaacggtggcgcgacgggcggtgcgtacgatgggacggtcagctcaggcggcggtatccacgatggatacgccgccgttgccggcatcgagggtggcgcagaaacgtacgtgcggtggccctggttgcgcatgttatcgagactgctctgcaccatggtcagaaaggtgctcttgggaatggtgatgttgtcctccgggagggcgctcatggttcgggtgtcgcggttaccgcagtgctgagcgcagggaaactggcttgcctttaaatacgtacttctcgcgactctggccacgcctttatctgttttgagcaggtctgtgcgatctcggataaatccggcgtcgatcgccttggccatcagtgtctcaagaggggccgcgaattctggcggtgtcacctgacgttcgcaaagatctaaacaattgaccgttattttaccggcttcctctcgcgtaagagaatcaaacttggagacgacccatgtagggtctggtccgtaaacggcgattgaccccctacgcctccccagtgcgcacagagagacgtgctgaaacgcgggtgggtgattggggtcttgcagctcttctgggtggagggacgagagggagagctccgggagccatgtgtgtaacatctccaacagggggtcccgaggcatggggtttagctggatctgggccgctcgcgagtcccccgcgagccgtgacgccagggccaagaaatcgtggctcgttagcttgcccaggcaaaatatcccgtgtgttacttggaagagaccgattgtgtgacctacgtgggcttcggggacgtgctctatgtttatcggtagagggtccgtatacgggagcagggtcgccacgattgagggctctaaatacagctccttttctatctttggtaggctgaccacgtccacgtatcccccaacgtacacgggagtcatgttgggtcggtgtctgcgtgagccgaaggagatgagcgccggtcttcgggggctgatgtggcgcgttatccgttgtgagaatttaaatacgtttctccctggcgagctcaggtttcttcacctggtgctgtgcgaaatgtacaactacggtcttaacgtgtaccttctgaaggaggcaattgccaataccggaacgagggacgatattgtcctcgggcgtaaggttccggtggagttctggaaaatcgtttacgatgggttaaaggagatgggggtttctgacgcaacgttgctctcagaaaccaaaaggggggctctgtggctttacttcaacgggcgtccgtgcctgttaaaaggtctaggcgactacgttttttgccaactggggttaagccacagcgtacgcgtggttccggaaaacctaacggatgggaattatctctataacctaggcagcgtcataccatgtcgcctgcttgtggcactgagctactgtttggcgttttgggggcacgctgaccacgagccctgggtcaggttgttcgccggcaagatttttatactgtacttaataatttccggccacattatgccacggaagtcaatcttagaacaggtgggaacgtccgggtacggtggcttcgtcgaggcagtgtgtcgcgatgttcgcgcggtacacgggattccggcgtgggattttgcgtccgcggctccggcgttaacgtctcaacagacggactacttgtttgcgttcaataacagcgtagtgtagggtcgcctcacactaccgacgtggcgaccggcaggtatcccaggaccaaaaaataaactctgtcgtagtcgtcgtaaactccgaattggcttttcattatctcgtgtgccagaccggggggcgatgggtgcgaaaggatggcggccaggccttcctcggccgttaaacgtagcgacgtttgagcctgaatcaggctgccagcgtccctgaacgtgagcttttggttcaggatgtcaaagatttcgttaaatctggaaccggtcaggttgacgaacggacgagtgtgttttagatgggatatggcctttgcctccagcgccgctagatacgcacccgggaacaaaaacgacgtgggggcgtctggcgcgcgcgaaagaattggggagatgtaattttctgtcaggaaatcaaaaagttgtccctttttaaggtattgtttggacccggacgggtcttctaccttggaaaatctcccacccaggagcgaggccaggttgaggcgccggtggggcgatctgggtgtgatttgggccagctccaatagcacgtaaaccaaaactaaacagcctagtgccgagaggccggctcggaggttccattggtacgcttgaaagggaggcacgtcttcgctgaacaaagtggtcgcaaggatgtatacgagatctggtccaggtggctgaccggtaccggggttcttggaaccgggtagcagtccgctctgtgccaacgtcgcgtatatcacgtggtttgtaaagaagtccgcgttgtactttgtttctttgtttaggggcgctagagaggcgcgttgcctcggattcgaatatgtaaactcgaaacccaggcccttcgtgttttgttttaggtgagagacaagcgtcgccagctttgtgccgaggttatcccagaggccgtttcgagttggcggcaacggggtacctgtccggtgacagtagtacccgttgaggaggcacagcgcggcctgcgtgtccgatacgttgcggacatccgcgtaaaagtcttgtgtgatcgaggcggctgttgagattacggcagacattagctttagagacgtggagttttgaaagtttgttgtgcctcgcaaatctctggggaacaccctgcgttgcatggcgttatctgtgaggctactgtaccacggcccgtattgtggcagccattggttttggttcatgtacaggcttggcaggagctccagacggagatcctggtttaccgtgaatcccgggtctccgggggctattgtgacaacgaacgcgtctccatgtgtctgcggggcgattttgggttgcgggtttagtgatgtttgggcattggcggtttccctgcccggggaccgcagatccccgaccatgttttgaattgtttctaggtcgcgcatgaccacttctgtttcccgttggtgcgtttgcagcacgttgtctagttctagccggagcaggttttttcgcagcgtgtgaacccttagcgccagctgttgatttcgcatcacaaacgcgtttcttctttcttctatcagagatggtttgtgaataaacagaacgttgtttggatggggggcccaaaaagttgcggtaatttgccgcgctggttttcgtgggtatctcatacagcatttttctgacgtgcgcatcttcccattggctaaaaaacccgttaagcgccgccgacgtggtgttgataaatgtaggcggaaacgctgatgactcagtatgaattgttttaagtgttttctggttcttaaatagcagccagggccttaccgaccagttttcgtgtcctcttggcgcggctctgcctatatatgccacggaatctgtcagttggtgtaagccctgccatcgctgtgatatttttattgcgttgccgggaaatacagccgcgcttaggcacgtcttgagttcaattacgtagcacgttttttgatgttcgctattaacggtaagcacacaatctgctattcgcctacctagggaaacctcgaaaaaaatcctaaatttaacgtttttggttgccctgagtgttgggcgaattgaaagaaactttaaaatatcatcaagcgaccggtgtttgattaaccgtcggtatactgctaagtgggcgcgagttccagcccgtttgcgtgaggccggaagggcttcgagggtcgcgacgtgcttacattgatttgcaaacgccatggccgaaggagggtctggctttggcgacgagcttgtccgtcagatgcgggatcggaagcctagatgggacgagtcttccgacgatactgatgacgttgacaccgaaagcaccgatcttgagtatgatgatgtgttccccgtggtagatacgcacggcttaatgagccctggaagtcaaaactatgacgtacccacgtctccgtccggaacgccgtgggaattactgcacccggacgccctgtatgcacatccgaggtgcccgcctaaaagggcggtggtgccgggcggtggcgcgcgtcccaaggtgtccgcgttctcggctagacttcaatatgttggacgacagagttttggggatagagagacgcggcagcttaccggggctcagttttcttccgagagcgagcacgaatatgcggaaataccggagcgtactactacacgcccggttgaaagcggtgacaagagaaatttcacctccgggcgtaggggtgcaatctcgggaccctcgtcgacaaaacctagtcacggtgcggggttaacacgtaagactaaaacgtcgctgagtgttagtcttaaaaacttgctgcggataaaggacgatgatgctaaggttgatgtacccaggccggttaccgtgccggttcatctcatgcagccgcatcctatgacggaatatagaaatgcctttttaatctatctagagggggtcatgggtgttggaaaaacaacgctattgaattcaatgactggcatggtgccgcaggaaaacgtcttaagctgtcccgagcccatgaaattttggacgtgtgtttattcaaattgccttaaagaacagcgcagcatagttaagcaaggcacccacgggaaattgatcacttctgctcgcgtatacgcgtgtcagagcaagtttgcgctaccgtttcgtgcgacagccgccggcatcggtcgcaacctgcaaccgtggctggtgggaaacggtagcacaaagccagcgaattggattgtttttgacagacacctactgtccgcaacggtggtttttccgctggttcacgtaaagtacaacaggctaacgccggatcatctgtttcaaatcctatctcttttctcggcacacgacggcgatgtggtcgttttgctaacgctcaacagttcggaggcgcacaggcgcattcaaagtcgaggccgtaaggaagaaaaaggaatcacgcaaaactacttgcgacaggtagcgtgggcgtaccatgccgtgttctgtacgtgggtgatgatgcaatatctcacaccggagcaaatggttcagctgtgtgtacaaactgtgtccatagaggacatatgcaatatgaattccagattgactcatcggtttctaaccctaacaaaactacatgaacagagtatgatacccatggtggcagaaatgttagtatcggttaaagaacacgtgaccttaatggaggtctgtttgggactctttaaagagctacgaaagcttcaaattttaattgttgacgcaggagaacatctagatgatgcgtgtggcctctggggaaatatttatgggcaggtgatgtcaaatgaggctattaaaccacgagcagtgaactggccagctcttgaaagctacattcaaacgctaaccaaattggaaggcaatggcgcgtattagttttatatttttttttactataataaggtgttctgtaacggacaaatacgtgtatgatgaaaaatcaaatgtagagttggaatttaatggaacaatttaccaaattaattggagaaacgtaagcaaagaattaacatctattgtaatggaagatgcgtggtacgattctttactgctggaaccattgtctgtgactttggaaaaaagaaaaagtttgctgcgatcaagtatagttaatgtgcataataatgactatacattttgtaaatcttcaagtgaccatgtaattaaccttacggtggattttaattacagttctttgccaggttttactgggaattttaatgtaatgacacatgctttaacacaaggcgttttattaacgaaacgtgaattatttaccaactcaacaaacattatggatcttttttacgcggaaaaaataaatgcagaaatgttcaagattacttttgattattccaatgtaataatttccggcattataacagaaaattggatattagtatcagttacaaattcaagtgtaaaatcaaatatgcaatgtgttgcactgttgtttggtgttcctagcacatttccagcgttaaaaggctatgtatcttatcgtgatttattggtcgttaaaaattcaaactatgctctgggtgtaattgcgccgaaatcttataatactcttgatttggcatttttaccaaaaaattttacagaaatgtttgtttcagttatagatagtccgcttaatgccattgattatctcaaaggaaaacttttggctattgaagcaaagggtgcctgtcaaaatccttcaaatgaaaatgatatcttatctttcttttttgaagttactgcagtaaattttttatttataaaaaatcttcaaaagcagcaattagttaacgttgggtgtgttgtacggcatgttgcagcgttagaaagtttaatgcatttattaaggctctgctacccaacgtttaagttatatgagctaaatttggaaacgttgtcgcatattgcagagtcccaggtttttaacttgccggcaaatagcatgttatcgttatcagtaaatgatcaagaggtcgtattttcaatgtttaaaattgtatataatacacctaaggttggtggtaaaattctaaacgaaattgtatatattactaattacatgtacaccaaatacagtgaaaattatcagctaaccaacacatttaggcggaatgtaatgaacatgtatgaagttttaacaacaattaaattaaacgtcacagattcttctgtgttttatccgtatatattgttcacatccatgtgtaataatgtagagatatcatatatgattaatcaaatagcaaagcccgatgacatcaccatatttcgcgtgttttcgccttgttttttaagcttaaggtttgatttagatgaaaacaagctacgatccgacgcaccgcagacgtcaaaaagaaccggctcggaactggcacaaggagcgtctggtttttggcggctgttgcacgcttttcacgccacgcgtataaacgagttttcagtcataaattgtaccaggctggcatggaagcaagtaaccgcgctgatgccgcttacgaacatcacgtacgtcataagctccgtgcgccctgatcacgctcgcgtttacgaggtttcggaggtgttccttaacagcgcgatgtttgtctcggccgtttatccaaattgttctcattttacaccgcccggtaccgccctacacattccgatattgtataacttttccgccccgaggataggatgtccgctgtgtgattcgattgttttgagctatgatgagaaccagggactacagaccatgatgtacgtgtcaaaccccacagttcaagccaacctgttttccccgtattctcctttctttgacaacgataattttcatatccattatttgtggttaatgaacaacggtaccgttgtggaaattcggggattgtacagaagacacgcactcagtgctattgcattagtatttgcttttattggaacaatgtctgctctgtattttctgtttaagctgttttccatcctggcctaaactgttaataaagcgtaaaactttaaaaggttgtttcccgtttctttttggatggttacgcggggactgagattagcgagaacctcggatagaggcgtgccgtcacatattgattcaatcacctctgcgccaacttgaatggccagctctcgattttggggaggaaacattaaaaaatatttgtcggtcgcggttaataatcgctgcggagcccgcgacaaaactcgaaaatccttctgaaagtcgtgggactctatggagtttaaacaaaacaccatgtcttccaggcgctgttttacgcgcgagagcatggattcgaagtattctctctgatacgggcccgccgccttacacgtattcgcgtatagcattttaaagtgaaacagaatattttccaaacccaaacgctgcaataggacgtgctttaaaaaaaggtgttggataggctttagttttaaattaggtaaaaacattatagagttgttggcgcgctgcataattgtcacggtgttaaacgcggtaatcgggctctcgcctatttgtggctggcgcgaacccatggcaaaaaatatgtctgccagtaaacagtccgactgtacatttgactcgctggtcgctggcggttgcttgaggatggatcgaagttttttattatccggcggaagcgcgctacataaggtcggtggcgcatttggatcgtattcttgcgtggtgtttgccagctcgtttatggttatctgtttgttatttgtaagagttaccggtggggtcttgacggtcgccgggacggaaaatggcctgcagtgcaaaaaaagagagttactcgcaatcactaattttgagtttacggtaattggtgtgtcctcaaagtcagaacttctaattactgggtctctgaacaatgatacgcctctctcgccttcgtccgttcccacgcacaagcagctgtttagcgcgtagggatggggtgacaccataattcgcagcattgataattccggacgttttggggcgtacaatggcatcatgtttgaatagatggtgctgaattttttaaacaacaggggaaggggtacggccggtcccggaagtccgaccattgcggcaagtgccgacgagtacacgacatgacagacgtactcgccgttggatagctgcaccgcccccccaactaagcgagcctttaaatctgagatttttatcattgattttcctttggtagtttcaggaaccgcagcggccttggcttcgcggttatatttttgctttatgtctccagtgaagtcccaaacgtctaatagtgtgtcgaattacctgcggttttaagtttgaaaacgcgtgaccggtcgtagctatgaaagacataaaattggcgttgcgtcctaactcgtctaaccgtatttttttacttcgcacgtatatctttggaatgaggttgcattttttatatcccagaatagcgcattcgtgtttgcttttggtgtgactaatgatgcgttttgaaaagtcaaacgtaccaaggcgggcggcgcggttcagggctcccagatccaaacgcaaaaacgaacaacattccaccaaacactcgataaaacgcttgtgtagagtttgtatttgcgctttatttcttgtctccagcgttgtgaatattgtgtttttcacctggtctattctagcatatggaacgcttaatattgggttcaattcatccgaaagttttttaagtaagggcctaccgtatattttagaaattgtcgcagttgccgccttaaaccaggaaacgttgcatccgtctttactagaaaccacagaagggaaattagtggtccagaaaacgtctcgccgcccccgtctaactatgtattggttttcaagcccgtggaggtctaacgtagagttccagttcgcaatcgaaaccgggaaaatgtgcttcaccggtaaaaagcacctgaggtttcccatagcgaacataaagtgcagcatgtccccgcttatgttaacgttaattgcagccccgttgcacaggttgtcggagtagaacacactcacggtgaatgacggttctttcacggagaattttttcattacaaagttattagtgagcctggggatgtcgataattgactcgaaggtaccggcgagagaccagcacgtgattttggtattaattaccatattggtattgaaaactgagattttgaagttgtgtaaactcatgtgcttaactggatcgaacatgtctggaagaggttcggtctggtggtcgtgacgcgcttgttcatatcgatctgggttttgatcggcgttaaaaataaaattgtaatctctatcttttggtgcgtattgaattcgcgaaaacatggcctctaggacgcgaggcgtttcgcctggccccagaaactcggtgttaggatacagcctgtcgcacgaatggcacacgtgttccgccacagacatgcacattgcgacgcggaatattggtacctcgggtggaaacgttagatagtacgttgactttggcgtcacaacaactaccttgtacggttttttgccgggatcctttttgttaattttcacctgtagatttacgtcaagcgattgatttactatgttttggtagtctatgattaattcgctcgccactttgatgtcctgaaatttgttttgctttgagaggttttttaaaagtaccggattggattcgatgttgtgaataattaccgtttggacgcgatgcagttcgggatgcgaaatccaatccgaagtaaaacccaaaatgttaaattttaagtaattcagatgcagttttgcacacgggggcggacaccgagctagcgtgaggattttgaatggctctaaaagtttaaagtaattctgataacacgtgccgatcagatgttccggcagtgtgcgcgtaaaggcagcaaagcattcctcgaattcccatatttgaaggtaacgacagaggattcgaaacatgctttgataatttacgccacataccattaatggcgaacagtacagcgaccgtaggattcgttcgtcgctttgaaccaaaaccgtgtcggacagttcgtaggtgaaaacggtgggtaaaaacgtatgcgtctcgatatgggaaaatgtacatccgatagcatcatcaattattaaacccggtcccgcgtactcgttaccggtcttgccaacgataccaccttggcggtgtataaaagggtatagtccttctcccaataccagacgccggagcgcacggcacgcgtaaaaggtggtctctgtttgcatagccctgtccaaggcctctagcgagacgttttcaagaatctcattattggtggccaggcagcaaaaaattgccagttgagttttaaaatcggaattggccggtgacggggcgttcgctgccaccgtagcaggacacgcgggcagaagaacagggccctgcaacagcataatggaggcggctctggaggtacgaccctttccatatatggccacggaggccaatctgcttcgtcaaatgaaagagtcggcagccagcggactttttaaaagttttcaactgctgttgggaaaagacgccagggagggaggagttcaatttgagggtcttttgggcgtgtataccaacgtaattcagtttgtgaagtttctggaaacgtcgctagcggttgcgtgcgttaacaccgagtttaaggacctcaaacgaatgacggatggaaaaattcagtttaaggtatctgtaccgaccatcgcgtatggggacggcaggcggcccacaaaacaaaaacaatacattatcatgaaggcctgcaataagcatcacattggtgccgagatagagctgtcgactgatgacatcgagctgctattcattgacagagaaaccccactcgattacacagaatacgccggggccgtaaagacgattaccgcctctctccagtttggcgtggacgcgctggagaggggcctggtagataccgtattgaatgttaagcttaggtccgccccgccgatgtttattctaaaaacactatcagacccggtctacaccgaacggggtctaaagaaggctgttaagtcagacatggtgtccatgttcaaaagctaccttatggataactcgtttttcctcgacaaatcagacatcgccgtcaagggaaagcagtacgtgctgtcggttctctccgacatggtgggggcggtgtgtcacgaaacggtttttaaggggacgaatacgtatctgtctgcatcgggagagccaattgccggagtcatggagaccacggaaaacgtaatgcgaaaactgttaaacatgctaggtcaggttgacgggggcatgtccggtccggcgtcttacgccaattacgttgtcaggggcgaaaatctcgtaaccgccgtgacgtacggtcgcgtcatgcgaacgtttgaccagttcatgaaacgcatagtggaccgtcccaacgcgcagcccagcgtcgacgatgatcgggacgcggtggcggacgggcaggactccctcgccaaaacaccgatcgcggcggctgttatccagattggagataaactggtggctttggaaagtttacagcgaatgtacaacgagacccagtttcccttcccgttaaacaggcgtatgcactatacttattttttccccattgggttgcacatgccccgtcctcagtattccacgtcggccacaattaagggggtcgagcacccagcggaacaatccgtcgaaacgtggattgtaaataaaaacaacgtactgctgagtttcaattatcaaaacgcgctcaagtccatctgtcatccgcgcatgcacaacccgatgccgtgtgggcaagctttgggtcaggcgtttccagatcccggacacgttcacaggtacggacaaagatctgagcaccccccgaacatgaacctatacgggctggtgtacaactattaccagggcaaaaacgtggcacacgttccggatgtcgccctaaaggcgacgatgaccacagatgagttgctgcacccaacctcgcacgagacgcttcgtttggaggttcacccgatgtttgattttttcgttcatcagcagcctggtgcgcaagccgcgtatagggcgacccacaggactatggtgggtaacattccacaacccctggcgcccaacgagtttcaaaacagcagaggcctgcagtttgacagagcggcggccgtggctcacgtgctggaccagtcaacgatggaaattatccaagatacggcgtttgacacgtcgtacccactactctgttatgtcatcgaatgcctcattcacggacaggaagacaaatttttgattaattctcctttaattgcattaaccattgaaacttactggaacaatgccggaaaactggcgtttattaacagcttccctatgctgcgatttatctgcgttcacctgggcaacggtagtatttctaaggacgtgtacgcccattaccgaaaagtttttggcgaactcgtggttttgcagcaggcgctctcgaaaatcgcgggccacgaggtggtggggcgcaggcccgcgtccgagctgattaactgtcttcaggaccccaatcttttgccgccctttgcttacaatgacgtttttaccaacctgctcaggcagtcctcgcggcaccccatggtactcataggcgacgaggggtacgaaacggaaaatgacagggatacgtacatcaacgtcagaggaaaaatggaggacctagtcggtgacatggttaacatttacgagaccagaaacaacgcggatcatgacggccgccacgtccttgacgtcggtccctttaatgaaaacgaacagcacatggctgtgctggaaaagcttttttattacgtggttctgccagcctgtaccaacggtcacgtctgcggcatgggcgtcgattttgacaacgtggccctggccctgacgtacaacggcccagtgtttgctgacgtcgtgaaccccgacgatgagattttggaccacctggagaacgggacgctccgcgagatgctcgaggcttcggatatacaccccaccgttgacatgattcgaactctttgcacgtcgtttctcacctgcccgtttgttacccaggcctcccgtgttgtgactcagcgggaccccgcgcaactgttgaccactcacgacgacgggagatacgtgagccagactgtcctcgttaacgggttcgcggcgtttgctatcgcagataggtctcgtgacgttgccgagaccatgttttacccggtgccgttcaccaagctgtacagcgatcccctggtggcggccacgctccacccgctggtcgcaaattacgtgacgcgcctgccggcccagcgcgtgccggtcgcgtttaacgtccccccggccctcatggccgagtacgaggagtggcacaagtctccaatgctggcctacgctaacacctgcccgatgacgcccacgtcgttgagcaccctggcgagcatgcacatgaagctgtccgcgccggggttcatctgccacgcaaagcacaagattcacccgggctttgcgatgaccgccgtccgaaccgatgaggtgttggcggagaacttgctatttagtgccagggcctcgacgtccatgtttttagggcagccatcggttatgcgtcgggaagtcagggcggacgcagtcacgtttgaggtgaatcatgagttggcatcgctggacatggcgctcggttattcttccaccatcacgcccgcccacgttgcggcgattacctcggacatgggcgttcactgtcaggacatgtttctcatgtttcccggggactcgtaccaggacaggaccctcaacgactacgttaaacaaaaagccggatgccaacgattcggtggtcctggccagattcgtgagcccgtcgcttacgttgcgggggtgccgcactcggacaacataccgggtctcagccacggacagctggccacgtgtgagattgttttgacgcccgttactgcagacgttacctattttcaaacccccaacagtccccggggacgggcatcctgcgtgatctcgtgtgacgcgtacaacaacgaaagcgcggaacgtttgctctttgaccactccatcccggattctgcctacgaataccgcactacggttaacccatgggcgtcgcagcagggctccctcggagacgtgctgtacaactcaacctcgcgccaggtcgcagtgccagggatgtacagtccgtgtcgccagtttttccacaaggacgctattttgcgtaacaatcggggcctgaacacactagtcacggaatacgcggcccgcctcacgggaacgccggcgaccagcgcgacggacctgcagtacgtggtggtcaacggaacggatgtgtttctagaacaaccgtgccagtttctacaagaagcgtttcccacgctcgccgccagtcacaggtctctgctggacgaatatatgtcgaataagctcacgcacgcccctgtgcacatgggacattatatgattgaggaagtggcccctatgaaaagactattaaagatcggaaacaaggtcgcctattagtttagctcagacggtctggagctaacgagagatggccctcgataagagcatcgttgtctcggtgacgtctagattattcgccgacgagatagcaaatcttcagtcaaagataggatgcattttgcctctcagagacgcccaccgtctgcagaatatacaggcgctgggtctggggaacctgtgctctagggattccgcggtggattttattcaggcatatcactatttggacaaatgcactctcgccgtgttggaagaggtcggtcccaacagtttacggctaacgcgcattgatcccatggacaattatcaaataaaaaacgcgtaccaaccggccttccattgggataactactcagaattggtagttataccaccggtctttgggcgcaaagatgcgaccgtctcactggagtctaacgggtttgatgtggttttccctgccgtggtgccagaaccactggctcaaacagtgcttcagaagctgctgctgtataacatatactacagagtggcggagacgacgcccaccgacgtcaacctagccgaggtgacgctgtacacgaccaatatcacttacatgggtcgcaactacgccctggacgtggaccccgttgggtcgagctcagctatgcggatgctggacgacctgtccatttacctgtgcgttttgtccgcgttaattccgcgcgggtgcgtaaggctactgacctcattggtgcgccacaacaaacacgaattagtcgagattttcgagggggtggtgccacctgaggtacaggccctggatctcaacaacgtaagcgtggccgacgacataacgcgcatgggtgccctcataacctatctacgaagtctcagttctatatttaatctgggccgcagatttcacgtttacgcgttctcatcggacacgaataccgcttcctgttggtgtgcatataactagaaacgggcctccctgtgctttgacatgtcgatccccaaaattatgacggtgtccagagacaacgagggtacggtgtgtgaagtcgcggtggacaacggacgacacagagcgatgatttattaccctaagaccaccaacttagcaaacgagcgcgcggacgttgttaaggaagcttttgataccgaaaccccagtggacattgtaaagcaaattgttaacgagggcctagctatatccaaaaaaaattgcgtccgtttggcgttgtatttatatttttatttgcagtacgtgtgctttgctctgctcctcacttggcagttaaacccgtacatggacccaccgggtctggtgtttgcggttaaccccatgggtccaaaacatgtcacgaaactaccgcacccggctattgttgcggtaggttgtggggcagacgccatctgtaagaactgtagcgtccccgatatcaaaacggagcttggaatggtttaccacaacgggtctagcgattctggtcagcgcgcacactatgggctggccctgttaaaggcggcctggcttgtcatgggaaatgtgtgtccggaaccagtagtgcggcaaggcgctgcattacttggtccatggaaccggacggagtggtcggattttaaatcggcaatggcggcaaccacgttttgcggatccagaggcgttctgtggtcaccgattcatgaaaaaaacctctgtcgccccacctggaatgatgtaattaacacatcagtttttacaaatgaatcactctgtccaaatatacctgtggtgcccgaaagtgtaatagtgcttaatggtgatgcatgagaacaataaacgtattcccacgcacttcatgtacgttgtttttattggtctggtaaggtattaacagtaatgggaggttccgctggtgcctataaagcaaaacggtcatagagtaacaatattgtgaatggggaaaatagtctgtaaatagtttatccgaacagtatgactgcacacacgaatggggttttaaccacgacgggcttttcaacaagtcagccggaatcggttcaagtttctccattttatcgcgtaattacaaaacctcccgttatgggcttgtttttttgcgtggctatgtgcgttatcgcgttggtatggtacgtgatgcggagggtgtgttgtaaggggcgcgttgttgccgattcgtgtcgcgacccgcgtcaacccgcgtatgagatgttgaatgttaggttgcgtccccacggaaccaatccatagagaactttcacgtacatagccaaagcgcacgacgtgctctctagctcgtttattctagcgatttaaaaacaaatttatcgttggttgccatgtagtgggccatgacggcggcgacaagcacgtcgtccgacatggttttttgtttcgcgcagtacgtgtgacctccgtcttttagtggaatgcaacgtattgccttgatttgatctattagatacgctacgggatcaaacgatagcttaatagtgtgagacaccacagtttggctagcgctaaacgttcccgagttcaatgcgtaaataaaagattcaaacgccttggctttctctggccccaacatgtaaatgggcgaacgtataagagtgttcttgtccacgtggtgtaaaaaactaagagggaccgagcaaatctcgtttaacacggttgctatggccactccggcatcttgactgctgttgccttccacggcgacgttaacgtgcgtgatctgcgggtgaagggtaacgatcgctcgaattaacgcggcggcacatgacgctatctggtacgcggcggtgccggttagatctcttagaaaaaaatgttccacgcctaataaaatgcatttaatcaccttgtgatttaccgcaatcaccgcaccgatgccggtgccggacgcgtctgtgttgttggtgtacgccgggtctatgtaaacgtggagacacggcttcatcgcgccctgtatatcttgtgacgtgctctttacgcggcacagatccaattgtgacagcgaagagtcgctaacgattttatgcatgctttgagcggacgtggcagcgtcccccattagctccgtggagaatgcgccttccaaaaaaagattagtcgtgctccgtaccgtctcatcaatcgtaatataggcaggaatatgcaggcggtagcacgggcacgccacgacagtgtcttgcaagttaaaatcatctttatgatccggacatacgtagttcaccacgttaagcatcttttcgtgggcgtccttcaaattaagtaaaaaactagttgatttgtcagatgagttacttgaggatataaaaatcagttttgcgtccttttgaagcataaaacccagaatcgcggggagggcatcttttttaatgaagttcgcctcgtcgatatataggaggtggaacgtctgtccccggatgctctaaaggcaaagaaaaacacaaaacgagttacgcgggtatggagaacgatacgcctaaggacaaaatctcggaagctgactttcaacagtgtcaggcgttctttcaccgtcccattagagatctaatttcatctggagctgacgctttaaaccactttagcctatctgaatcagacggacataaattggaacggattgttcttctgcttgacctggtggggacagaatgtctctcttataccacgatcgctgcaaagaatgtcaaatgacgcgcgtcaacagcccaatatgtcgatttcataacgtctctaacttataccagtgtttggattgtaagcgctatcacgtatgcgacgggggacgcaactgcgtgatcgtgtacactcgcgaaaatctagtgtgtgatttaacgggaaactgcgttttggataatgtgcaggacgtatgttcgtacggtcctccagaacgccgcgtacccgacgccttcatcgatccgctcgtgtcacacggcacgagggaatgtcttaaaagcgatatactgaggtactttgagacggtcggtgtgaaatctgaggcatattctaccgttgtcaagaatggacaattgaatggcatcataggtagattaatagacgctacgtttaacgagtgccttccggtaatgagcgacggcgaaggtggcagagacctcgcggcgagcatttacatccacataattatctccatatactccactaaaacggtatatgataatcttctatttaaatgtacgagaaataaaaaatacgaccacattgtaaaaactatcagagcgcaatggatgcgcatggtctcaaccggcgatccgtcgcgggtcagtgcgacgggttgtttcacgtgatacttccgcgagggtttatcctcgcgaacaatattacgtgcggtgaacggcagcggttttttgcacacacttggttcgctgcatctggacgcacgtctaagactttatacgtgtggggacgggtatttcaaaacaccgacccgggccgcggggacggtccgtccgggccgtggtccggactggcgattagtctgcctctgtttaccacaaatggaaaatttcatccgtttgatgtagttatactcaaggccgatacgcctgactctggaagctcgtggaccgtgaagttcttgtatatgtcattaattgcggcttacagaaacgcaatgcgaggtttaaaagataaagtttcgcaatgtaccgatgccgccgttgacggtgaggttcatcctctaaccgtcttaaaagaagcgttggtatcaccggacactgctacgcgacccgtgtccgcgtgcaaccctctacagatgttgaccggactcttacagtctagggtacgggacgactacgtgacacaccaccgtgcgctcgaacgcccaggtaatgtgaggggacaagtaatcgccccgacgcgcaccgagatgccaaacggatcgccaagtcgtgtaaggcttggattccgccctcccaaacaagccaactatccaaagacgtgggcgcaggcgcgtcacgttttctcgtctcgcgcatattacgtgtgcgtatatgataacgaagaactagataccaagtggcagcggcaagatccgcggccgttgccgctagattggtccgatccggtcgcgtacctgttagagggcgatttgtttttaggagccaagcagaatgcgtttgtcgattctatagaaaagacgtgcaggtgtcagaactataccattaagcaattttttccggttttgataaatagggacaacgaaacagtcgacttaattaaggagcattttatagaggcgtgcttcgtgattagaaaccaggtgtcagagaggagcgcttgggtaaaggcggcgctgtttcgcaacgatagtaacacgtattggaaggatgttttgggattatgggagcatgggcctcataagctgggtacggctataaaactaccaacatcggaaccttgcaatgccgacgtaaactggagctggctcctgtgtgacgaggatataactcggtcaattagcgggcagtctactgtttgcttagttgtctcgcctaccctgaccgcctggctggtgctcccggggggctttgttattaaaggccgctacgacctatcaagcgaggatttaatgtttgtggcttcgagatatggccacccagcgtcgtcacattcttaaatcgtttttaaacaaggaatgcatatggttgcgacacccgggtacgtccgcgtttgttcgggtatacaccgcgaccactgcgcattctgccgtttttgacccgccggtaactagcgaaaatgcgatgtcacttaactttttaaatgttatgatcgtaatcatgaaaccaaaagaatttggcccgtgcgttaccgtgtacatgaatggagatattctagatttttgtgccacggaatctgtcgccataagggacgtgcctggtagggcggacctgtgtttaattcgttttggtaccctttctaatgcgccgaggagcgttccgatacccgggccgttgaacccacatccgcgagaaaccgtgcccgggctaacaaaacaggaaattatatacacttcgcaaacagtgccaagaggacagataccagatgccataaaggggaaagagttccaccaaataaatccgtttttgtggtttgacggaggggcgttttggcaactgttcctctctgtggattttatgctgctctgtcccgcactcgacacagttccgtccctggccagaatcgttgggcttcttacacagtgcgataagagcacgtgtaaaatttgtacgggggcccacgtacacgttaacccgtatcgcggatacacgccacctgactcgcaagggacctcaccctcgtgcccctgccttatctcgtgcggggccaggcgcgcggcggatgtcctggttaccggacacgttaatcttttgggcctgctctttgaccccaaagcctcccccaaagtgaccaagctgcgtttaaaaagaaacccacgcccggtaccgatagaggacgccatgtcgggcgtcacggccgaagggaccgaggtgcaacccacttcgctaccgtgggccctcattcgcctgccggatttagccagtcgcgtgatgctatacggctgccagaacttaaaaagcatctgcttacgttcttattgaagcacgtcgcacacatcagcgaactcggccgccgtccggggctcgcgtacacgatggttccgttttccttctttatgttcaggttcttcgccggaaaccacctggatagcgtggcgatgacctcggagaaaacggcattggcgacgtgtttttggtgggccacgtaacccacgtgaacgttttcaaccgacgagagaagaacgctgataatggcgaccacgatccatgttttcccgtgacgacgcgggataacaaaaacgctggctttctgcttaaacgtctgtaacacgtcgtccgtggtttcaaataaaccaaagtgttgcttaaacgtggcaaataactggtgtgtcttttcgggtgctttgatagacgcgataaaataaaacgtgtgcatgattaattgttgttgaaagggctcaaggcaacaaaccccgggcacgtaactcccatttaaaaatgacgacaggttggctaaaaactgccgcagttctgcataaacggggcactctaggaaagcttcgtgggtccgttgggccgattggtattccatgtactggtccttggcgtttgccatttggacccgacaacatgtttgtttaaggtagtttgtgagttcctcggttaaccggggcaacgtagcgtggctagaaattcggggctttcccaacgtaggggccggcatgggttcttgaactctgcggtacgtttgaagataatcgtctaaaacagaactatatgcattaaccgcgtgaataactcccaagcatgggtgggccattcgttcggtcttgtcgttgcccgatataataacgggcggttgccgaaaccagttttcgcaaccaccgtccgtgaccacgcgcaagttattttgaagacgttcgcgatagctggttaacaacatgttcccttcaagttttttgaataacgggcaccccgaaacggaacgccgcttcgttaaaggcgttcagttagctttagacctgtgtgacaacactccgggacagtttaaactagttgaaacacctcttaatagttttcttctggtatccaacgttctgccggaatcgcgcccggttagagactgtccgcagccggaagggtttgactttgaacacattcacctcccaaaactaacacgcatgcagcgtgtcctggggcgatactgcgaccatgttaacaacgacgacacgtgcgttaacgtaaaggcaagttcctcgaattcacagggtgccttgttttatctgccgtatggacaggacgaatggaattgggcgctcacgttaaggaaagacaagttggttaaaatggctgtagagggcttgtcaaatcccacgacctggaaaggtttagagcccgtggatcctttaccgctcatatggcttctgttttacggttcccggtcgttctgtcgggaaccagagtgcctatatgaacgcaattttggtatgaagggacccatactcttaccgccacatatgtatgccccccaaaaggacgtaatgacttttgtccatcatgtaattaagtacgttaaatttttatacgtgaacgccggtgggggtcttgaaactgaaccgtccccgccgttcgaggcctcgcggttgcgcgcagccatcgctcgtctcggggacgtggaagcggatgacgcatacctgtccgcaaagtgcatgttgtgtcacctgtacaagcaaaacgatacgatttcgattcatgaaacacacgtgggcggagtcatcgcattaggcggagacggtgcgagatatataacgtctagtgttcgggctcaacggtgcacgagtcggggagattttgttttaatccccctgtacaacattgaagggctcgtaagcatgataagggaacatggcctcggcagcagctaaaaaaatgttaattaagtcggagctcgagtcggaaatcaacaaaaaactgtccatctccgtatttgacaggtttggggccgacagtgccgtgtttaacgcgcagtataagggaaccagggaatcgctgcggtcgtacaacagcctaaaaaagaaggacgatctggcgaccgttgtcggaacgctagaaacgtcgctgcgtgaaaaacaaagcgaattgggattactaaaggggtttaacaggaaaaaaattgaagagtttgacgctgtggcggacgcggttcgcgacctcaaggacgagctgtacggagaactggagattctaggtacgcttgacaatgaatctgttcccgtggaagaagagtcccccaaggacgacattattaggtggaaattggagcgtctgcccagagtgtgccccaaaagcccttgatcccattcccaaggttcagactgacgtcgacagaacagcatcgtcccatataaccgtcattaaaacacgtaagacgatcgcccaactgaagatacctaacaactggggccagtgtagtcaccaggcgacggactggaccgccgtgctcggacgcggctcgtatggtgtggtgaggtccatgtctctcggccgctgcgttaagcattttggcagccggcgtgagtttttttacgagtgcatttttaacgatatagtacgcgcctgccgggagaaacatcccctgaaccgcgggggtgaccgtatactatgtttcctagagccgtgcgtaccatgtcgcgccctgatattcccgcagttaacagggaatctgctaaacgcggatcttaaacacgtgaaccctgaacggctggccgttgaattctctgagctgagggaaggcgttagttttctaaataatatatgtggcatcgttcactgtgacatcagtccagaaaatatactgataaagggggaactgacaactgcgtacgggagacttatgatcggagatctagggtccgcctctttacacacgggaaccccttggaccggagtgatggtgacctccaaactcgggttcgtgcagcacacgtaccattttaaggcaccggccagatttatctgtaagcacatttaccggccgtcgtgtctcctctaccggtgtctgctgtcgtgcgccgggggcccgcaggcgcatatgctaaatcagccgttccaaatcactccacaactcggtctcacaattgacatatcgtccctgggttatagtttgctagcatgcctagagaaatatcttcagccagctgacccatttccccagcagggagcgttggcggacgcttcctccgaatccgcccacccattgttctatttgcgttgcatggtgccaagagtagtcatcgccgagattttttctgttgcctgggacgttccactcgatttaggcattgactcatctggccacgcgccagctattcccctgagagaagcgtacaggcggttttttgccaaccagtgtagtttatatagggcgcaatacaaagaggatgcgttagaaaacgcatcctcgcggctgtgtaactcaaaacttaaactagttctccagaagctgttggtcagggactactttagtcattgcggaaactgcggagatcatggattttttctcagatgagccgatggttcaggagatggcgcttctcgacatcgatgagcagcagcggctcctctcgaaaatgagcctggccaactttttaaaacatgagcgagtaagggcgttttttagcgataacaaaaaggaaataagcatgccggctatacggttcgtgtataatttttatctattcgccaaggtgggagattttatcggcaacaccgacgtgtacgatttttacgtcacttgcgtgttcagggggaggcgcctgacgcgcctgtcggaagtgtacgacgcgtgcctaaacatgcacccgcacgatcgacaccacgtgtgtgcattgatagaacaggtcacgcgcggccaaaacatcaatcctctgtgggacgctctgagggacggcataatttcgtcgtcaaaatttcactgggccataaaacaacagaattcgtccaaaaaaatttttaacccgtggcctatagtcaacaatcactttgtagcgggcccgctcgcgtttggactgcgttgcgaggaagtggttaaaaagatactggcgacgttgctgcatccaggcgaggcgcactgtgaaaactacggattcatgcagagtcctctcaacggggtttttggcgtctccttggattttggaattaacgtcaggtctgacccaaaagacggtttggagtttcacccagactgcaaaatctatgaaataaaatgccggtttaagtacactttttccaagatggagtgtgacccgatttacgctgcgtatgctaaactttatcagaagcccagcatgcagacgcttaaggggtttttgtactccatatctaaaccggcgatcgagtttgtcggagaggacaggctccccagtgaatcggactatcttgtggcatatgacaaagaatgggaggtgtgtccgcggaaaaagagacgcttaactgcagtacaccatctagttaaaaagtgcatgattcacaactctacggcgccttctgatgtgtatatattgtcagatccgcaggaaaccggaggccaaattaatattaaagctcatctgagcgccaacctatttataaacgtcaggcatccgtattattatcaagtgttgctccagtctctcgttgtacaggagtacatcagtctctccaagggaactaaaaatttgggaacccagaaaaactttatagcgactggcttttttagaaaacggcagtttcaagacccgagctgctgcacgatcggtgaatttgccccgttggatccacacgtagagataccgacccttttaatcgtgacaccggtgtattttcccagcgtggccaaacaccaactggtgaagcaggcgaccgaattctgggcggctagtgctcgtgaggcatttcccgagctaccatgggatttatcctctctgtgtgcaaacgccccaccaacaccgtagatgtgaagggggagcccatagatgtatccaaagaattcgatcctattataggagaagaaagcattgtcttgttaacggcagatgggactgcccccgcggcgctgtacaaacccaaaaccaagccatccaaacataaaaacaataaattgtcagattttgtttaagcattcgtatctttattggagtggggtggatggtgtggggttgggaagggaatgggattgaggggaggatgaaatgctagaatcatatgtattttggatatgcatcctcgtaatcgctcacgtcttcttctgtttcatataacgcgttcagttccggggagtacttaacttttgtcgtcttctctttagggccttgaggaaggggtgcgtattcgggcgactttgcgcaccatttccaatcgtttctgataattcttaagatgatggctaggatgcaagtgatcggaatcatagagatgttcacggctatgactttgtgcaggctggcagatatgaataccttgtcgtatcgaacgacgggaagaagcagaatcaggtaactgacaaaaacgccaacgtagaatccgatctggtgctttaggtactttactagaaatagttctgtgttgatataccaagctattgtaaggacaaaaaacaagtttattgaaccaaagacaatatcggaaacgagcatatagaaactgtttccaatagccatcatcgttccgagggagaatactaacatctccatggctattaaggacagataaagattggcgccaatcggttttccgtaacgcaacagggtgtctagcaagctattctcaggaatctgctgctctaagacgcgtagagaccaggtggccgaattgcacgatatcatagcctggacgtgcgtaaacgataggcaaaaatgtatacagtatacaaacgaggctaaaagtatatgtttgtacgacagaacgtgaataaataactggatggtccacagcctcaaaatagacataaaaactacactgtgggagcctatgagcacgatccatgtctggaggctggtcattgttgtcgcgtgcacgcgcttggccttgaatatggccaggatggcccacgcatagtaacacagcagatatccgtccaccaaaaacgcccacgagatgtagacaaacatttctggtgcttccaaaaacaaagtcggcgtcagatgtttagcagaacttctgaccgttaggtttaacgcgctgtagttgaccaacgtgttaaagtagcacggaaatccaagtcccgggaatgtggcggtcagtggcactaccgctgacattataaacataagtccaagaataaccagtagttttacccatgacgataagataaacgagtcgctccgtgaaattttcatagttcaaagaatctgctcgcgggtgcgttcggcaaactagcagtaaaatcagcaagtggcttacatgttacggatttaaataggctccgcttttaaatatcacattctgtgcaaaggagttgagccacggcaccatgaacgcccgggaggtggcactcacgggacatgttttgcacatatcgctgcatagcacgcacgagcgcgagaaattaataatctggcaggttcatttacttgtatgtcaacaatgcggaattcagggagatgccgcatatctatttgtcaccgaaacattaagcaatactgactggggaaatataccggcgataaaccgtcacgcaccgtccataaatgagcacggccgtaattatatgcagtgggaactccgtactcgtttacggaatcccatcattcaattgttaagtcgccagcccggtgcggttaacgtaagggtcagcgagccgaatatggtaatagtgggctgcgaacgagcgttggatcactcgtgttcggtgcgcgtgactggagcctatcttcattgcgataccactatggactttagtttggattctgttgtgtccccaacccgcgaattttggttctcagagatgttttctcactgtttagtttccaacattgaagtttaccttaaaacaacgggcgggttatactatagggcatcgagtgccacgcaatgccgaaaaagggcgaaagatggcgcattgggtattcttgatatctttaattgcgaatctcgtgaaatacaagttgccgggcagaagtacaccttgagtatcgccaccgcaacatttcacgttctctgggtggacgaggcgtgtatgtggaacggggccctggccgaattttttagggcgctgcacaataagttgttcggcgaccgggaaggcgtagcgccaacgttaacgtacgtgtgtccgggggccactccggagggaacccccttccccccctacttttccgcgtttccacacctcccgctcgtgtttggaagaccgcgaaggctcgacgtaaccgcggtccaagaactcccaaaagcacaaattgcggtacactggcccccgtttaaagattcaatcttaggggatcagcttctcatacctggcatttcacctaaaaagccaggtaccgtacccgttcgttggccgctttgggtggaggatgttaacttgagtctctgcgagacgacagaaagcgttgcccgcatagtcgacccacattctatagtaatcataaaattttcatcactgttgtgccagcacctaaaatgccaccgtgcgtttgttaaaaatgagttagaatacatagcaaccatctgttccagcgaccttcgcctcttcatccaagaggaatacaaccggttacttgccaccatttttacgtgggccgcggcgagcgggtatacctgggcggccattgataaaacaacagtattcatcaaggctccccagctcagcgcagctgtaagtggtttctgcccatcactaaatagttgccgtaggaaacaatgttacgaaggttaaaaataacagttcatttcctttcacaggaacagcaaaaggtcgtgacccgtcttgaggcgcatttgggacttcccgtacaggaaacttcccacccgcctgactggctcaagtgtgaggtctgctccgcgtccgtgtttttaaaaataccagccggggttttgtatgccggactcgcaagagaccccaccagggaagcaaaacgggactcgtggctggactgtctagtagaaggcgcgacgttgttgcttaacaactcagtgttaccgattggggcgctggcgggtatcttacccaccctttttgccaacaggcggtgtgttaatttttggctgctgccacgcgcgtgggtaaaatcggcgcccatatgccctcccctaccgattgactgtgttacgcctccacagtttgtcgtgacaaagcgtggaccaatctgctggtacaaggaatggccgttaccggttgacgttgattttatgtactacctacaggaggcactatgtgtttttagtgttgtgtccaacggggagggtacggagagtcacgcggacaatatacgacaattagagaagtttgaaaaggtactatgtttattttaaaacagagtctgtctggggatgctgtgtaggctggttatttgaaaatattttaataccgatgcgttatgaaggtgtctggtaaaaattgtgccgcaataaaatatgttaaaagtctccgcgtggccctcttcttctccaacgttctctgttagacgaaaggattccaaataaatgggtttcagtaagcctttcatgtcgtctgcgttttcgtccaacagcttatggagtttgaaccgcactaggggcggaacgcgcaaaatgccttggggtgggtttaactcgtggtatgccgccaaggtcgacgctatcacttcgatcccatacaatacaaacgcggaatcattacacgtatacctctcgtgtaggtacctggcccggtcaagcgcggacaggataaatgatagggggggcttatcgtagttttccagcatagaaagaatgcagcagagttctgttacgctcgcgctcgccggcttaaacagctgcacgcggtcagaaaaaacgtgcggatatatgtccagtgcgtcgatcatgtcctcgtgttcgaccacgcacgcaaagacaaacccagtaaacgttgccgactgaagcctggtgcgcacgtgcacgcctaaatcaggaacgtttagtcgtcctcgaacacacagcttaggcgtattggaagccatgcaaatgctattttttttatgtacttccaacatcagtcgcggaaaaactggataattatagggccctctggacgagcattctgtttcaggcattaccgcctcagatcggtgctgctcccccattagacgcttcagtatttggtccatcggttccaggctggctaatttgttcgcaatatttgtggcctatgtcatttatatacacagacagcctgctgtccacatacagctggtctgcaatatcgtgtaaacttaaggaaatgagagagtgatttatgaggtccatgtgagtcagtttggcgattatgtacgtaaacgggcctaagagaatggaaaccgtgtcctgcgagtaggcgatgctcgttcgctgcccctgattgtcgaccacgggtgtgaggttgtacgtttgaaacatttccccttcccacaggctagttagctctttgagcatctccataaacggagggacgtattgtgaaaaaaagctgtttactacggagctgcctttagggatgtgacattcggataggttaatggttgggtcgcgcagaccggataaagcctgcaacgtattctgagtgagtatattaatatcgtgtctgggcctgcctccctctggtaaactctgaagcaactgcgtttcaatgcattgaatcttctttacgtagtcgtctcgttctttctcaaggttagaaattgtatcgaactgctcgttgatctggttcgtcaggcatttgaacacggtatttgaaacctgtttccggagaccctgggtcgtactttccgtaccggtcccaaagagcttgtttttgtccacaatgttttgagagacgtctgtgacgaaatcctcaacaacgtctgttatgccgctcactgttttgttctccgcgagctttatcagcaagtttaggagctccctcttggggtcggttttttgagacgattgctctaagcgagccaatatgtctttatacatgtcgttgcacgtgctaccgaccagcgcttttagaggcgcggtatttagtaactgacataactttgcgtgctccgcggtcctgtgacatgtcataatcgcggtaaataaccggtacagagggctatcaaaggcgacgcgtccattgtaaattataggccgtgaaacaacttcaactgttatatctttttgtctgtaggtgccgagataagcagccttatctccgttaaactccacagacacctcggcgtagtctggaatataaatagacgtcacatcggtcattatcgaggtaatctcctgtgccagtttgtcgacaatccgcgatatcgtaacgtctccggaagcgtacgttttcctcatctgttgacagtgtgcttgtatcctggcgatcgtgtcgccgtcgggggtgcgcctgacaataggcaccaacccaagggcgaccacccagtcgacgtatcgttcgtacgataaaacagtcctggtgtgtagtacctgattaatagtgttcagtaacagttgatcgactgttagacgaacggtacgtgcccacgattcaaacgtagaccgtttgaattccggcgtcaggtgctcgacaccgcgccaacgggtcttcagggtggtctcgaagttggtccagtcctttaagacgttgtcgtatgtgcagctccccaatgccgtcttgtacaggtgtatgaacagctgtcttccaaagatccctggatttcgaagactggagtgtagcgtttgaccgcgaacgtacgcgtacttgccctgtaggatctcgaaaagcgagatggagagttccgtcggatggaccaaacacgtcgaaccaaatccggggttcatcttgaacatgacgtccgatgccaaagtcaggggtgtcgtggatcacgtcagtcgcctgtcaaatataactaccagcccaccggaaatgggttggtacgacctggccttcgatccggctgaagactccgggccgttcttgccgtttaccgtttatctaattacgggaactgctggtgctgggaaaagtaccagcatatcggccctgtaccaaaatttaaactgcctgatcacgggcgcgaccaccatagccgcacagaacctatcgcgtcgcctaaagacgttctgtcccacgatcttcagcgcttttggctttaagagccgacacatcaatatagccgtcagaaaagctcatcagaccggagccgtatccatagagcaaattcagcaacaggagctatcgaagtattggccggttatagtggacattatgaaagaggttatggcgaaaaaacccaatggcatgtacgggactatatccaacgcgaattttgaaaccctctcgagaatgaccggaccgtgtttatggacttccaatattattgtaatcgacgaggccggaaccctgtcctcttacatactcaccaccgtcgtgttcttttactggttcctaaacagctggctaaatacccctctttaccgccagggggcggttccgtgcatagtatgcgtcggttcgccaacgcagacaaacgcgttccagtcaacttacaaccacgggacgcaaaagacggagatatcgtcgtgcgagaacatcctaacattcatgatagggaagaaggtcgtatctgagtacgtacacttggagagaaactgggcgctgtttataaacaacaagcgctgcaccgatctgcagttcggacaccttctaaaaattttagagtataatcttcccattcctgacgaagtcatgagttacgtagacagatttgtcgttcctaaaagtaagattatggatcctttagaatacattggctggacccgactctttttgtcacatagcgaggtaaaggcgtatctaacaaacctacacacatgtctaacgctagggggcgataccagggacacgaagctctttacctgtcccgtggtgtgcgaggtgtttgtgaagccgtttgaggaatacaagcgggccgtcaacctcaccaacctcaccgtgaccgagtgggtgacaaaaaacctctttaagttaagtaattattcgcagtttgtggaccaggacatgtccatagtcgccacggaatccactgaacgttcgacccaggttacctttatcaccaagtttgttaaaaacagccacgtgtctttaaatggaaaaacaaaaaaatgcatatgtgggtttcagggtacgtactttgagttcaaaagaatcctagacagcgaactcttcgtggagacgcattcgcaagaccgtccggaatacgtatacggtttcttaaacacgctactgtacaacgccatgtactcgtttcacgcgtacggcgtgactaggtcacacgagaaatatctgcaagacctaaaatttgcacccctcccggccgctctggcaaccgggcgtgtagaccttcaaacggttcgtgaagagttaaacctggaagacgacatcttctaccacgtgtgcagtcccccgccacccgcgggtatcacctccctccaggttttggtcgacacgtactgcgccctaaaggacgtgttcgcctccagaataaaggtggcgtgtcgctggtttggcggggagtttgagaaggaaacgttttccgcgtttacggttaacatggtcgtaagggacggagttgactttgtctccccttcagaacgtctcaacgggctgttggcgtttgcatcgaccgttgaatcgtataaaattaaggggtacacgtttttaccggtagcgttcggtcgctgtcagggtttgccactcagtgatgacctcaggaagaagatgccctccctggtcgtgcaggactctagcggttttatcgcgtgcctagagaataacataaccaaattgaccgaaaccatggaggacgggagcgttttccaagtgtgctgtgcgggggactatggggtcagctcaaatttagccatgaccatcgtaaaggcacagggaatgtcgttggagcgagtagccgtagtatttgggtcccacaagaacgtccagacaagccacgtgtatgtagcaatatcaagggctgttaactcaaattatttggtcatggacagcaacccccttaaaaccctcctcagagaaccagtcgataacacctccgccaagcatatagtccgcgccctccacaacccaaacacaaccctcatctactaaataaataaaaacaagcaaatggaaaacattgtgtttttattcagtccaaccacggccacggatagttgtcatttccacacaccgggggggtggccgccatagtttgacgaccagaaacgcccgggccctctgggttagaagtagccccgccccccgtggtcgaggtcatacgtctcttagggccctgggttgtaccaacataaatcacgtcgtcatcaccgttgcagtccctgcgtgacgcgcgcctcttgcaggcggcacgaacaccgccgcttgtaacgtgatctgcatcgtcgtcgctgtctgaagatgaacacaggtcaatcacatcagtggcgcccgtgccctcatcggcgtctgattcgtcccatgtctcaacagagcctcggtcgtccacttcgtcgtcatctgaaataataatggtctcttgtcctgcagttgatccagcaacaacaggttcttcttcatcggaagaccatccgccctctgccggtgatctagatatatcacctgaccaggcgggcgaggcccgtggcgaaaagccgtcagcttctccgccactttcatcagtgtcctcgtagtcatctataaaaaaatcgtcttcgctgtctgattcagatgactggggtgcgcggtgttggccagtaaccaccgcaacgggccgcggaccaccaaacgcggtcagtggcgcgggcgcgctcggtggtgtggtggcacctgtcgaagtaatgacggtaggtagccggccgctctggttaaaggactcaccgtcccgtgcgtgcggatctattctaggtggggaggagaccggcggttcgtcttcttcatccacatcactgtagtcgctgttctccgaatgagagtcggtttcgtctggcgtttttggagtagtgggaggggaatcttggtcaaaaacatcctcaggtgcagtcatgtcaatcactggggggcctccgtacccgggcggtaccggcaaccccgtacgggacccggcgaacaaaaaacgtcttgttctctttttcctaggtgccccgggaatcggcagcatcctgggagtggccggcggggtccttgacgggtcggataagaacatagccatggccgaaacgttcacctgtaaaacgcactgcctatccccgatcagttaatattccagtcaacggcacccctccgattctgaactagatagtcattcgcaagtttaaaatggttgcagcctagaaactgcggccagggagaaccggttgccgcgtgggcggtagccaggggagacggatggcgcgccttgaggacgagatgtctctgtccgtttatcagcgtcgccttgtctatagcctttcttccccacagcataaaaacgcacctctgaagcttggcagaaaggcagcttattatgtagctggtgaaccaatcccagccaaggttggcgtgtgaccccgccttccctctttccaccgtcagaatggtgtttaaaagcagaactccccgttttgcccaacagtctaagcacccgtgagaaggagcggtgaacccaggtacagtattggctatctctttaaaaatatttttgaggcttggaggtatactgtagtccggagcaacgctaaaggctagtccggtggcttgaccgcgatggtaaggatcctggccaaggatcacgactttaatatcctccggttcgcaacaatacgaccaccacataattctatctatcggtggataaatcacggttacgttgctcatatccataacgcgcttcagcagagccgcgagcttctgttttaaaaatggagaaagatttaaaaattcaagccatgagtcgctaagtagcaacgtttgtgtggagggttcttctaacacttctggtgacattttactccaaactattgttttaagccaaccctccataaatccccgcgtctggtaattactttacgtatacgtgaaacttttgtacttctgtctgagaaatccaacagaggtggccgcgtataagcagttccgctttcactaccgtttatgtgaaagtcttcaaactcggctaaaaactcatcaagactatattgcatataattcaacaattgaagctcttctctggggacactgtaatttaacttgtacaaaatccttataaaaaacgccctaaggtgaaatccatttacacatatttctgtggtagagcgatcagctttatatcgcaatgttgctgtacacagccctggacataaagctaaatttgttaaatgaaaagaagcaatctcaaacggctccggaaatataaagtcactaagtggtattattttacaacatggatttactacaagcccggtacaacatgtaattaaaaaaaatccacatgtaataaataaacttaaggtatacatacttctcatgttttatacataatatgaacgctaatgctgtacttatataatatgtatagaattatgccaatacagtcatggtaagtatattttaaggcataatgcaaacatcaaataatataacacaaaatgcacgcttccggatatccgcccagctgttaacccgaaaatacagaaatgactacacaaacacacctgaaaccaattttattctcaacatatgcaacaatatttcagggtaacaccatgttaataaaatacgcagcatgcacatttttagctaagctcctcaaagacaatttcctcttcaattgatgactcatcaacctcggtttccgtgttaatgtcggaaatatatgagtccaggataccctcgtcatcgccacaaattaattccagggcttgtaaaatatcatccagtgaatcagctgctaatgtcaaggatgttgtttctaattttagcccatccaaatggtgagtgggtgacactttgggacaagaccccgtaaaattggcacgctccacacaaacggaaggatgcatttgatcgttcacgaggggcagcatatttatttttataacatcaggaaccggacccgtcaatgacgcacatatgtccattaagattggagtcgtgacactggttgaattgacggtgaaaaaaaatgtattcgtgtcacatgcgtaactattttttaaccacacgagggcggaataaaacgggtccccggcgctaaggccgtgaatgcggcaggcacggctcattgccatttccagatttgttaaatctgttcgaacatacgagtctaaaatcaagtataaccacgacaagttcaaacaggaggcaacgcggctggtttccacccggacatccccgctcacaaacccgaactctctaggaacgcacatcaaagcgttcagacaaaattccgaaaccgaaaccctagttctgagcaccatctggttataaactgcggttaagagacgagcggccacacactgctgcttgtgaggattcagcttaggtggcctgcaggcttgctgacaggcccgtagctgcctggcggcgctcgcgcacctttgacagccgcacgccagttccagagcgaggcagaagcgctctttgcagtcgcgccatatctcggccataggagattcgctcgcgtgatcttttaggtgcatatatatctgttgggtaacaaacctcactccctgtaaaaggggaataaggtccgttctttctatcgttttctctgaatctatagttacatctttaagtataaccaaagacgctaaaaacccaggatggtcgacacctttaaaaaatcgcaaaagcgaccggatggctctatccgcgtttccatgtcgttcaaaggtagtcacgatggatctccagttagactctgtctccctgttaacacccttaactggaatggaaacagccatgacaccgtgaacttcctgatgtctcctaaaactaaccccgaaacagagctaaataccaatgactgtcacccctaccccaagccacgcccccgtactattagaccagggtgagtaaccacgctatcctttaaaaacccatacgtggagtttgtaaggtaaacagctcgtgtatatttcagacgcctgtcataaaatggataccgacgacaatcaggtaattaaactttttttattcaagattcaggagggcgtgtttacagtggtgtaggtgggagcatatctcgccaatgggaatggctgatgaatccacacttagtgctcggatcaggtgctctgtgtactttactagtgccgatgaacctaaatttggcgtgtttaggtcgtaccaaccaaaaaactgcctggcctcgccttttagaagctctatgcaagctttaatcgtgtccaatagcttgtctttaaaaatgcaactctggtacatctttatgacagtggtccaaaaaaaacaaagatttaaaaacacgttaaactgcgtgtcctggtagtcctcgtatataacctcctcaacaagaaaaaattttttaaccaaactcgccaggtactgaaacgatggtacggacaggtcgtgaaaagtgtctatcatctccttcacctcccctttgaaggttttggttacaccgaccatgtgagataggcaccagctcaggggggaggtcggatcgtgagggggtaacaattcgtgggcgtgggggtactccagttttaggatctccggcaggacgcgtacgagttcttcgtcaaatcctacgcagcccgcatccaccaggggcagtactgagttaataagtccccgaattctatcgttcgtaattttgtggagctcctttagacagtaaaccatgttgctccgacactgcggtacgatgaaccgctctccttgtctgtgttgcatgagcacctggagtatcttagacaggtatagggcccgttccactctaagggttgccgcttggtagagcggattgcgggcattgacggtcagtgtgttcatttcaccgactatcatgccgagtggcggatagtggaacttgtacatatgatttaaaagatggtctttcccgtaatggcgactcatggtcgtgttttaacgttcacagggtgttaaaaaactcagaaggtcctgcgtggacaacttttgcggactctccgaaccgctcagggataagctgtaccacgcggtgcttcttataaacgaagcatacgtcaaaaaacatgacccacgtgacctaaataagctcacagaagaagtctgtcgatgtattgtaatggaatgtgcctccctgggtcccatctccgggctcatcgcggatctgaatctctttaacctgttctgcctctaccggggttcccgggttaaaacccgcggggccgccacctgtaacgtcccgtgcgcagaatgcgcccagggcatcgtgagaattctcaccgagagggccctttgttgtaccgaaaagatgttcatagcgtctgcctgcagcggggtcgttataccgccccagttggccagagttctgcacgacgtctacgcggaaatgaaggccaagtgtttgggggcgtggcgtcgtctcatatgttgcaggcggcctatcatggctattgccgactcggtcctcgtcacttataataccctggatgccgagggaaaactggaacttagactcaaagcactgtgcaaacttgtttttcaacccatctttcttcaaagaatcttagcccctatgcagctactggccaacgggaagatggttcctgacaattattttaccatcaccggtacggccgagaagaggcgccctgtcgtgactggaagtactagcgggatgacgtgtccgggaagcagccttgtccccgattccttaatcctgccagtatgcgaaccggggcttctcccggcacccctggttgacctcagtaatgtcttagaaaatccagaaatcatcctcagcgccccacccctgagtcaatttgtcatcacaaacacgcaccccagtctgcctcagtcagtcagcattattacgccaacccagggcgttgttcccggccaatgttttatggacacgtggaaagcggtgtcacagagcattcaccaccaggcacagacgcctattttggccgccgcactaaccggttcgacatctgcggcccctggcccgcatatcgcatgttccccagttgccggcacgtctcggcaggtggaagggtccgcgggcgtcgattgcgggaaaccagcatgcgttccgcagcccgcgttaccgcccaatgtccccgccaagaggatggaaacggtagcacagttgggaaacgctcccgtaaaaaacgtccacatcggaggccgcgtatacgctccactggttaatataccaataatagacttaacgtccccgtcagggtccggccagagtccggccgatatcgccaacactccagagtcccgcatggcggccggctctccgcccttcgccgaaaccgccgcaacggtccccgctaagagaaagcagccacgcgaggacgtggcagacaaaagactgaagggcgacgttcggggcgccgcaacagtaaaccaccctttcccgggaccgtccgggatgcgcgttcgcgagcagggcttattcgatttaatcgaaagctccacggatgtaaccgcgaacgcatctggacccaaaaacgacgacgacatgctagcggctatcctacaggacctgtatggactacagtcccccccggccatcgattccccctccagcaactcggacaatgaggagatatttccagaggttagtccgccatctagcggccacggatcgccttgaaagatgaccccaagacgcaagatcacaccaccgagacagtcccacgcaacccggcggatgtctaaccgtaaaaatatcaccggttggaaagtcccttggtgcgacaaaaatgagcttcaaacttacccattttctgtgctatttcgggacactaaaaaattgtcgccagtggcgacaatttttaagtctgtgtaaatgtcactctttctcaagtttctgtcctagaattttcacatccagtttcatatcatataaagacacctgttttgttttcgtgaatctggcgtgtttttatcttgagaggccccctaaaaacatgcctcgtgtgaaaacacaacccaagagaccccaagtgcttgaatttatgccattagatctccacggtggaacacacacggagatggattctcaaaacctgtgtcctgacggccaggatctgctcgggtcttatatctatacggagaataacggcccgttttcccaaataatgcacaatggacagagcaataccgggacaggtgaaagcttcggcagctacgctgccggcgacggttttctgggcggttctgtgtcagggatgtatggaaacaacaccggagagggcgcgtgttctaaaagaccgtccgcgtgccgtaaacgctcggctgcactaattcacgcggcgtccgaggcgtctgtggccgagcaaggcacctcacagggggcacatgccgtatctgaccggataggcagagacggtggcgctgacaatagactactcaaggtgagtgcgcggctgtcggacaaaacaaagagcgcccttcgcagccatccttgcttgcgttgctattctttgatgtttaacacgtaatgtctgtttatattaaccctgccatagcccgaaccgaacactgtgcgatgcgattataaaacgcaacactcggacacgtcagatggacgcgcaaaacgcgcgcggcagtgcaagacctcaggaaaaaataaagcccaacagctgcaacaggtacaataagcaaatttagtaatttgtattttttaagtttccgaggttttcccgctggcctgcggcaccgtgtccgtgctttagttggaatacctctcctaacaagcacgtattccaacaggagctggaaagtaaaacaacgcaactgaccgtcctcaccgctcaacacgaagcccttcaaaagcacgcatcatttttacagaagatgatgatattgatgtgcaaaaacgggaacagtaaaaaaagcacgtcatgatttaaagccacctcgggttactagtttaaaactgcctaattgattctattcacccttcaataaaataaaattttaatacgtttgcggtttgtccatttcctgtcttaaaactattaaacaattcatttgggaataaggatttatgggattaagggattaaatttttttggatcatgggatttgggaacatacgtctgggatggaggttatgcttcatggtctgggtggcgtggattgcacggggacggtcggtgtgcccaacctggcacctgacagatgggaaatacgaggcggtatacaggcactacctcgaagagtgccgcaaacatgaaggctcggggagcctggacggttccggacagacaaaggggtctggaaccaaagcaaccaccgaagctaatatatcgataagacctaacgttgtcacatcaggtcaaaataaagagccgcctgggacagcaccgagggccgaatcatcacacgacctgccacgcatcaagcaggttaacgctctccgattatcaaccccggaattggcgcaaccactcccggtagtaaaatcgactccgcgcgagtcacagtcaggtgggacaccctggaacgcgcgcccccacgcgttcattatgcacacaaacgacatgctcaacccatctgtggtcctgtctttcagagccatccgtgcgcggtccacacgcgataccgagcagtccgttcgcgatcggaacacggtcacgaccagctatcgtacccctggccgcccttccctctttcaagccagaccctcgtctcacggtgcgcgtctacccccttcgccccgaacgatggcaagatacgccgagtcgcgaacaatatgcgaccaaaattgaccgcaaagaataccacgccgtccgttcgacgcagtgcccgtatggacgctgcacgggacctcaagcgtccgtgtcagacccattcctcccttcgccgacgccgtggcacctggttcaccccctgtgggctatggccggaatattgggcctcacgatcgccttgtacacgatatatcaaatatatgcacatagaaactacacgacgcttcactgcgaataaaggtttatttattttgcacactagtccgcgtcgttatttctggtcctggactgggcgcgcctcctcctgagcgccccgtccgcgctggcagcgattccgcctctggtggtctcgtccattgacacgtccacgcggatactaattccggcgagggcgtcttccatctgcccgcgcgtcacggccttggccgtggccgccctcaccttagcttcaatcttacgggttgccacgttacataaggccgaaacggcagctgcgattttggcctccttttcggtgttagagatgacggggtcggagccggacctggtgggatccccagaatttatcagctttttgagctgcttgttttcaacggacaatttttgcacctgagcggctaactcctccattgtcagctcttttttgggggcgcgcgtcttgggacgcgtggaagacatggcagctactacgcattacagagtacagcgggacgtttaaatactttcaggcggcccctgaggtcactcgtgcgccacggtttcaacaaatttaaaaaagcacatatatgttaggtacagaaatgttgcaaccgcgaccagaacagagttgatcaccgcccatatactcgaaaaactagacagcacgggctggtacgtgtccgccgagcaatcatagtcataaaaagagggcgttacggagggagccgcggaggggtttgtagtcacaggaggcgtgcccgttcctgtagtcgaggaggtggtgacggtggcgcacaccggcagacacagatataagcacgccaacagggctaacgcgagcacaattgaccctgtcattttcaagccggcgttcggcaggcaaccctccactttggcacgcgcgctctccttatatactaccagttaaacattatggcagaagtgaccgcccatacggttccgtacgcctttgactcctgtaagtttgaaataataccaaaaaacaactcatctcgtatagcgttacgcaacaaatttcccgtcgtggtcaaaccgggagaacctctagtcgtgcccctgggactaaaaattatccgcgcgccccagtgcgcattctttctcagcggagcgccgacggatgaggtgtattaccacaccggacttatagatcaagggtatcgtggagaaataaaactcatcgttctcaataagactaaacaggtcgtcacactttaccggggagaagtcaacgtctcactcatcgcgtttatgtacgcctcgcccgggcccctaaaatgcccgatactgaacctaccacattactctctggacgctggatttgatgttacgtcgccacatgccatgaccatacccccaaccgacagaaccccgtttacgttgagtttgtattataagagtccacagctcagcaccccccacgtcccactaatcgtggggcgctctggactggcgacaaaggggcttaccgtcgacgcgaccaaatggacccagtcgttggtacatctgaggttttataactttaccaaagagccaatagatataccagcaaacagccgcatatgtcaggtagtgtttatccacgaagaccacgtcccaagcgggtggaacattcttagatcccgcgtacaactcggcagcaccctccagatatcgtgggccaaaatcaggtttaccgacgtggccacgctccccaaaacccacccgctcaactcccgccacactcaaagccaaaccgaaccggagaccgcccgcggcgcaaaggggttggggtcgtcagggttataattttaaaacatggcattattttaaagttgtttatttttaataaaacaatccaaatttacgttaaccatcagcttgcgcatcctgtattattttgtctggcacgatattatccgccaccgccaactggggcgtcggttgtcgaatcaccggatgggaggggttggaggacgcttcggtgtctaccagcacattattaatttccgcagcccacgtgacagaatcatcggacgcgatcgtcgtagccagtttttccatttccaggagtgacacggtctcgtcggcccgtttattcaaaagcgcgtgaaaggcgctctcgtccaccgtaatatcccctatagccagggacaacatggcgttccagataacgttctgtatagactttagcttagatatttgaatcctaaccgtctcctggcaccccgcgggtaccctagccgcgtcaaaacgacgaagatactcggttattttattggattgaacggccaacagatacgtctgccgaagggcggcggtgcacatctctgcctgcttcacatctcccatatccactcccgggggcagacccatggctatttcggtccgccagtgagtaggacactcgttaagaggatctggcaggggacgatattgacatcccacgcgtccaaacggactaaacccgcaacacgtccaaaacgaaccaggcgcggccattgccaaacaaaatggtagatgaaattagggcaattttctctactagtggagatatggccgaagtaattacggatatactgactgaaacgcaagcaacggcgtccttcttctgcgtgctccacgatcggggcgacgcgcctataaatactccacatgccgtaattaaactctgcctgcccgccaagcgcccaggcggcgggccaaggtgtttaccgttgatggtgctgaacctaccggcgtggcaggttaatctattcctaacaggtgacgcaccattgacctcggataacattaaagaccgcattgacctggctcagaccgaggaaatactcgaacccatattaagcgtactggcatgcaaacggtccgcgcagcagaccaaacatgactcgtttaaatctaaggtggcctggttcagggcaaagttcgtctcggccctaagaaaagtgtacaaaatgaccccatccccctattggatgataacgctgctgggctcgttcgaggcatcgtttgtactggccggcacgttttattttttccagtctcatatatgcacggcggagacgctggtgcatctaacgaggttattcagctccagtcagggccagagcttggtcaccgtaaacacctatgacgaattagggcgcgtattcgggcggtcagatttccttggaattgttccaaatttttgggcgtatctaaaatacaaaatgcaacaggacgacgtggagtccagggccatcgatcaaacaatcaactccatacgtggggggttgatgctgtcacctcaggacctcgtacacttcatctacctgtcattttacgagtgcatgaacgcacagacgttcctgtcgtactctcgtaccacgtccagtttaccgacccccgcaacggttaaccctccgcagctgtgtcggcgcttagaagcggattttaaggagcacgtgatggcatattacaataaagcaagctacctgagtacttacataaccattttaaccgtacccgctcctctcccggacggatacgaaaactttcaggaactagcatgtcaatactggtgtggacaatcgagagacgtggcggaaatcatgactagaattaatgaccagtatccacagctgaatctaacaaaagatttatccggtctccttgacctagcggccctagatcagtattccggaggccccaaggaaaacctttttacggtggcttcgagaattcccacatacaggtgcgagtttttgaataaacagtattttgttctaatgcacgcagactgtatagacgcgtactggaaacaaaacattatcgtgcccgaagacgcccaattgcaaggcctgacggatcaagatctcacctccaggatattttactgcgacctcggcctatctctacctacatttaaacaacaaattctagtttcacgccacgagtatttcaacccacgactcccagtttatagatgggtgttggattttgatctaaaggtaaccgaaggtagacggactctaaacgatatctacaacatatgcgtaacgctgcggcaggtcatattagaaacgttgcagttaataggtccactaaaaccgaaccaccccgtgtatttttttaagtcggcctgtccagccgtaacctggccggatgatatctcagacaccgcgttctgtcactgtgacgcaaaaatagggatgagaattgtaaccccgtttccgagcggatactgtttggtggggtccgctccactcgtgtccctgaccgacattttaaaccgcgtggttaaactggacacgcggctagcctcagagtatcctgggatcttagaagataaaggaccatttgactctggcatttacgccaaggggcgatgcgtgcgcgtacctcactgctataaggttggcccggggggcgagctgtcacggctcctaaaaattattatctgtcaccccgaagagtcagataaatcggcgtatttgaaaaacgcgtttaaggtttctaatctgctacatcacgcccctggagactctgtcaccaaaaacggccacctggtgtacgcgatcactgacgaaaacgagggctttctagaaagcaaaaccaagaataatcttcccaaaacaatcacggacctggccgaaaaaattgagcgaaccacagaaaaaccactaattgattgggcagcgaccgcagtgtggccaaaactacacgataccatacagcgtttctttccggatgaccgcatcggccaatttgcatctgtgagcttcatgcactccggagacaacattatacaagttaaaccacaaaaaggaaacaactttttctgtattaatcataagcaccgaaaccacactcagacagtccgtgtatttttaaccctacattccacaaaagaaagcgaggtcacggtgacctttatgagtcagtgctttgctgcaaaatgtaatcataatagcccaactgcacatttttcatttatggtacccataaccggcacgtaagagacacgaataaaatcatccgtgatgggccagctctccggctactttgttaattttaacaccgcgtaaccccaaaaacaacatctggacgagttttgttcgcgaacgtgaacatggcacaggcaatggtgagcctggaatacatgaaggatattttggacggtaagaagaccccttgtggctcttacgactcccatttaaaaccgcaacttatcaaacggctctttttatacgatttacatgcggtatgtgtttcacgcactaatttgttttatagggggcatatcatcgtcggacttcgacgactcgtcctcggacgaaatggacgacctgtccccaacgccggagccagaaccgtccacaacgccgaacagctttccggaggggcccaaatcacaagtggtggcgttacctaaaatccgtaaaagatctcgatctgagacgccggtaaaaattgagcacagatctccacttaaccgctcgcgatctcgatccagaacgcggtccgggtccggtcaacgatcaaaccagtcaggcagatacgtcaagagattcaaaccaacggttgatgcaccccgtcatcgagaaccgtggcacaggggcggtaagggaaaggccccgtttatccgcagagacgcaatggctggccgcggtcgacgcacatacggccacgactatcgcggaaaagccgctttaacgcggagcattaaagagtctattaaaaagatgcaccttccatccaccatgctctctcgtgcgcacgataaaaaggtattcgagggactgttgccacgacacctgggacagtgctttcaggtgtgcctgccggcgccgccaccgctgcaacccgaggtgttcacggatcgacagcttaccgctatagtcaagtctggcgggcgcagagacgcgctggtggccaaaaaagttagcctggctaaactaacaagcctatacaaacccctgctcacgtttgtgacggggagaaacaaccaggcccactggttggcgacgcgcaaaaacacgctagcgtctgcgggactcgaggctctcgcggcctttatcgaggaaggtctggcgtgggcccaggtgtgcgtatctcaaaacagatcgttgaacgacagcaacctagatatcattctagacagtagtcagagcgtgtgcacctggttcatctccaaaatcaggcatttgcacatacagtgcttcttggagaatcagggtgaggttagcctggtgaagcagctaacttacctcgtgtgtataaataaccgcctggcggaggcggcgaacctggcgggagaggtgaaactgaattttaaactcggaatgctgattgggttcgccctaaccttgccggcgctccttgcagagcataagttgtctggcgagagcctgtacctgtttaggtctttcctggagaaatatagaccgggagatgtcatgggactcttaaattcaatagtggtggagcactacaccaaatgtcgtagcgcggagtgtgtcattacaacccacgccatggtaggatctggcgaaaacaacaagggactttttttctttccagtgtaatatataacccatgtgtgtaagtagttacggtatattattcacgggcgtttaaatgcaataacccacataacaaaataaaaatgtgtaaaaccaaacacgcgtcaagctttttctgcaagggttctgtgctcggaacaaataggcagagttcgccacctagcggcacgttctctatagccttggtatacagcggatgttatttacggaagtgccacatgaaaccgtggccgggtgcgtaacagtaaaccgggtccaatcgcgcagtttacacaacacataaaataggcgaccgtttatagcggtttatttgaacatacaacacacatttcaggagtttcggtctttataaaaatcttcattcattcaaagtgccgatatatttcacgctggtggacaacaatacatatcggcgcctgtgattgatcctcctcctcgtatagggaccaggcagacacccaaatctgtgcaaacggatcgggtggcccgggatcagagggtcgtggtgactgggctaactttaacatgtattcatcggtatcaaaaacgcgatgtcccaccccagatggcacgcgtcttggttcagaggtgtttgaggtattgccacggaaccataaattaagaaatccatagccattcatcataatcccatactgacaactgcacaaaacaatgccgtcctccagggcgtgtaacgcgttcacaagtctgctcaacataaaatcgtcatccatataaggcgccgggggcaaccacaactgaagcggcccgggagccatgcagacgtggtgttcatttttatccgttggtctcgtggataaacgaacgcccatcccagaatgggtaaaagtctgtgcttgaaggaccccataataataaacctgaatatgcataaatccccacgggtcgtcaacagatggcgccaaacacgcagaaagaccgctcgatgcagatagcggcagggcgtgttccccgggcaacgcaaccgcgcccccttgactattgtcctgcctaccgtcttcaccggaggcggttgcggttgcagacgcctcggtggccggggcggtcatggtgaaggtaccggcgggacgggtacgaagtagcccgagcagctgctttaccgtgtattcagataccctgcccgcctcgtcggcgcccttgcgcttaaacatccgaaacctcaagccccgcagttctgccgctaccccagaggcccgagggcacaacctgcataacatctcttcatcggagcgcaacctgaagaccacgtttgcaaatgaaaaactccgtctcaaaacgtccttttcttctatgaaataggcactctttcttatcgcacccaataggcgcgccttggcctggcccagcctagacctcccggacggaagggggatgtttctctggttgcagtagtcctcgtacatcttcccatcccgaaggtaatcaaatcccacagaccacggggtcgtagtcgccagcacgaccctggttttttcgtcgtctgcccaaaacagtcccgaatacgttgcaagatttgcgtgagtaacgatccattctcgaagtcccatggcggacggaccaccgctctgaggtgggcgccaggtagccatggctgtatatgccaggttgatactcaccgctttcagttcgtgttaaaataaccaaactccgcccatgcggctaccgccgtgggcgcagatgtgtgcagccgtcacaaaagaatcccacctgcggcaactaattccctcagcgcccagtcagctggctaccggttcatttcactcacgagcacgccaccccatcatcttcaccgctcgttcccgcgtccggatcgtccattgaatctggtccggagtcgcccagctctactgccgggtcatgcgattggctcgtcccctctcctggatcttccatctggaatgaacagtcggtgcgaccctctgatgttacagtcgtcccggggtaaaaacaaccctctgtaccgcatacagacgaggtttcgcacacgtaacgcagtctcatggcgatgggcgcatcgacgggattcggcgactggaccgacagacacagggcgatctgtgcataatctctaggtggttcgtctccagggttcggggaccttgcaagagctctaagataatccaccatatcaaaaacctgaacgggttgcctggatggcaggtccttatactcctcctcattgttagtaagcaccttcatttcccggttctccaggttcttcacgcaaatgccagtatccgtaagggtaatcaacacacccttctcgcactttttgtgcataacccttagatcttctctggcaaactgacagtcaacgacccccggtgacggtaaaaacaaagtcctctcatcttggaaacaacacgcgtggccagcgaggacgggacatggtcgactgcagacccgtattccagcatgaacacgttcgaggccaacggtctctccaaaatatagaaacgttaattggatggcccctggcagccgatgctcgcgcacacgcttgtttttgtccatccgactccgagccaataccgttgcggtaaaaacccgtcttctttcacgtccagacccacccccctggtcgtattggaactcactgattaaaccaaggcactgagtttcggtggaagacatcagattacacattgcacaacttaccaccggttccggcaacaggcgaaacagtctatatcgagcacccggtcgcccataggccttattaataaatttaagctctcgtatacttttgtggtgtctcatgtcgtacaagaatctcattttcctgaatctgccatagtgggacggtttttcgccctgacatatgcctcgcatgttgcagtagtcatcaaaaaacttctcgctattccatacggaccgatctgcagttactttgttccacgggacccggacaacggtcttgtcctcattctcccaaaccaatccgcggtactcgcccgattcgaccgcggcaacgagccaggccctgatatcgactccacggcccgccattccgcgtattgggagcttgacggatcagctctcaagacaaatgaaacccttgcgttcacggcaccagttcataaatgtatctaaatgacggagtcatgtgccctaaacatatggctaagactactcaaactgtgcttaggggaagagtgacaaacacctggcactcaataaaattttgaggtcgggcattgacccgtgccttcctctggttagtattaaccccatcgccaccatattcaaggaacaaaacactccagatacaaaacactttattacagatagaaggcgctcaccggcctcacaaaaccggtcgcgcgcacaggtaacattaaacattagcggcggacaacgcccgccacagacactcgtgccacagctgaataattagcggtacagtgctttgcactccgccctcgggtgtaggtacgcccccaaaatataggtaaacacaagcttgtggaagcgccgctccgtccggagatgttctggccatggctgtcatatagtcaaccaagttaaatgcacgacacgcgttggcgcacgacaggaccgcaaccgcccccgtagattcgacgttcccttgaacgaacgccagggaatgccccaaccatctaatgtaaatgccggaggggtcggcccaaaacgccaacccgcgcaaaagatctttatccaggaagcgcaacgcctcggcctgaaactctgctaagtggcctggaatctccggaaccagtgcctgctcggcgatcggtgcggcgcatatgtgcccttgagggcgacgttcggacaatgggagcacacggatgccgttggacgattcagtcaccacttcaccaactctgtccccataatagtaaaacattatacgtagcatccaacactgttcacccccggctcccaccgcaccagtgtaccgaacaccggcgccaaccggaccaaacttattcacggcctcctcgcgcaacccccgtaacatagcagtagtgccggaggcctgatcgcaacgagcacacgacctcacggtaattggtaaaaggcgaatgattcggcatctgtccggatatggagtcgaaaggttgtccttagttgacacgtccgacacggtttgactatgccgtatggcgctggatagccagtttttgcattccttagcggttaattccctgccagccgcgtgcaagatgcccctggatcgacagtagtcaataaaaatgtttttttccgcctcctcgactcctctgctccctctgtcgtgattccacggtatgcggattaaagttctttcctcgtcaacccaccgcattccgggatgacgacctgtctcgcagcattccaccagccatcctcgaaggctggatggtttggtcactcggaccggccgctccatatcctcgccgcgtgcacggcaatgccaaaccttgtccaagttacctcctgacaagcacctgcgaccctcacagacctacgcgtggcaaacgggaacttgtggtctagtctgctgggacagtaccttagcgttatttttatctgtcgtgcactcctgcggcggaagtttgtggtttatgagcaccaccgggaaggattcatgtgtccctgcaccagctcgtcctgacagaccaccacagaaataggaacttgtgggctctcttgggcaccaggctgctgtgagagatacagtgttgccccgttgcacggcgcatggctctctggtcccggagagcgggcaagctttctcatgaaacccatgaagttaaaaattttctgtttagagaggaacatgacctctcgttggataatatcaccattacagtcgttacccgccgcgtacagcgtttccctgttcctattatcgacgtacactccggtttcgttgccgtgtacgtagatgccctcgttatacgcgaccagcatcgtttttatttgcgttatctgttctgcggtcagtttttcacgatctggagatggaaaccagacagtgaacccggtgccatagaaacacagatgaccgggatggggaaccgggcgcggatgcaaacgaacacctctccgatcttgggacgtgtgatccagtgccagaacgccaaaataaaacacctgaattctcaaccgccaaaacggcacaatttgatgaggcagcggcgcttctgggaccccgagacgatgtgctcgggcgtacgctgatctcgtcggagggaccaacgtgggcaagggggcgggcagcggcattcccggctctaacccgatgccacacacctgctccaaataatttataatcccttcagagttctcatcaaggatacacgcatagcatccagaatccatcaaaggttttacccggaaggcggtccagccgccaccgtgcgtccgcacgaccccatgctccacttcttgaaatccagcattctccctcaaacccctcaaaaatcgtctcttacactctagcaggtttccatctagcccaacctgcagacctctctccacgcagtaggcgacaacggctggagttccagctgcccccttcttccaagacagcttaaagctttttttccctcatcgcaccaagttaggtctgaataggttttctcgttgagatggtaaagggtccacgatcgtagatggctgtagttagaatctcgattcgccatgacagcgtacacctatacaataaggcgcgatacgagtgtacttttcccctgatgatatttgcagggctctatctcctcaaaacgtgcacctggctaagcaagcagctctatttctgccacttcctcatttataaaaccatattgtttctggtctcacgttttgcagctcacctgcaacgacacggtgaatgatcccgagtttcgctcctccagtgccttgacaaacactggcccagagttaaaaagtgaagcaaaaggcatagctttcgatttcagtcaccggcagtgagtggtggacattagaaagtgtttggcccacacattcagctgtgcactcaagcccgaccaatatggcacgtgaccattcccctatcacaacactagtacagaaaaaacgaaaccacacgcgaggtgacattttccaggttagagaaatttattaagccggcgaatccatctaacaaaacagcaaatgtttattcaaagtgcctatagatttctgtttgataaacaataatagatagtggagccctcgaagagtttcgctcctcgcccagggaacaacccgaaacccagagtttaacaaaggccgcggggggacccgtgtcgcgcggggagggggacctggccatatcaagcaaataacgatcagtgtcaaatgcccacacgtgcatcccggtgtaggcacgatgtggcaaactgtacggttccacggtatttccctgaaaccacacgttaggacccccggtaatcgtgaacataattcccctgctggtgctgcagaaaattattccatccacgagccatttcaaagcgtcctcgagtctctgcaccacagacgactcagtcgtctgtggtatcggaggaaaccacgcctgaaggggtccatccaaaacacacacgtggtgtcccgccttatttgttggttttgaagatagcctgattccctgcccggtggctgtcaacagctcagcctgcaattgcccatagtaataaatctttatatgcatgtgaccccatggatcgccggcagatggcgcagcacacgccataagtctggcgcgcagaaggcccgaagaatacgctgcatcgtcgtcctgcgcgtcgtgtccatcttgatgaccttcccgataccgtctctcttgatcacttacggacggtcccgaaagggcgtgaagcattccgcgcccgagcatggcaaaccgcatattgcgcaactcaaggcggagcgcgcacacgcgcggacacaccggacaagacatctcttcggatgaacgcaacctaaagaccagcgccgtgaacggaaattgatcggtagggaattccttatcttcctcaaagtacttgctctttcttacggctcccagcaggcgccccctggcttgacacagcctgctccttccagacggcagtgggatgtgtcttaactcacagtaggcatcatagtgttgaccgtcgcgttgataatcataattgggaaacgacggtggtgtagctgccaacaccagcctcgtcttttgctcgtctgcccaaaacaggcccgggtacagaccactatcggctttgaacgtcagccattccttgagggccatgccataacgagatgggccccttctcgattccccagcggccatgattcagtcggcaacgtttaaaacccatgtaaagtttcagttcaatttaaaatgacacaactccgcccatgctgtgacaaacataaagaaagtggcacacgtgtgcgctttggccgtcctgtcagatgaaccccgccggtaacatttacttcctcccatacggtgcgagggtagatacagatgtgcttcccgttgttcccgaagcaccgacacccgctccagacggtccctcgtccccgctgtcatctcccgcggcaccgttgctgacatcatctgacacatcatcattgcacacgtagcgaagtttcagggcaatgggagcatcccatgtattttcggactgagtagacgggcacatgaaaatccacgcacagtcacgcggcgggacatctcctggagttggcgatctcgccagctccctcaaataatgcaagagatcgaacgcctggagcggctgcgaaggccgcagctcatagtaattttcagaatagttggtcaatgccgtcatgttgcgttcttccaggtttttcacacagattccagactccgtcataacacagatcaaacctctctcgcatgttttctgcagggcctgcaaatctgccctggcaaactcgcagggaatcaactgggacgacggcaagaaacacgtcaatggattatcggcacaacacaggtgtccgggacgtttgggatcaggaaggccacatatacgcacacccgagccggctcgcaaaatttgaactccgacgttctctccaaagtaaaaaaaggatacctcaacagcccctagcacaggttgcgcagaagagtcctggagtcgtttggatttccgtttataaacggagactctcagtggcgccttagccctcggccgccgtgtcctctcgaaaacatcctctcgaacgcaactgcccatggaacagctatgcaacgatgtggtcgcgatgaggttacacagatcgcagcccaccgtaacctcaggaagcaattgaaagagtctgtatctttccccagagacgcccccggccttgctcggcggtgtcagctccctcaaatacctgtgagatctcactgcatataacatcctaattttcttaaatttctgggcatgtgaaccggtttgacacacgccacgcgctgagcaaaaatcgtcaaatatcttgttataatcttcatcgacgcgactgtcggtgcaccggttccacgggacacgtataatggtcttgtcctcgtcatcccattccactccggggtatcgcttagactccacggcctcgatgaaccaagcttttaaatccatatcgcgttccgccatctcgcgtattcaatacctctgaccgagcggctcagttaaaataccgagcccccagaaccccaggcctccgtaacaaaaaggatctagatctgaaagaagcctaactatatacgcccacgggaaaactctgtaacaaatgaaaaaaatgttctcagaaaacgaagagaggaaaactgacgacacgcaatctagtcaaaccagaacctaaaggccgtcactgacccatatctttccccttcccgagcgagtgcggtttctcacatgccgtgagcaaacgaaaacgctacacataataagacacgtgtgagaggaaaactttattgcagggacagggcaaaagcaagctgtgcacggtaacagtatgtgtcactgggccgcatccccgcacgcccgccacagacactcgtgccaaatttgaatggacagggggattgtgggacacggctcgccttccggagtgggaacgcctcctaggtggagagacgcataggcggctggaaatcgcgttccgtgtggcggggtggctgacaccgcactcagatattgggcgagcgaaaacacgcgcagaactcgcgcgttgttaattatatccaccggctccagcgaacctccgttacccagcaggaaggctggaacgtgacccatataccgaatgtaaatcccagacccgtctgcgtaaatcatcaacccgcgtatgatctccttccccagccactttagcatgctcgtctggtgaggaaacatatcctgtggggtatgcggcaacagggcctgttccggccgaatgcccgcacaaacgtgtccttgcagtggtcgctttggcgccggcaacaggcgaacgccgttgggcgacccgacctccatgttcccaaccaaccgcccatagtaataaacaaccagccgtagcatccaacactgctccccgtcgcctcccacggcgccttcgtactccatcccggcaccgagataccggaatctggcggttacctcgttgcgtacgtccaggagcatgccgccggtggcatccgcttcgttacagagcgggcaagatcgtacaaagatcggcaacaggcgaataacgcggcacctgttaggcgccggcgccgacaggttctcctcagtggatacatcttcgacggtctgactgtggcgtatagcgctggccaaccagttcttacattccctgacgctcaatcgtctgttaccggtctgccgtatgcctttaaattgacaatagtccagatacatgtttctctcatcgtcagagacgccacccgcgccctttaaatgattccaaggtaaacgaaccagagtcttttcctcatccagccaatgcattccaggatgcttatcattatcgcaacaatcgagcaaccatgccctcaggccagacggccggtttacgcggatcgaccctgcccgtccttccgccatctctgagggtccgctcgcataaactggcttaaaaatctatccccccccgtgcacctgtactttacagtcacaccttcgggcacacgccgtagggctggcagggacggcacccagcagctattttcatctctagcgccgcctactagcggaaatctgcggtctacaaaatccaccgagccgcccgaatgtcaccatgagtcaattcatcctgacagatcaccaaagtgacagggacctggggtcttttactggagccagggcgacccccaaggtaaagagtgactgcgttgctgggaacagtgtcgccgggaacgggtgaccgggcgagtctgcgcagtagacccataacatagaagatttgagatttgacagaggatcgcacagcccgttgtgcgtggttcccctctgcgtcgttccctgcgcagcgaaccgtgtgcctggttctgttatccacatacatccccagctcattgccatgcaaaaagacgccctcctcacacccgtttataatctcacacacggtttctctctgctccggcgttagcggcccatccaactgaggttttggaagccacatcttatacgctgtcccatagaaacactcgtgacccggtttggggtcttggcgcttatgcaatcgtacccctctacggtcctgggacgtgtgttccgctaccagcgacccaaaataaaaaactcgtatacgtagccgccaaaatggagtgatctggtctggatgcggtggtgcaaccgtcgccaacctggcggccctggcatatacggaccgcgccgcaccgtcagcctccgccggagcgggaggaggcaggggcgtccctggctcaagcccatggacgcaggccgcctctaagtaatttaggacggtctccgaatgactatctaagatgcaggcataacacagcggatccacggcgggtctcaagtaaaaaaatctttctccgccaaaacgcgttgttgttgcccgggcatcgtcctgttcaaacccagcgttttcccgaagcacccttagcagtcttttccggcactccgacacgttgtgctggcgtccacattccagatccctatccaggcagtacgcaaccacaggttgcatgccactcatacctcggtgccatgatatcctaaagctcctcttttcctcgtcgcaccaacacaagtgttcgggaaacgtgttcgcttccaaattggaaataatccatcttcgcaggtgattgtgagtgatttctatctcggtcattgtgaacttggtccacgaaaactaactcaaaagccctcaccactcccctaaactgtagctgaattttatgtccaccttatagggtgcacgtgtacgtatatgccccgtgaaaacctcgcacttcctcataaattatccccaaagcgtttcgggtcgctcacctaaactaactcgagcattacacggttcatagtgtcccgggtttcgccactcccccaccgcgacagagaaacgaaacgttcaatttaaagggcaccaagctcaactttatttaaggagaaaaaaaccaccgccatcagaaggtttccgcaggcacacacccccaagactattcttggcgagttcgcggcacgctgtagtttttttctgattatttgtaattcgcatccaatcgccccaaacatagtcaataaaaaaaccgtaaacacacacactctggtgaacaaattatgcgttcctggccccaagacgtgcgtgagcgtcagcagcacagcggctgctaaaacataaaccagggctaaaacgtttgaccgaaacacgagtccgaggcaacaaaaaagcccaacgtggaagacaaaaaaatagtgtacaagtccgaggagcagaggcgtcgtcaggccaacagacagcgtacccgcggccgcccctccaatcaaaacagtcacgagataaaagtcccatccacacacctcaagcgcttgctttatagagtatgtgatgtaccgcctagaggtgagcgaaaaaattgcatggcgtcgctgcaggcccgcctcataaagcggatgtgaccgcagcgctctgagtgaacaaacgccacctactataaaggccaccaggggcagcaaaaacaccggggctaaaaaaaggttcttaagaactaaaaaataatacaccgagaacgctgctaaaaatccaagttcgtagtatctgtgggcaatcgtggggcacaaatataccacgtcattcgccgcaacaacacagagcagcaggcacaggcacgaccccacaaaaatggaaacgtgaacccatggaaggtcaacgccgagggtggatgccatagaaccggcggtaataagaaacgccgagatggtgcacggtacatccacctgccgcgagagccaggccggtagcccaaatatacagaatagcaaaagcagcccatgtaagccaaccgcgccccaggagaatataatgttctcaacgctaatcaggggctctacaaaatacagcgagtgataaataaaacacagaataaacggggaagccgagaccgtcccgctcataaaagatagccaggcgagggacgcctccgacgtgtacgtgcccatggcgcggcgtttaaaacaacgggttgaacgtcgcgcgaggcgtcttggtttttcctttcccgcgactgggtgagctgctgtgttttcgcttgccgtgttttttttccggtcgagagccgctcgtccgcagtgatttggcgcccccgtctgtcggctcggcaccgtcctcggcaattgggacgcactgctcttttaagtccaggtcaggcgttctcggaggggacgggagctcaggaaccccctcgggatcggcaccttccgagagattgcccgggacggcattattccggtccgtcggatctacctttaatagcatgacgcccaacctgacgtcagcaaggctatctgtcagcggcaggccctcaacttccagaatctcattggcgtgccagcggaaaccaggcaaaaaaaatcccggaatcttacatgcgttcaatgcggccaccagagactctaaactcacatgagtcgtcaggtcgctagaaaccacacccacgtccagctttcttcctctagcgcaagtccacttggcgtccggggtgtcgggtttaaaatcaataattttactaaccccgtcgaccgtaaacgtggcaactgccagcgtttcgcttaacgagacggtgaccacctgcttggccctggttttctgctgccttagccactttaacaacaaagtcagcgtcttaaccgtaaggaacaggcgattccccgtcccggcctgctctagcctgtcgtggaacgagggtaggttgggttcgtccacgacgctcttgtgcacggtgctcgtgtgattgtccccgtaggcgatggtcgtcctaacaaactgcggctttccttccgcgtccgactcgtccccgtggtggtagaacgccacgttcatttccgcaatgttaactccaaacatctcacgggtgtgtgtgaacgtgttcccaagggaggtgtttcgaaagctaaagctaacctcgtccgtcatgtcagaatataccatcagcggcgccagtgcatttttaagcctgagaccgagaacgcccgcgtcccccacgctggagagcacgctaaggacgggggccctgccctggccgctgatctgtataacgcctttcttaacgatgcccttaatgtggtcgtaaaccctgctaatgctgccaagcgcatcgacgtccaccctggcgccgtaatgaaacgagacaggcatggctaaggcgaaattaacaccctaaaagacgagaagagaatggcaaacacgacagacacagcgttcaatgttcaaccttttaatagagcctctggcccggaaaatgagtaacatgggacacatggggactacagatcattatgaacggatgtgacatattcagaattatcacgctcaaaaaagttcacactcttggcactcgtatacgacaagggacacgcgtttggggggcgggcaccgaacaggggcggtatgtttattgactttagaatcctgtcggccgtggcctgcaaaaactgctcgatgtctccgacgttcaccatggtcacgtctcggctcttggaacgaataaacagagtctctatctccacggcttcgcgaaagaggccgtggatgtatgcgacggagggggattcgtctcgggagatcatggtgttgtacagcaagctggcggccctggtgtgtagcagctcgtccctggagatgtagtcgttagccaggcacaccccgcgcatgattccgcgaaggcgaaaaagtcctatagagtaaaatgaacttataaagtatatcccttcgataaccaggaacagcagcaccttctcggcccgcgtggtggccttgcgcactcgggaatgcagccagtcaatcttcttcgccagggtagcgtcctcgactatttttttggcataaatcattaagtccccccgattaccgccaaaaaataagttcagtatgttggcatacactttgccgtggatgttttccatggctacctgttcggcgtaatagtgggtcacgtcgtgattgctgaactctttaactaggtcttcgatgttaaagtttaccagactctctgccatacccaaaaatgtgaagagaaactggtaaaactccctatcggtgggactcaatagtcgcacctcatcggagtcctcgtgcagggaaatttgactggggaaccatctgttctgccatgtctcctgggtgagtgctaaaaagccagggtggtcgctcgcgtacagaaaccgatctggggactccatggcggcggttactattgacaggccaaacataccttggtccgctcaccgtcggccttttgtacgcagttagactccgccccctctgaagagatgttgtcacccacgacctgatctaagcactgcagggcattaagttttgtctgtttctgaattcgacaataatacagcatggtttttaaccctagcctgtatccggtcaggagcaagtctcgaatataactcgcccgcgtggcctgctcctcctttaaaaagaaattaagcgactgactctggtctacaaatggagcccgcgcaccggcccgctccaactgcttaattgggcagtaatcaaaggccgtcaaaaaaatcttgtatctgtccttcaggggttctggaaatgaagccacgtcacctccgtaacggcgcaccgttcgcaggtcaccaggcttcactctcttaaaaaaagtcacgtttggcttcaagatctcttctttgctggtgaccttcgatgcgatattggcaaagaacggataaaatgcttcggtgtaccccgtaagctgagaggttccggccgttggcatcagcgccaaaaactggctgttgaaaattccatgctgggcaatgctgcgccccagctgctcccatctctccaagggagggtgggacggcttaacgccgtcccacgtttgccagtgaaacacaccctgagccaatcgactccgctcccatccacgaaacggagtcccctcgccgagtaaaacaatctcatgactggtttgcaccgccgtaaaatacattgcctgaaaaatctccacgtccagcttggcgctctcggcgtcaaggtagccaaagcccagttcggcaaacacgtcggccagtccttggacgccaatgcccattgatcgttcctcctggccgcgcctaacgctctcggtaggcgccgtcccgcccaaaatgcacgcattgacaatgataactgcggcctccacggcatcatcgagcagttcaaaaccaaatgttacatcccccttcccaggctccacaccagactctcccctgtggggtctgaggcactttggtaggctaatgtttgccaagttacacacggaagcctgaccctcgggttgctgcacgatttccgcacagagattagaacagtttatggcgctgccttgcgtctcacaccagtggtgtttgttgagcgcctcctttagcaggacgtagggactgccggtcttaatgacagtgttaataagggcatacatcatcgattttaacggcaacgagctagagtgtttgccagcggccactagtctgttgtattcaatctcaaattcggcaccgtagagttttaggagatttggcgccacctctggcgcaaacaggtgccactggccatctgggtttgtttcgtacagtcggaaaaaaagctccggcacacacacgccctgaaacaggttgtgacaccgctcctgattctccggcatcttcgcgttcaaaaaatcacaaatctgatgatgccatagttccatgtaggcgctggcaccaacaggacggatattgttatcgttgaaatacccaacgtgggcgtttattaattttaaacagctggtgatgttcttgtgttccgcaaaagacgagacatctatccccacgcctgacttgctagcgagaaggggggacatttcctcatgaagtgctttgagggttttgtcttcggtcgccatggacggctttaaaataaaacagctagaaagctgacctccgcgaagcccggctgaccttaatacaggcgttgcgcagcacacaatctgtgacgagatgtaatggaacgcgtaaccaaccaggtacatctcatccagctccgtttcgctctccaccaggtgtctgagggtctcccgcaaacacggaaattttatacactgacaggccacaaaaacagccaccctcataaacatctgggccacgctttcaaaaatgggtgaagaaccctgggttctcagcacgtacgtatcgtaaaacctaacggcagacaggtagccgcagttaacgaaatttgtgtatgccttgctctgcttaaagtcctgtaaaagaccgtcaagggccgcttcgtgttttgacataaacgcgcgaacctcgtcgcttagcctttccccgaataccgcgagatagtctcgcaccgtaaccaggcacctgtcctccattattctgtgccaaaggagaccggttagtgagttggcctctatgtcccatccaagtgttgcctttaaagaattcacgagtgattcggcgcatcgttcagggtcaaggcttgagctacaccccccagttcccgcgtcggtaactagggttaaaggtttggcagattttgccgcagagaaagaagtttcggtgttcattgttaaagaaatagcttccaagaccccggattggaaacaaactccgtaaattttaacaccggtaaagcagcgcctttaaagtgaaggctttgaaaagatggttgtaaaccggaaggcacgcttccaagtctgcaaactacgccgaacgcaagcctatttatatacaggtcattctgcagctgaatgtatttggtgcgaatcacgccgctgtaaaaatccctcaattgggcagctatttcacaatatcctttaccagacttaaaaaacccaagtcctagcgccggtctattatttaccctaatgtaggttaagcaaacataaatagacgagtgggcgggcaaaggctcgtcgcgcaggctggacgggcatgagtcatctaaatccacaaacatgtcactaggaagcgtaaggccaatatgtgtaacaacgggctctctggcgactacgttgccctttaacgcagacgtcaccttggtgacaaacgtactgtggaccgtttgaaccaacggcccgaccggcgcaagaaactgatgaagcgagccggtttccaacaattcttcaaaattgggtatggcgtcaagtagaccgctctcgtggccgtaccaaacacacgctattctgttggtctggggggcagagtccgcgtccatcctagacagtcgcgccagcgacgtaggcgtgaataacatgtcaatggaggacccagtgtcagtctgtttaaaggaaaacaggtaggtgccccgaggttcctgtgaactcatggtctgagaataaatcaaaaaatctccatacgtttgacatgtaggcgagacagataaaaatccatctttgatggcctccaccccagtggtggtcgacaccacatatttagagagcagatcacgaacacccttagaaaagtcgcgaccgcgagataacgaaacccggtgaggaggcggcggcagtagacgcatcaaactatcattcagcttgttcacgtttgcgtccctggtcttcatggcgtcgtcaattcctgccgcacgggctgataacggtgacgagaacaccggcgggctttacaaattaactgacaacctcctaacgtgcaccggatcgctacaacagcttaaactcctgatggagttccaactaaaaccactaccaaccgcacaccttttaagcatgcccaccgtgacccggtttttaaatactgcattcaaaatagacaaccccctggtttcatttattcaaaaacaccctgtgttttttttaatgagagtcgccaggcttccggagccagtcattacagaccaccaaagcgcagaaacgtcaacaggcatactatccgaggttgtgaatgttcttaatacagctattcgaaaaccacacgagtccccagcggctaaagacaacgattatctcgacaaccgtgccatattggccatgattacagaatacatccatcacgtaacttcacgtacgccctcggggatcccaccgacaccaccaatgggtatcagccatctaccgtgcgtagagcaaattttacacgaaacccaccggcaatactggaacctaaccctcccggagtcgctatttatcgacatcggcgaagtcgcgtctccgcttcagacgtggctaatcctatcgtattgtaaaaaactgcaactggcaccaccgcccctattcccacctgtcgacgaactggcccgtcgtctggttaccggccaccacgaattgtttgtccccctatcgacgtcactggaaacgtacatcaccatgccagtatcaaaacagcgagcgtttgagatatacagcgtctttgccaaatcaaaaaacatagttgacggcacaccaattttagcgttcaccgacacagaacttacaacgttcacccccgaactgttattcctgtacgacttcgtaatcgagtccctgtgcaaaaaccaggcatacgggtgttcgcgcaacgcaattgagcattttattaagaaaggtatcgattttatggcggagttgggggcgttcattgaaaaaacgtgtggctatcgatcaaccgtaagcctgtccaacgtgagagccgttaaggccaggctcgcgagctgtggcctatccaaagaggcgtgtgaagatttccgcgcaatgatactgatgacaccgcacgagacgacgccaaagtgggaaaattttacggactttttagaaatggtaaaccaattaacactatacgggttctatttttacgagtgtcttaaccagtacagccccactagcatatcgctggccaaaattcaaaatatcttaaaccgagtcgatgccgaacagagcgatcgcgccctgtggcgcactccattaatcgggtctttcccgttcccctggaaattgaacaacgtgctggcgttttttaagcctagcacccccgtggcaacgctacaaaaaatctacaaggcaataccgtcgtacctaatgaggtctttgttcgaaatagcggccaacaaatcgtggggtaacatcgcgctcgcagagagcgccccgctaaccgacatacagaccgccgaaccagaccagggtcccgtgtccgcccaggttatcgcaaaatattgcagccgtctccaaataagcgcgacagattatgacgcagccatcgtctccagcccgggattcgccgccgagttcattaaaacaaaactgtatccgatccttagcgaggtgctccgaaacacgtccaaaaaaaatcgatcgctattccaaattagatggctcatagtcttcgcggcagaggacgccagagacctagcccctatcagacgctcgctggccctggcgtattttcaaataatggacattttggaagaaaagcattctccggagtcgttttacaacctgctggactatcttcaggaaacatttaggtgcatacgacaggtgataccggaagccacgtgcccacaagaatttctacaatacttgtttacttttcaaaacattccaatagcagcgagcttcattcaaacatccatgacctttgtagacgacctgaaaaacggcatccccggtatactggaccttgtctccttaggtgccgcgttttataacatgaaactactgtacgattcaacgctagacaccgtcgagattccaacggaagaagggcaacccatcgtcgtgagcatgttcgtattcaaatccacgattcgcgtcctcgagaagctcttacaggaagccgttatcgcgttaactcaaacgtccgaaccgatgtacgcagcgcacatccgtctgatgcaacacctcacgtacatgcaaaagatcgccggacacgaaataatgaccacacaacttccatccgtgtttcacgaaatacacgagggatatttgcagtgttttaagcgttttaaacgtctcatgttacacgttacgggaagctgctgctactcactgacgcgatactttggattcctatatcaaccccccctaatacccgataccatcgtacaaaaaattttaaactttaacgacaaaacggacacaaccgacgacatcttaaagagcctgtcacagcccgtgagacaaggacctctatcggctgaaaacgaaagtagcagtcgactctcaaaaaacaacgttgagctgcttcaaaaactgtacgacgactttcggaccgcctccacaaacaataaccccacctctattaaacttgaatattcgggtaattataacgaaacacaagtgtccgtagattggagcacatataacctggtgacatacaccgcacccgacgatacgttaaaattcaccccggttaacacggaggcactagatcgcatgtttgcagaataaacgatggaattgccgccaattttttcgaaatttaaaatagagggcgtggcaacgacgcaccaggccgactgcaggttcggacaatacgccggctcgcagtgcttaagcaactgcgttatctacctcgcacaaagttatttcaatagagagtcccccgtgacggacactaacgacctcgacgacgttttacggcaaggggcgacgttggactttatactgagacggtccgggacgctcggctataaccaatatgcccaactgcaccacatacccagctttatcaagaccaacgagtggaccgcggccatctttcaatctcaggagtactttggactaattggacttgacgcggccatccgcgaacctttcatcgaatccctaaaatcgatcctaacgcgaaattacgccggcaccgttcaatactttctgtttatatgcggcgacaaagccggggccgtaattatcaaaaacaaaacgttttacctgttcgatccccattgcgtaccccacgtaccaaatagtccggcacacgtaatcagttcgtcagaccccaccgccatactagagtacgtgtcaccgccagacagagaatacactggcagttttctatacattatgcccagcgaatatgtcaatccagagcactacatcaccaaccactatagaactataacgttcgccaaagtacacggcccgcacatagatatatccaccggcatagagccgtgcaccatcgaagacatcccaagcccgccgcgatcgccggatgtgacgtcaaaatcatccaacctcgcacgcgtgccgagaaccaccaccgacacgtcgagcgccaaacccccaccggcgacgctatccggtttacggggcgcggaaccaccgacaagctacccagacccggcaaccaacgacgcggacacaaaactcttaactcccgctccagcgcaaaccgccgtcgaccaccccgaattccaaacaacacctggagccacgctacttctttcagaactgtcggcatccaggggtcggaaacgcaagctttccagccttcagcgatattcggattccgacgaagcgtcgtctgacgacgaaggggccccacgtaggcgcgtacacgacgatgcgatatccgccgaggtgatctggatggacgacgatatatctcctctatattcaccgtcggcgactccatcgtttgacgacgtgttcgacagccccccaatgagcccggagtttacatacgaagacgcgacagaggacacggatggcgcgtttctagaacagatcgctcgagacgcggaaacgccgttctctgcgttcgacgaccttataacggaccacgacttttcttcccttgataaaaaaatagaacagttaatcaagtacgaagcgccctcgcagcacctgccaaacatctcggacaaacaaaacgggcgagccgtccgagaagcggcggccctccaggcgatggacaaaattatgattaatatcatacttgaacacggtctaattacagacgcgcaggcccggggaccgtccgcgtgcaaaaacgttcttcaatttttcatcctgtggggagaaaaactcaacataccaataagcgacgccaagcaggtcctagaactcgatctgcaactgataccgttacatacggctatcagcgaaggaaaattcaaacagggggcgttcaaaaaacacctaacaactaaaatcaaccggtgtctggcgtcaatgagggccacgcacgcagatgcgcaaaaaaaactggcgtcggctttcaacgtcgaaggctcacagatttcgtccagcgaagcgaaaatatcagtccgggcgctgaaggaacagatcgccaaccacctgagtccaggctttttagcggtctactccgcggacgaggtaaaacatctacgggataaaattcaggacctaaaaacaggcatcgagcagcgcaacaaagaaatccaacaggaagaactgttttttgatgccatgctcacagccctggacacgttccaaccccctccgaaaacggcatttccaatggagatctttccgcaccgtaaaaccgaagttatgctcgaccacctggcgtccataaccaccaggttaaccgaggacgccaccgaagccctcaacaattacctggagaccccgcccgaccaaggaacgcacattaccaacattccaaacttttcatccatcgtggcaaatatcatatccacgttaaaaatcctaacgtacgcagaaaacgacatgcaattaaacgtaacgcccatggcaacgtacaggcgtcagctgttgtacctcggaggcgagttagcaaccatctttaatttagagtggccatacgaaaccgtgccaccggttcaagaactgcccctcgtggcgcgggcgaaagcaaaaatggaatcggtaacaaaaatggaaaagaaccaacaggctctcgaccaaatactgggagacgccgaaacgttacttgacacaataaccgcaacatccggagatgagaacccggtccgcgccatgtccataccgatactggagacctacattacaaacgcaggcgccctgataggcagttctcgaaaccagcggttcgaaaaactcaaggccgccatccacgacctggcatcatcggagtcgttcataataatgctgctaaacaacacgcggctcgataacatatcagacaatctggccaagatcgacggcatcctgaccaacaacacacgttttctttcaaacgccactgttagcaaaacgctccaaacgctgggaggcagcctaatacgcgaatgcgtagaagcgctaaataaaaggagcccctcttccctcaacaacgcgcgtctcctcgcggttcaaaccatactggggcacgcgtccgttccagatcacgagacgctgacgcgaatcgtttccggcgtcgccagcgcacaaaaggaatccgctggcgatgatccagataggtggacgcgagtaaccggtcacctaaacgagctgaagctcgtaactacccaatcgcgtgtcgacaaagccaccaggcgcaaactgttaatgataataacccgtgacctcaaggaggcggaggtgtctcaggaaacggtcctggaaacacggtggcaagaaaacgtgctaaagtttcaaccgtcgacgtccaaagaaatcgaagactttttacagtcggcaccgtcagcaaaggcccgaaaattcgcagaaaaacacctacggacgctgatcacccaattcaacggccacgagcgaccgccgtccgaggccaccgccgttcccatggactacacgccgacgcccatacccacgccacaggccgtttctacggctaccgcggaaaagggaaaggccgcatggaataaaattcaacaggcctttcaggatttcaactttcacctcatcgacgcttcggattggcaagagatggcatcagaatactccagacacggctcgtcccttcctggtacggttggaccaaagctggtgcgcttcatggagagcatctcaaacaccctggacgacatcctcacgcagaagctggcatctctgcttccaaacgggcccgcgttcagacccccagcgtttgactggatcgcgccctatcaaacacgcgtaaacgcgtttctaaaaaccataggcctgcccatggtgcgcaacctggcggacaagatccatcaccaatgccaaactgtcagtcacgcggtgcaatccgcagaccttcaacaggccacggtgggaacaagtttagaacgacccgcggccgaatactgtcgaatactctctgacatgcaagtcgcgttcaacgaccacggaatcgccgtaagatcggaggccgcggcgtacacggacgcaatcaactcgccggccaacgtcgtgactcccccgaaacccaacctagaagcccccaagaagctaataacggcaactgacgccctaaccgtcgaggactttccagatttcctaaaaacgtcaatccttcaacaggagcagcgactcattgcgctccagagagcggaatttcagcaactagaggccagcatctcggcggccgaacggctccgccaatccacccgtgacgagatcgcaggcaagatggcaaccgctatcacgcaactcttaccccgcgcccccgtcgcaatatcatcgagaccgttgaacttatcaaaacctatagactttttgagttcaacggtatacgacaaaatcctggacaaggagccttacgagacagccatagcgggattcgcgtggctggaaatcgcgacaaaatccgtaatggtctacagtcaacaaaacgaaacgcaacagttaaacgtactgctgagcgaggtagaaaaacagagcaccgtcgcgcagcgtctacacgatttggaactgtcggcgaaaaacacggacgacgtaaaggtgctgaagcaggcgctagacgaactcgcgcccctcagggtaaagggcggaaaaaccaccgtagacgcgtggaaacaaaaactggaaagcatagaatccctgcttcgcgccacgaggacggcaggcgaaatatcatcggagcttgaacgcatcggcacacaggcggttggcaccatcaccgtccgcgatttaggaacgctctccgatcaatgccgggaagccgcaaatttcctcagacaggccagtctacccgaaggcttctcggacataggcacaaaactcagcgagcttcaggcgtacattaagtacaaaaaacagtttctggagcattttgaaacaacccagcctaacgtctttcaacgcttcccgctatcccaaaacataaccgaaaacgttccggcgcgcccggcgatggactcggtggccagactgaccaatcaccttcacgtgcgcggcagcgcgccccactttacaacgtggatagaaacgctaccgaccgtcgatccggaaaaaccaactcacgtcccggcgcacggaggagcccctctgcaccgccagatcacgtactcaaacgtcctagaggcgttgttttcattatgttccaccacgctaaccccggttccgacggcccccggtctggaaatcgcaaccagggcacgccgcggggcagaggccgcaacgtggatggacagacagtggcccgacatcgctcagacgctccaagacgttctcgacacgtacgaacacaccaccgcccacgcaaaccgggacgcggcatttaacacattcttggcgatgtgcgtttttacgcaaatcatcaggggcgctagcagagccgtgacgctcccgaagttaccgagcaccgccgtcgattttccagaagagatcgttctaacacccagggaatgcacaacactggtcaccgccatgtggcccaccctggcggccgcaatcttacgattaaaatcctactcggaagccctaggactaatgagtcgtttcctcccgctaatgttccaggcgctgccgcacctaacgctagaggcccaggtcaaaaacggcccacataacaccccgcctcagttgagatgctttgccaaaacagaggcaattccgtatttcccggcgcaatggcagtcagcgaacctagagcagagcctgtggggacagacggactttttgcaaatctgcgataacaatcaacgcaaggccagggtggcggccgtcacctgggcgctcacgacgatagacggcgtggttttggaccaactgtggtccacatttaaacccatgacagccgcgtcagacgacacgtacgtcgacctagtcgagaccctacacctgaccacctttggcccgcgcggtccaacgccgaggcgagaaacgaccaccgagcacccgccgtacgagtacggacagcccacgggctactgcatctcgggtcaatcgacgacgccggtccaggcttcaaacacaccggtatccgctttcgaggcggtgctcggagcaatggtgtttcacgtaccgatcagaatatttttggcggccacgcccaagcgccttggccaggcgcgcggcggcatggggctcctcacacccatcctggaatgcgtccccgacgtcgagcccttcaaaagcctgtataacgcaccccgcaaacccgtgcccattgaaacgctacccgcatccctccacccgcacgacgagcgacaggtctttctgagacaggcacagtggctatcctaccgattcacaccacacgaagccgcccggtcgtcgactccgccgcttctggtggtcatagaccctgaaaacctcgtaacggcaacgtactccagtggcgggcctgcaaatttcgagagcaggccgttttacgtgatgcccggaccataccccccagactggccaaaaacgctgtcggtaacatcaaacacgtccgtgacgcacctcagccacgacgagatatgtaacctctttactacgctatcccgagaacacgggaccgtgcaaggcagggatatcttcgcagcggctccgacaaacgtcacaccggaacaaaccgccaatcctccggcatgggaaacggataaccgattaataacgcaaacagaaaccgccaaaaaacctcatataattcctgcgtctcctaaagcgcggacagatccaccggtggaaaccacgacccaccattcacaagggcaagcgtcgcaacacgcaaacagcaacgtaaaccagcccggtcaaattacttcacacgcgtcacgtaacacaccgtcaaccgcacctcaggcctcatcttcaccggaaaaattcaacacgcaaacggtgcctcgactaatatctcaaacgtcggaaacggcccatataaaccagccagcctccggccaggtcaccgaaccaaagggaatctttgggacgtataaaccccgagtgctcaccgaacccgccaaacccgcaaacgccggcgtagcctctcgccaaccagaggcaaccaccacggtccccaagttaccgattaatccacccaccgctagggtctttatagggaccgcgtccaaactctcgccagccgtcgaagagagccacggcgccacacccgacgcacatcagtcgaagatagatcgggaaaaatacgccgagagtcggcctcgccgcaccccacacctcgaagaggggccacgggagcctcacgtcaacactccaaccagcgcacacataaacgtcccctctagccaaggtcaaaaaacagtacacgggcgcgaaaatcccggccttcaaacagcaactcccagcgccccccaaccaaccgcatcaaacccgcgcattcaatacacgctccccagaacggacggccggttgcttcacgacgaatcggaggtggaatcgaccccaaccgaggaggtaaaacgatcgccaaaaacacaagatgtgtctcacgggcccgaaccggacgactccaggtggaccgccccgctcggtccaaccatagagattcatcgactggaacacccccaaattctcaaaaatataacatcactcaccgtccccactcccagagtcaccccaatccctcccactaacatctggatacccctatcccacgtcaacatccaacacgaagaaatcacacgagccaagaatgtgttaatgcgatttattcaaaacgtacgaagaaaacttcaagcgtcgtctgacgctctatccgaggctattgccagaataaagtttttatatctgtaacgcgcccatctcacttgctttttttattttgagacgagcgtcttgtgtccagagtagttgcgtcgctaggtgataacgaagtggaccctatgccagaagacgccacgctcccgggtccgccgccaccgggcgcggggcccatcgagcctatcaatgaatggggcccgctggagatcgtagtaaagctatttgacccgagggtggaggccaccggggcaacgctcggggcggccgagccggacccggacaaaacgcccgagatactagaactagcgtcctttttgccgcgaaggccccggaggtggtctttgcgcagaattccattcttcttttgcatatacatgtcgtaatgatgtttggccgttaaaaacaccagataattacgtttcgcgatggcatactgggcgggagacatgtcaccctggggaaggttgttcatctcggcaaccagcgggtgatttgggtaatcgtgctcaaggcgtccctgaacgattggctccttaacccgcaaggacgacatctcttttaacttctattacactttccacaggacagggacgatatagacgaggtcaaataaaacagctcggccacacgcaaatgctttaataggccggtcgcgcagtccggcgacgccaaacagggcacgacgacgctaaccagggagttcgcgtctcgtatggcatgtgccgcgttgttggcgagcacgcaccgtaaatagggatctccaacacacgtgatctcgaatagagatataacccgcatgtgcgatccgccacaataagagcaatatacgcgcccggtggtagcacagatcgaaagctgcttctctttttggtcgcgactgaaaaacacgttggtgggtgggaaatttacggtttcaaatttaccccgtccgaaattcaaacagtaaccgcactcgaggcacaccaccaccctcggagctggcacggtcttctccagtacgctcctggccaccacctgggaccaaacaggtagagagatacacggaaacagtacgttatacgccaatactttttgacccaggtcgcgggatatctccgtctcggtcgactcccctatgggcaacacaacacgggacatgctcagcagggccctaaacgtcaggctcctcagaagggcgttaaacgggttgccgcacgggacggtcggcgccagttctcgcagcgaggccagaagtcccgcgtccgaagggcccgggacactctcattcaggttagctcccagacgtctggaaatggacggaacgttcaactgcatcgagacacaaccgcccccgttccatttcttccgcaaaacgggcagatccaacgcgtgctgtggcaaacaggtaaccaggggaaaccgctggcgacagttaagggttttgcacacgagacaacacgccctctgaaacgacacaacaagatacctggaccacgcgctcctgggaacggccgttaaacttaaaccttcgtcgctgggacagcccacgccggtgcagatacacctcagcacccacgcgtacgcctctaacaacgaccggccgatatcgtgcagccgcgatctcacgtcgccgtttttaggtgggttatccggcctccagccggtagcaatctcgttcagggcggtctgaaaggatggggcagaaattaacgtgcaagcccattttgggggtcgtccgtcccaggcaccgagcccgtacgtcacaaaacacacgtagcattcggggcatagtccgattgagcgtatagaggccgcgagatctaagcccagccgagaaccatccagccaacgatgggcaggataagaaccgcgcccatgacaggcagcttcgtcttcagccaggttaggcaagcggcccgaggccatcccccaattgtaccgattgaattggttaattggtcatcgccgcacgctctccgcgcccaatatccttcaactccgaccccgaagggcgggccacggagcccgttcgcctcaacgcaccgaggccgtcggagtgacccgtcagggcaaaaaccgttctaagaagggtttttaaccgtttagcgctctttggagtcacgaccaaaaactgtaaaacctgtcggtgctccgtaaagtaggtgcggcatatgaccatggagctgtaaacgtttaggtctccggagaaaaccagacgtgccttaaatttcataaaatcgtcctggcccagggacacggacgagttcctctcaagatacacgtccgaatttataggcaggttttttccaaactgggcatcggcgtcacgtggcttacacaaaaaacatttcagcgtggtggccaaaccgttgttgataattacaaaacacggggcaaacgggtaggccagtctctctagtttgtggagccaaaacttatacacaaactcgagatgatagacgcagccgtgctgcaggcgcacggtgcacacggggaccgccccgcctttagcgtatacgggagccccgtcctgacacctctccaagtccagggagattccagagggtcccaggtaagagacaactaaatcgcacagctcgtcaactaaacgttttccggaactcatcgttataaagatcctttaggtgctgtgcgtggctcccgtaaaaaccgcgtccgtgctaacgattttgtgaatgacctgttttacggcgtttaccttggcgtccaggaccatgcagtgctcacagtgagctgaccgcgtctgagcacgatgacagaggaaagtttttaaatactgacagtagttaatggcgttgagcctggaatatatggtgggaaacataattttcatgtcatcgggcagcagggactcgaacgccaataaatcgtcaccgaacatcacgtgagacagaggtaaaagatgctcaccgccggtaccgcgtaacacgcgaccagccaccccttcaaatattttagccttaaaaagcgggccccctaaagtcgtccaactcagcttaaaaactcctacccattatgttcgtcccgtggcaactggagaccctcatgagacactggccctctctgcggggactcgtagaacaatccttcctccccggtaccccggacggagcttttaacagcccggtattaatccacactcaggactctctacaacccgcctcatcgtgcagggtgtgtagcctcctgttcactctggtccggacattcccaccccccgactctttcttcgaagactacggctggttgtgcctcacctgcctatacgccccccgatcatggacggctaccctcatggtggctgccgaccttttggaactaacgcacgtgtacttcccgcaatgcgtgaaagatgggccagtatacaccgcccaaagcatcctcggaatcgacgtccagctgcacttcttcgcaacccgctgcttccgacccatcgacagagaacaaatactccacacatctcatttaaattttttacaaaccgagtttattaggggcatgttagaaggcacgattccgggatcgttctgttttaaaacgtcctggccgcgcacagaaaaggacgaccaacaacctaccgttgcgtgttgttccgttggccgcggaagtcacaccaaccgggataaccgcctacccgaggacctggaagaggcgttcaactccacgaacgccgaggaaaagcccagcctcctcggcgtcttttcggcaacgtgggcagaatcccagcttcttggctccgacacacaacaggcagatacccatttacaaccctccgccttcccaaccccagaagatgctgaccaatcacagggcccctgcctgatgcacccaacgctcaacctaaaaacaaaaaaccacaccgcatccatatgcgttctatgcgagtgtctggccgcacacccggacgccggtccggttctgaaagatctgcgtcgcgacattctggaaaacatggaaaacaacgttaagctcgtcaatcgcatatcgtacatcctaaacgatccggactcactgtcacacgtgcgcgacgaacatctgcgcggcctaattaaacggtgctcggcacaagaaatccacaagcattttttttgcgacccggtgtgcgtcctgaacacgtactcgcactgtcccgcggttttatttaaatgcccacctcccgaaaagtataagaagctcaaagctcgtctggcaaccggagagttcctagactgcaacagaatatttgactgcgagaccttacagaccctggccgtcctctttaaggggtctcaactggccaaaatcggcaaaaccacgtcgctcgagataatccgtgaactcggatttcaactgcgtcgacacaacattcaaatcacccacccgtttcaaacctccaacctatacatttaatcttcagaagcgcaccagacaatgccaaaacagcccagaagtcgattggcgtctcgagcgccgtacgcacctagcgtcaggcgaccggacgggccccagtccacgcgaccggcatccaggcacggcagctgcaaaagcgaaatcatgcagtggaaaaagttagtttcagacacgcagtttttttctgccctaacgcgccgccacgagctgggggtggactttttaagagaaatggggaccccgatatgcacctcaaagtccgttatgttgccgttaaacctaaaaaccatcgccccgggtcggtgcgtctctctctcatcattcggacactcgtcaaacatggggttcaactgttcgtcgtgcacgccaactgacaggtcagcggtgtctctggacgcaaacgcgctcggcgaagattccgccaggaaaaacagcgagctgtgttcagtggcgttaaccttttaccaccacgccgaaaaggtcgtgcagcacaagggcttttacctgtctctgctcagccactccatggaagtcgttaggaaaagcttcacgcaacccgggttgctctacgcccacctagtcctaaaaacctttggccacgatcctttacctatttttacagtcgatgccgatgagagactcgcactctgggcggtgttccacactagagacctacacctgggggaaaccagtctgcgactcattatggacaaccttccaaattatgacataacggtggactgcatcaagcaaacgtacataatgaagtttacaccctcgcgaccggacaacgcaaccgtgacggttcctgtcaacagcatttgcgaggccgtggccaccctagactgcaccgacgagtttcgagaagaaattcaaaggggcacggccatcataaactcccaggggctattgtaacctttccctagacggaaacagatgtaattcacctattcaaatgttaagtttttgttttgaacatatcaccaataaaaacaacttatgtttacacaaattgaagagcgtttcaattttaccataaacatagcaaaaaccacggtaactaaaactcaatagcgatattcggatgggatccctagagagcaatcgacgtgcgccggtattaattgacgggaacgcacgttaactgctctccacgaatcgcaaactccgcgtttttaggaccattcgacgccgttaataattggcagtaaactgcgaaagggcgtacatcgcagtagtgattttacgatgtgcacacctttccgcggttcaccgccaacaacaaaagcgggtcaccaggcacctaaatcgcccgggccttttatgccaaagtacaaaagggaccggcgttctacgttcacgaacaacactgttgatcgcaaatacaatagtgttgttggtgaccgcaaactgccaaatcctaaaatgcatggacatagtcctcaaaatctagaacgaacttccaaaaacatgcggcaataaacaaccccttgcagttaatgcacatcgtaacgagacggaatccaaagaacacgcccgatcttaagaccacgggcacgtgtctttgggttccgggggcgttacgatggaagttcatcaccaagtatcccatcaacctgttacaaaagcgtaaagataatcggtttacaataaacaataaaaaatagtggacgaagttacattccgacatacaagcggacccaaaaaaacaccccaacgcaaaacaactggagactgtgtctttgggtcccttccacgtcgcaatgagatttctggtgaccgtgtgctgcttaacgcgttacagttgtgttttatggctacgtaaacacaccaatctagaatgctaacacgtgcaccgcgcttaggcgcgtttgtgcaactaactgctgtcaggttactactcttatttttaacaagtagcataccaactgcaattaattgcactaaacccagtttagtctttttagtcgagtcggcccatagtcttcatggagaaccatcattcatctccgtcaacggcgtcggtgttaccgttgttattgcgggcaaaacaggctcgggggaaaaaagagcaacatatcgtcagtttaaaacaactcaagttctaaaatctcacccactcgtaaacaaaaaacatcgcagttaaggtgtatgtaccgaacataacaacacaagttttttaaaacacagctgcggtaagtaacccccattgccacgcgtgtcgcgtgctaagtgtttttaaaattacattgtgcgttttacacaccgagcagtaatctcaggagggcggttaacgagcgatatacatattccctaaacacgggaacgcgcgctgaccgccctccccaaatcacaacacgggactacaaagcctagtgttaatataatcaaattaaaaaaccacagaaacctttagtcgtgcgcaaacactagcaaaggtacctagagctttcccctatacttcaaaaaacagcggtgggttatttgaccacacgttaagtaaacacccgtaagaattattcccgtttatcaaaatggaaaaattaaggctttgcgttaaaatctgctaacgcaaagggcacttaatttttccagtttggactcggaactttaccgctaacgttaaaatttaataatgcaaaaggcacataattttttttgtgtaacctcaaaactctacagctaaagttaataacggcgactttgggcagcgaatcagtgtctgtcgcaaaccctgttaaatttaaacacaacagggagcgcgactaaacacggactaactgctcatcgggccctgttgaagaagttggtgttgcaatgcattagggaatctaaaaacgaaatcctgctcggtaacacggaaattgtctttagttccctacagcatcacaacaattgtaaaccataaacgtacgcgcgtggggttggtgtggtgtgaaatgcttggtgcaactgttacatgggcggattgtaaatgtggtgcttggtgcaacggtggtggggtgcaagtcccccggtggggtgcaagtcccccggtggggtgcaggtcccccggtggggtgcaggtcccccggtggggtgcaagtcccccggtggggtgcaagtcccccggtggggtgcaagtcccccggtggggtgcaagtcccccggtggggtgcaagtcccccggtggggtgcaagtcccccggtggggtgcaagtcccccggtggggtgcaagtcccccggtggggtgcacgtcccccggtggggtgcaagtcccccggtggggtgcaagtcccccggtggggtgcaagtcccccggtggggtgcaagtcccccggtggggttcaagtcccccggtggggtgcaagtcccccggtggggtgcaagtcccccggtggggtgcaggtcccccggtgggagcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggctcggctccggggtggctccgggtgggggcggcctaaaatccttaccggtaaatttagcagtaaatccaacgcagtaaatccgcaagctagccgcacagaggtgcgactgcctgccaaggctcctggcgcctcttttatagcgctaaatgccctccccaaatggttactatggtttagttatgcaaccataataccaataaatgaatcaccaagaatacggccaacgcgccagcgtccaaccgccctccaaccgtgcagcgccgtatattcgaattgccttccgcacgaatacggggcgcagcatggtcgcgggaggctggcattgcgcaatattcccagcattccaacgggcacgcaagggtccaggctaccggggattccccaaacatcatccagcgcatagaggttgtggctgagctgcctatctggggattccccaaacccgagacccactgagatgctattcagtgggactgcaggctgggtcctcccaggacaggtgcctgcgtgtgtggtaagggagttcccctaattaaaatattatattaataaataaaccaggctaagctgtaattaatgaggcacaggctgccgacctaggtacctggggaaccccctaacgggatgtaattaattcaaacctatataaattccaccctgttagggggatccccacgtgtacctgaattaatgcgggatacaggctgtaggtttggtaatgggtaccaggctggagtgactcataccatgggattaaataccagggatacaagctgtaggttcggtaacgggtaccaggctggagtgactcataccatgggattaaataccagggatacaggctgtaggttcggtaatgggtaccaggctggagtgactcataccatgggattaaataccagggatacaggctgtaggttcggtaatgggtaccaggctggagtgactcataccatgggattaaataccagggatacaggctgtaggttcggtaatttaaacctatataaattttaccctgttgggggaatccccgtttgtacctgaattaatataggaatacaggctgtagaatcggtaatggaaaccaggctgtaggactaggctggagtgactcataccatggaattcaattaacaaggaaaatataatagaatatatatatatatagggaacttgtaaacaaaacccaactcgcggattggctgcctgtttgggccaaccagcagcgagaattccctgattgacaggcggactggccaatggtttgcgagcatttttgattgacagaacggccggccaatcgcaaccgagaactcggcagcgaagcaaaagacagacggccgcggcgaccaatggccgccgcgggttagtttgattgacggcttggccggccaatgggaaacgtccctgccggcggcccctaatccccctggcttaagggattacggccgtaaacatcgctggcgcggtgccaccgccgccgaacccccgcgcccggttcggcgtgacctcgccgcgaccccgccgccctcatgcgcaccatgggtgcgatgggcaccggcggcaacattattcgctgaccgccggacgaccccggcgccaaagagcggccgatgggtacgcgcggcgaccatcgaaacatttttcaataaccccagtctgaccccgcggcccaaatctggggcccatggtggcgcgcggcccccaccgtccccatttcccacggtttgctcatgggcaccgacggtgcccgtggtgatatgtttcagtgaccctcggatgacccgggcgacgttttgcgcaccatggtcccgaattcccccatgagtacatgtttcggtgacccgccggcgaccccgggcggcaaaagccgccccatgggcaccggacggcaccatgtgcacatctttcagtgacccgcgggcgaccccggcggcggcaccgcgggcgctgggggtgatatgttctgatgaccgccgccttacgtttgcagcggtgtcagcggaacatattctccccggctcccgagcccggggcccgcggacccgagttgggttattaaagttacgttagggattaattttaatccactaccccccttaatgtgcggctcggcctgcggcgacgctgacgccggcgggttcgacccattgaccgcatcctcggtggccgggcagaggccggccggccaaaggtgcgatcgcggggtctgacccacccgtgggagaaacccctccgttccgttgtcttctgcctgcggtccctggccccctggcccaccggggatgtcttgtgagcactttccccggtgagaatagacaggagagtgcccgcaagacatcgacggggttgccggtcggtgaacgggaggcgcgccgcttgcgtgcgtgaggtgtcgccggtgggccgtatgacccccgcaacgcgaggggccgctggcgcggaaatttccccagaaccggccgcgccgcttggcggcgcggcttcctcccccaacgacccctaatttggtttttaagttgttatttaaagctccgctgtgtttgtggcacgcgcttaagcgttgccacctgttgcggagatcccccgtgcgcatcgccgccctcctcgcctcggtgcacgctgccgagcgtgccacctgtttggttgtgtcatcccatgtgcgcatccccccatcttatttccccgaccaggcaacataaacgtcacgtcgtttgtaagaaataattgctttttttattttcgttaaccccgcaaccgcgatacagtctctcgcctttcacccgttgttttgttaccagcccctcttggcggccggcaccggcgttaaccgggtgcgttggcggccacgaccttggccacgtcgcgcctggagacggcctgcagcaggtcacttaaaaccatgtagtttgaggggctgaccagggccgccttttccatttgcgagagccaccgaaaaaaggtgggtgactggttgcgcctgcgaccaagctggtcgcccgttagaaaaactaaatttttcacgtccctttctgagagctggcggtcaatggagagcatcaggtgtttgtatggactaaagtaactggggctggaccggagcctctcgataataaatcccacgtctagtaaaaaaaaggtggagaccaggtccaatcgcccaacgagaaacacggcctccagcaacaccgggaggggaataccgggttctccagttgaggggcacagcccgtttgcaaacccctcgggcgtatcgtcggacgccggccggtcaaaaagccacagcacggcctctctgtcgtctgcctctaggtggcgaccgaagtccactaaccgcttatgcgggaacatgggtccggtgcggcgctcgccgacaccctcttctacctacactagagcgtttgataaatcacgacgaaccgcgggcgttacttaaaatgtggtcttcgtcaaattcccgcaaggtggtggcaaccgactcggcggcggccgcgagggttgcggccgaaacgccgacggcctccgccaatctggccggaggcggcgcagcggcaccgaggctaaacagcgcgcaggcggccgccaccagggacgggggcagaccgccggtggccgggttcacgatggctttgtgaacagactccaccacctggctgtgaagggcaaagagctgctcttttgtaaagccgcttttaaacagggtgggcccgatggcgtccgttggtaagacggcctctagtttccaccttaaagccttaaggattgatttttcctgtttcagtaaatccgccacggaaaaatcctccgccgcgcaaaagcataaaaaggcggccttgaaaggattcaggtctctaatcttgccggctaaaaatagacaggcggcccccagcctctgaaaccgccgacgggggatgctgcgacactctagatagcgatccagaatactaacggccagtgggaaaacgctagcatccgcctggtgggccctggcaacagaccgcatccacgtccccaagataactcgcatgcccaccgtcacctccgtttgtatcgtcccatagcaggccgtggatgttacaaaggacgcttcgtgggccaggaggttagagagggcccggtcctggtacaggacggggtcaatggtcccggtgggcactgggccaacagaagccattgtcaaggcaaggtaaagaaagcgatataagtcctacctgcggctcacggaggcttaatacgaacgtccggcttaacacacaatcttctttgcgctctatgtccgctccacactggtcccatgtcttctgcagttcagccctgcctgttcccggaaagcggcggactagatgcaacgcggaagcggctgttcatttaaggtgagtcacaggcggggcaatttgccaaccagtgacgcgtttcctgcagtctgggttttcacctggcaagcgccactggttcttggcagccggccggtggggatttttttagtgctgaattggcagtcctctgtccatgcgcactatgctggcctggaagtggggggcggatgggtgatgtcgttcccaccggcgacgggcctttttgacggccttggcggttccgcggctttgacagaacaccgtaatgcaaaaagccgggatgacgggactgccaggtgcgtccggcaaaacgcggacgctggatgctctaatgccgggaaactgtaggcaccttgccaggcgatacagtgaataggggtctgagttacaaaagatgactccgtgacaccacgggcccggccgaggggggtcgatcctgggtcccaatacctgacgataaaatcttctctgtgggcggtattttctgggtacctcttcctctgatgagggatacggtatgggaatggggtatcgcggcgggcgcggggggcgcggggggcgcgccggttgctgctgccgctcctccccctcttgctcctgttgtctttcaggagcttcgtcctcgtgggagttgtgactcgtgcatctgagacgtaaacaaggaactcctccggcacgcgtgggtggaccccaaccccctacggtgtacgagtgggatgtatggccaggaaacgggggtcctggccgagacggcccggtggggtggtctgtgtggctcaccgcctgttgcgtgttaggggatggggcctgttgaggcgttggcgattggggtggtcccggtgagtcgggcggggattgcggagggggcgaggacgtcggcgttggcgattgtggcggtccaggtgagtcgggagggggcaaggacgtcggaggctcaggcggttcaggtgagtcgtggtgcgatggttgcaaaaacgacgaaggctgctgattgggcgatggattatgcgacgacggcggtgcaggtgagtgggggccggggtgtgacgggggtgatggtgggggcgaatgcgatgcgggggatgccggggacgaccctgacgtgttttgaggtgacaggcctgaagctaacaccggcgcagacccgcttggcgacggcgttggagatcccaacacgatgacggactcagaccctgaccctgatggcgacccttgggatgacccgtgcctggattcggtggccgtttcggtgtccgatccgtgcccttgttctgcaaccgcccgcacttgcggccctgatgttgttggagcggggggcgcgggtgcggcggctggcgcggccgtgccacgccccctgccgcgcctcccggcctgttctctagtgccgccgcgtcttccgcaatgtcccctgcaggaacttcgcaacccgtgtcccgatactatgccggaacgatgttgccggctgccccacatgttcgcggtttactgcgtgtaaaaaacggcagattaagtagattgccctttccacgacccgcagccacctgagcgcgcgcacgcagaacctatgtttagtgcgattgcgattatgtccgctaggtggcgggctattttgatcctgccgcgcacgcattgaccgttaggtggcgcagagcgccgttaaccgcagcgagcaccgcacggcggttttagtttcgttactataacgtttagttaaaatccgtggccgatattgggtggcagtgtgtaagtcacgcgcgccgcggcccccttaccttgtcgccggcgaaccgcggaaagtctgttccgccgcgcagcgcgcggccgctgccgaaaccaacgccgcttgaatttcgcgggcggctgggcttggtgtcagtgattcacacagatgcgatgtgattgggcgtggtggtcaacgtcacttgttaacccgtaaatctggaaattcttgtccgccgcgccggtgagccgcggccctaagccttatagtgctgtgcagcgatcctgaaactcgaaagctggccatataaaccccagtatggcgttgcgtgtcggtggcaacctttttgaaaaagatttattgccgccgggtgttaagcaccaacaccgaccgtgcgttttcaattatgtcgggaggaattacattaacgctgctgctggcgacgcttgccacggttcggtgcgctcttcaaacgcactatgcggcggtccccgtgcactctaccgcgtccctggggtgcgtgttaacgacaccccacgacgttcttatcgttacctggcaaaaacaggaatcgcctagtcccgttaacgtggccacatatagttccgaagcgggcacggtggttcagcccccgttcgccggtagggttgacattcccgaacacaagttgaccagaacgaccctgaagttttttaatgccaccctggaggacgaggggtgctacctgtgtatctttaacgcgtttggagtgggaaagctgtcgggaaccgcctgcttgacggtttacgtccccctgtccatgtccgtcacgttttaccccccgattaacccgacgcagctcgtctgtcgggccgaggccagtcccgcaccgtcggtcaactggaccggcgtgccgcccgagctgtgcagcgaacctgaagtgtttccccggcccaacggaacaaccctggttgtgggtcgctgcaacgtaacgtcggtggaccccgaagaccttgagaacgccacgtgcctggtcactcatataggcggtttggccgcggcgcggcccctggaccccgtgttttcggatcccctggaagggacgagccactacgtggtgggtgtggtggcagcggccgccgttttaggcatttttttaacgggtgtttttttgtataggtctatgtgagcgcgcgtgtcccccgtgtctagtgttttgttccccagtgagtgtctccatgacaaatacaaatttgaggctggctttttagggtggtttcttgtgcgacgcttcctgtgtaactgcatacaccggggtgtcgccaggaaaccgcgtctcccctttatgtccgctcgccctcccagagcgaaagtgagaatggttcctggggcgttttggcgttgagagagtcgggcgatgttgccgtagcggcgtctgcaaaggctcacccgcttctgttttttttctttttgtcagacaacaacatggacgccttgaacaataaccttaacctgctgatggattttctgtctaactattcgaatagctacagtagttatgacgacaatatgtcttacaccttagacacggaatccacgctgtgtcggctgacggtggttttcccacctaccgtttatgcgattatatgcttttttattttttgcattacgctgtttgggaacgcgttggtgctatatattttttttaaatttaaagcgctcgccaactctgtggatgtactgatggctgggttgtgttgtaactccctgtttctgtgcgcgtcgtttttgttcagctggctgctgtacgtcgcgccacagatgctcacgtccgcgacgtgcaaggtggaaatctttttcttttacctgtacacgtactttggcgtgtacattgtggtgtgtatcagccttatcaggtgcctgttagttgtgttttcccgccgcccgtgggtcaagcacggggcctccggctttctctgcgtgtgtgtgtctttaatcgtggcgctggcgctgtctgccaacgcgagcctctataggacggccctgcgtcacccagagaccagcgagtggatatgctacgaagatgccggggaagataccgtcaactggaagctgagaatcagaaccaccagcgcgatctgcgggtttttggttccgtttgggctgatggtgctcttttacggacttacgtggtgtatggttaaaagcacgaagctggccagaaagggagccgttaggggtgtaattgtgacggtggtggtgctgtttttaattttttgcctgccctatcacctgtgcaacttttttgacaccctgttgaggaccggttttctggccgaaacgtgctacctcagggacgtgatcagcgtggccatgcacatatgctccctgctacagagcatgtatagcgcgttcgtgccagtcgtgtattctggtcttgggtctctgtttaggagaagggttagggatacctggtccgtgtttaggtgtttttccacttcaggtagtttatgagacactcacgcgacacttggttggattgtttgtgtacatttattttcattttgtgtacatttattttcattaaagcgatctgacctgcagaccttacctgacgtttactgtctgtttcttatgcaccagaggaacagggactggaaggccagcgcccacggggaaactgtcatgtccgcaagctcgggcggcacgtacgcccactgccaggggtggaacgtcacggcggggtcgaccagggaggccaggtggcgcccgtccggtgacgtgatggcggccacggccgagttggcagacgggttgcgtgggtagtgctgcgcgaacatcctcgggtcggcgttgcccgtgtggtagttcagggcgatgcgctgctgctggttgagatggtactccatggcgtcgcgcgggtatctcacgcccaggtaccggccgttggcccaccctgggaggacgaggccccgaagaaccctaaacatgatgctgatggtggtctgggggatgtggaggtttagccagaggcactcgtggttccctgatgcgttctcctccaggtggatgtcccactggtcgggggttttgggtccgggcgtgtcgtgaggggtctctctaagaagaccgagcgcccccaggagctggaacccaaactccccgcagcatagtgaaaatgtatccgctcggcggaagaaggccataaggcccccatagcacccagggtcgttgagaagacccatgatcgcgcatcgggcccccacgtagctgtcttcgatgcccacggttccaccgatggtcagaccggagaatccccggaggatgttccctcctctaaggtcgtcggtggagacggccgcgacgtcgaacccgacgttggtgaaggcggccatcagcgccctgggaagcggggcaccgggggtgaccaaggcggccactgctggcggcctcgacggcgttgcaaacagagtcagttcgctgtttctgcaaacctcggcgaggtggcccaggttgtgttggttgcacccgtagtcctttctgtagagttcctgcgcgggcgtgaagctgggcccccatgagtaccactgttcgtccgagaacgaggtccagtttgccgccaccgaagtgagggtctgtgaatagacttcatcgttgttgtgtgagatgacgattctttctgacagaccctcctgacccacggtgccgcacacggtggcccggcagtcaaagttttggcacgcctggcgcacctcgtcgacgtgctggggctggatttcgaagatgacgcccggggtttcggacaccagccactgcaggggcgtttcctctgacgggatgcgaatctgtaggcctcggttaccggccagggccatctcgatagcggtaaccaccatgcctccgtcgctgacgtcgtggccggacaccaccagaccccgggagagcagggcctctgttagcatgaagaggttggccaggtgtgtcgcctgaacgtccggtagggtgggactcggcaagagacacaggtgctcaaaggtcgatccctgggtcaggtgcggcacggggaaggacaccagcacgatgaggttaccggtggccttcaggtcaggtgtgacgcgttgcctggacgacttcacctcggcggtggccgtgatgacgactgcgttaaacggcacgggggctaccgtctgatgctgttgggtggcgctgatcagttcttccgacaggcacggggtgctgccggccgacgtgatggcgaagttgatgctcaggtccctgcatagttccttgcaggcaaacagtgtgtgctgcaggagccaggcctggtcgtcctccgggttccaaccgaccgacgccgtcagcgtgatgtcgcttagacgccgcacgtgcgctagcatgatgttggtgaccgcctcgcagatggcgtatctcgcgcccactgccgcgttgatgcccatcttgtatgcctgttctccataagccacgcactgtccgacccaccggttcctgcccctgccctgggtgacccaggtttctgggctaaccaggcgttccgtcgcgtcctgcctggtgatcggctgggggcgatatatgggcacccgcgccattctgtccgtaaacaccgacgtgtggttgacaatgtggtaatctgacagcggccggcccaggggtcccacttcgcactgctgagccacgagtccgttggagcatctgtccacgtgtcgcgtgacaaactctttgctgccaaccgtcggacatcgcagaagctgatcgaccacggaggccaggcggaataggccccagtcggtcggcgagggtgcctgttcgccaccatcctcaggttcttctgggcgcaccagccagttggaggaaacgggctgctcgtcgaacccaaaagttgcgatgacttcggccgatgttggctgtctgatcggccacgctcccgggttccacagctccaggtcgttcacgaactgaattccaagctcttcgcaggtttccccaacgacggcgaaagggcatccgcagatgcgtgcggccctcctgatggcatccaacgggcccggttccggctccgtgtcgcgcgggaggacggcaaagaccacgcaggcagacacgttgagaaacttgttaactaggatgtcgttcacggtttcccgtgagatgtctggcgtggcggtcagttggctcagaatggttggtggaagctgagagacaaagaggcgcaggccgccccgcgagaccagggctcccaggtggccgatgacagaggcgggttcatgggcgcgattaatgctggtaaataggggcccttccaaaatctcgtaagccagcgacaacgtttgggtgatgcgggtaatacctgcggcgctccccaggtaaagcgagcgctggttgccatcgccgtcaacgggttcaaaagacccaagacagatgataggctgactgaataaagacctcctgaggaatgtgtatgcttgaggcacgcacgataacagcgatgtggtggtaataacgttcgcctcgctggtggtggcgctgtggacggttttaagaaagccaccgaggacaggcgtgccggtgcgcgccagctgctggcagtggagtgtcgctgttgtgttaatagctctgtggcgtgcgcgagaaaatgcgcccactgacggcgtagtggcgtaaaatccacacagggccgatctgatttcgcccccgggcagcagcgtggcatggaacagaatgttttgcagcgcacctgcacctgtctgaatggcgctgtgcaggtgtgtaaatgagtcagctatgtgggttacgttttcgcacgggaggcgctggaaaacgcagctgtagctcctgcgggccatgacggcgtatctggacaccgtatagctgttgccgttggaggcctgcacggccagcggtaatatgttccgttgtaacgggagcatcaccgcggcgcatattgggtctctctcgggcaacggtctccaggcggtggattgcaatgtcaccgggagctggcgctgcacgcgcacatcatattctactagtgtgccaagtgtcagggcggccaaggtgcccgggggtacccgatcggcgacaaaccgtctcgtctggcagctcagatgtctgcccacctctaaccgcaacgttctatcgaggccgctgtctctaaggcactgcactatttcgctagatcttgtggtaggttggcgcatcaggtctgggccatagggcagggatatagtatgatggccctggaggtgcgccgaccgagtttccagaggtgcataattaaccagaggcgccagaagatgccagataacgcccagcgctgggtcgtcatcgtcgttgtcaaagttatcagaggggcgtgttacaagccaaagtaaaaattcactaaagcactgttctggcacaagcgccaaaacactttctgcgtccgaattgtcatgaataaaagcctcctcttctggactaagggctgcagcggcccagcgagggttcgtgcgttgggccatatttgctaagtctagccggcgcaaaagcaagcgggtgggactttaatagggcgtataggggttgttaggtatgcggtggggagccgtccatgttacatggacttcaatattaatttatgttagttttgggcaccttgggatgttacacttataacttcccccatgtcacagtaatcacttgacagatccccttctaactcataacaactgtttcggggtgttgaggttacaggtaagggtgggaggggaatgttaggtggcgaaccaaagcataacgtcgcggggcgggagtgggagcctgtaaccccagagatgtcacttacgcttgttaggcgatcgacaactacctccgaaagtgtgtcctgttggatgttttgtggattaggtaaaacagaaagttgtggagaaagtaaccttatagatgacctcctgctggcgcgtggctgggtgacagctggttgttgatgaatgaacacttcctcgggcgttcctggggtggtgggtgtggtctcactaaggacggccaactcaacactaacttcctcccgttcagaccgtgagcggtttcgtctgtagcgtcttgtggagggggttgcctgaacccttgttggtgtaaacaatccaggtaaccggtaaatgcacgcggtcagggcgatttgtaacaaacctaaaaacgcataataagcttacaaacgcatttttggtaacagtttgtataatttagccccaaagtttatacaaagcgaaactacttaccgattagtaaaatgcaaaccgcaagaaggataacactcactgattctgttccaattggttcgaatttaagaatggagagtaaatttgccccgctgagaatgcctggaaataagaggcgcgtattttacatgtggtacagggtagaaaaccttgtaggtttcagttgagtcaccctgccacgtaaacccgcctagagtgctgcacgcggtgttggttgcaggaagtacatcctaattttaactctaaaaggggttccttgtaaaattatttaagttatggtgtacttacataatatccaatgagtagttacactccaaaaagatactcgataaaatgcgtgatatgtgaagtttgcaacaaatgcagcggtgagtaggcccaagaaaatgataaaccctatatgaggaaattttattagtacgtttgtgtaactgcaaaataaaattttaaataaatactttatattcatgttaccaagtttaatttgtggtggttgttcttgaattatatgtactcctagttctgaggcaactgtatgtagtgcaagcacgctgccttgcaacataaccaataggcatgttccatgattcgatattcgccatgccattttaagtgccacgcagattgcagcaatagctataaaaaaacaaaagtatgttaatagtctataaagtataaaacattaaaacaaaattaaaaaattaaatacctgtatttgccgtaactatagagtatgataatagtaaggtgtaagactgtgttgttgtagtttctgttaatgcataaagtcccacaagtgctaaaataaaaaaataaatgttagttatttaatttttaaaagtttatactattcaagtaattcaaagttgtgtcatacctgcgtaaattgcaaaccaagatccaattattcttagtatgtttgtttttccacatagccacacataaccataaaacacaatgggcattaaaattactggcaacagcgcagatactttgtagtttggagtccacataagaaatgccgtaagaataacagtgcctatccatgttggtcccacaagaagacatgatgttgttttactagaaacaattttcggtggttttataacaaatattaatatgcctgaaaaatataaacaaacgattaatttaaggccaatatcatagtaaaacaaactttaaacaggtaaaaagttacgtcttacctatgtaaaattgagatacgcatgcaatagcactacacgtctgtaccaaaatacttgcgttgtccaaggccaatgccgatagtaaaacacaacatgctgcaaacaagtacacaaaatataagtttaatttgctaattaaaattaatatttcaaatgtattataaaatcatacctgtaatccatgggaaaaaaattgaaggcaacggagttaattgacctgctctgcttcgattacaatacaaactaatagttttccataaaagagaacaacacgtaacaaatgaacaaataattgtgactgcttgattagaagtgtccctggcatgttgtaccaaggctattatccaaaacgttaatgtaagaaggtttgtaagtaaccaactactccaaataaaaaaggtatagctacacgccattttaaaataatcaaaactgtattatcaagttaagggggagtttgagctttaaaaggttggttacatcgtttccaggcaaacctcacactgagtcacacaaaggtttctgtgtgcccctgtacgggaatctgccttattgctgattttgttggcaaccaaccaaagaaatcatggaaaagcagagtgggaatatttgtttttgttcgttggcctggatgcttggaaacattcctgttattttgatgttaggccaaactgtggaaaacattaagagttgttattgaaaatattttttggtaacaaaaccatgttttaggtcccaaaaggaacataaaaatgtttaatagaaacatgcctagtacagaacattttgtttggtacgtaaactaaaaatatgtatggtaagggctaggggtaagggcaaggggtaagggctaggggtaagggcaaggggtaagggcaaggggtaagggcaaggggtaagggcaaggggtaagggcaaggggtaagggcaaggggtaagggcaaggggtaagggcaaggggtaagggctaggggtaagggctaggggtaagggctaggggtaagggctagggtgagctagggtgagctagggttatagttatagtgtaaaataataggcctacgataaaatgtcaacatatattttttaatttgtgtttcattaacagccatggtgtattagagtaaaggttaacgcttcaatacaatatacaagtaaaagtaaaccacacgaatttataacatatttacaaaagcgaaccaagctggcacatgtaacttcacgctaccctaaacgtacaatactgggactagaacccagaggtagttagaatatacggtagttacagaactttgcagttcccttaggccagcagggctctgcggttaattaaacaaagtttaaagataactgaaactttaggaagtgcgtatgggtgcaatgtgttccaaatagggcaagggttacataaactgttgcctagcggccgggcccggaggcgcccggccggcgccgccgccgggccgcggccgccatcttgcgcccggggcgagggtcccccgcgcgccccccgggcccgcgcgccgggcgccgccggcctcccccggctcccggccctccgccccgccggcccgggcccgcggccgccgcccccggccgccccgcgccgcgcgccgggccgctttcggttcgcggggccgggggtcccgcggggggccggggccgcccccgggtgccgctccgccgggcccggccgactcccgggagcgccccggtccggccgagcccggcggcgccgcgccccgacgccccccgggcccggggccccacaagccgcggcgcgcggctcccgatgccgggcggccgccgcccggcatggcggtcctccgccggcctcccctcccccacgccgccccgaaaggtggtctccgcgccgccgggaggggggccggggcccggggcgcctcggcggggcccggcgcggggcgcgaccgagggccccgggagaacgggggatcgggaaaacgcgaggggagcgggggacaggggacggcgtgtgcgtgcttgtgagacaccgggtacggctgcctgcctgctcgctggcctgcttgctgaggggacagtaggcctgcttgctcgctggcctgcttgctgaggggacagtaggcctgcttgctgaggggacagtaggcctgcttgctcgctggcctgcttgctgaggggacagtagggctgctggcttgctagtagggctgctcgctggcctgcttgcttgctcgctggcctgcttgcttgctcgctggcctgcttgcttgctcgctggcctgcttgcttgctcgctggcctgcttgcttgctcgctggcctgcttgcttgctcgctggcctgcttgcttgctcgctggcctgcttgctgaggggacagtagggctgcttgcttgctaaggggacggtacgcctgcctgatggcttgatagtagggctgctgggctgctagtagggctgctgggctgctagtagggctgctgggctgctagtagggctgctgggctgctagtagggctcctgggctgctagtagggctgctgggctgctagtagggctcctgggctgctagtagggctgctgggctgctagtagggctgctgggctgctagtagggctgctgggctgctagtagggctgcctgctggcttgcttgcttgcttgctagtggggccgcttgcctgctactagggctgctgtgcagctgggagaacagagtagggctgccggccagctgcgtgcgagggcgtccgagggccagacgaggacacgggacccgggcctctcccccgcccggaccgccgggcacccggcccagatct

SEQ ID NO: 2 - DNA, 426 bp, Macaca fuscata herpesvirus 17792
gaattcatattgcatatgtcctctatggacacagtttgtacacacaactgaaccatttgttccggtgtgagatattgcatcatcacccacgtacagaacacggcgtggtacgcccacgctacctgtcgcaagtagttttgcgtgattcctttttcttccttgcggcctcgactttgaatgcgcctgtgcgcctccgaactgttgagcgttagcaaaacgaccacatcaccgtcgtgtgccgagaaaagagataggatttgaaacaaatgatccggcgttagcctgttgtactttacgtgaaccagcggaaaaacaacggtggcggacagtaggtgtctgtcaaaaacaatccaattcgctggctttgtgctaccgtttcccactagccacggttgcaggttgcgaccgatgccggcggccgttgca

SEQ ID NO: 3 - protein, 141 aa, Macaca fuscata herpesvirus 17792
ATAAGIGRNLQPWLVGNGSTKPANWIVFDRHLLSATVVFPLVHVKYNRLTPDHLFQILSLFSAHDGDVVVLLTLNSSEAHRRIQSRGRKEEKGITQNYLRQVAWAYHAVFCTWVMMQYLTPEQMVQLCVQTVSIEDICNMN

SEQ ID NO: 4 - DNA, 524 bp, Macaca fuscata herpesvirus 17792
gaattcaatgactggcatggtgccgcaggaaaacgtcttaagctgtccagagcccatgaaattttggacgtgtgtttattcaaattgccttaaagagcagcgcagcatagttaagcaaggcacccacgggaaatcgatcacttctgctcgcgtatacgcgtgtcagagcaagtttgcgctaccttttcgtgcaacggccgccggcatcggtcgcaacctgcaaccgtggctagtgggaaacggtagcacaaagccagcgaattggattgtttttgacagacacctactgtccgccaccgttgtttttccgctggttcacgtaaagtacaacaggctaacgccggatcatttgtttcaaatcctatctcttttctcggcacacgacggtgatgtggtcgttttgctaacgctcaacagttcggaggcgcacaggcgcattcaaagtcgaggccgcaaggaagaaaaaggaatcacgcaaaactacttgcgacaggtagcgtgggcgtaccacgccgtgttcttgt

SEQ ID NO: 5 - protein, 174 aa, Macaca fuscata herpesvirus 17792
NSMTGMVPQENVLSCPEPMKFWTCVYSNCLKEQRSIVKQGTHGKSITSARVYACQSKFALPFRATAAGIGRNLQPWLVGNGSTKPANWIVFDRHLLSATVVFPLVHVKYNRLTPDHLFQILSLFSAHDGDVVVLLTLNSSEAHRRIQSRGRKEEKGITQNYLRQVAWAYHAVFL

SEQ ID NO: 6 - DNA, 630 bp, Macaca fuscata herpesvirus 17792
agcgagtcmgtgagcgaggaagcggaagagcgcccaatacgcaaaccgcctctccccgcgcgttggccgattcattaatgcaggttaacctggsktrtcgaaattaatacgactcactatagggagaccggcctcgagcagctgaagcttgcatgcctgcaggtcgactctagaggatccccgggtaccgagctcgaattccagacaggtaagattttagctcctaatgtttgccttgccgcctagctcctaatgtttgccttgccgcctagctcctaatgtttgccttgccgcctagctcctaatgtttgccttgccgcctagctcctaatgkttgccttgccgcctagctcctaatgtttgccttgccgcctagctcctaatgtttgccttgccgcctagctcctaatgtttgccttgccgcctagctcctaatgtttgccttgccgcctagctcctaatgtttgcccacgtttatgtttaagcacactaaaatttaaaaacgtttgtgttggtttttatgaccggcttggtacaaaacctgctggtgattttttacccaacaaaaataataaataaaaaagttaaaacttatttctgttgtctgtggtaattgcgttccccgagggt

SEQ ID NO: 7 - DNA, 406 bp, Macaca fuscata herpesvirus 17792
gaattcgcagcccctcttgaaacactgatggccaaggcgatcgacgccggatttatccgagatcgcacagacctgctcaaaacagataaaggcgtggccagagtcgcgagaagtacgtatttaaaggccagccagtctccctcctctcagcacggcggtaaccgcgacacccaaaccatgagcgccctcccggatgacaacatcaccattcccaagagcacctttctaaccatggtgcagagcagtctcgatcacatgcgcaaccagggccaacgcgcgtacgtttccgcgccaccctcgatgccggcaacggcggcgtatccctcgtggataccgccaccagaactgaccgtcccgtcgtacgcgccgcccgtagcgccaccgttcccttttcagtcggcgtttg

SEQ ID NO: 8 - protein, 135 aa, Macaca fuscata herpesvirus 17792
EFAAPLETLMAKAIDAGFIRDRTDLLKTDKGVARVARSTYLKASQSPSSQHGGNRDTQTMSALPDDNITIPKSTFLTMVQSSLDHMRNQGQRAYVSAPPSMPATAAYPSWIPPPELTVPSYAPPVAPPFPFQSAF

SEQ ID NO: 9 - DNA, 630 bp, Macaca fuscata herpesvirus 17792
  misc_feature (26)..(26): n is a, c, g, or t
grgcgcagcgagtcagtgagcgaggnaagcggaagagcgcccaatacgcaaaccgcctctccccgcgcgttggccgattcattaatgcaggttaacctggsttatcgaaattaatacgactcactatagggagaccggcctcgagcagctgaagcttgcatgcctgcaggtcgactctagaggatccccgggtaccgagctcgaattccagattgactcatcggtttctaaccctaacaaagttgcatgaacagagtatgataccaatggtggtagaaatgttagcagcggttaaagaacacgtgaccttaatggaggtctgtttgggcctctttaaagagctacgaaagcttcagattttaattgttgacgcgggagaacatttagatgatacgtgtggcctttggggaaatatttatgggcaggtaatgtcaaatgaggctattaaaccacgagccgtgaactggccagcccttgaaagctacattcaaacgctaaccagcttggaaagcaatgcagccaattaaccacggtttgtgtttgttggtaattacattctctgtaattaatggatatgaatataatgaagaaaatgtacctggacttgaaatagttctttttacccctgcc

SEQ ID NO: 10 - protein, 124 aa, Macaca fuscata herpesvirus 17792
SLHACRSTLEDPRVPSSNSRLTHRFLTLTKLHEQSMIPMVVEMLAAVKEHVTLMEVCLGLFKELRKLQILIVDAGEHLDDTCGLWGNIYGQVMSNEAIKPRAVNWPALESYIQTLTSLESNAAN

SEQ ID NO: 11 - DNA, 524 bp, Macaca fuscata herpesvirus 17792
gaattcaatacgctccaaaagatagagatcacaattttatttttgacgccaatcaaaacccagatcgacataaacaagtgcaccacgaccaccagaccgaaccgcttccagacatgttcgatccagtaaagcacctaagtttacacaacttcaaaatctcagttttcaataccaatatggtaattaataccaaaatcacgtgccggtctctcaccggtaccttcgagtcaatcatcgacatccccaggctcacaaataactttgtaatgaaaaaattctccgtgaaagaaccgtcattcactgtgagcgtgttctactccgacaacctgtgcaacggggctgcgattaacgttaacataagcggggacatgctgcactttatgttcgctatgggaaacctgaggtgctttttgccggtgaagcacattttcccggtttcgattgcgaactggaactctacgttagacctccacgggctcgaaaaccaatacatagttagacgggggcggcgagacgttttctgg

SEQ ID NO: 12 - protein, 174 aa, Macaca fuscata herpesvirus 17792
IQYAPKDRDHNFIFDANQNPDRHKQVHHDHQTEPLPDMFDPVKHLSLHNFKISVFNTNMVINTKITCRSLTGTFESIIDIPRLTNNFVMKKFSVKEPSFTVSVFYSDNLCNGAAINVNISGDMLHFMFAMGNLRCFLPVKHIFPVSIANWNSTLDLHGLENQYIVRRGRRDVFW

SEQ ID NO: 13 - DNA, 398 bp, Macaca fuscata herpesvirus 17792
gatcccatagagtccctgttctgcggcggtctttttaactctatagacgataccattaacgcactgagccgggattgttcggtaacgttctttcaacaggcaaactataccaacgtcatgcgaaaacaaaacgagctgtttaccagactcaacagcatattgtgtcaggggagcgcgggatcgcaaaaaccggccaccccctcggagccacggaccgccaccgtcgcggcgaccgcggcaagcgacgtcattaaagacgcacaatatcgcaaagaacagtacatgaaaaaggtggccagggacggctttaaaaaactaacagagtgtctgcagacgcaaagcgcggtgttggcaaacgcgctctgcatgcgcgtatgggggggcgtcgcatacggcga

SEQ ID NO: 14 - protein, 132 aa, Macaca fuscata herpesvirus 17792
DPIESLFCGGLFNSIDDTINALSRDCSVTFFQQANYTNVMRKQNELFTRLNSILCQGSAGSQKPATPSEPRTATVAATAASDVIKDAQYRKEQYMKKVARDGFKKLTECLQTQSAVLANALCMRVWGGVAYG

SEQ ID NO: 15 - DNA, 524 bp, Macaca fuscata herpesvirus 17792
gaattcacacccgaacaaatctaaaagcaaaattacacaaattaacaaaaatacaactataacacagttaaaaaattcaatacataattacaatggtaaaattcacgcgtacataattacaattttaaattcaatttaaaaattcacgtgtacacaattacaaaattcacacaattttaaaatccttttaaccataaaattagaacggcactgtatatatgcagcgtattgcaaactggcagctttgtgttaaagaccacggcattaaattttaaattcgccaggtcgccaaaagtactggttacgttagttcatctaaaagcttaacgtgcttgtgcttaaatttacgccgtgccattgctccattttaaattaaaatttaaaatgtagtgcgagcgagctagaaacagaaaccgcgacgcggggcaggttgcgtaggttcgccgtttttggctccagtgacaatccaaaagctgcggtttacgagccattgtttttgtcaaccactaaaccgaaagcatgcg

SEQ ID NO: 16 - DNA, 438 bp, Macaca fuscata herpesvirus 17792
ggtacccccacgactataaaaagagacaccctggtggccgccgccgcgccctgccccgtggtgcgagtaagctccgccgacgacgcgccgcgggatctcgtcgcgtcaccagacaccggggcgctctccattgacgcgttcacaatcccggtcggtctccccggggtggtctcggcggagtgtcacgtgtctatgcgcgacaacggggtccacgaacgcatgagccattaacggcgacaatgggaacaccggtgcgtttctttcgcggcgagtggcaaacctcgagtctggtggacaacggcacgccacggtacagctccctggtgtgggccgccactatccacgacggctacctgacactggtgaacaggtcggagctgtgcgtcacggagcggtctccgtgtctgccggcgtgccccagcatcgggagactggtcg

SEQ ID NO: 17 - protein, 76 aa, Macaca fuscata herpesvirus 17792
GTPTTIKRDTLVAAAAPCPVVRVSSADDAPRDLVASPDTGALSIDAFTIPVGLPGVVSAECHVSMRDNGVHERMSH

SEQ ID NO: 18 - DNA, 497 bp, Macaca fuscata herpesvirus 17792
  misc_feature (441)..(441): n is a, c, g, or t
ggtaccgagccgcttccagacggacattattccgagcggaaccgtgctcaagctcctggggcgaaccgagaacggcaccagcgtgtgcgtgaacgtgttccgtcaacaggtatatttctacgcgaaggtcccagccggcgtcaacgtcacccacgtcctccagcaggccctcaagaacaccgccggcagggccgcgtgcggcttctcgaccagacgagtaaccaaaaaaattctcaaaacgtacgacgtcgcggagcatcccgtcactgaaatcacgctatcgtccggttccatgctctcgaccctcagcgaccgcctcgtcgcgtgcgggtgcgaggtgttcgagtcaaacgtggacgccgttcgccggttcgtcctggatcacgggtttaccacgttcgggtggtactcgtgcgcgcgtgccacgccccgcctagcggncagagatgccagaacggccctggagtttgactgcagctgggaggacctcagcgttc

SEQ ID NO: 19 - protein, 165 aa, Macaca fuscata herpesvirus 17792
  misc_feature (147)..(147): Xaa can be any naturally occurring amino acid
VPSRFQTDIIPSGTVLKLLGRTENGTSVCVNVFRQQVYFYAKVPAGVNVTHVLQQALKNTAGRAACGFSTRRVTKKILKTYDVAEHPVTEITLSSGSMLSTLSDRLVACGCEVFESNVDAVRRFVLDHGFTTFGWYSCARATPRLAXRDARTALEFDCSWEDLSV

SEQ ID NO: 20 - DNA, 314 bp, Macaca fuscata herpesvirus 17792
ggtaccgttggggttgaataggcgcaggtgtgttgtgcaaccggaaagcaaaagtgttggggtgacgatgaataggggctccggtgccacgcccgcaactagaaacgccagatcgccccgagcggcgctccctgttggaaagattaactgaacccggacttccgcggagcttccaggttggatggttttttcggcgtcggcgtacacccccaatacgcggccgccggtgtgggtgggcaggacgggtccggactggcccaagactatctcggcgttggggtcccccggagcgtaaatgattttcatttgcgccg

SEQ ID NO: 21 - protein, 104 aa, Macaca fuscata herpesvirus 17792
AQMKIIYAPGDPNAEIVLGQSGPVLPTHTGGRVLGVYADAEKTIQPGSSAEVRVQLIFPTGSAARGDLAFLVAGVAPEPLFIVTPTLLLSGCTTHLRLFNPNGT

SEQ ID NO: 22 - DNA, 681 bp, Macaca fuscata herpesvirus 17792
ggtaccgcaaggtggccacctcggtaaccgtctatcgagggtggaccgagaccgccgtgaccggtaagcaagaggtcattcgaccggtgccgcagtacgagattaaccacatggacacgacctaccagtgtttcagctccatgcgcgtgaacgttaacggcatcgaaaacacctacacggacagggacttcactaaccagaccgtgttcctgcaaccggtcgaggggctcacggataacattcagcgatacttcagtcagccggtgctgtacacgacaccgggatggtttcctggaatctacagggtccgaaccacggtcaactgcgagatcgtggacatgatcgcgcgttcggcggaaccgtactcttactttgtcaccgccctgggagacacggtagaggtatcgccgttctgcttaaacgactcgacgtgctccgtcgctgataaagccgaaaacggcctcggcgtgcgcgtgcttacaaattacaccattgttgacttcgctacccgcacgcccaccaccgaaacgcgagttttcgcagactcgggagaatacaccgtatcgtggaaggcggaagaccctaagtcggcagtctgtgcgctgacgctctggaaaacctttcccagggccatacagacgacgcacgaaagccagctaccactttgtggccaacgacgtg

SEQ ID NO: 23 - protein, 226 aa, Macaca fuscata herpesvirus 17792
YRKVATSVTVYRGWTETAVTGKQEVIRPVPQYEINHMDTTYQCFSSMRVNVNGIENTYTDRDFTNQTVFLQPVEGLTDNIQRYFSQPVLYTTPGWFPGIYRVRTTVNCEIVDMIARSAEPYSYFVTALGDTVEVSPFCLNDSTCSVADKAENGLGVRVLTNYTIVDFATRTPTTETRVFADSGEYTVSWKAEDPKSAVCALTLWKTFPRAIQTTHESQLPLCGQRR

SEQ ID NO: 24 - DNA, 389 bp, Macaca fuscata herpesvirus 17792
ggtacctcggcgcacctgtccgccgcgtacgtcgtttcaacgacgtcgtacgcgtgaaacgtaatggattggtcttgggggtccgggacgactggccgttggccgcgcgccgtccacatggaccgttcgtgggcgagcagcacgtctccgcgcgcgccgttttcaaagtacgtcggcgggaacgggatcgtgaccgggatcatcccgcctgcccccggcgttcggaggcaggcgggaatgagcctgcagacgtctggtggcggctgaacggcgccggcgggggcgggcgcatcggtgcatttgtgtgagggggggcgtggtcctcgaggacccaggtacgggttaaaaaaatccatgctcgatacagaacgctgacgcccgggggcgcc

SEQ ID NO: 25 - protein, 129 aa, Macaca fuscata herpesvirus 17792
GAPGRQRSVSSMDFFNPYLGPRGPRPPSHKCTDAPAPAGAVQPPPDVCRLIPACLRTPGAGGMIPVTIPFPPTYFENGARGDVLLAHERSMWTARGQRPVVPDPQDQSITFHAYDVVETTYAADRCAEV

SEQ ID NO: 26 - DNA, 330 bp, Macaca fuscata herpesvirus 17792
  misc_feature (78)..(78): n is a, c, g, or t
ggtaccgcctaaccttaaagatgtgggggacgatattttttttaaacaccattagaatgccttcttggtgcgttttgncctcggtgccaggacacgtcttttccaggttaaacctgaagagttcgccggtggccgaggcgctgcacacgcgaaacttgaacgcctcggccctgggtgggttttcgggaggtgtgggcgtcgacgggcccggcgtggactttgcagtggtggtcacgccctttggggtggtgacgttttcgccaaccggcgcgccaatcgcgattatcacgacccacgcccgcagcagacgacgcgtccggttaggtatca

SEQ ID NO: 27 - protein, 109 aa, Macaca fuscata herpesvirus 17792
  misc_feature (84)..(84): Xaa can be any naturally occurring amino acid
IPNRTRRLLRAWVVIIAIGAPVGENVTTPKGVTTTAKSTPGPSTPTPPENPPRAEAFKFRVCSASATGELFRFNLEKTCPGTEXKTHQEGILMVFKKNIVPHIFKVRRY

SEQ ID NO: 28 - protein, 140 aa, Macaca fuscata herpesvirus 17792
NVAVEGNSSQDAGVAIATVLNEICSVPLSFLHHADKNTLIRSPIYMLGPEKAKAFESFIYALNSGTFSASQTVVSHTIKLSFDPVAYLIDQIKAIRCIPLKDGGHTYCAKQKTMSDDVLVATVMAHYMATNDKFVFKSLE